(19) United States
(12) Patent Application Publication
(10) Pub. No.: US 2010/0105054 A1
(43) Pub. Date: Apr. 29, 2010

(54) GENE EXPRESSION IN DUCHENNE MUSCULAR DYSTROPHY

(75) Inventors: Brenda L. Wong, Cincinnati, OH (US); Frank R. Sharp, Davis, CA (US); Lisa Lit, Davis, CA (US)

Correspondence Address: THOMPSON HINE L.L.P.;Intellectual Property Group, P.O. BOX 8801, DAYTON, OH, 45401-8801, US

(73) Assignees: CINCINNATI CHILDREN'S HOSPITAL MEDICAL CENTER, Cincinnati, OH (US); THE UNIVERSITY OF CALIFORNIA AT DAVIS, Sacramento, CA (US)

(21) Appl. No.: 12/579,038

(22) Filed: Oct. 14, 2009

Related U.S. Application Data
(60) Provisional application No. 61/105,577, filed on Oct. 15, 2008.
(60) Provisional application No. 61/105,565, filed on Oct. 15, 2008.

(51) Int. Cl. C12Q 1/68

(52) U.S. Cl. 435/6

(57) ABSTRACT

Gene expression in peripheral blood from individuals with Duchenne muscular dystrophy (DMD), compared to control individuals, demonstrated differential gene sets that could be used in a method to diagnose DMD, to evaluate effect of DMD therapy, and/or to evaluate propensity to DMD.

This application claims priority from U.S. patent application Ser. No. 61/105,577 filed Oct. 15, 2008 with a petition for revival in progress, and co-pending U.S. patent application Ser. No. 61/105,565 filed Oct. 15, 2008, each incorporated by reference herein in its entirety.

The terms normal individuals, individuals without Duchenne muscular dystrophy (DMD), control, control group, or controls, patients without DMD, healthy individuals, and normal patients are used synonymously. The terms individuals with DMD, DMD patients, and patients with DMD are used synonymously. Gene expression changes refers to changes in expression of a nucleic acid, also referred to as regulation. Nucleic acid refers to either the sequence that encodes a protein; the nucleic acid can be deoxyribonucleic acid (DNA) or messenger ribonucleic acid RNA (mRNA), or the non-coding sequence that can be either 5′ to the coding sequence (upstream) or 3′ to the coding sequence (downstream). Gene expression changes are not limited to changes in the coding sequence. The nucleic acid sequence can be coding, noncoding (e.g., regulatory sequence such as a promoter), or both coding and noncoding. Evaluation of gene expression encompasses evaluation of mRNA transcription from DNA, and translation into protein, as known by a person having ordinary skill in the art. A described mRNA includes the resultant protein, known by a person having ordinary skill in the art.

DMD is caused by gene mutations in dystrophin, resulting in muscle degeneration associated with chronic inflammation and fibrosis. Strong evidence for immune modulation of muscular dystrophies comes in part from studies of the mdx mouse model of DMD. Treatment with anti-tumor necrosis factor α (anti-TNF-α) protects dystrophic muscle from necrosis. Treatment with cyclosporin A improves function and histology. Expression of perforin, the cytotoxic T-lymphocyte and Natural Killer cell-derived, pro-apoptotic protein is increased in DMD muscle. A null mutation in perforin in the mdx mouse eliminates muscle apoptosis and reduces muscle necrosis. Data suggest an important role for the immune system in the pathogenesis of muscular dystrophy in the mdx mouse.

The immune system also has a role in DMD in humans. Multiple randomized trials have shown improved function and strength in individuals with DMD treated with prednisone (PRED), and with the oxazoline derivative of prednisolone deflazacort (DEFL). It has been argued that since azathioprine, another immunosuppressant, does not improve DMD in humans, the improvements seen with prednisone may not be related to immunosuppression. However, in some immune-mediated disorders such as myasthenia gravis, azathioprine was a less effective immunosuppressant compared to prednisone.

Gene expression profiling of muscle biopsies has been used to examine specific disease-related cell death and cell regeneration pathways in DMD and other muscular dystrophies. Gene expression profiling in muscle also reflects the effects of immune system-modulating treatments; e.g., intravenous immunoglobulin (IV Ig) treatment for inflammatory myopathy changes muscle gene expression.

Clarifying the mechanisms of immune modulation of muscle pathology in muscular dystrophies will provide pathophysiological, diagnostic, and treatment-related implications. Better understanding of these mechanisms at the level of gene expression in muscle is limited by the requirement of an invasive muscle biopsy.

It is known that genetic diseases, such as tuberous sclerosis and neurofibromatosis, and chromosomal disorders, such as Down syndrome, produce disease-specific RNA expression profiles in peripheral whole blood. There are specific RNA expression profiles in peripheral blood following ischemic stroke, likely representing an immune response to injured brain.

The immune response to dying muscle associated with DMD, in addition to the genetic changes associated with DMD, resulted in a specific expression profile in peripheral blood. The demonstrated changes of gene expression, as assessed by measurements of RNA levels from peripheral blood of subjects with DMD compared to controls, on whole genome microarrays, are disclosed.

Individuals with DMD administered the corticosteroids prednisone and deflazacort had improved muscle function and strength. DMD individuals receiving deflazacort had fewer side effects, in particular, less weight gain. Individuals with DMD administered the immunosuppressant azathioprine had no improvement. Benefits from corticosteroid administration may not directly relate or relate entirely to immunosuppression.

Gene expression profiling of muscle tissue has been used to classify muscular dystrophies and examine specific DMD-related cell death and cell regeneration pathways. IV Ig administration for inflammatory myopathy changed gene expression profiles in muscle, evidencing an alteration of the immune response.

Monitoring gene expression changes, e.g., over time, after treatment with an agent, etc. in muscle has, until now, required obtaining a muscle biopsy specimen from the individual. Using peripheral blood as a tissue source in which to monitor gene expression changes eliminated this invasive procedure.

Gene expression changes in blood were used to monitor the effect of steroid administration, resulting in a specific profile of gene expression. Deflazacort and prednisone generated distinct expression profiles. The expression profiles indicated similar mechanisms of action. Deflazacort and prednisone displayed differences in side effects. Overall effects of corticosteroid treatment on mRNA expression in blood of individuals with DMD, and expression specific for deflazacort and specific for prednisone, were identified in a case-controlled whole genome expression microarray study.

One embodiment is a method to evaluate an individual's propensity for DMD. The method comprises determining from blood of a test individual expression of at least one gene from SEQ ID NOS. 1-55; comparing the expression of the gene(s) from the test individual with expression of the same gene(s) from a control individual; evaluating the test individual's propensity for DMD by determining if the gene(s) in the test individual is either over-expressed ≧1.5 times or is under-expressed ≧1.5 times compared to expression of the same gene(s) from the control individual; and ranking the test individual's propensity for DMD based on at least one of (a) the extent that over-expression or under-expression exceeds 1.5, or (b) the number of genes that are over-expressed or under-expressed, where the test individual's propensity for DMD is ranked higher when (a) is farther from 1.5, and (b) is farther from 1 compared to the control individual. In one embodiment the gene is selected from SEQ ID NOS. 1-5, 6-11, 51-55, and/or 46-50. In one embodiment the method further ranks the test individual's propensity higher if at least one of the genes over-expressed or under-expressed is at least one of SEQ ID NOS. 1-5 or SEQ ID NOS. 51-55. In one embodiment, the method ranks the test individual's propensity higher if at least one of the genes over-expressed or under-expressed is SEQ ID NO. 1. In one embodiment, in (a), over-expression or under-expression exceeds 2. In one embodiment, in (a), over-expression or under-expression exceeds 2.5.

One embodiment is a method to diagnose DMD in a test individual. The method comprises determining from blood of a test individual expression of at least one gene selected from SEQ ID NOS. 1-55; comparing expression of the gene(s) from the test individual with expression of the same gene(s) from a control individual; diagnosing DMD by determining if the gene(s) in the test individual is either over-expressed ≧2.5 times or is under-expressed ≧2.5 times compared to expression of the same gene(s) from the control individual; and determining a confidence level for the diagnosis for DMD based on at least one of (a) the extent that over-expression or under-expression exceeds 2.5, or (b) the number of genes that are over-expressed or under-expressed, where the diagnosis for DMD in the test individual has a higher confidence level when (a) is farther from 2.5, and (b) is farther from 1. In one embodiment, the method ranks the confidence level for the diagnosis higher if at least one of the gene(s) over-expressed or under-expressed is at least one of SEQ ID NOS. 1-5 or SEQ ID NOS. 51-55. In one embodiment, the method ranks the confidence level for the diagnosis higher if at least one of the gene(s) over-expressed or under-expressed is defensin.

One embodiment is a method of ranking a gene as a target for ameliorating DMD. The method comprises (a) determining from blood of a test individual with DMD expression of at least one gene selected from SEQ ID NOS. 1-55; (b) comparing the expression of the gene(s) from the test individual with an expression from the same gene(s) from a control individual; and (c) ranking the gene as a target by determining if the at least one gene in the test individual is either over-expressed ≧1.5 times or is under-expressed ≧1.5 times compared to the same gene(s) in the expression profile from the control individual, where the gene is ranked as a better target the farther (c) is from 1.5. In one embodiment, in (c), over-expression or under-expression is ≧2 and the gene is a better target the farther (c) is from 2. In one embodiment, in (c), over-expression or under-expression ≧2.5 and the gene is a better target the farter (c) is from 2.5. In one embodiment, in (a), the gene is at least one of SEQ ID NO. 1-5 or SEQ ID NOS. 51-55. In one embodiment, the method further comprises administering at least one agent specific for at least one of the ranked gene targets, the highest ranked gene, for example, an antibody, an antisense oligonucleotide, etc. In one embodiment, the agent modulates the immune system of the individual with DMD and/or modulates iron utilization of the individual with DMD.

One embodiment is a method of monitoring therapy in an individual with DMD. The method comprises determining from blood of a test individual administered therapy expression of at least one gene selected from SEQ ID NOS. 1-55; comparing expression of the gene(s) from the test individual with expression of the same gene(s) from either (i) a control individual, or (ii) a previous expression result from the test individual; evaluating the test individual's response to therapy by determining if the gene(s) in the test individual is either over-expressed or is under-expressed compared to expression of the same gene(s) from either (i) or (ii), i.e., where the test individual's results after therapy are closer to the control individual's results; and ranking the test individual's response to therapy based on at least one of (a) the extent of over-expression or under-expression, or (b) the number of genes that are over-expressed or under-expressed, where the test individual's response to therapy is ranked higher when at least one of (a) or (b) is closer to either (i) or (ii). In one embodiment, the over-expression or under-expression is ≧1.5. In one embodiment, the over-expression or under-expression is ≧2. In one embodiment, the over-expression or under-expression is ≧2.5.

One embodiment is a kit for diagnosing DMD from peripheral blood. The kit contains at least one primer of a forward oligonucleotide primer and a reverse oligonucleotide primer that anneals to at least one of SEQ ID NO. 1-55; and instructions for using the primer to quantitate SEQ ID NO. 1-55 in peripheral blood. The diagnosis of DMD using the kit is made by determining from blood of a test individual expression of at least one gene selected from SEQ ID NOS. 1-55; comparing expression of the gene(s) from the test individual with expression of the same gene(s) from a control individual; diagnosing DMD by determining if the gene(s) in the test individual is either over-expressed ≧2.5 times or is under-expressed ≧2.5 times compared to expression of the same gene(s) from the control individual; and determining a confidence level for the diagnosis of DMD based on at least one of (a) the extent that over-expression or under-expression exceeds 2.5, or (b) the number of genes that are over-expressed or under-expressed, where the diagnosis for DMD in the test individual has a higher confidence level when (a) is farther from 2.5, and/or (b) is farther from 1. In one embodiment, the primer anneals to defensin. In one embodiment, the primer anneals to T cell leukemia/lymphoma 1a. In one embodiment, the primer is used in the polymerase chain reaction (PCR). In one embodiment, the primer is used in quantitative PCR. In one embodiment, the primer is used in a microarray. In one embodiment, the primer is used in analysis of a Northern blot. Other embodiments are know to a person skilled in the art.

One embodiment is a method of monitoring steroid treatment of DMD. The method comprises determining expression, from blood of a treated individual with DMD administered prednisone and/or deflazacort, at least one gene in Table 4; comparing expression of the gene(s) from the treated individual with an individual with DMD not administered either prednisone or deflazacort or any other steroid; evaluating the treated individual's response to prednisone and/or deflazacort administration by determining if the gene(s) in the treated individual is either over-expressed ≧1.5 times or is under-expressed ≧1.5 times compared to expression of the same gene(s) from the individual with DMD not administered either prednisone or deflazacort or any other steroid; and ranking the treated individual's response to treatment based on at least one of (a) the extent that over-expression or under-expression exceeds 1.5, or (b) the number of genes that are over-expressed or under-expressed, where the treated individual monitored has a greater response to treatment when (a) is farther from 1.5, and (b) is farther from 1. In one embodiment, the ranking is compared to the treated individual's previous expression ranking. In one embodiment, the gene is T cell leukemia/lymphoma 1a. In one embodiment, monitoring determines treatment efficacy. In one embodiment, monitoring determines compliance.

One embodiment is a method of ranking a gene as a target for ameliorating DMD. The method comprises determining from blood of an individual with DMD administered prednisone and/or deflazacort expression of at least one gene in Table 4; (b) comparing expression of the gene(s) from (a) with expression of the same gene(s) from an individual with DMD not administered either prednisone or deflazacort; and (c) ranking the gene(s) as a target for ameliorating DMD by determining if the gene(s) in (a) is either over-expressed ≧1.5 times or is under-expressed ≧1.5 times compared to the same gene(s) in the individual with DMD not administered prednisone or deflazacort, where the gene is a better target the farther (c) is from 1.5. In one embodiment, in (c), over-expression or under-expression is ≧2 and the gene(s) is a better target the farther (c) is from 2. In one embodiment, in (c) over-expression or under-expression is ≧2.5 and the gene is a better target the farther (c) is from 2.5.

One embodiment is a method of determining amelioration of prednisone side effects in an individual with DMD. The method comprises ranking a gene as a target for ameliorating prednisone side effects by (a) determining from blood of an individual with DMD administered prednisone expression of at least one gene selected from Table 5; (b) comparing the expression in (a) with expression of the same gene(s) from an individual with DMD administered deflazacort, and (c) ranking the gene as a target by determining if the gene in (a) is either over-expressed ≧1.5 times or is under-expressed ≧1.5 times compared to the same gene in expression from (b), where the gene is a better target the farther (c) is from 1.5. In one embodiment, at least one agent specific for the gene is then prescribed. In one embodiment, at least one of the determined targets is responsible for the side effects caused by prednisone compared to deflazacort.

One embodiment is a method of determining efficacy of treatment of DM. The method comprises determining, from blood of a treated individual with DMD administered a steroid, expression of at least one gene in Table 4; comparing expression of the gene(s) from the treated individual with expression of the same gene(s) in an individual with DMD not treated with a steroid; evaluating treatment efficacy by determining if the gene(s) in the treated individual is either over-expressed ≧1.5 times or is under-expressed ≧1.5 times compared to expression of the same gene(s) from the individual with DMD not treated with a steroid; and determining treatment efficacy based on at least one of (a) the extent that over-expression or under-expression exceeds 1.5, or (b) the number of genes that are over-expressed or under-expressed, where the treatment is more efficacious when (a) is farther from 1.5, and/or (b) is farther from 1. In one embodiment, the gene encodes T cell leukemia/lymphoma 1a. In one embodiment, the gene encodes defensin.

One embodiment is a kit for monitoring steroid treatment from peripheral blood in an individual with DMD. The kit contains at least one primer of a forward oligonucleotide primer and a reverse oligonucleotide primer that anneals to at least one of the genes in Table 4; and instructions for using the primer to quantitate the gene(s) in Table 4 in peripheral blood. Monitoring is by determining expression, from blood of a treated individual with DMD administered prednisone and/or deflazacort, at least one gene in Table 4; comparing expression of the gene(s) from the treated individual with an individual with DMD not administered a steroid; evaluating the treated individual's response to prednisone and/or deflazacort administration by determining if the gene(s) in the treated individual is either over-expressed ≧1.5 times or is under-expressed ≧1.5 times compared to expression of the same gene(s) from the individual with DMD nor administered prednisone and/or deflazacort; and ranking the treated individual's response to treatment based on at least one of (a) the extent that over-expression or under-expression exceeds 1.5, or (b) the number of genes that are over-expressed or under-expressed, where the treated individual has greater response to treatment when (a) is farther from 1.5, and (b) is farther from 1. In one embodiment, the primer anneals to the gene encoding defensin. In one embodiment, the primer anneals to the gene encoding T cell leukemia/lymphoma 1a. In one embodiment, the primer is used in PCR. In one embodiment, the primer is used in qPCR. In one embodiment, the primer is used in a microarray. In one embodiment, the primer is used in analysis of a Northern blot. Other embodiments are known to a person skilled in the art.

In one embodiment, a gene expression profile characteristic of DMD comprised at least one gene in SEQ. ID NOS. 1-55.

In one embodiment, a gene expression profile characteristic of DMD comprised at least one of nucleic acids 1-59, shown in Table 1.

In one embodiment, a gene expression profile characteristic of DMD comprised at least one of nucleic acids 1-191, shown in Table 2.

In one embodiment, a gene expression profile characteristic of DMD comprised at least one of nucleic acids 1-1467, shown in Table 3.

In addition to listing the nucleic acids, Tables 1, 2, and 3 disclose the relative extent of regulation and whether regulation is by over-expression or by under-expression.

In one embodiment, DMD was diagnosed by evaluating gene expression or gene expression profiles. In one embodiment, DMD was diagnosed by determining expression of at least one gene selected from SEQ. ID NOS. 1-55 in peripheral blood of the individual. Expression of the selected gene(s) was then compared to expression of that same gene(s) in an individual that did not have DMD. The individual was diagnosed for DMD by determining if the gene in the individual was either over-expressed ≧2.5 times or was under-expressed ≧2.5 times, compared to expression of the same gene in the individual without DMD. Confidence in the diagnosis, described as a confidence level of the diagnosis, was determined by evaluating the extent that over-expression or under-expression exceeded 2.5, the identity of the gene over-expressed or under-expressed, and/or the number of genes that were over-expressed or under-expressed. The confidence level of the diagnosis for DMD was higher, based on: (a) the farther the over-expression or under-expression was from 2.5, (b) whether the gene was from SEQ ID NO. 1-55 and/or (c) the greater the number of genes that were over-expressed or under-expressed. In one embodiment, the gene expression profile comprises defensin (SEQ ID NO. 1).

In one embodiment, DMD was diagnosed by determining an expression profile of at least one gene selected from Nucleic Acid Nos. 1-191 in peripheral blood of the individual. The expression profile of the selected gene(s) was then compared to the expression profile of that same gene in an individual that did not have DMD. The individual was diagnosed for DMD by determining if the gene in the individual was either over-expressed ≧2.0 times or was under-expressed ≧2.0 times, compared to the same gene in the expression profile in the individual without DMD. A confidence level of the diagnosis was determined by evaluating the extent that over-expression or under-expression exceeded 2.0, the identity of the gene over-expressed or under-expressed, and/or the number of genes that were over-expressed or under-expressed. The confidence level of the diagnosis for DMD was higher, based on: (a) the farther the over-expression or under-expression was from 2.0, (b) whether the gene was from SEQ ID NO. 1-55, and/or (c) the greater the number of genes that were over-expressed or under-expressed.

In one embodiment, DMD was diagnosed by determining an expression profile of at least one gene selected from Nucleic Acid Nos. 1-1467 in peripheral blood of the individual. The expression profile of the selected gene(s) was then compared to the expression profile of that same gene in an individual that did not have DMD. The individual was diagnosed for DMD by determining if the gene in the individual was either over-expressed ≧1.5 times or was under-expressed ≧1.5 times, compared to the same gene in the expression profile in the individual without DMD. A confidence level of the diagnosis was determined by evaluating the extent that over-expression or under-expression exceeded 1.5, the identity of the gene over-expressed or under-expressed, and/or the number of genes that were over-expressed or under-expressed. The confidence level of the diagnosis for DMD was higher, based on: (a) the farther the over-expression or under-expression was from 1.5, (b) whether the gene was from SEQ ID NO. 1-55, and/or (c) the greater the number of genes that were over-expressed or under-expressed.

In one embodiment, a gene expression profile from peripheral blood showing increased defensin SEQ ID NO.: 1 in an individual, compared to a gene expression profile for defensin mRNA in a normal individual, was used to diagnose DMD. In one embodiment, defensin mRNA was expressed greater than 2.5 fold higher than defensin mRNA was expressed in a normal individual.

In one embodiment, a gene expression profile from peripheral blood showing decreased utrophin Nucleic Acid No. 1378 in an individual, compared to a gene expression profile for utrophin mRNA in a normal individual, was used to diagnose DMD. In one embodiment, utrophin mRNA was expressed less than 1.5 fold lower than utrophin mRNA was expressed in a normal individual.

In one embodiment, gene expression profiles in an individual were used to evaluate DMD disease propensity, disease severity, disease subtype, therapy efficacy, therapy compliance, an individual's response to therapy, etc. In one embodiment, DMD was evaluated by determining an expression profile of at least one gene selected from SEQ. ID NOS. 1-55 in peripheral blood of the individual. In one embodiment, DMD was evaluated by determining an expression profile of at least one gene selected from Nucleic Acid Nos. 1-191 in peripheral blood of the individual. In one embodiment, DMD was evaluated by determining an expression profile of at least one gene selected from Nucleic Acid Nos. 1-1467 in peripheral blood of the individual. The expression profile of the selected gene(s) was then compared to the expression profile of the same gene(s) in an individual that did not have DMD.

The individual's disease propensity, disease severity, and/or disease subtype for DMD was evaluated by determining if the gene in the individual was either over-expressed ≧1.5 times or was under-expressed ≧1.5 times, compared to the same gene expressed in the individual without DMD. Propensity was evaluated by determining the extent that over-expression or under-expression exceeded 1.5, the identity of the gene over-expressed or under-expressed, and/or the number of genes that were over-expressed or under-expressed. The individual's propensity for DMD was higher, based on: (a) the farther the over-expression or under-expression was from 1.5, (b) whether the gene was from SEQ ID NO. 1-55, and/or (c) the greater the number of genes that were over-expressed or under-expressed. In one embodiment, the gene encoded defensin (SEQ ID NO. 1). In one embodiment, propensity for DMD was distinguished from diagnosis of DMD, where propensity for DMD was determined in an asymptomatic and/or undiagnosed individual. In one embodiment, disease severity was determined in a symptomatic individual. In one embodiment, disease subtype was determined in an asymptomatic individual or in a symptomatic individual. For example, an individual's propensity for a particular DMD subtype may be determined, or an individual may be diagnosed as having a particular DMD subtype.

The individual's therapy efficacy, therapy compliance, and/or response to therapy may be termed therapy monitoring. The individual's therapy for DMD was monitored by determining if the gene in the individual was either over-expressed ≧1.5 times or was under-expressed ≧1.5 times, compared to the same gene(s) expressed in the individual without DMD. Therapy was monitored by determining the extent that over-expression or under-expression exceeded 1.5, the identity of the gene(s) over-expressed or under-expressed, and/or the number of genes that were over-expressed or under-expressed. The efficacy of therapy, compliance with therapy, and or response to therapy for DMD was lower, based on: (a) the farther the over-expression or under-expression was from 1.5, (b) whether the gene was from SEQ ID NO. 1-55, and/or (c) the greater the number of genes that were over-expressed or under-expressed. In one embodiment, the gene encoded defensin.

In one embodiment, at least one steroid was administered as therapy for individuals with DMD and gene expression was compared to gene expression in individuals with DMD not administered any steroids. In one embodiment, corticosteroids were administered as therapy for individuals with DMD. In one embodiment, prednisone was administered as therapy for individuals with DMD. In one embodiment, deflazacort was administered as therapy for individuals with DMD. In one embodiment, deflazacort and prednisone were administered as therapy for individuals with DMD. In one embodiment, the efficacy or compliance to steroid, corticosteroid, prednisone and/or deflazacort treatment for DMD was evaluated by determining expression of at least one gene selected from nucleic acids 1-524 (Table 4) in peripheral blood of the individual. Nucleic acids 1-524 represented those nucleic acids that were either over-expressed ≧1.5 times or were under-expressed ≧1.5 times, compared to the same nucleic acids expressed in an individual with DMD not administered any steroids.

In one embodiment, the efficacy or compliance to prednisone and/or deflazacort treatment for DMD was evaluated by determining expression of at least one gene selected from nucleic acids 1-508 (Table 5) in peripheral blood of the individual. Nucleic acids 1-508 represented those nucleic acids expressed in an individual with DMD and administered prednisone that were either over-expressed ≧1.5 times or under-expressed ≧1.5 times, compared to the same nucleic acids expressed in an individual with DMD and administered deflazacort. In one embodiment, the efficacy or compliance to prednisone treatment for DMD was evaluated by determining if the gene in the individual was either over-expressed 1.5 times or was under-expressed ≧1.5 times, compared to the same gene in the expression profile in an individual with DMD and treated with deflazacort. In one embodiment, the efficacy of or compliance with deflazacort administration for DMD was evaluated by determining if the gene in the individual was either over-expressed ≧1.5 times or was under-expressed ≧1.5 times, compared to the same gene in the expression profile in an individual with DMD and administered prednisone.

In one embodiment, a therapeutic target for DMD treatment was determined by evaluating gene expression. In one embodiment, a therapeutic target for DMD treatment was determined by determining expression of at least one gene selected from SEQ. ID NOS. 1-55 in peripheral blood of the individual with DMD. Expression of the selected gene(s) was then compared to expression of that same gene in an individual that did not have DMD. The gene(s) was determined to be a therapeutic target for DMD treatment by determining if the gene in the individual was either over-expressed ≧2.5 times or was under-expressed ≧2.5 times, compared to the same gene expressed in the individual without DMD. A confidence level of the therapeutic target was determined by evaluating the extent that over-expression or under-expression exceeded 2.5, the identity of the gene over-expressed or under-expressed, and/or the number of genes that were over-expressed or under-expressed. The confidence level of the therapeutic target for DMD was higher, based on: (a) the farther the over-expression or under-expression was from 2.5, (b) whether the gene was from SEQ ID NO. 1-55, and/or (c) the greater the number of genes that were over-expressed or under-expressed. In one embodiment, the gene expression profile comprises defensin.

In one embodiment, a therapeutic target for DMD treatment was determined by determining expression of at least one gene selected from nucleic acids 1-1467 in peripheral blood of the individual with DMD. Expression of the selected gene(s) was then compared to expression of the same gene(s) in an individual that did not have DMD. The gene(s) was determined to be a therapeutic target for DMD treatment by determining if the gene(s) in the individual was either over-expressed ≧1.5 times or was under-expressed ≧1.5 times, compared to the same gene(s) in expressed in the individual without DMD. A confidence level of the therapeutic target was determined by evaluating the extent that over-expression or under-expression exceeded 1.5, the identity of the gene over-expressed or under-expressed, and/or the number of genes that were over-expressed or under-expressed. The confidence level of the therapeutic target for DMD was higher, based on: (a) the farther the over-expression or under-expression was from 1.5, (b) whether the gene was from SEQ ID NO. 1-55, and/or (c) the greater the number of genes that were over-expressed or under-expressed.

In one embodiment, a therapeutic target for DMD treatment was determined by determining expression of at least one gene selected from nucleic acids 1-191 in peripheral blood of the individual with DMD. Expression of the selected gene(s) was then compared to expression of that same gene in an individual that did not have DMD. The gene(s) was determined to be a therapeutic target for DMD by determining if the gene(s) in the individual was either over-expressed ≧2.0 times or was under-expressed ≧2.0 times, compared to the same gene(s) expressed in the individual without DMD. A confidence level of the therapeutic target was determined by evaluating the extent that over-expression or under-expression exceeded 2.0, the identity of the gene over-expressed or under-expressed, and/or the number of genes that were over-expressed or under-expressed. The confidence level of the therapeutic target for DMD was higher, based on: (a) the farther the over-expression or under-expression was from 2.0, (b) whether the gene was from SEQ ID NO. 1-55, and/or (c) the greater the number of genes that were over-expressed or under-expressed.

In one embodiment, an agent's effect on the pathophysiology of DMD was evaluated. At least one cell, referred to as the test cell, was exposed to the agent, and expression of at least one gene selected from SEQ ID NOS. 1-55 in the cell(s) was compared to expression of the same gene(s) selected from SEQ ID NOS. 1-55 in a cell of an individual without DMD, referred to as the control cell. The contribution of the agent to the pathophysiology of DMD was evaluated by determining if the gene(s) in the test cell was either over-expressed ≧1.5 times, or was under-expressed ≧1.5 times, compared to the same gene(s) expressed in the control cell. The agent's contribution to the pathophysiology of DMD was evaluated by determining the extent that over-expression or under-expression exceeded 1.5, the identity of the gene over-expressed or under-expressed, and/or the number of genes that were over-expressed or under-expressed. The agent contributed more to the pathophysiology of DMD based on at least one of the farther the over-expression or under-expression was from 1.5, the gene was from SEQ ID NO. 1-55, and/or the greater the number of genes that were over-expressed or under-expressed. In one embodiment, expression of at least one gene selected from nucleic acids 1-191 was compared. In one embodiment, expression of at least one gene selected from nucleic acids 1-1467 was compared. In one embodiment, the cell was a component of peripheral blood. In one embodiment, based on the extent that the agent contributed to DMD, agents that antagonized the action of the agent were predicted or used to treat DMD.

The patent or application file contains at least one drawing executed in color. Copies of this patent or patent application publication with color drawing(s) will be provided by the Office upon request and payment of the necessary fee.

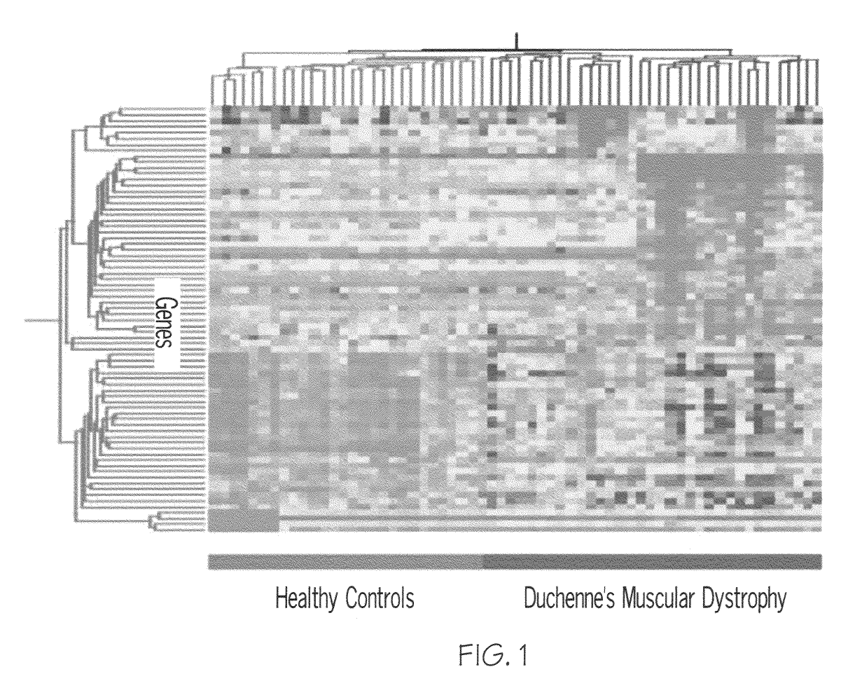

FIG. 1 shows cluster analysis of genes differentially regulated in individuals with DMD compared to control individuals.

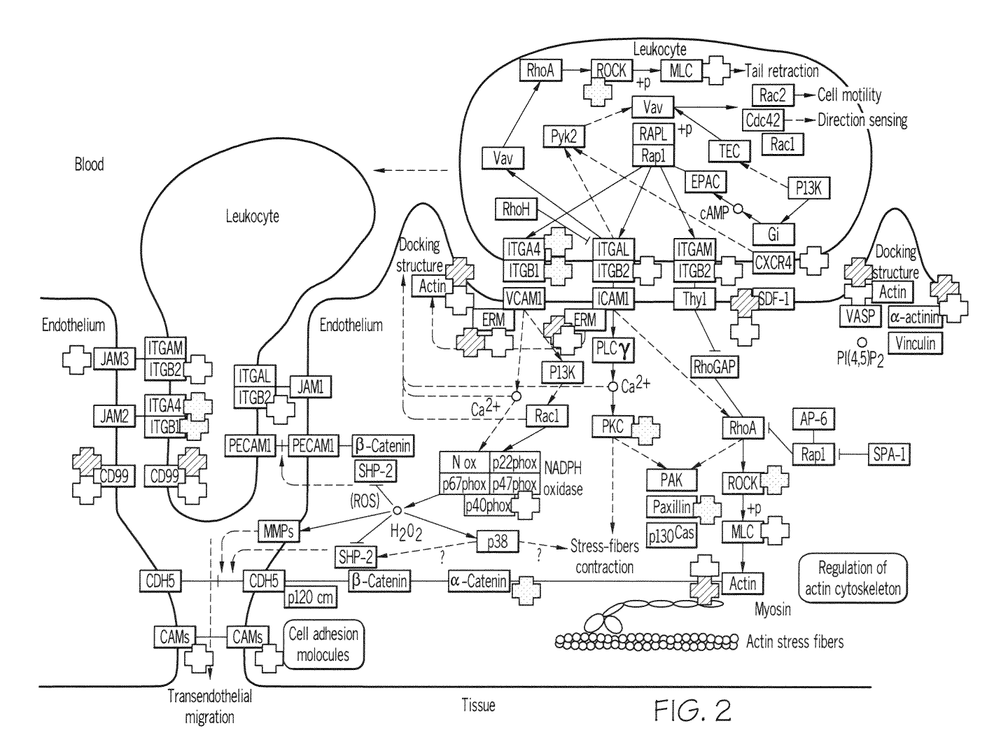

FIG. 2 schematically shows DMD gene expression and differential regulation in the leukocyte trans-endothelial migration pathway.

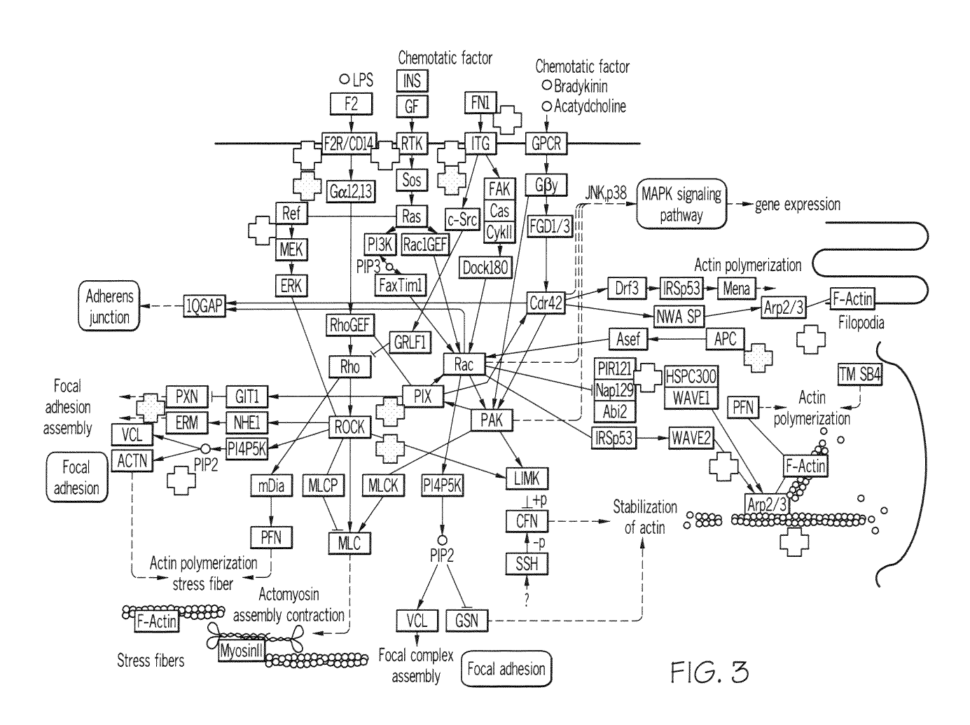

FIG. 3 schematically shows DMD gene expression and differential regulation in the actin cytoskeleton pathway.

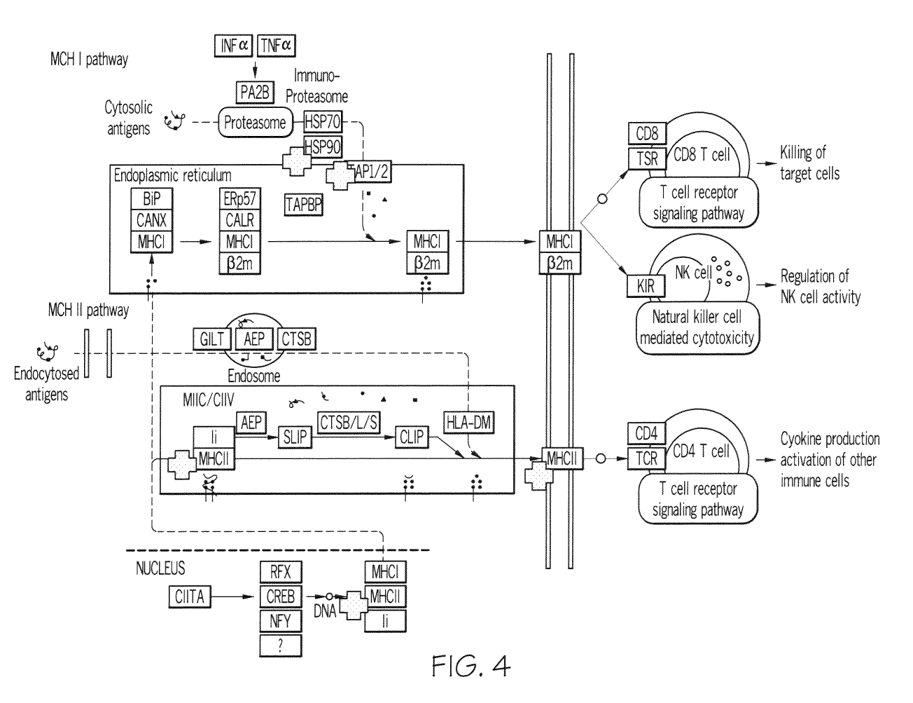

FIG. 4 schematically shows DMD gene expression and differential regulation in the antigen processing and presentation pathway.

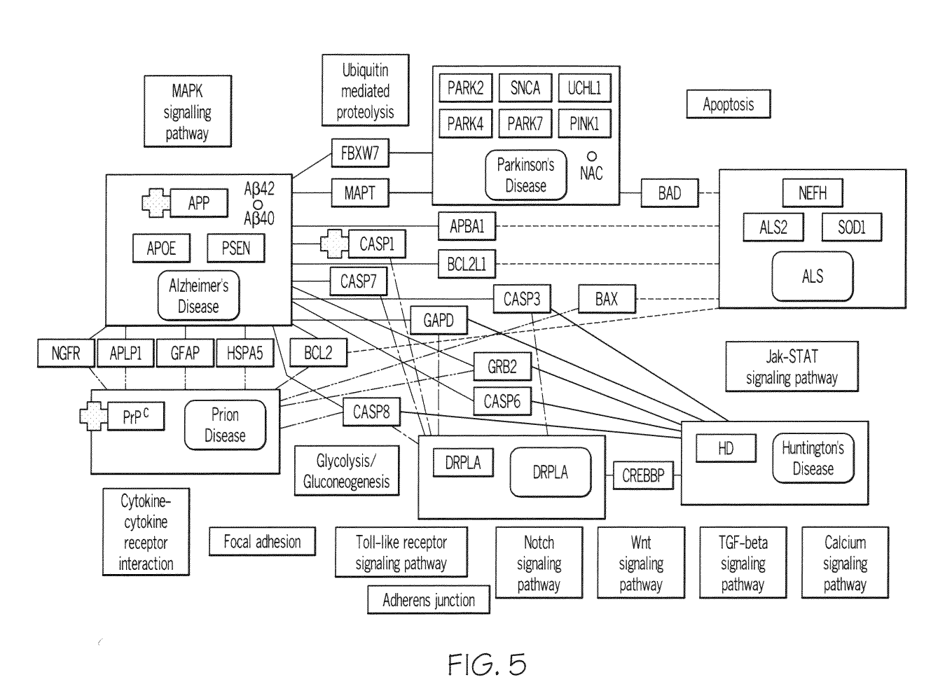

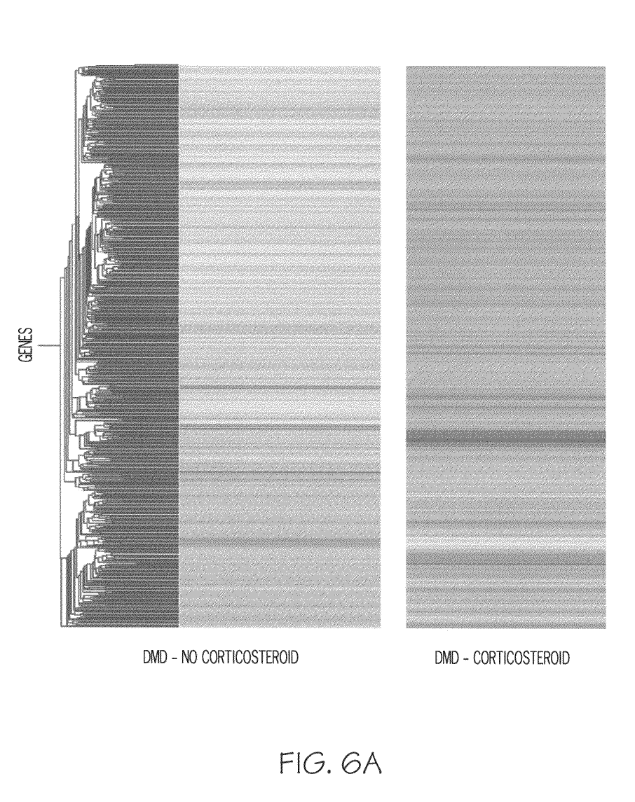

FIG. 5 schematically shows DMD gene expression and differential regulation in the neurodegenerative disorders pathway.

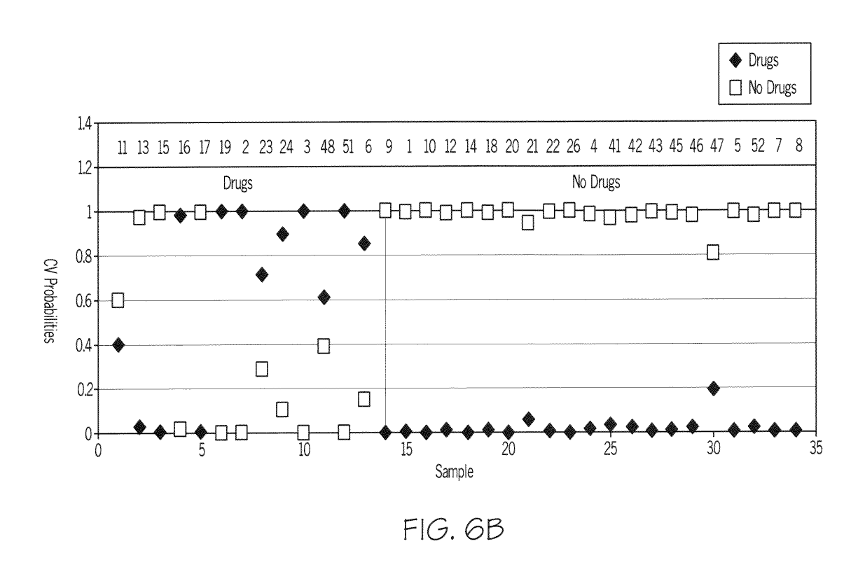

FIG. 6A shows cluster analysis of genes differentially regulated in individuals with DMD administered no corticosteroids, compared with individuals with DMD administered corticosteroids.

FIG. 6B shows prediction analysis of individuals with DMD receiving no corticosteroid compared to individuals with DMD receiving corticosteroids.

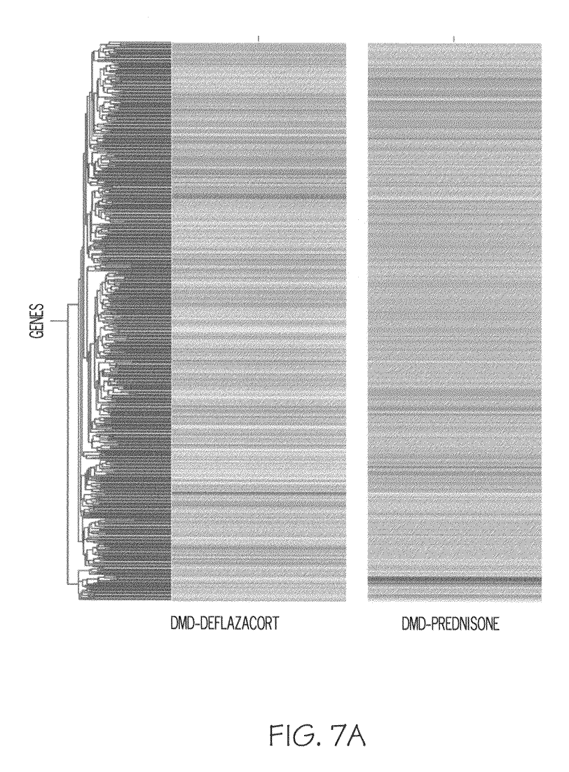

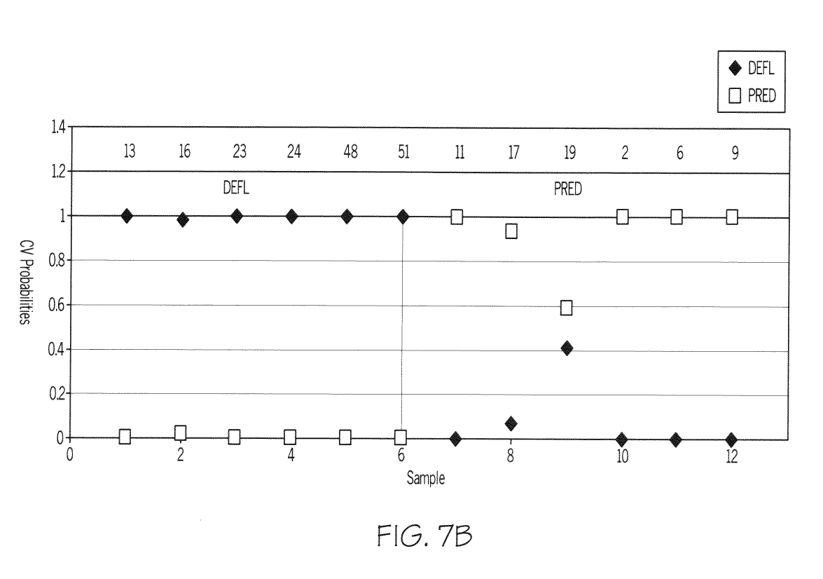

FIG. 7A shows cluster analysis of genes differentially regulated in peripheral whole blood of individuals with DMD receiving deflazacort, compared to individuals with DMD receiving prednisone.

FIG. 7B shows a prediction analysis of microarray of individuals with DMD receiving deflazacort compared to individuals with DMD receiving prednisone.

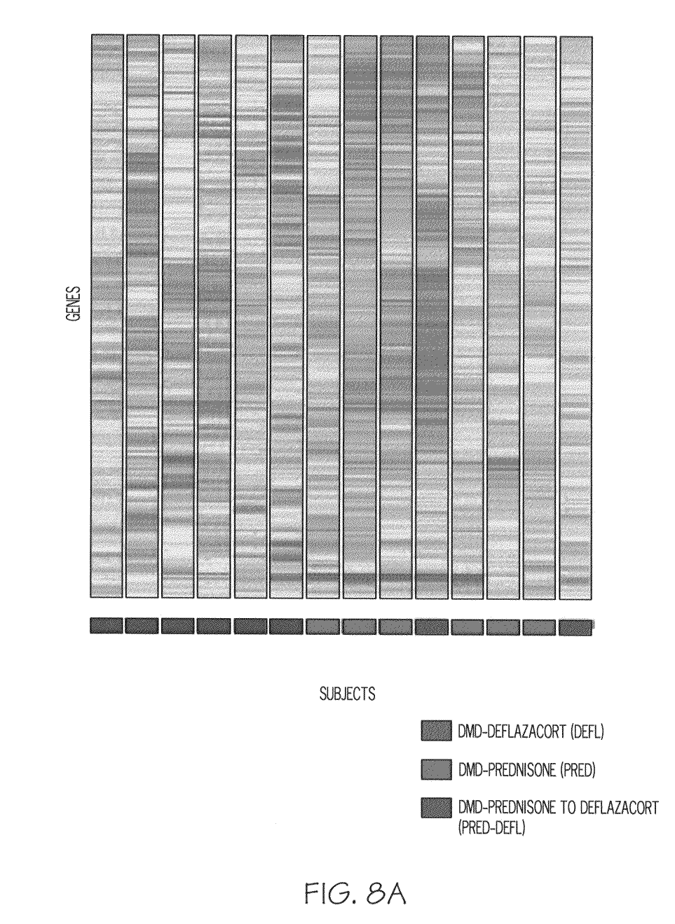

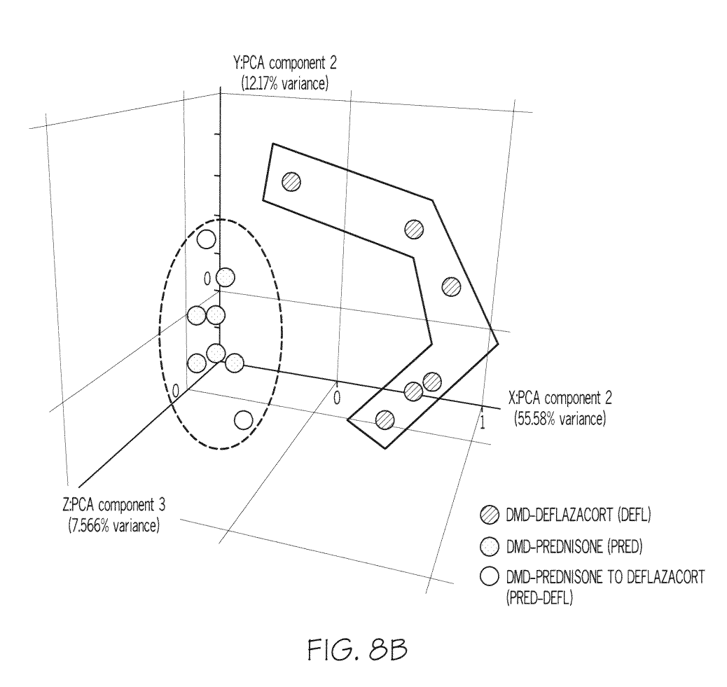

FIG. 8A shows cluster analysis of genes differentially regulated in peripheral whole blood of patients with DMD receiving deflazacort, patients with DMD receiving prednisone, compared with patients with DMD receiving prednisone and deflazacort.

FIG. 8B shows principal component analysis of individuals with DMD receiving prednisone and deflazacort, individuals with DMD receiving prednisone, and individuals with DMD receiving deflazacort.

Except where specifically indicated, gene expression in all individuals and all gene expression data were evaluated from peripheral blood.

Individuals with DMD, some of which had been administered prednisone and/or deflazacort, compared to controls, had significant differences in gene expression, which was the first demonstration of gene expression changes in peripheral blood on a whole genome level.

A total of 10,763 genes were significantly regulated in peripheral blood of these individuals with DMD, compared to individuals without DMD. Of the 10,763 genes that were significantly regulated, with regulation including genes that were either up- or down-regulated, the data were:

1467 genes had a fold change >|1.5|;

191 genes had a fold change >|2.0|; and

59 genes had a fold change >|2.5|, with fold changes indicated as absolute values.

The regulated genes in individuals with DMD were expressed mainly in neutrophils, monocytes, B cells, and CD4+ T cells. This mirrored to some extent the muscle infiltration of monocytes/macrophages and T-cells in individuals with DMD. Different genes were activated in the blood, compared to muscle, in individuals with DMD, though there were similarities in the functional pathways.

The gene encoding utrophin (nucleic acid 1378) was regulated in both blood and muscle of individuals with DMD. The utrophin protein is highly homologous to dystrophin (80% homology). Dystrophin is deficient in individuals with DMD. In blood, utrophin was down-regulated two-fold in individuals with DMD compared to controls. In muscle, utrophin is up regulated in individuals with DMD compared to controls, presumably as a partial compensation for the dystrophin deficiency. The two-fold down-regulation of utrophin in blood presumably relates to a change in immune function of the white cells in individuals with DMD, rather than as a consequence of the dystrophin deficiency in muscle.

In individuals with DMD, some genes that are normally expressed in muscle were regulated in blood, but were not regulated in muscle. Some of these genes regulated in blood, but not muscle, of individuals with DMD include myoferlin, sarcolemma associated protein (SLMAP), myocyte enhancer factor 2C (MEF2C), myotubularin related protein 1 (MTMR1), and phosphodiesterase 4D interacting protein (PDE 4DIP) (nucleic acids 1405, 1465, 1232, 1423, 1464, and 80, respectively).

Myoferlin was down-regulated two fold in blood of individuals with DMD but, although present, was not regulated in muscle of individuals with DMD. Myoferlin has an unknown function, but is highly homologous to dysferlin, which is mutated in some types of limb-girdle muscular dystrophy. Myoferlin protein levels do not appear to be dysregulated in subjects with dysferlinopathy.

Sarcolemma associated protein (SLMAP) was down-regulated in blood of individuals with DMD but, although present, was not regulated in muscle of individuals with DMD. The coiled-coil, tail-anchored SLMAP protein self-organizes in the cardiomyocyte and may be involved in the excitation coupling apparatus.

Myocyte enhancer factor 2C (MEF2C), myotubularin related protein 1 (MTMR1), and phosphodiesterase 4D interacting protein (PDE 4DIP, myomegalin) were regulated in blood of individuals with DMD but, although present, were not regulated in muscle of individuals with DMD. MEF2 genes control skeletal muscle development and MEF2C mediates the activation of induced cell death (AICD) of macrophages. Mutations of myotubularin-like proteins lead to myotubular myopathy and a form of Charcot-Marie-Tooth peripheral neuropathy. The myotubularin family of PI 3-phosphatases is a key regulator of two phosphoinositols that regulate traffic within the endosomal-lysosomal pathway. Myomegalin functions as an anchor to localize components of the cAMP-dependent pathway to the Golgi/centrosomal region of the cell.

Regulation of genes encoding myoferlin, sarcolemma associated protein (SLMAP), myocyte enhancer factor 2C (MEF2C), myotubularin related protein 1 (MTMR1), and phosphodiesterase 4D interacting protein (PDE 4DIP) point to pathways that are activated in white blood cells in individuals with DMD, and to pathways that are utilized within normal muscle cells but are not activated in the diseased muscle in individuals of DMD. These genes likely participated in functions of immune cells that were activated in response to diseased DMD muscle cells.

There was significant overlap in the signaling pathways that were activated in blood and muscle of individuals with DMD, including the leukocyte trans-endothelial migration pathway (FIG. 2) and the regulation of actin cytoskeleton pathway (FIG. 3). This similarity of signaling pathways in blood and muscle of individuals with DMD occurred despite the different genes activated in these pathways in blood compared to muscle. In muscle, the trans-endothelial migration pathway is more likely to represent muscle cell responses to infiltrating leukocytes, whereas in blood the response is likely in the infiltrating leukocytes themselves. The actin cytoskeleton pathway was also regulated in both blood and muscle of individuals with DMD. Regulation of the actin cytoskeleton in diseased muscle of individuals with DMD would be expected, because dystrophin forms part of the dystrophin-associated protein complex that links the cytoskeleton to the extracellular matrix in muscle cells. The reason for changes in the actin cytoskeleton of leukocytes in individuals with DMD is unclear. It is possible that other functions of dystrophin in muscle, including regulation of calcium, calmodulin, and nitric oxide synthase, affect leukocytes and their cytoskeleton during muscle infiltration. Leukocytes likely undergo changes of their actin cytoskeleton during infiltration into the diseased muscle fibers in individuals with DMD.

Amyloid precursor protein, cellular prion protein, and caspase 1 were up-regulated in individuals with DMD. Caspase-1, which cleaves interleukin 1 produced by white blood cells, could be involved in white blood cell apoptosis or regulating pro-inflammatory IL-1 levels in white blood cells. Caspase-1 up regulation may contribute to changes of oxidative stress and possible role of apoptosis in cell death in DMD muscle.

To identify pathways with the most differentially expressed genes, gene lists were searched against the KEGG database of human biochemical pathways using the KEGG pathway functional annotation tool in DAVID Bioinformatics Resources: http://david.abcc.ncifcrf.gov/. The top biochemical pathways identified by KEGG in blood of individuals with DMD included leukocyte transendothelial migration and antigen processing pathways; both are closely related to immune function. The monocytes/macrophages and T cell infiltration in muscle of individuals with DMD, and with dysferlin myopathy, is a well-recognized inflammatory response. Most individuals with DMD express a highly conserved peptide in the hypervariable domain of the T-cell receptor in cytotoxic lymphocytes, suggesting a specific immune response to a common antigen.

Immune cells, such as macrophages and T cells, promote the pathology of dystrophic muscle. Prednisone and prednisolone improve muscle strength in dystrophin deficient mdx mice, reduce muscle degeneration in dystrophin deficient Caenorhabditis elegans (C. elegans), decrease CD-11 inflammatory cells in mdx muscle, and improve function in individuals with DMD. Prednisone thus appears to improve function, at least in part, by decreasing inflammation and improving survival of diseased muscle fibers. The discloses data support a significant immune response in blood of individuals with DMD that involves several cell types including neutrophils, monocytes/macrophages, B cells, and T-cells. An agent that targeted one or more individually regulated genes would more specifically modulate the immune system and improve function in individuals with DMD. The agent may up-regulate gene expression. The agent may down-regulate gene expression. Examples of agents include, but are not limited to, an antisense oligonucleotide, an inhibitory RNA (RNAi), an agonist of the resultant protein, an antagonist of the resultant protein, an expression vector of the regulated gene, an antibody, etc. In one embodiment, an anti-human monoclonal antibody may be administered. In one embodiment, an antisense oligonucleotide for defensin may be administered. In one embodiment, a define RNAi may be administered.

The 1467 genes that had a fold change >|1.5| (Table 3, nucleic acids 1-1467), the 191 genes that had a fold change >|2.0| (Table 2, nucleic acids 1-78 and 1355-1467), and the 59 genes that had a fold change >|2.5| (Table 1, nucleic acids 1-45 and 1454-1467, and SEQ ID NOS. 1-55) were evaluated by cluster analysis to determine capability to separate individuals with DMD compared to control individuals. The results are shown in FIG. 1 where the red area indicates genes that were up-regulated, the dark blue area indicates genes that were down-regulated, the yellow area indicates genes that were not regulated, the light blue area indicates genes from control individuals, and the purple area indicates genes from individuals with DMD. The gene expression clusters indicated that individuals with DMD could be subgrouped based upon factors such as age, duration of DMD, and severity of DMD. These factors correlated with specific expression profiles. Some of the gene expression changes might correlate with an immune response to any muscle disease of any cause, such as other types of muscular dystrophy and other neuromuscular disorders. Some of the gene expression changes might be specific to muscle disease caused by specific gene mutations.

An RNA expression profile of individuals with DMD compared to control individuals was generated. The immune response to necrosis/apoptosis of dystrophic muscle likely accounts, at least in part, for the changes of RNA expression. RNA changes might provide an assay for monitoring targeted therapies for DMD and other neuromuscular disorders.

Gene expression and/or gene expression profiles were identified in individuals with DMD that were administered the agents deflazacort, prednisone, or deflazacort and prednisone. Gene expression changes common to administration of either deflazacort or prednisone related to the efficacy of deflazacort therapy, prednisone therapy, or deflazacort and prednisone therapy in individuals with DMD. In addition, there were gene expression changes that were specific for deflazacort or prednisone that might relate to deflazacort- or prednisone-specific side effects or therapeutic actions.

When gene expression in individuals with DMD not receiving deflazacort or prednisone, were compared to gene expression in individuals with DMD receiving deflazacort, prednisone, or deflazacort and prednisone, expression of 524 genes was significantly different. The results are shown in Table 4, FIG. 6A. Of these 524 genes, expression in 127 genes was down-regulated (24%), and expression in 397 genes was up regulated (76%).

Using these data in a prediction analysis of microarray (PAM) to cross-validate probabilities, 59 probes representing 50 annotated genes were identified that optimally distinguished patients with DMD receiving deflazacort, prednisone, or deflazacort and prednisone, from patients with DMD not receiving deflazacort, prednisone, or deflazacort and prednisone. Table 6. Ten-fold leave-one-out cross-validation demonstrated that these probes correctly classified 29 of 34 individuals (85.2%) administered deflazacort, prednisone, or deflazacort and prednisone. FIG. 6B, Table 16. The analysis accurately identified all of the 20 individuals with DMD not administered deflazacort, prednisone, or deflazacort and prednisone. The analysis correctly identified 9 of the 14 (64.3%) individuals administered deflazacort, prednisone, or deflazacort and prednisone. Of the 5 individuals that were not accurately identified, 3 individuals had been administered prednisone, and two had been administered deflazacort.

The individuals with DMD administered deflazacort or prednisone, compared to individuals with DMD not administered deflazacort or prednisone, demonstrated enhanced defense responses and up-regulation of genes associated with primary and secondary granules in granulocytes (neutrophils), iron trafficking, and chondroitin sulfate synthesis. The individuals with DMD administered deflazacort or prednisone, compared to individuals with DMD not administered deflazacort or prednisone, demonstrated increased expression levels of lactotransferrin and lipocalin, genes involved in iron and heme homeostasis. Lactotransferrin, an iron binding protein found in secondary granules of neutrophils, is important in the innate immune response against infections, and appears to be critically important in the oxidative burst during infections. These results confirmed a previous study that demonstrated that corticosteroid administration to normal individuals increased lactotransferrin blood levels. Lipocalin 2 is important in removing or preventing iron from entering siderophore bound iron in bacteria. Lipocalin 2 also binds and carries iron in neutrophils, and plays a role in resistance against tissue injury. Both lactotransferrin and lipocalin 2 traffic iron to late endosomes where acidification and reduction processes release iron stores. Steroids have previously been reported to induce lipocalin both in vitro and in vivo.

The individuals with DMD administered deflazacort or prednisone, compared to individuals with DMD not administered deflazacort or prednisone, demonstrated up-regulated expression levels of haptoglobin and CD163 nucleic acids (nucleic acids 1641 and 1526, respectively). Haptoglobin, made predominantly in the liver, binds hemoglobin and other heme proteins. It is specifically taken up by the CD163 receptor into macrophages, thus sequestering heme and iron and preventing oxidative damage. The up-regulation of haptoglobin could serve to bind and sequester heme proteins released from injured or dying muscle fibers in DMD and other neuromuscular diseases. Corticosteroids induce haptoglobin protein production in blood of mammals and in cultured cells. The results showing increased haptoglobin expression in response to corticosteroids in humans confirmed these previous studies.

The disclosed results are consistent with a previous human study showing that corticosteroids also induced CD163 protein on the surface of peripheral blood monocytes. That study suggested that corticosteroids facilitated CD163-mediated endocytosis of hemoglobin to monocytes/macrophages and thereby induced CD163 and heme oxygenase-1 (HO-1) synthesis. The HO-1 metabolism of heme from heme-containing proteins and sequestration of the released iron could account for some of the anti-inflammatory actions of corticosteroids.

Several of the disclosed up-regulated genes, including lactotransferrin, cathepsin G, elastase, and azurocidin, directly bound ceruloplasmin. Ceruloplasmin is an iron binding protein and an endogenous antioxidant that oxidizes Fe²⁺ to Fe³⁺, mitigating the oxidant effect of iron. While some of the disclosed corticosteroid up-regulated genes were reported previously in other systems, the disclosed results demonstrated the coordinated induction of iron and heme regulatory genes in human leukocytes in response to corticosteroids. This is particularly interesting in light of therapeutic effects of iron deprivation demonstrated in the mdx mouse.

The individuals with DMD administered deflazacort or prednisone, compared to individuals with DMD not administered deflazacort or prednisone, demonstrated up-regulated expression levels of genes in the chondroitin sulfate biosynthesis pathway. Biglycan and decorin, chondroitin/dermatan sulfate proteoglycans found in muscle extracellular matrix, have been shown to increase in muscle of individuals with DMD. Biglycan and decorin may act as regulators of fibrosis through inhibition of TGF-B1 profibrotic cytokine activity. However, reduced proteoglycan sulfonation in and at the basal lamina has been identified in cultured DMD muscle cells. The disclosed up-regulation of chondroitin β-1,4 N-acetylgalactosaminyltransferase (ChGn, GALNACT-2) in response to corticosteroids might normalize proteoglycan sulfonation and enhance anti-fibrotic effects of biglycan and decorin by contributing to initiation and elongation in chondroitin sulfate biosynthesis.

The individuals with DMD administered deflazacort or prednisone, compared to individuals with DMD not administered deflazacort or prednisone, demonstrated up-regulated expression levels of FKBP5. The up-regulation of FKBP5 in response to corticosteroid use has been consistently demonstrated, including in the mdx mouse, and has been associated with loss of corticosteroid efficacy. The FKBP5 protein inhibits calcineurin, which controls and regulates slow-twitch oxidative (type 1) muscle fibers. Manual curation comparing expression changes in the mdx mouse identified only one regulated probe in addition to FKBP5; this was KFL10. KFL10, up-regulated following six weeks of prednisone administration in the mdx mouse, was also up-regulated in individuals administered prednisone, deflazacort, or both, compared with individuals DMD not administered any agent. KLF10 is a TGF-β-inducible early gene that acts as a transcription factor regulator binding to GC-rich Sp1-like sequences. Thus it is possible that although downstream expression changes differ between muscle and blood in response to steroid administration in individuals with DMD, these cascades are initiated by similar mechanisms.

None of the genes having deflazacort- or prednisone-altered expression in individuals with DMD had glucocorticoid (corticosteroid) response elements (GRE) significantly over- or under-represented in transcription factor binding motifs. The most significantly over-represented transcription factor binding motif family was the myocyte-specific enhancer factor-2 (MEF2) family. The proteins in the MEF2 family bind to the MEF2 DNA sequence in regulatory regions of most muscle-specific genes. Other over-represented transcription factor motifs included CEBPA, Oct 6 and Brn 2. CEBPA (CCAAT enhancer binding proteins) are transcription factors involved in specification of myeloid lineages (i.e., all white blood cell types except lymphocytes and monocytes) from stem cells and up-regulation of expression for primary and secondary granule proteins. The shift to a predominantly myeloid lineage immune response, from lymphocyte defense responses, in response to corticosteroids is further supported by under representation of binding sites for Ikaros transcription factors, transcription factors regulating lymphocyte differentiation. The transcription factors Oct6 and Brn2 interact to promote the transition from promyelinating to myelinating Schwann cells. Since the short form of dystrophin is found in peripheral myelin, over-representation of Oct6 and Brn2 transcription factor binding sites is intriguing Enhanced proteolytic activity by matrix metalloproteinase 9 (MMP-9) in mdx mouse, suggested to arise from increased inflammation and subsequent macrophage activation, might potentiate myelin degeneration in dystrophin-deficient Schwann cells. Thus the over representation of the Oct6 and Bm2 transcription factors suggest a possible remyelination response due to corticosteroid administration in individuals with DMD.

The individuals with DMD administered deflazacort, compared to the individuals with DMD administered prednisone, demonstrated 508 genes nucleic acids 1-508 that were significantly different. The results are shown in Table 5, FIG. 7A. Of the 508 genes, 478 (94%) were significantly down-regulated in individuals with DMD administered deflazacort compared to individuals with DMD administered prednisone. Of the 508 genes, 30 (6%) were significantly up-regulated in individuals administered deflazacort compared to individuals administered prednisone. Using these data in a prediction analysis of microarray (PAM) to cross-validate probabilities, 496 probes were identified as distinguishing individuals with DMD administered deflazacort, from individuals with DMD receiving prednisone. The results are shown in Table 7. Ten-fold leave-one-out cross-validation demonstrated that these probes correctly classified all individuals according to type of drug administered. The results are shown in FIG. 7B and Table 17.

In the individuals with DMD administered deflazacort, expression of retinoic acid receptor a (RARa) was significantly up-regulated compared to individuals administered prednisone. Retinoic acid inhibits adipocyte differentiation through RARa, and increased expression of RARa has been associated with reduced obesity levels.

In the individuals with DMD administered prednisone, expression of a number of genes related to lipid metabolism, particularly in adipose tissue, was up-regulated compared to individuals administered deflazacort. For example, Cas-Br-M (murine) ecotropic retroviral transforming sequence (CBL) is involved in adipose formation and energy homeostasis. The nicotinic acid-coupled G protein-coupled receptor 109B (GPR109B), and the G-protein-coupled receptor 43, a short-chain fatty acid receptor, each inhibits niacin-mediated lipolysis in adipose tissue. Nuclear receptor co-activator 1 (NCOA1), a co-activator required for transcriptional activity of the steroid receptor superfamily, is involved in energy metabolism. The interleukin 1 and interleukin 6 systems are both associated with obesity. Individuals with DMD administered prednisone had up-regulated interleukin 1 beta, interleukin 1 receptor antagonist, and interleukin 6 receptor. These results suggested a mechanism by which weight gain, an important side effect of prednisone administration, might be ameliorated, as well as a mechanism by which weight gain might be inhibited with deflazacort, for example, by administering agents that perturb regulation. There were fewer changes in gene expression in individuals that had been administered deflazacort compared to individuals that been administered prednisone. The gene expression profile of individuals administered deflazacort is more similar to the gene expression profile of individuals with DMD not administered prednisone or deflazacort, that to the gene expression profile of individuals with DMD administered prednisone.

Corticosteroid administration fostered myeloid maturation, may suppress fibrosis, and appeared to up-regulate genes for primary and secondary granule proteins and subsequent iron trafficking functions in individuals with DMD. The disclosed results indicated pathways involved in weight gain and metabolism that are up-regulated in individuals with DMD administered prednisone but not in individuals with DMD administered deflazacort. The association of these pathways with corticosteroid administration points to mechanisms of corticosteroid actions in DMD that might provide specific treatment targets for other agents without side effects of corticosteroids.

### EXAMPLE 1

### Gene Expression in Control Individuals Compared to Individuals with DMD not Administered any Agent any or Administered Prednisone and/or Deflazacort

Gene expression from 34 males with DMD, ranging from 3 years to 20 years with a mean age of 9 years±3 years, was compared to gene expression in 21 control males matched for age. Table 8. Individuals with DMD were evaluated and had been diagnosed by a single board-certified pediatric neurologist. The diagnosis of DMD was based in part upon clinical history, examination findings, and selected muscle biopsies and was confirmed by molecular diagnostic testing.

Of the 34 individuals with DMD, 14 were administered either prednisone (PRED, n=6) or deflazacort, an oxazoline derivative of prednisolone (DEFL; n=8) at the time blood samples for gene expression evaluation were obtained. The mean duration of administration of either prednisone or deflazacort for these 14 individuals was 43.9 months. Of the 34 individuals with DMD, 20 individuals had not been administered either prednisone or deflazacort at the time blood samples for gene expression evaluation were obtained and details of clinical and genetic testing are available upon request.

There was a non-significant age difference (t-test, p>0.20) between the control individuals and the individuals administered either prednisone or deflazacort. One control individual and 3 individuals with DMD were not Caucasian. An unpaired t-test was performed when comparing results among all of the individuals (both control individuals and individuals with DMD), and a paired t-test was performed when comparing equal numbers of age-matched, male, Caucasian control individuals and individuals with DMD, as subsequently shown.

The control individuals were recruited from local schools with similar demographics to those from which individuals with DMD attended, and blood was obtained and processed using identical methods for both groups.

Sample Processing and Array Hybridization

Fifteen ml of blood was collected from each individual into 6 PAXgene® Vacutainer® tubes (PreAnalytiX, Germany) by antecubital fossa venipuncture. PAXgene® tubes contain a proprietary reagent that immediately stabilizes RNA, thus reducing RNA degradation and minimizing gene expression changes following phlebotomy. The tubes were then inverted 20 times, then remained undisturbed for 2 h at room temperature (about 19° C. to about 22° C.), and then stored at −70° C. until processed.

Total RNA was isolated using the Paxgene® blood RNA kit (PreAnalytiX, Germany) according to the manufacturers instructions. The RNA isolated is from all cells in whole blood. Typical yields of total RNA isolated from about 7.5 ml of human whole blood were between 10 μg RNA and 40 μg RNA. RNA quality and purity was analyzed by spectrophotometry using the Nanodrop ND-1000, and RNA integrity was analyzed using the Agilent 2100 Bioanalyzer. A₂₆₀/A₂₈₀ of purified RNA exceeded 2.0, and the 28S/18S rRNA ratios were equal to or exceeded 1.8.

Sample labeling, hybridization to chips, and image scanning were performed using standard Affymetrix protocols. For each whole blood sample from control individuals and individuals with DMD, total RNA was labeled using the One-Cycle Target Labeling protocol and hybridized to the arrays. Gene expression was assessed on the Human U133 Plus 2.0 GeneChip (Affymetrix, Santa Clara Calif.), which consisted of a single oligonucleotide microarray that surveyed over 54,000 probe sets, or greater than 30,000 possible human genes. Double-stranded cDNA synthesis, biotin-labeled cRNA synthesis, and cRNA fragmentation were performed according to the Affymetrix GeneChip Eukaryotic Expression Analysis protocol. The labeled, fragmented cRNA (15 μg) was hybridized at 45° C. overnight. After hybridization, the array was washed and stained. The arrays were scanned with an argon-ion laser at λ=570 nm, with a resolution of 3 μm/pixel (Affymetrix, Inc.).

Probe-Level Data Analysis

After scanning the array, the raw probe level values for each probe were saved in Affymetrix.cel files. The probe level values were imported into Genespring 7.2 (Agilent Technologies, Palo Alto Calif.), and then processed using GC-RMA, followed by a three-step normalization (data transformation, per chip normalization, and per gene normalization) (Genespring 2004).

Statistical Analyses

Statistical analyses, including t-test analyses, unsupervised hierarchical clustering, and principal component analyses were conducted using Genespring 7.2 software (GENESPRING® 7, Silicon Genetics, Redwood City Calif.). Significance was assessed using the criteria at least 1.5-fold difference in expression between groups and statistically significantly different level of expression (parametric t-tests, p≦0.05). Corrections for multiple comparisons used a Benjamini-Hochberg False Discovery Rate (FDR) of 0.05, disclosed in Tang et al., J Cereb Blood Flow Metab 26 (2006) 1089, which is expressly incorporated by reference herein in its entirety. This value was chosen because no more than 5% of the genes should represent false positives.

Results of the t-tests were reported without a fold change cutoff. Genes for which the fold change in the average expression values in individuals with DMD, compared to control individuals, were >1.5 (least stringent), >2.0 (moderately stringent), or >2.5 (very stringent). The gene lists were used for cluster analyses to determine how well the genes separated individuals with DMD from control individuals. The increasing degrees of stringency provided manageable numbers of genes; primary data are available upon request. Previous blood studies evaluating gene expression in neurological diseases had often used 1.5 fold changes. Approaches that combined significance value measures and magnitude of change measures provided good power and prediction, as shown for Significance Analysis of Microarrays (SAM) and Prediction Analysis of Microarrays (PAM).

Prediction analysis of microarray (PAM) software, which employs the “nearest shrunken centroids” method, was used to identify a minimum set of signature genes that distinguished individuals with DMD from control individuals. The accuracies of the classifiers were tested using a tenfold cross-validation.

Assessment of Possible Biological Significance of Identified Genes

Gene lists were searched against the KEGG database of human biochemical pathways (using the KEGG pathway functional annotation tool in DAVID Bioinformatics Resources: http://david.abcc.ncifcrf.gov/) to identify pathways with the most differentially expressed genes. A probability p value for the numbers of genes expressed in a given pathway was used to select the most regulated pathways in blood of individuals with DMD.

The primary gene expression data published for muscle from individuals with DMD, compared to control individuals, was re-analyzed using GCRMA. The regulated genes in muscle of individuals with DMD subjects were compared to the regulated genes in peripheral blood of individuals with DMD. The most prominent pathways regulated in blood were compared to those regulated in muscle for individuals with DMD (FIGS. 2, 3, 4, and 5).

When gene expression was evaluated from the 34 individuals with DMD, compared to the 21 control individuals, 10,763 genes were found to be significantly regulated (unpaired t-test and a Benjamini-Hochberg False discovery rate (FDR), (p≦0.05), no cutoff for fold change, data not shown). Of the 10,763 genes that were significantly regulated, 59 genes showed a fold change >|2.5| (Table 1), 191 genes showed a fold change >|2.0| (Table 2), and 1467 genes showed a fold change of >|1.5| (Table 3).

A separate analysis was performed on equal sized groups matched for gender (male), race (Caucasian), and age (3 years to 20 years). A paired t-test for individuals with DMD (n=20) compared to control individuals (n=20) resulted in lists of genes that overlapped the above gene lists, using a FDR of <0.05 and the fold changes indicated above (data not shown).

Using the data in Table 1 (which lists the 59 genes differentially regulated in individuals with DMD compared to control individuals using an unpaired t-test, FDR <0.05, fold change >2.5), a cluster analysis separated individuals with DMD from control individuals. The results are shown in FIG. 1. Red indicates genes that were up-regulated. Dark blue indicates genes that were down-regulated. Yellow indicates genes that were not regulated. Light blue indicates genes from control individuals. Purple indicates genes from individuals with DMD.

Control individuals formed a single group with a block of genes (lower left) that were generally expressed at higher levels in control individuals, compared to individuals with DMD (lower right).

Based upon the patterns of gene expression for individuals with DMD, different subgroups of genes were discerned. These subgroups appeared to further distinguish among individuals with DMD. where at least two subgroups of individuals with DMD and as many as five subgroups of individuals with DMD were detected. The clinical factors which subdivide these subgroups of individuals with DMD is unclear.

Prediction Analysis of Individuals with DMD and Control Individuals

Prediction analysis of microarray (PAM) identified 13 genes from Tables 9 and 10) as signature genes, with an overall misclassification rate of 6%. Signature genes were identified by adjusting a threshold of fold change ≧2.2, or fold change ≦2.2, which defined the minimum number of genes that best distinguished individuals with DMD from control individuals. The PAM analysis of these 13 genes showed that, of these 34 individuals with DMD, 2 individuals were misclassified as controls, while of these 21 control individuals, 2 individuals were misclassified as individuals with DMD. Data from the cluster analysis shown in FIG. 1 accurately segregated all individuals with DMD and control individuals. Table 16.

Signaling Pathways Activated in Blood of Individuals with DMD

Based upon the genes showing ≧2 fold regulation (the 191 genes in Table 2), the signaling pathways in which these genes operated were examined. The genes in Table 2 were searched against the KEGG database of human biochemical pathways, and the pathways with the most statistically significant numbers of differentially regulated genes are shown in Table 11 (p value=significance of the number of differentially regulated genes expressed in a pathway, compared to the total number of genes currently known in that pathway; methods for calculating p value are included within the KEGG package.)

The results are shown in FIGS. 2, 3, 4, and 5. Genes from blood of individuals with DMD, compared to control individuals are shown in stippling. Genes from muscle of individuals with DMD, compared to control individuals, are shown in cross-hatch (Haslett et al., Neurogenetics 4 (2003) 163, which is expressly incorporated by reference herein in its entirety), and results from re-analyzed Haslett data are unmarked. The re-analysis used GCRMA for normalization, and used the same criteria for muscle samples as was used for blood samples, namely, unpaired t-test, FDR <0.05, fold change >2.0.

The numbers of genes determined to be regulated in a particular pathway was a function of the number of genes examined. Thus, examining the 1467≧1.5 fold differentially regulated genes (Table 3) using the KEGG pathway program resulted in 3-7 times more genes in each pathway, but the pathways with the largest and most significant numbers of regulated genes for individuals with DMD, compared to control individuals, were the same as shown in FIGS. 2, 3, 4, and 5.

The genes regulated in blood of individuals with DMD, compared to the genes reported to be regulated in muscle of individuals with DMD, showed relatively few identical genes with the exception of utrophin. Utrophin was up-regulated in muscle in individuals with DMD (Haslett et al. cited previously), while utrophin was down-regulated in blood of individuals with DMD (Table 3). Although FIG. 3 shows regulation of integrin (ITG) in both blood and muscle from individuals with DMD (ITG gene both red and yellow), a blood-specific integrin is expressed in blood (integrin, β1-fibronectin receptor, β-polypeptide, antigen CD29 included MDF2, MSK12) and a muscle-specific integrin is expressed in muscle (integrin, β 2-complement component 3 receptor 3 and 4 subunit). Gene expression levels of amyloid pre-protein (APP), cellular prior protein (PrPc), and caspase-1 (ICE, interleukin-1 converting enzyme) were increased in blood from individuals with DMD (FIG. 5) but not in muscle from individuals with DMD.

Genes Regulated in Individuals with DMD were Expressed in Specific Blood Cell Types

Specific cell types from blood were not isolated in individuals with DMD or control individuals. Cell type assignment for differentially regulated genes in blood from individuals with DMD subjects was assessed using inventors' prior data Du et al., Genomics 87 (2006) 693, which is expressly incorporated by reference herein in its entirety. The genes showing ≧2 fold regulation (the 191 genes in Table 2) were mapped onto the expression profiles for different types of peripheral blood cells obtained from 3 control individuals. The genes showing ≧2 fold regulation (the 191 genes in Table 2) were expressed mainly in neutrophils, and to a lesser extent in monocytes, B cells, and CD4+ T helper cells.

### EXAMPLE 2

### Gene Expression in Control Individuals with DMD Not Administered Prednisone or Deflazacort, Compared to Individuals with DMD Administered Prednisone or Deflazacort

Gene expression from 34 males with DMD, ranging from 3 years to 20 years with a mean age of 9±3 years, was determined (Table 8). Individuals with DMD were evaluated and had been diagnosed by a single board-certified pediatric neurologist. The diagnosis of DMD was based in part upon clinical history, examination findings, and selected muscle biopsies and was confirmed by molecular diagnostic testing.

Of the 34 individuals with DMD, 14 were administered either prednisone (PRED, n=6) or deflazacort, an oxazoline derivative of prednisolone (DEFL, n=8) at the time blood samples for gene expression evaluation were obtained. The mean duration of administration of either prednisone or deflazacort for these 14 individuals was 43.9 months. Of the 34 individuals with DMD, 20 individuals had not been administered either prednisone or deflazacort at the time blood samples for gene expression evaluation were obtained.

Sample processing and array hybridization, probe-level data analysis, and statistical analysis were identical to Example 1.

Assessment of Biological Significance of Identified Genes

Differentially regulated genes were analyzed using The Database for Annotation, Visualization and Integrated Discovery (DAVID, http://niaid.abcc.ncifcrf.gov/) to examine co-regulation of gene pathways and biological significance. DAVID identifies over-representation of genes within particular pathways, indicating co-regulation of genes in the pathway, using the Expression Analysis Systematic Explorer (EASE) program to generate an EASE score, which is a modified Fisher Exact test score. The following DAVID default parameters were applied: minimum number of genes required per pathway or chromosome=2; maximum EASE score accepted=0.1. A DAVID Functional Annotation Clustering Report (FACR) identified similar annotations for identified probe lists. The FACR analysis addresses redundancy of different annotation databases by grouping similar annotations together, and calculates a Group Enrichment Score. The Group Enrichment Score ranks these annotation groups (in-log scale of group members' p-values in each annotation cluster) according to degree of common genes identified across annotation databases. Higher Group Enrichment Scores indicated increased level of biological significance (http://david.abcc.ncifcrf.gov/content.jsp?file=functional annotation.html).

To determine if similar transcription factors were involved in coordination of expression changes in response to corticosteroids, transcription factor binding motifs (TFBMs) that were wither over- or under-represented in differentially expressed genes were identified using the TELiS data base (http://www.telis.ucla.edu/). TELiS performs frequency analyses to compare the average number of TFBMs detected in promoters of differentially expressed genes, with the average number in non-differentially expressed genes, using a z-test.

RNA Expression Changes in Individuals with DMD not Administered Prednisone or Deflazacort, Compared to Individuals with DMD Administered Prednisone or Deflazacort

When individuals with DMD not administered prednisone or deflazacort, were compared to individuals with DMD administered either prednisone or deflazacort, expression of 524 genes was significantly different (fold change >|1.5|, unpaired t-test, p≦0.05) FIG. 6A, Table 4.

Of the 524 genes, expression levels were down-regulated in 127 genes (24%), and were up-regulated in 397 genes (76%) in individuals with DMD administered prednisone or deflazacort, compared to individuals with DMD not administered prednisone or deflazacort (log-likelihood, p<0.00001) (FIG. 6A). The 524 genes were analyzed using PAM, which identified 59 probes, representing 50 annotated genes, that optimally distinguished the individuals (Table 6). Ten-fold leave-one-out cross-validation demonstrated that these genes accurately identified 29 of 34 individuals (85.2%) according to whether the individual was or was not administered prednisone or deflazacort (FIG. 6B, Table 16). All 20 individuals with DMD not administered prednisone or deflazacort were accurately identified. Nine of 14 individuals with DMD (64.3%) administered prednisone or deflazacort were accurately identified. Of the individuals not accurately identified, 3 were administered prednisone, and 2 were administered deflazacort. Prednisone and deflazacort had been administered for a duration ranging from 2 months to 108 months to individuals with DMD ranging in age from 4.5 years to 14.4 years.

Promoter Sites Activated in Blood of Individuals with DMD Administered Prednisone or Deflazacort.

Using the 59 genes identified by PAM, TELiS identified 45 genes as present within its database. Using these 45 genes, TELiS identified 13 transcription factor binding motifs (TFBMs) that were significantly over-represented within the 45 regulated genes, and one TFBM that was under-represented within the 45 regulated genes. Table 12. Glucocorticoid response elements did not appear within the identified TFBMs.

Biological Significance Assessment

Using the 59 genes identified by PAM as input to DAVID, 50 genes were identified. The strongest Functional Annotation Cluster (enrichment score=5.9) identified by DAVID reflected antibiotic and antimicrobial response functions (p-value range 4.30⁻⁹ to 2.20⁻³). Table 13. Within this list manual curation identified genes determined to be up-regulated in individuals with DMD administered prednisone or deflazacort, involved in iron trafficking (lipocalin, lactotransferrin, haptoglobin, cathepsin G, azurocidin, elastin), and involved in chondroitin sulfate synthesis (chondroitin β1,4 N-acetylgalactosaminyltransferase).

RNA Expression Changes in Blood in Individuals with DMD Administered Deflazacort Compared to Individuals with DMD Administered Prednisone

Expression of 508 genes was significantly different in individuals with DMD administered deflazacort compared to individuals with DMD administered prednisone (fold change ≧|1.5|, unpaired t-test, p≦0.05) (FIG. 7A, Table 5). Expression of 478 genes (94%) had significantly lower expression levels in individuals administered deflazacort compared to individuals administered prednisone (FIG. 7A). Thirty genes (6%) had significantly higher expression levels in individuals administered deflazacort compared to individuals administered prednisone (log-likelihood, p<0.00001) (FIG. 7A). The 508 genes were analyzed by PAM, which indicated that 496 genes were capable of distinguishing individuals administered deflazacort form individuals administered prednisone (Table 7). Ten-fold leave-one-out cross validation demonstrated that these 496 genes accurately identified all individuals according to type of steroid administered (FIG. 7B, Table 17).

The expression profile of the 2 individuals that were initially administered prednisone and later administered deflazacort was examined to determine whether the expression profiles for these individuals would resemble either of the previous groups. Using the 508 gene list, these 2 individuals had expression patterns similar to individuals administered prednisone, using either unsupervised hierarchical clustering (FIG. 8A) or principal component analyses (FIG. 8B). The 496 gene list generated using PAM accurately identified 1 of the 2 individuals.

Functional Annotation Clustering

Using the 496 gene set, the strongest Functional Annotation Cluster (enrichment score=7.49) identified by DAVID reflected intracellular signaling and signal transduction (p-value range 1.70⁻¹⁰ to 1.40⁻⁶) (Table 14). Further manual curation identified genes in the 496 gene set involved in adipose formation, retinoic acid signaling, and NFKB pathways.

Overlap Between Comparisons

Eighteen genes were identified as differently regulated in both individuals administered prednisone or deflazacort, compared to individuals not administered prednisone or deflazacort (18/524, 0.03%), and in individuals administered deflazacort compared to individuals administered prednisone (18/508, 0.04%) (Table 15). Nucleic acids 1702, 1848, 1750, 1576, 1738, 1711, 1650, 1686, 1675, 1675, 1807, 1664, 1590, 1736, 1596, 1688, 1667, and 1660.

TABLE 1
Control vs. Duchenne muscular dystrophy (DMD) - ≧|2.5| Fold Difference

| | | Fold | Genbank Accession | Nucleic | SEQ ID |
|---|---|---|---|---|---|
| Common Name | Description | Change | Number | Acid No. | NO. |
| DEFA4 | defensin, alpha 4, corticostatin | 26.06 | NM_001925 | 1 | 1 |
| SH3GL3 | predicted; contains SH3 | 11.96 | AF036272 | 2 | 2 |
| | domain; member of the EEN | | | | |
| | gene family; Homo sapiens | | | | |
| | EEN-B2-L4 mRNA, complete | | | | |
| | cds. | | | | |
| ANKRD20A | hypothetical protein | 11.14 | AL136793 | 3 | 3 |
| | DKFZp434A171 | | | | |
| | Homo sapiens cDNA: | 8.979 | AK027226 | 4 | 4 |
| | FLJ23573 fis, clone LNG12520. | | | | |
| CEACAM8 | carcinoembryonic antigen- | 7.116 | M33326 | 5 | 5 |
| | related cell adhesion molecule 8 | | | | |
| | CDNA clone IMAGE: 5271374, | 6.775 | BC041865 | 6 | 6 |
| | partial cds | | | | |
| ANKRD20A | Similar to FLJ00310 protein | 6.628 | BC022380 | 7 | 7 |
| | (LOC388669), mRNA | | | | |
| AZU1 | azurocidin 1 (cationic | 6.272 | NM_001700 | 8 | 8 |
| | antimicrobial protein 37) | | | | |
| ELA2 | elastase 2, neutrophil | 5.796 | NM_001972 | 9 | 9 |
| | CDNA FLJ33010 fis, clone | 5.19 | AI379823 | 10 | 10 |
| | THYMU1000336 | | | | |
| | Homo sapiens full length insert | 5.063 | AF086134 | 11 | 11 |
| | cDNA clone ZA88B06. | | | | |
| RAB45 | hypothetical protein FLJ31614 | 3.964 | NM_152573 | 12 | 12 |
| ECHDC3 | enoyl Coenzyme A hydratase | 3.84 | NM_024693 | 13 | 13 |
| | domain containing 3 | | | | |
| | Transcribed sequence with | 3.824 | AI797678 | 14 | 14 |
| | weak similarity to protein | | | | |
| | sp: O14595 (H. sapiens) | | | | |
| | NIF2_HUMAN Nuclear LIM | | | | |
| | interactor-interacting factor 2 | | | | |
| RAB45 | hypothetical protein FLJ31614 | 3.654 | NM_152573 | 15 | |
| IL1R2 | Human soluble type II | 3.624 | U64094 | 16 | 15 |
| | interleukin-1 receptor mRNA, | | | | |
| | complete cds. | | | | |
| TRIM4 | tripartite motif-containing 4 | 3.573 | AF220023 | 17 | 16 |
| | Transcribed sequences | 3.52 | BE972639 | 18 | 17 |
| | Transcribed sequences | 3.509 | AW337833 | 19 | 18 |
| CTSG | cathepsin G | 3.437 | NM_001911 | 20 | 19 |
| | Clone IMAGE: 5519764, mRNA | 3.434 | BC035915 | 21 | 20 |
| | | 3.195 | NM_018572 | 22 | |
| | CDNA clone IMAGE: 6165535, | 3.114 | AW086077 | 23 | 21 |
| | partial cds | | | | |
| SLC4A4 | solute carrier family 4, sodium | 3.081 | AF069510 | 24 | 22 |
| | bicarbonate cotransporter, | | | | |
| | member 4 | | | | |
| SLC16A3 | solute carrier family 16 | 2.984 | NM_004207 | 25 | 23 |
| | (monocarboxylic acid | | | | |
| | transporters), member 3 | | | | |
| | unnamed protein product; | 2.978 | AK094974 | 26 | 24 |
| | Homo sapiens cDNA FLJ37655 | | | | |
| | fis, clone BRHIP2010444. | | | | |
| | CDNA FLJ41501 fis, clone | 2.948 | BF667120 | 27 | 25 |
| | BRTHA2006975 | | | | |
| | Transcribed sequences | 2.887 | AW665840 | 28 | 26 |
| | Transcribed sequences | 2.859 | AW665538 | 29 | 27 |
| | CDNA FLJ41762 fis, clone | 2.843 | AK096159 | 30 | 28 |
| | IMR322004768 | | | | |
| | Transcribed sequences | 2.725 | AW340015 | 31 | 29 |
| | | 2.686 | NM_014136 | 32 | |
| FAD104 | FAD104 | 2.663 | N32832 | 33 | 30 |
| | out-of-frame rearrangement, | 2.649 | AJ275453 | 34 | 31 |
| | stop codon in FR2, 1 bp | | | | |
| | deletion; Homo sapiens partial | | | | |
| | IGVH4 gene for | | | | |
| | immunoglobulin heavy chain V | | | | |
| | region, case 2, cell D 56. | | | | |
| | Transcribed sequence with | 2.603 | AI733564 | 35 | 32 |
| | weak similarity to protein | | | | |
| | pir: A40138 (H. sapiens) A40138 | | | | |
| | glycogen phosphorylase | | | | |
| | | 2.578 | NM_018575 | 36 | |
| FLJ36166 | hypothetical protein FLJ36166 | 2.563 | AL049437 | 37 | 33 |
| ZNF33A | Homo sapiens brain my041 | 2.562 | AF063599 | 38 | 34 |
| | protein mRNA, complete cds. | | | | |
| | Homo sapiens B lymphocyte | 2.553 | AF304443 | 39 | 35 |
| | activation-related protein BC- | | | | |
| | 2048 mRNA, complete cds. | | | | |
| | Transcribed sequences | 2.551 | AI003508 | 40 | 36 |
| | Clone IMAGE: 5299049, mRNA, | 2.542 | BC036241 | 41 | 37 |
| | partial cds | | | | |
| HP | haptoglobin-related protein | 2.534 | NM_020995 | 42 | 38 |
| DAZ3 | deleted in azoospermia 4 | 2.533 | AF271088 | 43 | 39 |
| | CDNA clone IMAGE: 5541269, | 2.533 | BC039490 | 44 | 40 |
| | partial cds | | | | |
| SLC4A4 | solute carrier family 4, sodium | 2.511 | AF157492 | 45 | 41 |
| | bicarbonate cotransporter, | | | | |
| | member 4 | | | | |
| FNBP1 | formin binding protein 1 | 0.398 | AA937109 | 1454 | 42 |
| DDX3X | DEAD (Asp-Glu-Ala-Asp) box | 0.398 | R60068 | 1455 | 43 |
| | polypeptide 3, X-linked | | | | |
| UBE4B | ubiquitination factor E4B (UFD2 | 0.396 | AW241715 | 1456 | 44 |
| | homolog, yeast) | | | | |
| C22orf1 | chromosome 22 open reading | 0.392 | BC034024 | 1457 | 45 |
| | frame 1 | | | | |
| | CDNA FLJ10256 fis, clone | 0.392 | AK001118 | 1458 | 46 |
| | HEMBB1000870 | | | | |
| IGHM | Partial mRNA for IgM | 0.39 | BG340670 | 1459 | 47 |
| | immunoglobulin heavy chain | | | | |
| | variable region (IGHV gene), | | | | |
| | clone LIBPM376 | | | | |
| | Full length insert cDNA clone | 0.385 | AF085827 | 1460 | 48 |
| | YF47B02 | | | | |
| MO25 | MO25 protein | 0.363 | AF151824 | 1461 | 49 |
| SIAT1 | sialyltransferase 1 (beta- | 0.361 | AV695711 | 1462 | 50 |
| | galactoside alpha-2,6- | | | | |
| | sialyltransferase) | | | | |
| TncRNA | MRNA; cDNA | 0.338 | AI042152 | 1463 | 51 |
| | DKFZp686L01105 (from clone | | | | |
| | DKFZp686L01105) | | | | |
| MTMR1 | myotubularin related protein 1 | 0.32 | AK001816 | 1464 | 52 |
| FER1L3 | fer-1-like 3, myoferlin (C. elegans) | 0.32 | NM_013451 | 1465 | 53 |
| TncRNA | MRNA; cDNA | 0.302 | AV659198 | 1466 | 54 |
| | DKFZp686L01105 (from clone | | | | |
| | DKFZp686L01105) | | | | |
| HIPK3 | homeodomain interacting | 0.248 | AF305239 | 1467 | 55 |
| | protein kinase 3 | | | | |

TABLE 2
Control vs. Duchenne muscular dystrophy (DMD) - ≧|2.0| Fold Difference

| | | | Genbank | | |
|---|---|---|---|---|---|
| | | Fold | Accession | Nucleic | SEQ ID |
| Common Name | Description | Change | Number | Acid No. | NO. |
| DEFA4 | defensin, alpha 4, corticostatin | 26.06 | NM_001925 | 1 | 1 |
| SH3GL3 | predicted; contains SH3 domain; | 11.96 | AF036272 | 2 | 2 |
| | member of the EEN gene family; | | | | |
| | Homo sapiens EEN-B2-L4 mRNA, | | | | |
| | complete cds. | | | | |
| ANKRD20A | hypothetical protein DKFZp434A171 | 11.14 | AL136793 | 3 | 3 |
| | Homo sapiens cDNA: FLJ23573 fis, | 8.979 | AK027226 | 4 | 4 |
| | clone LNG12520. | | | | |
| CEACAM8 | carcinoembryonic antigen-related cell | 7.116 | M33326 | 5 | 5 |
| | adhesion molecule 8 | | | | |
| | CDNA clone IMAGE: 5271374, partial | 6.775 | BC041865 | 6 | 6 |
| | cds | | | | |
| ANKRD20A | Similar to FLJ00310 protein | 6.628 | BC022380 | 7 | 7 |
| | (LOC388669), mRNA | | | | |
| AZU1 | azurocidin 1 (cationic antimicrobial | 6.272 | NM_001700 | 8 | 8 |
| | protein 37) | | | | |
| ELA2 | elastase 2, neutrophil | 5.796 | NM_001972 | 9 | 9 |
| | CDNA FLJ33010 fis, clone | 5.19 | AI379823 | 10 | 10 |
| | THYMU1000336 | | | | |
| | Homo sapiens full length insert cDNA | 5.063 | AF086134 | 11 | 11 |
| | clone ZA88B06. | | | | |
| RAB45 | hypothetical protein FLJ31614 | 3.964 | NM_152573 | 12 | 12 |
| ECHDC3 | enoyl Coenzyme A hydratase domain | 3.84 | NM_024693 | 13 | 13 |
| | containing 3 | | | | |
| | Transcribed sequence with weak | 3.824 | AI797678 | 14 | 14 |
| | similarity to protein sp: O14595 | | | | |
| | (H. sapiens) NIF2_HUMAN Nuclear | | | | |
| | LIM interactor-interacting factor 2 | | | | |
| RAB45 | hypothetical protein FLJ31614 | 3.654 | NM_152573 | 15 | |
| IL1R2 | Human soluble type II interleukin-1 | 3.624 | U64094 | 16 | 15 |
| | receptor mRNA, complete cds. | | | | |
| TRIM4 | tripartite motif-containing 4 | 3.573 | AF220023 | 17 | 16 |
| | Transcribed sequences | 3.52 | BE972639 | 18 | 17 |
| | Transcribed sequences | 3.509 | AW337833 | 19 | 18 |
| CTSG | cathepsin G | 3.437 | NM_001911 | 20 | 19 |
| | Clone IMAGE: 5519764, mRNA | 3.434 | BC035915 | 21 | 20 |
| | | 3.195 | NM_018572 | 22 | |
| | CDNA clone IMAGE: 6165535, partial | 3.114 | AW086077 | 23 | 21 |
| | cds | | | | |
| SLC4A4 | solute carrier family 4, sodium | 3.081 | AF069510 | 24 | 22 |
| | bicarbonate cotransporter, member 4 | | | | |
| SLC16A3 | solute carrier family 16 | 2.984 | NM_004207 | 25 | 23 |
| | (monocarboxylic acid transporters), | | | | |
| | member 3 | | | | |
| | unnamed protein product; Homo | 2.978 | AK094974 | 26 | 24 |
| | sapiens cDNA FLJ37655 fis, clone | | | | |
| | BRHIP2010444. | | | | |
| | CDNA FLJ41501 fis, clone | 2.948 | BF667120 | 27 | 25 |
| | BRTHA2006975 | | | | |
| | Transcribed sequences | 2.887 | AW665840 | 28 | 26 |
| | Transcribed sequences | 2.859 | AW665538 | 29 | 27 |
| | CDNA FLJ41762 fis, clone | 2.843 | AK096159 | 30 | 28 |
| | IMR322004768 | | | | |
| | Transcribed sequences | 2.725 | AW340015 | 31 | 29 |
| | | 2.686 | NM_014136 | 32 | |
| FAD104 | FAD104 | 2.663 | N32832 | 33 | 30 |
| | out-of-frame rearrangement, stop | 2.649 | AJ275453 | 34 | 31 |
| | codon in FR2, 1 bp deletion; Homo | | | | |
| | sapiens partial IGVH4 gene for | | | | |
| | immunoglobulin heavy chain V region, | | | | |
| | case 2, cell D 56. | | | | |
| | Transcribed sequence with weak | 2.603 | AI733564 | 35 | 32 |
| | similarity to protein pir: A40138 | | | | |
| | (H. sapiens) A40138 glycogen | | | | |
| | phosphorylase | | | | |
| | | 2.578 | NM_018575 | 36 | |
| FLJ36166 | hypothetical protein FLJ36166 | 2.563 | AL049437 | 37 | 33 |
| ZNF33A | Homo sapiens brain my041 protein | 2.562 | AF063599 | 38 | 34 |
| | mRNA, complete cds. | | | | |
| | Homo sapiens B lymphocyte | 2.553 | AF304443 | 39 | 35 |
| | activation-related protein BC-2048 | | | | |
| | mRNA, complete cds. | | | | |
| | Transcribed sequences | 2.551 | AI003508 | 40 | 36 |
| | Clone IMAGE: 5299049, mRNA, | 2.542 | BC036241 | 41 | 37 |
| | partial cds | | | | |
| HP | haptoglobin-related protein | 2.534 | NM_020995 | 42 | 38 |
| DAZ3 | deleted in azoospermia 4 | 2.533 | AF271088 | 43 | 39 |
| | CDNA clone IMAGE: 5541269, partial | 2.533 | BC039490 | 44 | 40 |
| | cds | | | | |
| SLC4A4 | solute carrier family 4, sodium | 2.511 | AF157492 | 45 | 41 |
| | bicarbonate cotransporter, member 4 | | | | |
| MIDN | midnolin | 2.473 | AL512725 | 46 | |
| HP | haptoglobin | 2.47 | NM_005143 | 47 | |
| AP2B1 | adaptor-related protein complex 2, | 2.465 | NM_001282 | 48 | |
| | beta 1 subunit | | | | |
| GOLGA1 | golgi autoantigen, golgin subfamily a, 1 | 2.463 | AW675473 | 49 | |
| LOC51152 | melanoma antigen | 2.421 | NM_016181 | 50 | |
| MGC40107 | phospholipid scramblase 3 | 2.383 | BF975929 | 51 | |
| KIAA1181 | KIAA1181 protein | 2.382 | AK000752 | 52 | |
| ORM1 | orosomucoid 1 | 2.358 | NM_000607 | 53 | |
| CA4 | carbonic anhydrase IV | 2.351 | NM_000717 | 54 | |
| OPHN1 | oligophrenin 1 | 2.342 | NM_002547 | 55 | |
| FLJ10618 | hypothetical protein FLJ10618 | 2.337 | AL136803 | 56 | |
| POLK | polymerase (DNA directed) kappa | 2.321 | BC014955 | 57 | |
| OVOS2 | hypothetical protein DKFZp434C0631 | 2.28 | AW594320 | 58 | |
| | xm39b03.x1 NCI_CGAP_GC6 Homo | 2.266 | AW197431 | 59 | |
| | sapiens cDNA clone IMAGE: 2686541 | | | | |
| | 3′ similar to contains element KER | | | | |
| | repetitive element;, mRNA sequence. | | | | |
| | | 2.262 | NM_018496 | 60 | |
| FLJ45803 | LOH11CR1F gene, loss of | 2.26 | BE176566 | 61 | |
| | heterozygosity, 11, chromosomal | | | | |
| | region 1 gene F product | | | | |
| FLJ12078 | | 2.259 | NM_024977 | 62 | |
| DAZ3 | deleted in azoospermia 2 | 2.251 | NM_020364 | 63 | |
| | Human full-length cDNA 5-PRIME | 2.234 | BG285837 | 64 | |
| | end of clone CS0DK007YB08 of | | | | |
| | HeLa cells of Homo sapiens (human) | | | | |
| TIMM8A | synonyms: DDP, DFN1, MTS, DDP1, | 2.222 | BC005236 | 65 | |
| | MGC12262; Homo sapiens | | | | |
| | translocase of inner mitochondrial | | | | |
| | membrane 8 homolog A (yeast), | | | | |
| | mRNA (cDNA clone MGC: 12262 | | | | |
| | IMAGE: 3682832), complete cds. | | | | |
| DAZ3 | deleted in azoospermia 4 | 2.211 | AF248483 | 66 | |
| SYCP1 | synaptonemal complex protein 1 | 2.177 | X95654 | 67 | |
| | Transcribed sequences | 2.174 | H28915 | 68 | |
| ASTN2 | synonym: KIAA0634; Homo sapiens | 2.159 | BC010680 | 69 | |
| | astrotactin 2, mRNA (cDNA clone | | | | |
| | IMAGE: 3872170), complete cds. | | | | |
| ALDH1B1 | aldehyde dehydrogenase 1 family, | 2.137 | BC001619 | 70 | |
| | member B1 | | | | |
| IL18RAP | interleukin 18 receptor accessory | 2.128 | NM_003853 | 71 | |
| | protein | | | | |
| MPO | myeloperoxidase | 2.126 | NM_000250 | 72 | |
| LOC125476 | unnamed protein product; Homo | 2.118 | AK095502 | 73 | |
| | sapiens cDNA FLJ38183 fis, clone | | | | |
| | FCBBF1000155. | | | | |
| | Transcribed sequences | 2.113 | BF954306 | 74 | |
| ORM2 | orosomucoid 1 | 2.089 | NM_000607 | 75 | |
| | predicted protein of HQ2221; Homo | 2.052 | AF116695 | 76 | |
| | sapiens PRO2221 mRNA, complete | | | | |
| | cds. | | | | |
| RBBP6 | Homo sapiens brain my038 protein | 2.037 | AF063596 | 77 | |
| | mRNA, complete cds. | | | | |
| ANKRD22 | wo31e07.x1 NCI_CGAP_Gas4 Homo | 2.023 | AI925518 | 78 | |
| | sapiens cDNA clone IMAGE: 2456964 | | | | |
| | 3′ similar to contains Alu repetitive | | | | |
| | element;, mRNA sequence. | | | | |
| JAK1 | Janus kinase 1 (a protein tyrosine | 0.5 | NM_002227 | 1355 | |
| | kinase) | | | | |
| LOC115294 | RC4-HT0276-100300-015-e11 | 0.499 | BE150929 | 1356 | |
| | HT0276 Homo sapiens cDNA, mRNA | | | | |
| | sequence. | | | | |
| ZNF24 | zinc finger protein 24 (KOX 17) | 0.498 | NM_006965 | 1357 | |
| TIA1 | TIA1 cytotoxic granule-associated | 0.497 | BC015944 | 1358 | |
| | RNA binding protein | | | | |
| NDUFA5 | NADH dehydrogenase (ubiquinone) 1 | 0.497 | NM_005000 | 1359 | |
| | alpha subcomplex, 5, 13 kDa | | | | |
| GOLGIN-67 | MRNA; cDNA DKFZp686O038 (from | 0.496 | AF164622 | 1360 | |
| | clone DKFZp686O038) | | | | |
| RHOQ | ras homolog gene family, member Q | 0.496 | BE551407 | 1361 | |
| CCM1 | Homo sapiens mRNA; cDNA | 0.496 | AL049325 | 1362 | |
| | DKFZp564D036 (from clone | | | | |
| | DKFZp564D036). | | | | |
| KLF4 | Kruppel-like factor 4 (gut) | 0.495 | BF514079 | 1363 | |
| FLJ31306 | hypothetical protein FLJ31306 | 0.495 | AW136120 | 1363 | |
| TCF8 | transcription factor 8 (represses | 0.495 | U12170 | 1365 | |
| | interleukin 2 expression) | | | | |
| UQCRC2 | ubiquinol-cytochrome c reductase | 0.493 | AI961429 | 1366 | |
| | core protein II | | | | |
| KLHL5 | Homo sapiens mRNA; cDNA | 0.492 | AL049385 | 1367 | |
| | DKFZp586M1418 (from clone | | | | |
| | DKFZp586M1418). | | | | |
| MGC46719 | hypothetical protein MGC46719 | 0.492 | AA923372 | 1368 | |
| ZNF160 | zinc finger protein 160 | 0.492 | AA701249 | 1369 | |
| CASP1 | CARD only protein | 0.49 | NM_052889 | 1370 | |
| RTN1 | reticulon 1 | 0.49 | NM_021136 | 1371 | |
| HSPCA | heat shock 90 kDa protein 1, alpha | 0.489 | AF028832 | 1372 | |
| | 7d75g01.x1 NCI_CGAP_Lu24 Homo | 0.489 | BE674143 | 1373 | |
| | sapiens cDNA clone IMAGE: 3278832 | | | | |
| | 3′ similar to contains element MER1 | | | | |
| | repetitive element;, mRNA sequence. | | | | |
| LOC374443 | 601556050F1 NIH_MGC_58 Homo | 0.489 | BE738988 | 1374 | |
| | sapiens cDNA clone IMAGE: 3825757 | | | | |
| | 5′, mRNA sequence. | | | | |
| P15RS | hypothetical protein FLJ10656 | 0.488 | AA778694 | 1375 | |
| | Transcribed sequences | 0.488 | AA223929 | 1376 | |
| | CDNA FLJ39805 fis, clone | 0.488 | CA444630 | 1377 | |
| | SPLEN2007951 | | | | |
| UTRN | utrophin (homologous to dystrophin) | 0.488 | NM_007124 | 1378 | |
| DLAT | dihydrolipoamide S-acetyltransferase | 0.487 | BF978872 | 1379 | |
| | (E2 component of pyruvate | | | | |
| | dehydrogenase complex) | | | | |
| HSPCB | heat shock 90 kDa protein 1, beta | 0.486 | BG612458 | 1380 | |
| PPM1B | protein phosphatase 1B (formerly | 0.484 | AJ271832 | 1381 | |
| | 2C), magnesium-dependent, beta | | | | |
| | isoform | | | | |
| ANKHD1 | multiple ankyrin repeats, single KH- | 0.484 | AK024189 | 1382 | |
| | domain (MASK) homolog | | | | |
| FLI1 | | 0.484 | M93255 | 1383 | |
| ARHGEF7 | Rho guanine nucleotide exchange | 0.483 | AI040887 | 1384 | |
| | factor (GEF) 7 | | | | |
| | Transcribed sequences | 0.483 | AI168187 | 1385 | |
| GNA13 | guanine nucleotide binding protein (G | 0.482 | NM_006572 | 1386 | |
| | protein), alpha 13 | | | | |
| C14orf43 | | 0.482 | NM_018678 | 1387 | |
| NEK4 | NIMA (never in mitosis gene a)- | 0.481 | NM_003157 | 1388 | |
| | related kinase 4 | | | | |
| ZF | HCF-binding transcription factor | 0.481 | NM_021212 | 1389 | |
| | Zhangfei | | | | |
| FLJ12716 | FLJ12716 protein | 0.479 | AK023390 | 1390 | |
| APG5L | APG5 autophagy 5-like (S. cerevisiae) | 0.479 | AF293841 | 1391 | |
| HSPCA | heat shock 90 kDa protein 1, alpha | 0.479 | AI962933 | 1392 | |
| | UI-E-EO1-aid-b-20-0-UI.s1 UI-E-EO1 | 0.477 | BM677619 | 1393 | |
| | Homo sapiens cDNA clone UI-E-EO1- | | | | |
| | aid-b-20-0-UI 3′, mRNA sequence. | | | | |
| DLEU2 | deleted in lymphocytic leukemia, 2 | 0.477 | BC006995 | 1394 | |
| T3JAM | TRAF3-interacting Jun N-terminal | 0.477 | AW963138 | 1395 | |
| | kinase (JNK)-activating modulator | | | | |
| PRNP | prion protein (p27-30) (Creutzfeld- | 0.476 | NM_000311 | 1396 | |
| | Jakob disease, Gerstmann-Strausler- | | | | |
| | Scheinker syndrome, fatal familial | | | | |
| | insomnia) | | | | |
| CCR2 | chemokine (C-C motif) receptor 2 | 0.475 | NM_000648 | 1397 | |
| TUBGCP3 | tubulin, gamma complex associated | 0.475 | NM_006322 | 1398 | |
| | protein 3 | | | | |
| OLIG1 | oligodendrocyte transcription factor 1 | 0.475 | AL355743 | 1399 | |
| | Transcribed sequences | 0.474 | AI032730 | 1400 | |
| | AV736963 CB Homo sapiens cDNA | 0.473 | AV736963 | 1401 | |
| | clone CBFBOH07 5′, mRNA | | | | |
| | sequence. | | | | |
| | MRNA; cDNA DKFZp564H023 (from | 0.47 | AL049273 | 1402 | |
| | clone DKFZp564H023) | | | | |
| | MRNA; cDNA DKFZp547L144 (from | 0.47 | AL390176 | 1403 | |
| | clone DKFZp547L144) | | | | |
| ZNF146 | zinc finger protein 146 | 0.469 | NM_007145 | 1404 | |
| FER1L3 | fer-1-like 3, myoferlin (C. elegans) | 0.468 | AF207990 | 1405 | |
| | UI-H-BI1-acb-b-12-0-UI.s1 | 0.467 | AW135176 | 1406 | |
| | NCI_CGAP_Sub3 Homo sapiens | | | | |
| | cDNA clone IMAGE: 2713679 3′, | | | | |
| | mRNA sequence. | | | | |
| HLA-DRB3 | major histocompatibility complex, | 0.467 | AA807056 | 1407 | |
| | class II, DR beta 3 | | | | |
| EPB41L3 | erythrocyte membrane protein band | 0.467 | NM_012307 | 1408 | |
| | 4.1-like 3 | | | | |
| PLEKHF2 | pleckstrin homology domain | 0.466 | BF439250 | 1409 | |
| | containing, family F (with FYVE | | | | |
| | domain) member 2 | | | | |
| | Transcribed sequence with moderate | 0.462 | AW452971 | 1410 | |
| | similarity to protein pir: I60307 (E. coli) | | | | |
| | I60307 beta-galactosidase, alpha | | | | |
| | peptide - Escherichia coli | | | | |
| SMC4L1 | SMC4 structural maintenance of | 0.461 | NM_005496 | 1411 | |
| | chromosomes 4-like 1 (yeast) | | | | |
| DDX42 | DEAD (Asp-Glu-Ala-Asp) box | 0.46 | AF147429 | 1412 | |
| | polypeptide 42 | | | | |
| SMARCA2 | SWI/SNF related, matrix associated, | 0.46 | NM_003070 | 1413 | |
| | actin dependent regulator of | | | | |
| | chromatin, subfamily a, member 2 | | | | |
| | CDNA clone IMAGE: 4402168, partial | 0.459 | BC038203 | 1414 | |
| | cds | | | | |
| TAP2 | transporter 2, ATP-binding cassette, | 0.459 | NM_000544 | 1415 | |
| | sub-family B (MDR/TAP) | | | | |
| HCRP1 | hepatocellular carcinoma-related | 0.458 | AK025343 | 1416 | |
| | HCRP1 | | | | |
| | Transcribed sequence with weak | 0.458 | AA740632 | 1417 | |
| | similarity to protein ref: NP_060312.1 | | | | |
| | (H. sapiens) hypothetical protein | | | | |
| | FLJ20489 [Homo sapiens] | | | | |
| TTC3 | tetratricopeptide repeat domain 3 | 0.458 | AU131711 | 1418 | |
| SMG1 | | 0.458 | AC003007 | 1419 | |
| SGPP1 | sphingosine-1-phosphate | 0.457 | NM_030791 | 1420 | |
| | phosphatase 1 | | | | |
| DDX10 | DEAD (Asp-Glu-Ala-Asp) box | 0.456 | NM_004398 | 1421 | |
| | polypeptide 10 | | | | |
| NEDD5 | neural precursor cell expressed, | 0.456 | AI191427 | 1422 | |
| | developmentally down-regulated 5 | | | | |
| MEF2C | MADS box transcription enhancer | 0.454 | NM_002397 | 1423 | |
| | factor 2, polypeptide C (myocyte | | | | |
| | enhancer factor 2C) | | | | |
| LNPEP | leucyl/cystinyl aminopeptidase | 0.453 | NM_005575 | 1424 | |
| TGOLN2 | trans-golgi network protein 2 | 0.449 | NM_006464 | 1425 | |
| TFCP2 | transcription factor CP2 | 0.446 | NM_005653 | 1426 | |
| | Homo sapiens mRNA; cDNA | 0.444 | AL353949 | 1427 | |
| | DKFZp761P1114 (from clone | | | | |
| | DKFZp761P1114). | | | | |
| CSPG6 | chondroitin sulfate proteoglycan 6 | 0.44 | AF020043 | 1428 | |
| | (bamacan) | | | | |
| PIK3AP1 | phosphoinositide-3-kinase adaptor | 0.44 | BC029917 | 1429 | |
| | protein 1 | | | | |
| JIK | STE20-like kinase | 0.44 | BE178502 | 1430 | |
| FLJ23091 | putative NFkB activating protein 373 | 0.439 | AL534095 | 1431 | |
| MSI2 | musashi homolog 2 (Drosophila) | 0.439 | BF435123 | 1432 | |
| DSP | desmoplakin | 0.433 | NM_004415 | 1433 | |
| SAMHD1 | SAM domain and HD domain 1 | 0.431 | AF147427 | 1434 | |
| BCOR | BCL6 co-repressor | 0.429 | AF317391 | 1435 | |
| ILF3 | interleukin enhancer binding factor 3, | 0.428 | AF141870 | 1436 | |
| | 90 kDa | | | | |
| H41 | hypothetical protein H41 | 0.427 | BG257762 | 1437 | |
| LOC284058 | hypothetical protein LOC284058 | 0.426 | BC006271 | 1438 | |
| UPF3B | UPF3 regulator of nonsense | 0.426 | NM_023010 | 1439 | |
| | transcripts homolog B (yeast) | | | | |
| PXN | Paxillin (PXN), mRNA | 0.422 | D86862 | 1440 | |
| CTNNA1 | catenin (cadherin-associated protein), | 0.42 | BG330076 | 1441 | |
| | alpha 1, 102 kDa | | | | |
| C6orf67 | chromosome 6 open reading frame | 0.42 | AK022883 | 1442 | |
| | 67 | | | | |
| ATRX | alpha thalassemia/mental retardation | 0.418 | AA826176 | 1443 | |
| | syndrome X-linked (RAD54 homolog, | | | | |
| | S. cerevisiae) | | | | |
| HYPB | huntingtin interacting protein B | 0.418 | AI868267 | 1444 | |
| TTF2 | transcription termination factor, RNA | 0.416 | AF080255 | 1445 | |
| | polymerase II | | | | |
| | Clone IMAGE: 5267013, mRNA | 0.415 | BI052176 | 1446 | |
| KBTBD2 | kelch repeat and BTB (POZ) domain | 0.414 | AF151831 | 1447 | |
| | containing 2 | | | | |
| PTPRO | protein tyrosine phosphatase, | 0.413 | NM_002848 | 1448 | |
| | receptor type, O | | | | |
| C14orf85 | CDNA: FLJ21234 fis, clone | 0.412 | AK024887 | 1449 | |
| | COL00841 | | | | |
| | CDNA FLJ35626 fis, clone | 0.411 | AA700631 | 1450 | |
| | SPLEN2011086 | | | | |
| ZNF267 | zinc finger protein 267 | 0.411 | AU150728 | 1451 | |
| HTATSF1 | HIV TAT specific factor 1 | 0.41 | NM_014500 | 1452 | |
| SP100 | nuclear antigen Sp100 | 0.409 | AF056322 | 1453 | |
| FNBP1 | formin binding protein 1 | 0.398 | AA937109 | 1454 | 42 |
| DDX3X | DEAD (Asp-Glu-Ala-Asp) box | 0.398 | R60068 | 1455 | 43 |
| | polypeptide 3, X-linked | | | | |
| UBE4B | ubiquitination factor E4B (UFD2 | 0.396 | AW241715 | 1456 | 44 |
| | homolog, yeast) | | | | |
| C22orf1 | chromosome 22 open reading frame 1 | 0.392 | BC034024 | 1457 | 45 |
| | CDNA FLJ10256 fis, clone | 0.392 | AK001118 | 1458 | 46 |
| | HEMBB1000870 | | | | |
| IGHM | Partial mRNA for IgM immunoglobulin | 0.39 | BG340670 | 1459 | 47 |
| | heavy chain variable region (IGHV | | | | |
| | gene), clone LIBPM376 | | | | |
| | Full length insert cDNA clone | 0.385 | AF085827 | 1460 | 48 |
| | YF47B02 | | | | |
| MO25 | MO25 protein | 0.363 | AF151824 | 1461 | 49 |
| SIAT1 | sialyltransferase 1 (beta-galactoside | 0.361 | AV695711 | 1462 | 50 |
| | alpha-2,6-sialyltransferase) | | | | |
| TncRNA | MRNA; cDNA DKFZp686L01105 | 0.338 | AI042152 | 1463 | 51 |
| | (from clone DKFZp686L01105) | | | | |
| MTMR1 | myotubularin related protein 1 | 0.32 | AK001816 | 1464 | 52 |
| FER1L3 | fer-1-like 3, myoferlin (C. elegans) | 0.32 | NM_013451 | 1465 | 53 |
| TncRNA | MRNA; cDNA DKFZp686L01105 | 0.302 | AV659198 | 1466 | 54 |
| | (from clone DKFZp686L01105) | | | | |
| HIPK3 | homeodomain interacting protein | 0.248 | AF305239 | 1467 | 55 |
| | kinase 3 | | | | |

TABLE 3
Control vs. Duchenne muscular dystrophy (DMD) - ≧|1.5| Fold Difference

| | | | | Genbank | | |
|---|---|---|---|---|---|---|
| Common | | Fold | | Accession | Nucleic | SEQ ID |
| Name | Description | Change | AFFY ID | Number | Acid No. | NO. |
| DEFA4 | defensin, alpha 4, | 26.06 | 207269_at | NM_001925 | 1 | 1 |
| | corticostatin | | | | | |
| SH3GL3 | predicted; contains SH3 | 11.96 | 211565_at | AF036272 | 2 | 2 |
| | domain; member of the EEN | | | | | |
| | gene family; Homo sapiens | | | | | |
| | EEN-B2-L4 mRNA, complete | | | | | |
| | cds. | | | | | |
| ANKRD20A | hypothetical protein | 11.14 | 224012_at | AL136793 | 3 | 3 |
| | DKFZp434A171 | | | | | |
| | Homo sapiens cDNA: | 8.979 | 232538_at | AK027226 | 4 | 4 |
| | FLJ23573 fis, clone | | | | | |
| | LNG12520. | | | | | |
| CEACAM8 | carcinoembryonic antigen- | 7.116 | 206676_at | M33326 | 5 | 5 |
| | related cell adhesion | | | | | |
| | molecule 8 | | | | | |
| | CDNA clone | 6.775 | 1560144_at | BC041865 | 6 | 6 |
| | IMAGE: 5271374, partial cds | | | | | |
| ANKRD20A | Similar to FLJ00310 protein | 6.628 | 1570255_s_at | BC022380 | 7 | 7 |
| | (LOC388669), mRNA | | | | | |
| AZU1 | azurocidin 1 (cationic | 6.272 | 214575_s_at | NM_001700 | 8 | 8 |
| | antimicrobial protein 37) | | | | | |
| ELA2 | elastase 2, neutrophil | 5.796 | 206871_at | NM_001972 | 9 | 9 |
| | CDNA FLJ33010 fis, clone | 5.19 | 231598_x_at | AI379823 | 10 | 10 |
| | THYMU1000336 | | | | | |
| | Homo sapiens full length | 5.063 | 1561754_at | AF086134 | 11 | 11 |
| | insert cDNA clone ZA88B06. | | | | | |
| RAB45 | hypothetical protein | 3.964 | 1553186_at | NM_152573 | 12 | 12 |
| | FLJ31614 | | | | | |
| ECHDC3 | enoyl Coenzyme A hydratase | 3.84 | 219298_at | NM_024693 | 13 | 13 |
| | domain containing 3 | | | | | |
| | Transcribed sequence with | 3.824 | 227921_at | AI797678 | 14 | 14 |
| | weak similarity to protein | | | | | |
| | sp: O14595 (H. sapiens) | | | | | |
| | NIF2_HUMAN Nuclear LIM | | | | | |
| | interactor-interacting factor 2 | | | | | |
| RAB45 | hypothetical protein | 3.654 | 1553185_at | NM_152573 | 15 | |
| | FLJ31614 | | | | | |
| IL1R2 | Human soluble type II | 3.624 | 211372_s_at | U64094 | 16 | 15 |
| | interleukin-1 receptor mRNA, | | | | | |
| | complete cds. | | | | | |
| TRIM4 | tripartite motif-containing 4 | 3.573 | 224159_x_at | AF220023 | 17 | 16 |
| | Transcribed sequences | 3.52 | 243332_at | BE972639 | 18 | 17 |
| | Transcribed sequences | 3.509 | 231688_at | AW337833 | 19 | 18 |
| CTSG | cathepsin G | 3.437 | 205653_at | NM_001911 | 20 | 19 |
| | Clone IMAGE: 5519764, | 3.434 | 1569664_at | BC035915 | 21 | 20 |
| | mRNA | | | | | |
| | | 3.195 | 1553498_at | NM_018572 | 22 | |
| | CDNA clone | 3.114 | 232800_at | AW086077 | 23 | 21 |
| | IMAGE: 6165535, partial cds | | | | | |
| SLC4A4 | solute carrier family 4, sodium | 3.081 | 210739_x_at | AF069510 | 24 | 22 |
| | bicarbonate cotransporter, | | | | | |
| | member 4 | | | | | |
| SLC16A3 | solute carrier family 16 | 2.984 | 202856_s_at | NM_004207 | 25 | 23 |
| | (monocarboxylic acid | | | | | |
| | transporters), member 3 | | | | | |
| | unnamed protein product; | 2.978 | 1563941_at | AK094974 | 26 | 24 |
| | Homo sapiens cDNA | | | | | |
| | FLJ37655 fis, clone | | | | | |
| | BRHIP2010444. | | | | | |
| | CDNA FLJ41501 fis, clone | 2.948 | 225062_at | BF667120 | 27 | 25 |
| | BRTHA2006975 | | | | | |
| | Transcribed sequences | 2.887 | 244539_at | AW665840 | 28 | 26 |
| | Transcribed sequences | 2.859 | 244532_x_at | AW665538 | 29 | 27 |
| | CDNA FLJ41762 fis, clone | 2.843 | 1560431_at | AK096159 | 30 | 28 |
| | IMR322004768 | | | | | |
| | Transcribed sequences | 2.725 | 237771_s_at | AW340015 | 31 | 29 |
| | | 2.686 | 220859_at | NM_014136 | 32 | |
| FAD104 | FAD104 | 2.663 | 242029_at | N32832 | 33 | 30 |
| | out-of-frame rearrangement, | 2.649 | 234415_x_at | AJ275453 | 34 | 31 |
| | stop codon in FR2, 1 bp | | | | | |
| | deletion; Homo sapiens | | | | | |
| | partial IGVH4 gene for | | | | | |
| | immunoglobulin heavy chain | | | | | |
| | V region, case 2, cell D 56. | | | | | |
| | Transcribed sequence with | 2.603 | 236439_at | AI733564 | 35 | 32 |
| | weak similarity to protein | | | | | |
| | pir: A40138 (H. sapiens) | | | | | |
| | A40138 glycogen | | | | | |
| | phosphorylase | | | | | |
| | | 2.578 | 220874_at | NM_018575 | 36 | |
| FLJ36166 | hypothetical protein | 2.563 | 215143_at | AL049437 | 37 | 33 |
| | FLJ36166 | | | | | |
| ZNF33A | Homo sapiens brain my041 | 2.562 | 224276_at | AF063599 | 38 | 34 |
| | protein mRNA, complete cds. | | | | | |
| | Homo sapiens B lymphocyte | 2.553 | 1555373_at | AF304443 | 39 | 35 |
| | activation-related protein BC- | | | | | |
| | 2048 mRNA, complete cds. | | | | | |
| | Transcribed sequences | 2.551 | 244114_x_at | AI003508 | 40 | 36 |
| | Clone IMAGE: 5299049, | 2.542 | 1561271_at | BC036241 | 41 | 37 |
| | mRNA, partial cds | | | | | |
| HP | haptoglobin-related protein | 2.534 | 208470_s_at | NM_020995 | 42 | 38 |
| DAZ3 | deleted in azoospermia 4 | 2.533 | 216922_x_at | AF271088 | 43 | 39 |
| | CDNA clone | 2.533 | 1561489_at | BC039490 | 44 | 40 |
| | IMAGE: 5541269, partial cds | | | | | |
| SLC4A4 | solute carrier family 4, sodium | 2.511 | 211494_s_at | AF157492 | 45 | 41 |
| | bicarbonate cotransporter, | | | | | |
| | member 4 | | | | | |
| MIDN | midnolin | 2.473 | 225954_s_at | AL512725 | 46 | |
| HP | haptoglobin | 2.47 | 206697_s_at | NM_005143 | 47 | |
| AP2B1 | adaptor-related protein | 2.465 | 200612_s_at | NM_001282 | 48 | |
| | complex 2, beta 1 subunit | | | | | |
| GOLGA1 | golgi autoantigen, golgin | 2.463 | 214997_at | AW675473 | 49 | |
| | subfamily a, 1 | | | | | |
| LOC51152 | melanoma antigen | 2.421 | 220771_at | NM_016181 | 50 | |
| MGC40107 | phospholipid scramblase 3 | 2.383 | 227063_at | BF975929 | 51 | |
| KIAA1181 | KIAA1181 protein | 2.382 | 224576_at | AK000752 | 52 | |
| ORM1 | orosomucoid 1 | 2.358 | 205041_s_at | NM_000607 | 53 | |
| CA4 | carbonic anhydrase IV | 2.351 | 206209_s_at | NM_000717 | 54 | |
| OPHN1 | oligophrenin 1 | 2.342 | 206323_x_at | NM_002547 | 55 | |
| FLJ10618 | hypothetical protein | 2.337 | 223698_at | AL136803 | 56 | |
| | FLJ10618 | | | | | |
| POLK | polymerase (DNA directed) | 2.321 | 1555317_at | BC014955 | 57 | |
| | kappa | | | | | |
| OVOS2 | hypothetical protein | 2.28 | 228245_s_at | AW594320 | 58 | |
| | DKFZp434C0631 | | | | | |
| | xm39b03.x1 | 2.266 | 236571_at | AW197431 | 59 | |
| | NCI_CGAP_GC6 Homo | | | | | |
| | sapiens cDNA clone | | | | | |
| | IMAGE: 2686541 3′ similar to | | | | | |
| | contains element KER | | | | | |
| | repetitive element;, mRNA | | | | | |
| | sequence. | | | | | |
| | | 2.262 | 220869_at | NM_018496 | 60 | |
| FLJ45803 | LOH11CR1F gene, loss of | 2.26 | 238701_x_at | BE176566 | 61 | |
| | heterozygosity, 11, | | | | | |
| | chromosomal region 1 gene F | | | | | |
| | product | | | | | |
| FLJ12078 | | 2.259 | 220900_at | NM_024977 | 62 | |
| DAZ3 | deleted in azoospermia 2 | 2.251 | 208281_x_at | NM_020364 | 63 | |
| | Human full-length cDNA 5- | 2.234 | 242881_x_at | BG285837 | 64 | |
| | PRIME end of clone | | | | | |
| | CS0DK007YB08 of HeLa | | | | | |
| | cells of Homo sapiens | | | | | |
| | (human) | | | | | |
| TIMM8A | synonyms: DDP, DFN1, MTS, | 2.222 | 210800_at | BC005236 | 65 | |
| | DDP1, MGC12262; Homo | | | | | |
| | sapiens translocase of inner | | | | | |
| | mitochondrial membrane 8 | | | | | |
| | homolog A (yeast), mRNA | | | | | |
| | (cDNA clone MGC: 12262 | | | | | |
| | IMAGE: 3682832), complete | | | | | |
| | cds. | | | | | |
| DAZ3 | deleted in azoospermia 4 | 2.211 | 216351_x_at | AF248483 | 66 | |
| SYCP1 | synaptonemal complex | 2.177 | 216917_s_at | X95654 | 67 | |
| | protein 1 | | | | | |
| | Transcribed sequences | 2.174 | 244359_s_at | H28915 | 68 | |
| ASTN2 | synonym: KIAA0634; Homo | 2.159 | 1554816_at | BC010680 | 69 | |
| | sapiens astrotactin 2, mRNA | | | | | |
| | (cDNA clone | | | | | |
| | IMAGE: 3872170), complete | | | | | |
| | cds. | | | | | |
| ALDH1B1 | aldehyde dehydrogenase 1 | 2.137 | 209646_x_at | BC001619 | 70 | |
| | family, member B1 | | | | | |
| IL18RAP | interleukin 18 receptor | 2.128 | 207072_at | NM_003853 | 71 | |
| | accessory protein | | | | | |
| MPO | myeloperoxidase | 2.126 | 203949_at | NM_000250 | 72 | |
| LOC125476 | unnamed protein product; | 2.118 | 1559716_at | AK095502 | 73 | |
| | Homo sapiens cDNA | | | | | |
| | FLJ38183 fis, clone | | | | | |
| | FCBBF1000155. | | | | | |
| | Transcribed sequences | 2.113 | 239811_at | BF954306 | 74 | |
| ORM2 | orosomucoid 1 | 2.089 | 205040_at | NM_000607 | 75 | |
| | predicted protein of HQ2221; | 2.052 | 224254_x_at | AF116695 | 76 | |
| | Homo sapiens PRO2221 | | | | | |
| | mRNA, complete cds. | | | | | |
| RBBP6 | Homo sapiens brain my038 | 2.037 | 223802_s_at | AF063596 | 77 | |
| | protein mRNA, complete cds. | | | | | |
| ANKRD22 | wo31e07.x1 | 2.023 | 238439_at | AI925518 | 78 | |
| | NCI_CGAP_Gas4 Homo | | | | | |
| | sapiens cDNA clone | | | | | |
| | IMAGE: 2456964 3′ similar to | | | | | |
| | contains Alu repetitive | | | | | |
| | element;, mRNA sequence. | | | | | |
| NPAT | nuclear protein, ataxia- | 1.997 | 211585_at | U58852 | 79 | |
| | telangiectasia locus | | | | | |
| PDE4DIP | phosphodiesterase 4D | 1.996 | 209700_x_at | AB042555 | 80 | |
| | interacting protein | | | | | |
| | (myomegalin) | | | | | |
| | CDNA FLJ41173 fis, clone | 1.987 | 1558404_at | BC015390 | 81 | |
| | BRACE2042394 | | | | | |
| SLC16A3 | solute carrier family 16 | 1.981 | 202855 s_at | AL513917 | 82 | |
| | (monocarboxylic acid | | | | | |
| | transporters), member 3 | | | | | |
| | Transcribed sequences | 1.98 | 239464_at | BF448114 | 83 | |
| BAGE | B melanoma antigen | 1.978 | 207712_at | NM_001187 | 84 | |
| DNCL2A | dynein, cytoplasmic, light | 1.975 | 217918_at | NM_014183 | 85 | |
| | polypeptide 2A | | | | | |
| | Transcribed sequence with | 1.974 | 222329_x_at | AW974816 | 86 | |
| | weak similarity to protein | | | | | |
| | sp: P39188 (H. sapiens) | | | | | |
| | ALU1_HUMAN Alu subfamily | | | | | |
| | J sequence contamination | | | | | |
| | warning entry | | | | | |
| | | 1.963 | 232953_at | AL137028 | 87 | |
| HIST1H4I | histone 1, H4j | 1.954 | 208580_x_at | NM_021968 | 88 | |
| | Homo sapiens cDNA clone | 1.944 | 1566862_at | BC040841 | 89 | |
| | IMAGE: 5310996, partial cds. | | | | | |
| KIAA0186 | | 1.925 | 206102_at | NM_021067 | 90 | |
| | Similar to | 1.921 | 226243_at | BF590958 | 91 | |
| | ENSANGP00000018456 | | | | | |
| | (LOC391356), mRNA | | | | | |
| | ot20h03.x1 NCI_CGAP_GC3 | 1.921 | 239959_x_at | AI147520 | 92 | |
| | Homo sapiens cDNA clone | | | | | |
| | IMAGE: 1615445 3′ similar to | | | | | |
| | contains Alu repetitive | | | | | |
| | element;, mRNA sequence. | | | | | |
| RPS21 | ribosomal protein S21 | 1.921 | 214097_at | AW024383 | 93 | |
| ANKRD18A | FLJ35740 protein | 1.919 | 1559406_at | AB095935 | 94 | |
| SCO2 | SCO cytochrome oxidase | 1.918 | 205241_at | NM_005138 | 95 | |
| | deficient homolog 2 (yeast) | | | | | |
| CXADR | coxsackie virus and | 1.91 | 203917_at | NM_001338 | 96 | |
| | adenovirus receptor | | | | | |
| | Similar to neurobeachin | 1.891 | 215801_at | AL133604 | 97 | |
| | (LOC388835), mRNA | | | | | |
| THEDC1 | hypothetical protein | 1.89 | 233126_s_at | AK001844 | 98 | |
| | FLJ11106 | | | | | |
| | human kpni repeat mrna | 1.884 | 217137_x_at | K00627 | 99 | |
| | (cdna clone pcd-kpni-8), 3′ | | | | | |
| | end. | | | | | |
| TFG | TRK-fused gene | 1.882 | 239385_at | AI150613 | 100 | |
| GZMB | granzyme B (granzyme 2, | 1.84 | 210164_at | J03189 | 101 | |
| | cytotoxic T-lymphocyte- | | | | | |
| | associated serine esterase 1) | | | | | |
| | Clone IMAGE: 5748207, | 1.836 | 1563316_at | BC042082 | 102 | |
| | mRNA | | | | | |
| GZMM | granzyme M (lymphocyte | 1.832 | 207460_at | NM_005317 | 103 | |
| | met-ase 1) | | | | | |
| | qb56c01.x1 | 1.824 | 241718_x_at | AI148165 | 104 | |
| | NCI_CGAP_Brn23 Homo | | | | | |
| | sapiens cDNA clone | | | | | |
| | IMAGE: 1704096 3′ similar to | | | | | |
| | contains OFR.b3 OFR OFR | | | | | |
| | repetitive element;, mRNA | | | | | |
| | sequence. | | | | | |
| | Transcribed sequences | 1.823 | 239140_at | AI741900 | 105 | |
| | CDNA FLJ42250 fis, clone | 1.822 | 227235_at | AI758408 | 106 | |
| | TKIDN2007828 | | | | | |
| H963 | platelet activating receptor | 1.813 | 207651_at | NM_013308 | 107 | |
| | homolog | | | | | |
| ZNF43 | zinc finger protein 43 (HTF6) | 1.808 | 1568646_x_at | BC038199 | 108 | |
| | UI-H-BI1-aeg-f-07-0-UI.s1 | 1.808 | 243934_at | AW139261 | 109 | |
| | NCI_CGAP_Sub3 Homo | | | | | |
| | sapiens cDNA clone | | | | | |
| | IMAGE: 2719260 3′, mRNA | | | | | |
| | sequence. | | | | | |
| FLJ20406 | hypothetical protein | 1.8 | 219541_at | NM_017806 | 110 | |
| | FLJ20406 | | | | | |
| | Transcribed sequences | 1.798 | 241769_at | AW962458 | 111 | |
| | Transcribed sequence with | 1.797 | 217569_x_at | AA017093 | 112 | |
| | weak similarity to protein | | | | | |
| | ref: NP_060312.1 (H. sapiens) | | | | | |
| | hypothetical protein | | | | | |
| | FLJ20489 [Homo sapiens ] | | | | | |
| | Transcribed sequences | 1.791 | 243648_at | AA280627 | 113 | |
| LOC338758 | Hypothetical protein | 1.785 | 238893_at | AI377324 | 114 | |
| | LOC338758, mRNA (cDNA | | | | | |
| | clone IMAGE: 5299152), | | | | | |
| | partial cds | | | | | |
| C9orf122 | Homo sapiens hypothetical | 1.774 | 1557541_at | BC036230 | 115 | |
| | protein LOC158228, mRNA | | | | | |
| | (cDNA clone | | | | | |
| | IMAGE: 5288595), partial cds. | | | | | |
| MGC3196 | | 1.768 | 220934_s_at | NM_024084 | 116 | |
| | Similar to RIKEN cDNA | 1.767 | 243610_at | AI768674 | 117 | |
| | 1700028P14 (LOC138255), | | | | | |
| | mRNA | | | | | |
| MGC23280 | hypothetical protein | 1.762 | 226121_at | AI076793 | 118 | |
| | MGC23280 | | | | | |
| MGC57827 | MRNA similar to RIKEN | 1.761 | 225834_at | AL135396 | 119 | |
| | cDNA 2700049P18 gene | | | | | |
| | (cDNA clone MGC: 57827 | | | | | |
| | IMAGE: 6064384), complete | | | | | |
| | cds | | | | | |
| SIAT7D | sialyltransferase 7D ((alpha- | 1.76 | 223285_s_at | AW044319 | 120 | |
| | N-acetylneuraminyl-2,3-beta- | | | | | |
| | galactosyl-1,3)-N-acetyl | | | | | |
| | galactosaminide alpha-2,6- | | | | | |
| | sialyltransferase) | | | | | |
| MGC27165 | VH gene family III; VDJ | 1.759 | 216558_x_at | AF044595 | 121 | |
| | rearrangement; Homo | | | | | |
| | sapiens lymphocyte- | | | | | |
| | predominant Hodgkin's | | | | | |
| | disease case #7 | | | | | |
| | immunoglobulin heavy chain | | | | | |
| | gene, variable region, partial | | | | | |
| | cds. | | | | | |
| FLJ20086 | Transcribed sequences | 1.757 | 1568951_at | AL530743 | 122 | |
| | Homo sapiens cDNA | 1.757 | 215859_at | AK022309 | 123 | |
| | FLJ12247 fis, clone | | | | | |
| | MAMMA1001397. | | | | | |
| | Homo sapiens , Similar to | 1.756 | 1570623_at | BC038672 | 124 | |
| | neuronal thread protein, clone | | | | | |
| | IMAGE: 5265833, mRNA. | | | | | |
| | CDNA FLJ30858 fis, clone | 1.754 | 1566658_at | AK055420 | 125 | |
| | FEBRA2003308 | | | | | |
| CR1 | complement component | 1.744 | 239206_at | BE552138 | 126 | |
| | (3b/4b) receptor 1-like | | | | | |
| UGCG | UDP-glucose ceramide | 1.74 | 221765_at | AI378044 | 127 | |
| | glucosyltransferase | | | | | |
| ENSA | endosulfine alpha | 1.734 | 228851_s_at | AV726322 | 128 | |
| PRKD2 | qv38c04.x1 | 1.734 | 241669_x_at | AI251399 | 129 | |
| | NCI_CGAP_Ov31 Homo | | | | | |
| | sapiens cDNA clone | | | | | |
| | IMAGE: 1983846 3′, mRNA | | | | | |
| | sequence. | | | | | |
| PCF11 | pre-mRNA cleavage complex | 1.72 | 239926_at | AI675753 | 130 | |
| | II protein Pcf11 | | | | | |
| CD8B1 | CD8 antigen, beta | 1.719 | 207979_s_at | NM_004931 | 131 | |
| | polypeptide 1 (p37) | | | | | |
| FLJ20641 | hypothetical protein | 1.718 | 220060_s_at | NM_017915 | 132 | |
| | FLJ20641 | | | | | |
| ZNF287 | Homo sapiens mRNA; cDNA | 1.717 | 216710_x_at | AL359578 | 133 | |
| | DKFZp547N163 (from clone | | | | | |
| | DKFZp547N163). | | | | | |
| GPAM | glycerol 3-phosphate | 1.712 | 225420_at | AV699379 | 134 | |
| | acyltransferase, mitochondrial | | | | | |
| DKFZp313G1735 | hypothetical protein | 1.71 | 239147_at | AI243677 | 135 | |
| | DKFZp313G1735 | | | | | |
| | Transcribed sequence with | 1.707 | 237442_at | AV699911 | 136 | |
| | weak similarity to protein | | | | | |
| | sp: P23961 (H. sapiens) | | | | | |
| | ALUC_HUMAN !!!! ALU | | | | | |
| | CLASS C WARNING ENTRY | | | | | |
| | !!!! | | | | | |
| | UI-H-BI4-apm-c-02-0-UI.s1 | 1.704 | 236732_at | BF510982 | 137 | |
| | NCI_CGAP_Sub8 Homo | | | | | |
| | sapiens cDNA clone | | | | | |
| | IMAGE: 3087746 3′, mRNA | | | | | |
| | sequence. | | | | | |
| | Transcribed sequence with | 1.704 | 228919_at | AA601031 | 138 | |
| | strong similarity to protein | | | | | |
| | ref: NP_001778.1 (H. sapiens) | | | | | |
| | cell division cycle 2-like 1, | | | | | |
| | isoform 1; Cell division cycle | | | | | |
| | 2-like 1; PITSLRE protein | | | | | |
| | kinase alpha; p58/GTA | | | | | |
| | protein kinase; | | | | | |
| | galactosyltransferase | | | | | |
| | associated protein kinase; | | | | | |
| | CDC-related protein kinase | | | | | |
| | p58; PITSLRE B [Homo | | | | | |
| | sapiens] | | | | | |
| C14orf2 | chromosome 14 open reading | 1.703 | 202279_at | NM_004894 | 139 | |
| | frame 2 | | | | | |
| C1orf40 | chromosome 1 open reading | 1.703 | 226674_at | AL522395 | 140 | |
| | frame 40 | | | | | |
| | Homo sapiens cDNA | 1.697 | 234087_at | AK022343 | 141 | |
| | FLJ12281 fis, clone | | | | | |
| | MAMMA1001745. | | | | | |
| KIAA1530 | KIAA1530 protein | 1.696 | 233893_s_at | AB040963 | 142 | |
| C4A | complement component 4A | 1.693 | 214428_x_at | K02403 | 143 | |
| | Homo sapiens cDNA | 1.692 | 233779_x_at | AK022046 | 144 | |
| | FLJ11984 fis, clone | | | | | |
| | HEMBB1001348. | | | | | |
| MLC1SA | myosin light chain 1 slow a | 1.686 | 204173_at | NM_002475 | 145 | |
| LOC400581 | CDNA clone | 1.685 | 1559688_at | AK000842 | 146 | |
| | IMAGE: 30337642, partial cds | | | | | |
| | | 1.684 | 220452_x_at | NM_021031 | 147 | |
| FLJ10420 | hypothetical protein | 1.684 | 230123_at | AI608836 | 148 | |
| | FLJ10420 | | | | | |
| | Homo sapiens pregnancy- | 1.683 | 234425_at | AF233648 | 149 | |
| | induced hypertension | | | | | |
| | syndrome-related protein | | | | | |
| | (PIH3) mRNA, partial cds. | | | | | |
| MRPL36 | mitochondrial ribosomal | 1.682 | 224331_s_at | AB049654 | 150 | |
| | protein L36 | | | | | |
| MGC72104 | Transcribed sequences | 1.678 | 243689_s_at | AI681945 | 151 | |
| PIN1 | ubiquitin-like 5 | 1.672 | 202927_at | NM_006221 | 152 | |
| | Transcribed sequences | 1.668 | 237096_at | BF938956 | 153 | |
| DKFZP564O243 | DKFZP564O243 protein | 1.668 | 210006_at | BC002571 | 154 | |
| MGC26885 | hypothetical protein | 1.668 | 214965_at | AF070574 | 155 | |
| | MGC26885 | | | | | |
| | Transcribed sequences | 1.668 | 244612_at | AW117181 | 156 | |
| | MRNA; cDNA | 1.667 | 233853_at | AL117552 | 157 | |
| | DKFZp564C0170 (from clone | | | | | |
| | DKFZp564C0170) | | | | | |
| ZNF272 | Homo sapiens clone | 1.666 | 233175_at | AF113692 | 158 | |
| | FLB4848. | | | | | |
| | CDNA: FLJ21358 fis, clone | 1.664 | 233377_at | AK025011 | 159 | |
| | COL02842 | | | | | |
| | Transcribed sequence with | 1.663 | 227931_at | AI823917 | 160 | |
| | moderate similarity to protein | | | | | |
| | pir: I60307 (E. coli) I60307 | | | | | |
| | beta-galactosidase, alpha | | | | | |
| | peptide - Escherichia coli | | | | | |
| ATF7IP | Homo sapiens cDNA | 1.656 | 216198_at | AK021569 | 161 | |
| | FLJ11507 fis, clone | | | | | |
| | HEMBA1002160. | | | | | |
| | Homo sapiens , clone | 1.652 | 1562474_at | BC043601 | 162 | |
| | IMAGE: 5228040, mRNA. | | | | | |
| CHD1 | chromodomain helicase DNA | 1.652 | 204258_at | NM_001270 | 163 | |
| | binding protein 1 | | | | | |
| MGC10084 | hypothetical protein | 1.645 | 1557836_at | AK093087 | 164 | |
| | MGC10084 | | | | | |
| | CDNA: FLJ22733 fis, clone | 1.641 | 233784_at | AK026386 | 165 | |
| | HSI15907 | | | | | |
| SLC2A11 | solute carrier family 2 | 1.64 | 1558540_s_at | AK055523 | 166 | |
| | (facilitated glucose | | | | | |
| | transporter), member 11 | | | | | |
| KSP37 | Ksp37 protein | 1.64 | 223836_at | AB021123 | 167 | |
| MPO | myeloperoxidase | 1.638 | 203948_s_at | J02694 | 168 | |
| | od19h05.s1 | 1.636 | 244125_at | AA837131 | 169 | |
| | NCI_CGAP_GCB1 Homo | | | | | |
| | sapiens cDNA clone | | | | | |
| | IMAGE: 1368441, mRNA | | | | | |
| | sequence. | | | | | |
| | Transcribed sequence with | 1.635 | 1557437_a_at | AW273830 | 170 | |
| | moderate similarity to protein | | | | | |
| | ref: NP_060219.1 (H. sapiens) | | | | | |
| | hypothetical protein | | | | | |
| | FLJ20294 [Homo sapiens ] | | | | | |
| LOC150678 | helicase/primase complex | 1.633 | 226845_s_at | AL036350 | 171 | |
| | protein | | | | | |
| | CDNA FLJ38525 fis, clone | 1.633 | 237509_at | AI733348 | 172 | |
| | HCHON2000851 | | | | | |
| LOC205251 | hypothetical protein | 1.633 | 228614_at | AW182614 | 173 | |
| | LOC205251 | | | | | |
| ECGF1 | endothelial cell growth factor | 1.632 | 217497_at | AW613387 | 174 | |
| | 1 (platelet-derived) | | | | | |
| HIST1H1C | Homo sapiens histone 1, | 1.631 | 209398_at | BC002649 | 175 | |
| | H1c, mRNA (cDNA clone | | | | | |
| | IMAGE: 3608862). | | | | | |
| | CDNA FLJ31683 fis, clone | 1.63 | 228315_at | AI632728 | 176 | |
| | NT2RI2005353 | | | | | |
| MGC33382 | hypothetical protein | 1.629 | 229442_at | BF059556 | 177 | |
| | MGC33382 | | | | | |
| MPL | CDC20 cell division cycle 20 | 1.629 | 207550_at | NM_005373 | 178 | |
| | homolog (S. cerevisiae) | | | | | |
| | MRNA; cDNA | 1.629 | 233152_x_at | AL049979 | 179 | |
| | DKFZp564C142 (from clone | | | | | |
| | DKFZp564C142) | | | | | |
| MRPL11 | mitochondrial ribosomal | 1.626 | 219162_s_at | NM_016050 | 180 | |
| | protein L11 | | | | | |
| MRPL12 | mitochondrial ribosomal | 1.625 | 203931_s_at | NM_002949 | 181 | |
| | protein L12 | | | | | |
| | 601823004F1 NIH_MGC_77 | 1.621 | 1556021_at | BF105980 | 182 | |
| | Homo sapiens cDNA clone | | | | | |
| | IMAGE: 4042801 5′, mRNA | | | | | |
| | sequence. | | | | | |
| CKLF | chemokine-like factor | 1.618 | 221058_s_at | NM_016326 | 183 | |
| | Transcribed sequences | 1.617 | 243149_at | AI467945 | 184 | |
| SEPW1 | selenoprotein W, 1 | 1.613 | 201194_at | NM_003009 | 185 | |
| KIAA1411 | Human DNA sequence from | 1.605 | 233730_at | AL078591 | 186 | |
| | clone RP1-198I9 on | | | | | |
| | chromosome 6q12-13, | | | | | |
| | complete sequence. | | | | | |
| | CDNA FLJ41455 fis, clone | 1.604 | 244395_at | AA018404 | 187 | |
| | BRSTN2012284 | | | | | |
| | Homo sapiens cDNA | 1.601 | 217052_x_at | AK024108 | 188 | |
| | FLJ14046 fis, clone | | | | | |
| | HEMBA1006461. | | | | | |
| AD7C-NTP | contains expressed Alu- | 1.601 | 208014_x_at | NM_014486 | 189 | |
| | containing domains; | | | | | |
| | go_component: integral to | | | | | |
| | membrane [goid 0016021] | | | | | |
| | [evidence TAS] [pmid | | | | | |
| | 9399956]; go_component: | | | | | |
| | extracellular space [goid | | | | | |
| | 0005615] [evidence TAS] | | | | | |
| | [pmid 9399956]; go_process: | | | | | |
| | central nervous system | | | | | |
| | development [goid 0007417] | | | | | |
| | [evidence TAS] [pmid | | | | | |
| | 9399956]; go_process: | | | | | |
| | apoptosis [goid 0006915] | | | | | |
| | [evidence TAS] [pmid | | | | | |
| | 9399956]; Homo sapiens | | | | | |
| | neuronal thread protein | | | | | |
| | (AD7C-NTP), mRNA. | | | | | |
| | Uncharacterized gastric | 1.601 | 233744_at | AW668616 | 190 | |
| | protein ZG33P mRNA, partial | | | | | |
| | cds | | | | | |
| CDC27 | cell division cycle 27 | 1.6 | 238876_at | AA584875 | 191 | |
| | qs72h01.x1 NCI_CGAP_Pr28 | 1.6 | 229498_at | AI197932 | 192 | |
| | Homo sapiens cDNA clone | | | | | |
| | IMAGE: 1943665 3′, mRNA | | | | | |
| | sequence. | | | | | |
| PRDX4 | peroxiredoxin 4 | 1.598 | 201923_at | NM_006406 | 193 | |
| MRS2L | MRS2-like, magnesium | 1.596 | 224101_x_at | BC001028 | 194 | |
| | homeostasis factor (S. cerevisiae) | | | | | |
| UBE2S | ubiquitin-conjugating enzyme | 1.594 | 202779_s_at | NM_014501 | 195 | |
| | E2S | | | | | |
| YT521 | splicing factor YT521-B | 1.592 | 214814_at | BF592058 | 196 | |
| LOC339005 | 7n19a10.x1 | 1.591 | 244105_at | BF195207 | 197 | |
| | NCI_CGAP_Lu24 Homo | | | | | |
| | sapiens cDNA clone | | | | | |
| | IMAGE: 3564955 3′ similar to | | | | | |
| | TR: Q9QXM1 Q9QXM1 P300 | | | | | |
| | TRANSCRIPTIONAL | | | | | |
| | COFACTOR JMY.;, mRNA | | | | | |
| | sequence. | | | | | |
| HKE2 | HLA class II region expressed | 1.59 | 233588_x_at | BE561798 | 198 | |
| | gene KE2 | | | | | |
| FLJ30435 | Hypothetical protein | 1.586 | 1554741_s_at | AF523265 | 199 | |
| | LOC286286 (LOC286286), | | | | | |
| | mRNA | | | | | |
| D15Wsu75e | DNA segment, Chr 15, | 1.581 | 212527_at | BF057059 | 200 | |
| | Wayne State University 75, | | | | | |
| | expressed | | | | | |
| | Clone IMAGE: 5313053, | 1.58 | 1560540_x_at | BC042815 | 201 | |
| | mRNA | | | | | |
| UBE2G2 | ubiquitin-conjugating enzyme | 1.577 | 209042_s_at | BC001738 | 202 | |
| | E2G 2 (UBC7 homolog, | | | | | |
| | yeast) | | | | | |
| | Homo sapiens cDNA: | 1.577 | 1566749_at | AK024520 | 203 | |
| | FLJ20867 fis, clone | | | | | |
| | ADKA02259. | | | | | |
| P24B | integral type I protein | 1.577 | 208837_at | BC000027 | 204 | |
| CLGN | calmegin | 1.576 | 205830_at | NM_004362 | 205 | |
| | | 1.574 | 214984_at | AC003007 | 206 | |
| ZSIG11 | putative secreted protein | 1.57 | 233350_s_at | AU158474 | 207 | |
| | ZSIG11 | | | | | |
| | AU144530 HEMBA1 Homo | 1.57 | 215607_x_at | AU144530 | 208 | |
| | sapiens cDNA clone | | | | | |
| | HEMBA1002189 3′, mRNA | | | | | |
| | sequence. | | | | | |
| | Transcribed sequence with | 1.569 | 235543_at | AI928184 | 209 | |
| | moderate similarity to protein | | | | | |
| | ref: NP_060265.1 (H. sapiens) | | | | | |
| | hypothetical protein | | | | | |
| | FLJ20378 [Homo sapiens ] | | | | | |
| LOC253982 | hypothetical protein | 1.568 | 214993_at | AF070642 | 210 | |
| | LOC253982 | | | | | |
| MGC14141 | hypothetical protein | 1.565 | 225568_at | BE728983 | 211 | |
| | MGC14141 | | | | | |
| | Transcribed sequences | 1.565 | 231495_at | AA744508 | 212 | |
| FLJ21736 | esterase 31 | 1.563 | 220335_x_at | NM_024922 | 213 | |
| EML5 | echinoderm microtubule | 1.563 | 242443_at | AW026978 | 214 | |
| | associated protein like 5 | | | | | |
| | Transcribed sequence with | 1.561 | 236209_at | BF446127 | 215 | |
| | weak similarity to protein | | | | | |
| | ref: NP_055730.1 (H. sapiens) | | | | | |
| | KIAA1074 protein [Homo | | | | | |
| | sapiens] | | | | | |
| KIAA1720 | KIAA1720 protein | 1.561 | 223265_at | AL136569 | 216 | |
| | MRNA; cDNA | 1.56 | 1562281_at | AL832038 | 217 | |
| | DKFZp313A0310 (from clone | | | | | |
| | DKFZp313A0310) | | | | | |
| BCL3 | B-cell CLL/lymphoma 3 | 1.557 | 204908_s_at | NM_005178 | 218 | |
| RPL13 | ribosomal protein L13 | 1.556 | 229590_at | AI369389 | 219 | |
| | hh20e02.x1 | 1.556 | 1555926_a_at | BE045368 | 220 | |
| | NCI_CGAP_Lu24 Homo | | | | | |
| | sapiens cDNA clone | | | | | |
| | IMAGE: 2955674 3′, mRNA | | | | | |
| | sequence. | | | | | |
| SULF1 | sulfatase 1 | 1.554 | 212354_at | BE500977 | 221 | |
| | EST374778 MAGE | 1.553 | 239154_at | AW962705 | 222 | |
| | resequences, MAGG Homo | | | | | |
| | sapiens cDNA, mRNA | | | | | |
| | sequence. | | | | | |
| | novel ORF; Homo sapiens | 1.55 | 216702_x_at | U82303 | 223 | |
| | unknown protein mRNA, | | | | | |
| | partial cds. | | | | | |
| PPP2R2A | protein phosphatase 2 | 1.548 | 236492_at | AI934447 | 224 | |
| | (formerly 2A), regulatory | | | | | |
| | subunit B (PR 52), alpha | | | | | |
| | isoform | | | | | |
| | Full length insert cDNA clone | 1.547 | 1560926_at | AF085924 | 225 | |
| | YR43G06 | | | | | |
| FLJ10276 | hypothetical protein | 1.546 | 1559971_at | BC037860 | 226 | |
| | FLJ10276 | | | | | |
| SITPEC | likely ortholog of mouse | 1.546 | 218225_at | NM_016581 | 227 | |
| | signaling intermediate in Toll | | | | | |
| | pathway-evolutionarily | | | | | |
| | conserved | | | | | |
| C6orf157 | chromosome 6 open reading | 1.545 | 1569262_x_at | BC036049 | 228 | |
| | frame 157 | | | | | |
| MYST4 | MYST histone | 1.545 | 1562236_at | AL832065 | 229 | |
| | acetyltransferase (monocytic | | | | | |
| | leukemia) 4 | | | | | |
| MMRN1 | multimerin 1 | 1.543 | 205612_at | NM_007351 | 230 | |
| TTN | titin | 1.541 | 1561230_at | BC043155 | 231 | |
| | 602295566F1 NIH_MGC_86 | 1.54 | 235058_at | BG028320 | 232 | |
| | Homo sapiens cDNA clone | | | | | |
| | IMAGE: 4390216 5′, mRNA | | | | | |
| | sequence. | | | | | |
| AGRN | agrin | 1.54 | 217410_at | AK021586 | 233 | |
| KIAA1458 | KIAA1458 protein | 1.539 | 233874_at | AK025264 | 234 | |
| SPAG9 | sperm associated antigen 9 | 1.537 | 212468_at | AK023512 | 235 | |
| MTVR1 | Mouse Mammary Turmor | 1.535 | 32209_at | AF052151 | 236 | |
| | Virus Receptor homolog 1 | | | | | |
| SMARCD3 | SWI/SNF related, matrix | 1.534 | 204099_at | NM_003078 | 237 | |
| | associated, actin dependent | | | | | |
| | regulator of chromatin, | | | | | |
| | subfamily d, member 3 | | | | | |
| TNRC6 | trinucleotide repeat | 1.532 | 243834_at | BF507964 | 238 | |
| | containing 6 | | | | | |
| VEST1 | vestibule-1 protein | 1.531 | 1562301_at | AL834454 | 239 | |
| FLJ11218 | wn03f05.x1 NCI_CGAP_Ut2 | 1.531 | 238634_x_at | AI924246 | 240 | |
| | Homo sapiens cDNA clone | | | | | |
| | IMAGE: 2444385 3′, mRNA | | | | | |
| | sequence. | | | | | |
| CD8B1 | CD8 antigen, beta | 1.528 | 215332_s_at | AW296309 | 241 | |
| | polypeptide 1 (p37) | | | | | |
| SLC15A1 | solute carrier family 15 | 1.527 | 211349_at | AB001328 | 242 | |
| | (oligopeptide transporter), | | | | | |
| | member 1 | | | | | |
| AF5Q31 | ALL1 fused gene from 5q31 | 1.527 | 1555435_at | BC025700 | 243 | |
| MSTP9 | | 1.524 | 213382_at | AL137798 | 244 | |
| KIAA0540 | KIAA0540 protein | 1.524 | 212443_at | AB011112 | 245 | |
| KIAA1833 | Homo sapiens cDNA | 1.522 | 232498_at | AK023386 | 246 | |
| | FLJ13324 fis, clone | | | | | |
| | OVARC1001745. | | | | | |
| | Clone IMAGE: 5581630, | 1.52 | 1569665_at | BC035773 | 247 | |
| | mRNA | | | | | |
| SCEL | sciellin | 1.519 | 1554920_at | BC020726 | 248 | |
| FKBP11 | FK506 binding protein 11, 19 kDa | 1.519 | 219118_at | NM_016594 | 249 | |
| CCDC2 | coiled-coil domain containing 2 | 1.518 | 61732_r_at | AI610355 | 250 | |
| | Transcribed sequences | 1.517 | 235952_at | AA521504 | 251 | |
| METAP2 | methionyl aminopeptidase 2 | 1.516 | 202015_x_at | NM_006838 | 252 | |
| FLJ20186 | hypothetical protein | 1.516 | 219646_at | NM_017702 | 253 | |
| | FLJ20186 | | | | | |
| | MRNA; cDNA | 1.516 | 1564580_at | AL050059 | 254 | |
| | DKFZp566H153 (from clone | | | | | |
| | DKFZp566H153) | | | | | |
| FBN2 | fibrillin 2 (congenital | 1.515 | 203184_at | NM_001999 | 255 | |
| | contractural arachnodactyly) | | | | | |
| PTK9 | PTK9 protein tyrosine kinase 9 | 1.512 | 243033_at | AA827894 | 256 | |
| MAF | v-maf musculoaponeurotic | 1.512 | 1566324_a_at | AA442149 | 257 | |
| | fibrosarcoma oncogene | | | | | |
| | homolog (avian) | | | | | |
| RPL10L | ribosomal protein L10-like | 1.511 | 217559_at | AI001784 | 258 | |
| C20orf38 | chromosome 20 open reading | 1.511 | 220456_at | NM_018327 | 259 | |
| | frame 38 | | | | | |
| DUSP16 | dual specificity phosphatase | 1.51 | 1563505_at | AL833073 | 260 | |
| | 16 | | | | | |
| | Clone IMAGE: 5299520, | 1.51 | 1562431_x_at | BC041911 | 261 | |
| | mRNA | | | | | |
| C6orf163 | chromosome 6 open reading | 1.51 | 242446_at | AI033106 | 262 | |
| | frame 163 | | | | | |
| | Homo sapiens cDNA: | 1.509 | 234219_at | AK024998 | 263 | |
| | FLJ21345 fis, clone | | | | | |
| | COL02694. | | | | | |
| STRA13 | stimulated by retinoic acid 13 | 1.506 | 209478_at | U95006 | 264 | |
| MPRA | membrane progestin receptor | 1.505 | 242123_at | BF764937 | 265 | |
| | alpha | | | | | |
| MGC9712 | hypothetical protein | 1.505 | 235817_at | AA890722 | 266 | |
| | MGC9712 | | | | | |
| RANBP2L1 | RAN binding protein 2-like 1 | 1.503 | 211324_s_at | AL136868 | 267 | |
| PDE4D | phosphodiesterase 4D, | 1.502 | 204491_at | R40917 | 268 | |
| | cAMP-specific | | | | | |
| | (phosphodiesterase E3 dunce | | | | | |
| | homolog, Drosophila) | | | | | |
| C19orf24 | hypothetical protein | 1.501 | 221587_s_at | BC000890 | 269 | |
| | FLJ20640 | | | | | |
| | Clone IMAGE: 5259584, | 1.501 | 231193_s_at | BE326569 | 270 | |
| | mRNA | | | | | |
| FLJ11292 | hypothetical protein | 1.5 | 220828_s_at | NM_018382 | 271 | |
| | FLJ11292 | | | | | |
| | Clone IMAGE: 5259584, | 1.5 | 224778_s_at | AK025902 | 272 | |
| | mRNA | | | | | |
| | Transcribed sequences | 0.667 | 239124_at | AA002064 | 273 | |
| ESDN | endothelial and smooth | 0.667 | 230175_s_at | AA805633 | 274 | |
| | muscle cell-derived | | | | | |
| | neuropilin-like protein | | | | | |
| HSPA1A | heat shock 70 kDa protein 1A | 0.666 | 200799_at | NM_005345 | 275 | |
| PPID | peptidylprolyl isomerase D | 0.666 | 204186_s_at | AI014573 | 276 | |
| | (cyclophilin D) | | | | | |
| HRB2 | Transcribed sequence with | 0.666 | 232441_at | AU147079 | 277 | |
| | weak similarity to protein | | | | | |
| | ref: NP_060265.1 (H. sapiens) | | | | | |
| | hypothetical protein | | | | | |
| | FLJ20378 [Homo sapiens ] | | | | | |
| ELL2 | elongation factor, RNA | 0.666 | 226099_at | AI924426 | 278 | |
| | polymerase II, 2 | | | | | |
| MAP3K2 | mitogen-activated protein | 0.666 | 221695_s_at | AF239798 | 279 | |
| | kinase kinase kinase 2 | | | | | |
| | MRNA; cDNA | 0.666 | 232835_at | AA533080 | 280 | |
| | DKFZp667N064 (from clone | | | | | |
| | DKFZp667N064) | | | | | |
| C14orf138 | chromosome 14 open reading | 0.666 | 218940_at | NM_024558 | 281 | |
| | frame 138 | | | | | |
| | Transcribed sequence with | 0.665 | 239866_at | AA705933 | 282 | |
| | weak similarity to protein | | | | | |
| | ref: NP_055301.1 (H. sapiens) | | | | | |
| | neuronal thread protein | | | | | |
| | [Homo sapiens ] | | | | | |
| ARL10C | ADP-ribosylation factor-like | 0.665 | 222442_s_at | AW055237 | 283 | |
| | 10C | | | | | |
| PLEKHA2 | 602068320F1 NIH_MGC_58 | 0.665 | 217677_at | BF542055 | 284 | |
| | Homo sapiens cDNA clone | | | | | |
| | IMAGE: 4067357 5′, mRNA | | | | | |
| | sequence. | | | | | |
| | EST379822 MAGE | 0.665 | 235360_at | AW967747 | 285 | |
| | resequences, MAGJ Homo | | | | | |
| | sapiens cDNA, mRNA | | | | | |
| | sequence. | | | | | |
| TMF1 | TATA element modulatory | 0.665 | 235566_at | AW591660 | 286 | |
| | factor 1 | | | | | |
| NUP54 | nucleoporin 54 kDa | 0.665 | 218256_s_at | NM_017426 | 287 | |
| MAPK9 | mitogen-activated protein | 0.665 | 203218_at | W37431 | 288 | |
| | kinase 9 | | | | | |
| SLC38A1 | solute carrier family 38, | 0.665 | 224579_at | BF247552 | 289 | |
| | member 1 | | | | | |
| RAB21 | RAB21, member RAS | 0.665 | 226268_at | AI309554 | 290 | |
| | oncogene family | | | | | |
| PRKAG1 | protein kinase, AMP- | 0.665 | 227527_at | AI394529 | 291 | |
| | activated, gamma 1 non- | | | | | |
| | catalytic subunit | | | | | |
| KIAA1972 | KIAA1972 protein | 0.665 | 225774_at | AA062971 | 292 | |
| TRIB2 | tribbles homolog 2 | 0.665 | 202479_s_at | BC002637 | 293 | |
| FLJ20273 | RNA-binding protein | 0.665 | 229440_at | AI830823 | 294 | |
| USP13 | ubiquitin specific protease 13 | 0.665 | 205356_at | NM_003940 | 295 | |
| | (isopeptidase T-3) | | | | | |
| | Transcribed sequence with | 0.665 | 229249_at | BF511212 | 296 | |
| | weak similarity to protein | | | | | |
| | sp: P11369 (M. musculus) | | | | | |
| | POL2_MOUSE Retrovirus- | | | | | |
| | related POL polyprotein | | | | | |
| | [Contains: Reverse | | | | | |
| | transcriptase; Endonuclease] | | | | | |
| KPNB1 | karyopherin (importin) beta 1 | 0.665 | 208975_s_at | L38951 | 297 | |
| FEZ2 | Homo sapiens mRNA; cDNA | 0.665 | 215000_s_at | AL117593 | 298 | |
| | DKFZp564G082 (from clone | | | | | |
| | DKFZp564G082). | | | | | |
| MBD6 | methyl-CpG binding domain | 0.665 | 226076_s_at | AI174548 | 299 | |
| | protein 6 | | | | | |
| OSBPL11 | oxysterol binding protein-like | 0.665 | 218304_s_at | NM_022776 | 300 | |
| | 11 | | | | | |
| CD47 | CD47 antigen (Rh-related | 0.665 | 213856_at | BG230614 | 301 | |
| | antigen, integrin-associated | | | | | |
| | signal transducer) | | | | | |
| HYPB | huntingtin interacting protein B | 0.664 | 212493_s_at | AI761110 | 302 | |
| TIEG | TGFB inducible early growth | 0.664 | 202393_s_at | NM_005655 | 303 | |
| | response | | | | | |
| VAMP4 | vesicle-associated membrane | 0.664 | 211760_s_at | BC005974 | 304 | |
| | protein 4 | | | | | |
| LARS | leucyl-tRNA synthetase | 0.664 | 222428_s_at | D84223 | 305 | |
| KIAA2024 | similar to F10G7.10.p | 0.664 | 234982_at | BF577193 | 306 | |
| FBXO21 | F-box only protein 21 | 0.664 | 212229_s_at | AK001699 | 307 | |
| SNAP23 | UI-H-BI2-ahj-d-03-0-UI.s1 | 0.664 | 229773_at | AW293341 | 308 | |
| | NCI_CGAP_Sub4 Homo | | | | | |
| | sapiens cDNA clone | | | | | |
| | IMAGE: 2726837 3′, mRNA | | | | | |
| | sequence. | | | | | |
| UBE2J1 | ubiquitin-conjugating enzyme | 0.664 | 217826_s_at | NM_016021 | 309 | |
| | E2, J1 (UBC6 homolog, | | | | | |
| | yeast) | | | | | |
| LLT1 | lectin-like NK cell receptor | 0.664 | 233500_x_at | AF285089 | 310 | |
| MED6 | mediator of RNA polymerase | 0.664 | 207078_at | NM_005466 | 311 | |
| | II transcription, subunit 6 | | | | | |
| | homolog (yeast) | | | | | |
| NFATC3 | nuclear factor of activated T- | 0.664 | 210556_at | U85430 | 312 | |
| | cells, cytoplasmic, | | | | | |
| | calcineurin-dependent 3 | | | | | |
| RBL2 | retinoblastoma-like 2 (p130) | 0.663 | 212331_at | X76061 | 313 | |
| LASS6 | hypothetical protein | 0.663 | 235463_s_at | AI081356 | 314 | |
| | LOC253782 | | | | | |
| LOC149705 | chromosome 20 open reading | 0.663 | 228031_at | AW444778 | 315 | |
| | frame 121 | | | | | |
| C1orf8 | 7q91g02.x1 | 0.663 | 241018_at | BF446758 | 316 | |
| | NCI_CGAP_Lu24 Homo | | | | | |
| | sapiens cDNA clone | | | | | |
| | IMAGE: 3705794 3′, mRNA | | | | | |
| | sequence. | | | | | |
| DDX21 | DEAD (Asp-Glu-Ala-Asp) box | 0.663 | 208152_s_at | NM_004728 | 317 | |
| | polypeptide 21 | | | | | |
| MAP3K7IP2 | mitogen-activated protein | 0.663 | 212184_s_at | AL117407 | 318 | |
| | kinase kinase kinase 7 | | | | | |
| | interacting protein 2 | | | | | |
| | Clone IMAGE: 5272105, | 0.663 | 1557738_at | BC041344 | 319 | |
| | mRNA | | | | | |
| HS2ST1 | heparan sulfate 2-O- | 0.663 | 203284_s_at | AW151887 | 320 | |
| | sulfotransferase 1 | | | | | |
| MAN2A1 | mannosidase, alpha, class | 0.663 | 235103_at | AA029155 | 321 | |
| | 2A, member 1 | | | | | |
| KIAA0232 | KIAA0232 gene product | 0.663 | 232366_at | AF143884 | 322 | |
| LPIN1 | lipin 1 | 0.663 | 212276_at | D80010 | 323 | |
| C10orf137 | erythroid differentiation- | 0.663 | 213410_at | AL050102 | 324 | |
| | related factor 1 | | | | | |
| TMOD2 | tropomodulin 2 (neuronal) | 0.663 | 226186_at | AW207699 | 325 | |
| | CDNA FLJ38745 fis, clone | 0.662 | 1566001_at | AK096064 | 326 | |
| | KIDNE2012291 | | | | | |
| MTO1 | mitochondrial translation | 0.662 | 233665_x_at | AK026073 | 327 | |
| | optimization 1 homolog (S. cerevisiae) | | | | | |
| CASP8 | caspase 8, apoptosis-related | 0.662 | 207686_s_at | NM_001228 | 328 | |
| | cysteine protease | | | | | |
| PTGS1 | prostaglandin-endoperoxide | 0.662 | 205127_at | NM_000962 | 329 | |
| | synthase 1 (prostaglandin | | | | | |
| | G/H synthase and | | | | | |
| | cyclooxygenase) | | | | | |
| RASA1 | RAS p21 protein activator | 0.662 | 202677_at | NM_002890 | 330 | |
| | (GTPase activating protein) 1 | | | | | |
| RABGEF1 | 602506825F1 NIH_MGC_79 | 0.662 | 235407_at | BG435723 | 331 | |
| | Homo sapiens cDNA clone | | | | | |
| | IMAGE: 4604046 5′, mRNA | | | | | |
| | sequence. | | | | | |
| STRN3 | striatin, calmodulin binding | 0.662 | 204496_at | NM_014574 | 332 | |
| | protein 3 | | | | | |
| DUSP6 | dual specificity phosphatase 6 | 0.662 | 208892_s_at | BC003143 | 333 | |
| | Transcribed sequences | 0.662 | 231205_at | BF055351 | 334 | |
| IPLA2(GAMMA) | intracellular membrane- | 0.662 | 223982_s_at | AB041261 | 335 | |
| | associated calcium- | | | | | |
| | independent phospholipase | | | | | |
| | A2 gamma | | | | | |
| BCLAF1 | BCL2-associated | 0.662 | 201101_s_at | BE963370 | 336 | |
| | transcription factor 1 | | | | | |
| SHMT1 | serine | 0.662 | 227304_at | N25727 | 337 | |
| | hydroxymethyltransferase 1 | | | | | |
| | (soluble) | | | | | |
| SSBP1 | single-stranded DNA binding | 0.662 | 214060_at | BE220360 | 338 | |
| | protein 1 | | | | | |
| ARNTL | aryl hydrocarbon receptor | 0.662 | 210971_s_at | AB000815 | 339 | |
| | nuclear translocator-like | | | | | |
| | Rho-associated, coiled-coil | 0.662 | 213044_at | N22548 | 340 | |
| | containing protein kinase 1 | | | | | |
| RAB5A | RAB5A, member RAS | 0.661 | 209089_at | BC001267 | 341 | |
| | oncogene family | | | | | |
| VPS41 | vacuolar protein sorting 41 | 0.661 | 210849_s_at | AF135593 | 342 | |
| | (yeast) | | | | | |
| 6-Sep | septin 6 | 0.661 | 212414_s_at | D50918 | 343 | |
| PDK3 | pyruvate dehydrogenase | 0.661 | 221957_at | BF939522 | 344 | |
| | kinase, isoenzyme 3 | | | | | |
| CDYL | chromodomain protein, Y-like | 0.661 | 203100_s_at | NM_004824 | 345 | |
| KIFAP3 | AV712694 DCA Homo | 0.661 | 227637_at | AV712694 | 346 | |
| | sapiens cDNA clone | | | | | |
| | DCAAJG07 5′, mRNA | | | | | |
| | sequence. | | | | | |
| | Hypothetical gene supported | 0.661 | 226596_x_at | AK027125 | 347 | |
| | by AK027125 (LOC402460), | | | | | |
| | mRNA | | | | | |
| RPGR | retinitis pigmentosa GTPase | 0.661 | 207624_s_at | NM_000328 | 348 | |
| | regulator | | | | | |
| KIAA0261 | KIAA0261 | 0.661 | 1554441_a_at | AF479418 | 349 | |
| | Transcribed sequences | 0.661 | 236474_at | AI797677 | 350 | |
| KIAA0196 | KIAA0196 gene product | 0.661 | 201985_at | NM_014846 | 351 | |
| TOB2 | transducer of ERBB2, 2 | 0.661 | 222243_s_at | AB051450 | 352 | |
| | DKFZp686E1983_r1 686 | 0.661 | 1558937_s_at | AL711520 | 353 | |
| | (synonym: hlcc3) Homo | | | | | |
| | sapiens cDNA clone | | | | | |
| | DKFZp686E1983 5′, mRNA | | | | | |
| | sequence. | | | | | |
| APC | adenomatosis polyposis coli | 0.661 | 203526_s_at | M74088 | 354 | |
| KIAA1221 | KIAA1221 protein | 0.661 | 1553725_s_at | NM_016620 | 355 | |
| MYCBP2 | protein associated with Myc | 0.66 | 1557370_s_at | AK000968 | 356 | |
| PSIP1 | PC4 and SFRS1 interacting | 0.66 | 222286_at | R43279 | 357 | |
| | protein 1 | | | | | |
| UBR1 | AV715153 DCB Homo | 0.66 | 226921_at | AV715153 | 358 | |
| | sapiens cDNA clone | | | | | |
| | DCBBNE07 5′, mRNA | | | | | |
| | sequence. | | | | | |
| UPF3A | UPF3 regulator of nonsense | 0.66 | 214323_s_at | N36842 | 359 | |
| | transcripts homolog A (yeast) | | | | | |
| | Transcribed sequences | 0.66 | 243915_at | AW130385 | 360 | |
| LOC91137 | om73g04.s1 | 0.66 | 226831_at | AA928542 | 361 | |
| | NCI_CGAP_GC4 Homo | | | | | |
| | sapiens cDNA clone | | | | | |
| | IMAGE: 1552854 3′, mRNA | | | | | |
| | sequence. | | | | | |
| EAF1 | ELL associated factor 1 | 0.66 | 226952_at | AU149385 | 362 | |
| CPR8 | cell cycle progression 8 | 0.66 | 221156_x_at | NM_004748 | 363 | |
| | protein | | | | | |
| ZNF567 | zinc finger protein 567 | 0.66 | 235648_at | AA742659 | 364 | |
| RECQL | RecQ protein-like (DNA | 0.66 | 210568_s_at | BC001052 | 365 | |
| | helicase Q1-like) | | | | | |
| SCFD1 | sec1 family domain | 0.659 | 215548_s_at | AB020724 | 366 | |
| | containing 1 | | | | | |
| HIP1 | huntingtin interacting protein 1 | 0.659 | 205426_s_at | U79734 | 367 | |
| TSBF1 | tumor suppressor TSBF1 | 0.659 | 227801_at | N90779 | 368 | |
| MAP3K5 | mitogen-activated protein | 0.659 | 203837_at | NM_005923 | 369 | |
| | kinase kinase kinase 5 | | | | | |
| MGC27165 | Homo sapiens partial IGVH3 | 0.659 | 214973_x_at | AJ275469 | 370 | |
| | gene for immunoglobulin | | | | | |
| | heavy chain V region, case 2, | | | | | |
| | cell E 172. | | | | | |
| APOL6 | apolipoprotein L, 6 | 0.659 | 241869_at | AW026509 | 371 | |
| CD6 | CD6 antigen | 0.659 | 211893_x_at | U66145 | 372 | |
| RHOB | 603295907F1 NIH_MGC_96 | 0.659 | 1553962_s_at | BI668074 | 373 | |
| | Homo sapiens cDNA clone | | | | | |
| | IMAGE: 5315136 5′, mRNA | | | | | |
| | sequence. | | | | | |
| MAPKAP1 | mitogen-activated protein | 0.659 | 222426_at | BG499947 | 374 | |
| | kinase associated protein 1 | | | | | |
| SFPQ | splicing factor | 0.659 | 221768_at | AV705803 | 375 | |
| | proline/glutamine rich | | | | | |
| | (polypyrimidine tract binding | | | | | |
| | protein associated) | | | | | |
| TXK | TXK tyrosine kinase | 0.659 | 206828_at | NM_003328 | 376 | |
| KIAA1228 | KIAA1228 protein | 0.658 | 1558511_s_at | AA187963 | 377 | |
| LOC155435 | Hypothetical protein | 0.658 | 225839_at | AW290882 | 378 | |
| | LOC155435 (LOC155435), | | | | | |
| | mRNA | | | | | |
| | Transcribed sequences | 0.658 | 239358_at | BE645256 | 379 | |
| SH3BP5 | SH3-domain binding protein 5 | 0.658 | 201811_x_at | NM_004844 | 380 | |
| | (BTK-associated) | | | | | |
| PDCD4 | programmed cell death 4 | 0.658 | 212593_s_at | N92498 | 381 | |
| | (neoplastic transformation | | | | | |
| | inhibitor) | | | | | |
| ATP6V0A2 | synonyms: a2, TJ6, J6B7, | 0.658 | 205704_s_at | NM_012463 | 382 | |
| | Stv1, TJ6M, TJ6s, Vph1, | | | | | |
| | ATP6a2, ATP6N1D; | | | | | |
| | go_process: immune | | | | | |
| | response [goid 0006955] | | | | | |
| | [evidence E]; Homo sapiens | | | | | |
| | ATPase, H+ transporting, | | | | | |
| | lysosomal V0 subunit a | | | | | |
| | isoform 2 (ATP6V0A2), | | | | | |
| | mRNA. | | | | | |
| | Transcribed sequences | 0.658 | 235786_at | AI806781 | 383 | |
| KIAA1228 | | 0.658 | 1555830_s_at | BC001224 | 384 | |
| HBP1 | HMG-box transcription factor 1 | 0.658 | 207361_at | NM_012257 | 385 | |
| TRA1 | tumor rejection antigen | 0.658 | 200599_s_at | NM_003299 | 386 | |
| | (gp96) 1 | | | | | |
| HGRG8 | high-glucose-regulated | 0.658 | 217812_at | NM_016258 | 387 | |
| | protein 8 | | | | | |
| PAI-RBP1 | PAI-1 mRNA-binding protein | 0.658 | 210466_s_at | BC002488 | 388 | |
| DR1 | down-regulator of | 0.658 | 209187_at | AW516932 | 389 | |
| | transcription 1, TBP-binding | | | | | |
| | (negative cofactor 2) | | | | | |
| FRMD3 | FERM domain containing 3 | 0.658 | 230645_at | BF110588 | 390 | |
| SEC10L1 | UI-H-BI4-aoy-b-12-0-UI.s1 | 0.658 | 228418_at | BF509391 | 391 | |
| | NCI_CGAP_Sub8 Homo | | | | | |
| | sapiens cDNA clone | | | | | |
| | IMAGE: 3086542 3′, mRNA | | | | | |
| | sequence. | | | | | |
| TLR4 | synonym: Lps; encodes most | 0.658 | 232068_s_at | AF177765 | 392 | |
| | common amino acid | | | | | |
| | sequence in humans; | | | | | |
| | membrane spanning | | | | | |
| | component of the human LPS | | | | | |
| | receptor; human homolog of | | | | | |
| | the mouse Lps gene product; | | | | | |
| | Homo sapiens toll-like | | | | | |
| | receptor 4 (TLR4) gene, | | | | | |
| | TLR4A allele, complete cds. | | | | | |
| SRP72 | signal recognition particle | 0.658 | 208800_at | AV702627 | 393 | |
| | 72 kDa | | | | | |
| DKFZp762C186 | tangerin | 0.658 | 1557749_at | AK092750 | 394 | |
| | Transcribed sequences | 0.658 | 235347_at | BF435591 | 395 | |
| TTC3 | tetratricopeptide repeat | 0.658 | 208662_s_at | AI885338 | 396 | |
| | domain 3 | | | | | |
| | yh87a04.r1 Soares placenta | 0.657 | 1560171_at | R34775 | 397 | |
| | Nb2HP Homo sapiens cDNA | | | | | |
| | clone IMAGE: 136686 5′, | | | | | |
| | mRNA sequence. | | | | | |
| | Transcribed sequence with | 0.657 | 227682_at | BE645154 | 398 | |
| | weak similarity to protein | | | | | |
| | pir: A45973 (H. sapiens) | | | | | |
| | A45973 trichohyalin - human | | | | | |
| ZCCHC8 | zinc finger, CCHC domain | 0.657 | 218478_s_at | NM_017612 | 399 | |
| | containing 8 | | | | | |
| SUGT1 | SGT1, suppressor of G2 | 0.657 | 223330_s_at | BC000911 | 400 | |
| | allele of SKP1 (S. cerevisiae) | | | | | |
| HNRPDL | heterogeneous nuclear | 0.657 | 1554678_s_at | AB066484 | 401 | |
| | ribonucleoprotein D-like | | | | | |
| RW1 | RW1 protein | 0.657 | 212507_at | D87446 | 402 | |
| CHC1L | chromosome condensation 1- | 0.657 | 204759_at | NM_001268 | 403 | |
| | like | | | | | |
| PAPD1 | hypothetical protein | 0.657 | 218947_s_at | NM_018109 | 404 | |
| | FLJ10486 | | | | | |
| SMAD2 | MAD, mothers against | 0.657 | 203077_s_at | NM_005901 | 405 | |
| | decapentaplegic homolog 2 | | | | | |
| | (Drosophila) | | | | | |
| KIAA0431 | | 0.657 | 201855_s_at | NM_015251 | 406 | |
| IDN3 | IDN3 protein | 0.657 | 242352_at | AW272262 | 407 | |
| | Transcribed sequences | 0.657 | 241164_at | AA047225 | 408 | |
| RABEP1 | yh12b01.s1 Soares infant | 0.656 | 225064_at | R60018 | 409 | |
| | brain 1NIB Homo sapiens | | | | | |
| | cDNA clone IMAGE: 42905 3′, | | | | | |
| | mRNA sequence. | | | | | |
| | Homo sapiens mRNA; cDNA | 0.656 | 1560680_at | AL833513 | 410 | |
| | DKFZp686F0137 (from clone | | | | | |
| | DKFZp686F0137). | | | | | |
| FLJ32731 | yb33d05.s1 Stratagene fetal | 0.656 | 222491_at | T56470 | 411 | |
| | spleen (#937205) Homo | | | | | |
| | sapiens cDNA clone | | | | | |
| | IMAGE: 72969 3′, mRNA | | | | | |
| | sequence. | | | | | |
| KIAA0648 | KIAA0648 protein | 0.656 | 212138_at | AK021757 | 412 | |
| C19orf6 | chromosome 19 open reading | 0.656 | 213986_s_at | AI805266 | 413 | |
| | frame 6 | | | | | |
| CHS1 | Chediak-Higashi syndrome 1 | 0.656 | 215415_s_at | U70064 | 414 | |
| HSPD1 | Transcribed sequence with | 0.656 | 200806_s_at | BE256479 | 415 | |
| | strong similarity to protein | | | | | |
| | pir: A32800 (H. sapiens) | | | | | |
| | A32800 chaperonin GroEL | | | | | |
| | precursor —human | | | | | |
| | CDNA FLJ27162 fis, clone | 0.656 | 236134_at | AA769995 | 416 | |
| | SYN01352 | | | | | |
| DDHD1 | DDHD domain containing 1 | 0.656 | 225970_at | AA029818 | 417 | |
| ZC3HDC7 | zinc-finger protein AY163807 | 0.656 | 226897_s_at | AW169959 | 418 | |
| MGC8721 | hypothetical protein | 0.656 | 200847_s_at | NM_016127 | 419 | |
| | MGC8721 | | | | | |
| GCL | hypothetical protein | 0.655 | 239355_at | BF675979 | 420 | |
| | FLJ13057 similar to germ | | | | | |
| | cell-less | | | | | |
| SON | SON DNA binding protein | 0.655 | 201086_x_at | NM_003103 | 421 | |
| KIAA0893 | KIAA0893 protein | 0.655 | 203855_at | NM_014969 | 422 | |
| SOD2 | superoxide dismutase 2, | 0.655 | 216841_s_at | X15132 | 423 | |
| | mitochondrial | | | | | |
| NCOR1 | nz73d11.s1 | 0.655 | 243682_at | AA721355 | 424 | |
| | NCI_CGAP_GCB1 Homo | | | | | |
| | sapiens cDNA clone | | | | | |
| | IMAGE: 1301109 3′ similar to | | | | | |
| | gb: U01120 GLUCOSE-6- | | | | | |
| | PHOSPHATASE (HUMAN);, | | | | | |
| | mRNA sequence. | | | | | |
| MCM3AP | Homo sapiens cDNA | 0.655 | 215581_s_at | AK022303 | 425 | |
| | FLJ12241 fis, clone | | | | | |
| | MAMMA1001274. | | | | | |
| | yI97c03.s1 Soares infant | 0.655 | 224582_s_at | H09085 | 426 | |
| | brain 1NIB Homo sapiens | | | | | |
| | cDNA clone IMAGE: 46374 3′, | | | | | |
| | mRNA sequence. | | | | | |
| MME | membrane metallo- | 0.655 | 203434_s_at | AI433463 | 427 | |
| | endopeptidase (neutral | | | | | |
| | endopeptidase, | | | | | |
| | enkephalinase, CALLA, | | | | | |
| | CD10) | | | | | |
| PTPN11 | predicted protein of HQ1847; | 0.655 | 209896_s_at | AF119855 | 428 | |
| | Homo sapiens PRO1847 | | | | | |
| | mRNA, complete cds. | | | | | |
| DNAJC10 | ER-resident protein ERdj5 | 0.655 | 221782_at | BG168666 | 429 | |
| HNRPM | heterogeneous nuclear | 0.655 | 1555844_s_at | AL713781 | 430 | |
| | ribonucleoprotein M | | | | | |
| TDE2 | tumor differentially expressed 2 | 0.655 | 208671_at | AF164794 | 431 | |
| SMURF1 | E3 ubiquitin ligase SMURF1 | 0.655 | 1559426_at | AL042818 | 432 | |
| HIC | I-mfa domain-containing | 0.655 | 217599_s_at | BE910600 | 433 | |
| | protein | | | | | |
| LOC91526 | hypothetical protein | 0.654 | 235778_s_at | AI985590 | 434 | |
| | DKFZp434D2328 | | | | | |
| WASPIP | Wiskott-Aldrich syndrome | 0.654 | 202664_at | AW058622 | 435 | |
| | protein interacting protein | | | | | |
| | MRNA; cDNA | 0.654 | 1563621_at | AL713724 | 436 | |
| | DKFZp667O0416 (from clone | | | | | |
| | DKFZp667O0416) | | | | | |
| 13CDNA73 | hypothetical protein CG003 | 0.654 | 204072_s_at | NM_023037 | 437 | |
| GBF1 | golgi-specific brefeldin A | 0.654 | 201439_at | NM_004193 | 438 | |
| | resistance factor 1 | | | | | |
| FLJ35036 | hypothetical protein | 0.653 | 1558733_at | BE386445 | 439 | |
| | FLJ35036 | | | | | |
| SMAD5 | MAD, mothers against | 0.653 | 225223_at | AI478523 | 440 | |
| | decapentaplegic homolog 5 | | | | | |
| | (Drosophila) | | | | | |
| ITGA6 | integrin, alpha 6 | 0.653 | 201656_at | NM_000210 | 441 | |
| | Transcribed sequences | 0.653 | 230387_at | AL038450 | 442 | |
| SMC6L1 | SMC6 structural maintenance | 0.653 | 218781_at | NM_024624 | 443 | |
| | of chromosomes 6-like 1 | | | | | |
| | (yeast) | | | | | |
| MGC4126 | hypothetical protein | 0.653 | 214739_at | AI357539 | 444 | |
| | MGC4126 | | | | | |
| BMI1 | COMM domain containing 3 | 0.653 | 202265_at | NM_005180 | 445 | |
| CUL4B | cullin 4B | 0.653 | 215997_s_at | AV694732 | 446 | |
| PBEF1 | pre-B-cell colony enhancing | 0.653 | 217739_s_at | NM_005746 | 447 | |
| | factor 1 | | | | | |
| XPO5 | exportin 5 | 0.653 | 223057_s_at | BC000129 | 448 | |
| MGC39820 | MRNA; cDNA | 0.652 | 238461_at | AA228031 | 449 | |
| | DKFZp686J24234 (from | | | | | |
| | clone DKFZp686J24234) | | | | | |
| RPS6KA3 | ribosomal protein S6 kinase, | 0.652 | 203843_at | AA906056 | 450 | |
| | 90 kDa, polypeptide 3 | | | | | |
| POLR2B | 601504169T1 NIH_MGC_71 | 0.652 | 1555837_s_at | BE614461 | 451 | |
| | Homo sapiens cDNA clone | | | | | |
| | IMAGE: 3905776 3′, mRNA | | | | | |
| | sequence. | | | | | |
| LOC339745 | MRNA; cDNA | 0.652 | 225659_at | BF516590 | 452 | |
| | DKFZp686O22117 (from | | | | | |
| | clone DKFZp686O22117) | | | | | |
| UHRF2 | ubiquitin-like, containing PHD | 0.652 | 225610_at | BF511410 | 453 | |
| | and RING finger domains, 2 | | | | | |
| PPP4R2 | hypothetical protein | 0.652 | 225519_at | AA206408 | 454 | |
| | FLJ10213 | | | | | |
| | Transcribed sequence with | 0.651 | 231366_at | AI190575 | 455 | |
| | weak similarity to protein | | | | | |
| | ref: NP_062553.1 (H. sapiens) | | | | | |
| | hypothetical protein | | | | | |
| | FLJ11267 [Homo sapiens ] | | | | | |
| CNOT8 | CCR4-NOT transcription | 0.651 | 202164_s_at | AF180476 | 456 | |
| | complex, subunit 8 | | | | | |
| SLC11A2 | solute carrier family 11 | 0.651 | 237106_at | AI051244 | 457 | |
| | (proton-coupled divalent | | | | | |
| | metal ion transporters), | | | | | |
| | member 2 | | | | | |
| JAK2 | Janus kinase 2 (a protein | 0.651 | 205841_at | NM_004972 | 458 | |
| | tyrosine kinase) | | | | | |
| MBNL2 | muscleblind-like 2 | 0.651 | 232138_at | AW276914 | 459 | |
| | (Drosophila) | | | | | |
| POLR2A | polymerase (RNA) II (DNA | 0.651 | 202725_at | NM_000937 | 460 | |
| | directed) polypeptide A, | | | | | |
| | 220 kDa | | | | | |
| | Clone IMAGE: 5175565, | 0.651 | 228465_at | T79942 | 461 | |
| | mRNA | | | | | |
| KIAA0478 | KIAA0478 gene product | 0.651 | 203959_s_at | NM_014870 | 462 | |
| IGHM | Ig VH4 heavy chain; Human | 0.651 | 216491_x_at | U80139 | 463 | |
| | immunoglobulin heavy chain | | | | | |
| | variable region (V4-4) gene, | | | | | |
| | partial cds. | | | | | |
| BIRC2 | baculoviral IAP repeat- | 0.651 | 202076_at | NM_001166 | 464 | |
| | containing 2 | | | | | |
| RAB11-FIP4 | 602400349F1 NIH_MGC_15 | 0.651 | 225746_at | BG248313 | 465 | |
| | Homo sapiens cDNA clone | | | | | |
| | IMAGE: 4545685 5′, mRNA | | | | | |
| | sequence. | | | | | |
| VPS35 | vacuolar protein sorting 35 | 0.651 | 222387_s_at | BG476669 | 466 | |
| | (yeast) | | | | | |
| | Transcribed sequence with | 0.65 | 217540_at | AA721025 | 467 | |
| | moderate similarity to protein | | | | | |
| | ref: NP_060265.1 (H. sapiens) | | | | | |
| | hypothetical protein | | | | | |
| | FLJ20378 [Homo sapiens ] | | | | | |
| C6orf69 | chromosome 6 open reading | 0.65 | 214849_at | AW500220 | 468 | |
| | frame 69 | | | | | |
| KIAA0092 | translokin | 0.65 | 209862_s_at | BC001233 | 469 | |
| | Transcribed sequence with | 0.65 | 235679_at | AI598222 | 470 | |
| | weak similarity to protein | | | | | |
| | ref: NP_071431.1 (H. sapiens) | | | | | |
| | cytokine receptor-like factor | | | | | |
| | 2; cytokine receptor CRL2 | | | | | |
| | precusor [Homo sapiens ] | | | | | |
| TA-PP2C | T-cell activation protein | 0.65 | 225213_at | AW300598 | 471 | |
| | phosphatase 2C | | | | | |
| TMOD3 | tropomodulin 3 (ubiquitous) | 0.65 | 223078_s_at | AF177171 | 472 | |
| CUL4A | cullin 4A | 0.65 | 201423_s_at | AL037208 | 473 | |
| TCERG1 | transcription elongation | 0.65 | 202396_at | NM_006706 | 474 | |
| | regulator 1 (CA150) | | | | | |
| HECTD1 | HECT domain containing 1 | 0.65 | 224481_s_at | BC006237 | 475 | |
| KIAA0962 | KIAA0962 protein | 0.65 | 212911_at | AB023179 | 476 | |
| KIAA0220 | hypothetical protein | 0.65 | 221501_x_at | AF229069 | 477 | |
| | LOC339047 | | | | | |
| FLJ46365 | np29d10.s1 NCI_CGAP_Pr22 | 0.649 | 235964_x_at | AA603344 | 478 | |
| | Homo sapiens cDNA clone | | | | | |
| | IMAGE: 1117747 3′ similar to | | | | | |
| | contains Alu repetitive | | | | | |
| | element; contains element L1 | | | | | |
| | repetitive element;, mRNA | | | | | |
| | sequence. | | | | | |
| VAMP3 | vesicle-associated membrane | 0.649 | 201337_s_at | NM_004781 | 479 | |
| | protein 3 (cellubrevin) | | | | | |
| MDA5 | melanoma differentiation | 0.649 | 219209_at | NM_022168 | 480 | |
| | associated protein-5 | | | | | |
| FLJ13386 | hypothetical protein | 0.649 | 222151_s_at | AK023738 | 481 | |
| | FLJ13386 | | | | | |
| ACADM | acyl-Coenzyme A | 0.649 | 202502_at | NM_000016 | 482 | |
| | dehydrogenase, C-4 to C-12 | | | | | |
| | straight chain | | | | | |
| CREBBP | CREB binding protein | 0.649 | 202160_at | NM_004380 | 483 | |
| | (Rubinstein-Taybi syndrome) | | | | | |
| DOCK11 | dedicator of cytokinesis 11 | 0.649 | 226875_at | AI742838 | 484 | |
| ANAPC5 | anaphase promoting complex | 0.649 | 239651_at | BE671583 | 485 | |
| | subunit 5 | | | | | |
| HERC1 | hect (homologous to the E6- | 0.649 | 218306_s_at | NM_003922 | 486 | |
| | AP (UBE3A) carboxyl | | | | | |
| | terminus) domain and RCC1 | | | | | |
| | (CHC1)-like domain (RLD) 1 | | | | | |
| | Clone IMAGE: 4250282, | 0.649 | 1569181_x_at | BC017896 | 487 | |
| | mRNA | | | | | |
| SETDB1 | SET domain, bifurcated 1 | 0.649 | 203155_at | NM_012432 | 488 | |
| CDA08 | T-cell immunomodulatory | 0.648 | 221449_s_at | NM_030790 | 489 | |
| | protein | | | | | |
| SLC23A2 | match: proteins: Tr: Q9Z2J0 | 0.648 | 209236_at | AL389886 | 490 | |
| | Tr: Q9UI39 Tr: Q9UHI7 | | | | | |
| | Tr: O95191 Tr: Q9WTW8 | | | | | |
| | Tr: Q9WTW7; Human DNA | | | | | |
| | sequence from clone RP1- | | | | | |
| | 237C24 on chromosome 20. | | | | | |
| | Contains the 3′ end of the | | | | | |
| | SLC23A1 gene encoding | | | | | |
| | solute carrier family 23 | | | | | |
| | (nucleobase transporters) | | | | | |
| | member 1, ESTs, STSs and | | | | | |
| | GSSs, complete sequence. | | | | | |
| OGT | O-linked N-acetylglucosamine | 0.648 | 207564_x_at | NM_003605 | 491 | |
| | (GlcNAc) transferase (UDP- | | | | | |
| | N- | | | | | |
| | acetylglucosamine:polypeptide- | | | | | |
| | N-acetylglucosaminyl | | | | | |
| | transferase) | | | | | |
| EIF5 | eukaryotic translation | 0.648 | 208290_s_at | NM_001969 | 492 | |
| | initiation factor 5 | | | | | |
| KIAA0329 | KIAA0329 | 0.648 | 204307_at | AB002295 | 493 | |
| CSNK1G3 | casein kinase 1, gamma 3 | 0.648 | 220768_s_at | NM_004384 | 494 | |
| DKFZP434C212 | DKFZP434C212 protein | 0.648 | 212804_s_at | AI797397 | 495 | |
| | Transcribed sequence with | 0.648 | 225157_at | AW245631 | 496 | |
| | weak similarity to protein | | | | | |
| | ref: NP_060312.1 (H. sapiens) | | | | | |
| | hypothetical protein | | | | | |
| | FLJ20489 [Homo sapiens ] | | | | | |
| FLJ22344 | 602362764F1 NIH_MGC_90 | 0.648 | 235740_at | BG250585 | 497 | |
| | Homo sapiens cDNA clone | | | | | |
| | IMAGE: 4471166 5′, mRNA | | | | | |
| | sequence. | | | | | |
| STK38 | serine/threonine kinase 38 | 0.648 | 1553117_a_at | NM_007271 | 498 | |
| FLJ25286 | tf96e06.x1 NCI_CGAP_CLL1 | 0.648 | 241734_at | AI391443 | 499 | |
| | Homo sapiens cDNA clone | | | | | |
| | IMAGE: 2107138 3′, mRNA | | | | | |
| | sequence. | | | | | |
| | Clone 24653 mRNA | 0.648 | 244650_at | AA581439 | 500 | |
| | sequence | | | | | |
| | CDNA FLJ10131 fis, clone | 0.648 | 215024_at | AK000993 | 501 | |
| | HEMBA1003041 | | | | | |
| ING5 | inhibitor of growth family, | 0.648 | 228287_at | BG054893 | 502 | |
| | member 5 | | | | | |
| LLT1 | lectin-like NK cell receptor | 0.647 | 220132_s_at | NM_013269 | 503 | |
| CR2 | complement component | 0.647 | 205544_s_at | NM_001877 | 504 | |
| | (3d/Epstein Barr virus) | | | | | |
| | receptor 2 | | | | | |
| EML4 | echinoderm microtubule | 0.647 | 223069_s_at | AF177377 | 505 | |
| | associated protein like 4 | | | | | |
| | Transcribed sequences | 0.647 | 236280_at | AI225238 | 506 | |
| KIAA1982 | hypothetical protein | 0.647 | 228029_at | AW513477 | 507 | |
| | FLJ14297 | | | | | |
| SFRS15 | splicing factor, | 0.647 | 222310_at | AA648521 | 508 | |
| | arginine/serine-rich 15 | | | | | |
| ADD1 | adducin 1 (alpha) | 0.647 | 214726_x_at | AL556041 | 509 | |
| LOC161527 | Transcribed sequence with | 0.647 | 213212_x_at | AI632181 | 510 | |
| | weak similarity to protein | | | | | |
| | ref: NP_061122.1 (H. sapiens) | | | | | |
| | golgin-like protein [Homo | | | | | |
| | sapiens] | | | | | |
| ARHGAP17 | RhoGAP interacting with | 0.646 | 218076_s_at | NM_018054 | 511 | |
| | CIP4 homologs 1 | | | | | |
| ZC3HDC8 | zinc finger protein | 0.646 | 223506_at | AF334161 | 512 | |
| CPR8 | cell cycle progression 8 | 0.646 | 222156_x_at | AK022459 | 513 | |
| | protein | | | | | |
| | Transcribed sequences | 0.646 | 230391_at | BF439449 | 514 | |
| MALAT-1 | PRO1073 protein | 0.646 | 224558_s_at | AI446756 | 515 | |
| MTRR | 5-methyltetrahydrofolate- | 0.646 | 203199_s_at | N29717 | 516 | |
| | homocysteine | | | | | |
| | methyltransferase reductase | | | | | |
| LOC157378 | hypothetical protein | 0.646 | 241342_at | BG288115 | 517 | |
| | BC017881 | | | | | |
| SHMT1 | serine | 0.646 | 227305_s_at | N25727 | 518 | |
| | hydroxymethyltransferase 1 | | | | | |
| | (soluble) | | | | | |
| | zh97e12.s1 | 0.646 | 237586_at | AA007336 | 519 | |
| | Soares_fetal_liver_spleen_1 | | | | | |
| | NFLS_S1 Homo sapiens | | | | | |
| | cDNA clone IMAGE: 429262 | | | | | |
| | 3′ similar to contains element | | | | | |
| | MER26 repetitive element;, | | | | | |
| | mRNA sequence. | | | | | |
| GARNL1 | GTPase activating RANGAP | 0.646 | 214855_s_at | AL050050 | 520 | |
| | domain-like 2 pseudogene | | | | | |
| TAB3 | TAK1-binding protein 3 | 0.646 | 227357_at | BF593914 | 521 | |
| USP48 | ubiquitin specific protease 31 | 0.646 | 229812_at | BE645018 | 522 | |
| SS18 | synovial sarcoma | 0.645 | 202816_s_at | AW292882 | 523 | |
| | translocation, chromosome | | | | | |
| | 18 | | | | | |
| | cong2.P5.a4 conorm Homo | 0.645 | 226341_at | AI535737 | 524 | |
| | sapiens cDNA 3′, mRNA | | | | | |
| | sequence. | | | | | |
| LARS | leucyl-tRNA synthetase | 0.645 | 222427_s_at | AK021413 | 525 | |
| | Transcribed sequences | 0.645 | 241595_at | BF223007 | 526 | |
| ADORA3 | adenosine A3 receptor | 0.645 | 206171_at | NM_000677 | 527 | |
| APG10L | APG10 autophagy 10-like (S. cerevisiae) | 0.645 | 1559820_at | BC018651 | 528 | |
| ZNF605 | hypothetical protein | 0.645 | 227822_at | AI341321 | 529 | |
| | LOC90462 | | | | | |
| EIF2C4 | eukaryotic translation | 0.645 | 227930_at | AI669957 | 530 | |
| | initiation factor 2C, 4 | | | | | |
| NPHP3 | nephronophthisis 3 | 0.645 | 235432_at | BE865779 | 531 | |
| | (adolescent) | | | | | |
| INSR | insulin receptor | 0.644 | 213792_s_at | AA485908 | 532 | |
| PRMT3 | AL551971 Homo sapiens | 0.644 | 213320_at | AL551971 | 533 | |
| | PLACENTA COT 25- | | | | | |
| | NORMALIZED Homo sapiens | | | | | |
| | cDNA clone CS0DI060YG02 | | | | | |
| | 3-PRIME, mRNA sequence. | | | | | |
| C6.1A | H. sapiens c6.1A mRNA. | 0.644 | 231913_s_at | X64643 | 534 | |
| PPP2R5C | protein phosphatase 2, | 0.644 | 1554365_a_at | BC016183 | 535 | |
| | regulatory subunit B (B56), | | | | | |
| | gamma isoform | | | | | |
| ZC3HDC7 | zinc-finger protein AY163807 | 0.644 | 218348_s_at | NM_014153 | 536 | |
| HCCR1 | cervical cancer 1 | 0.644 | 242424_at | AA345855 | 537 | |
| | protooncogene | | | | | |
| HIST1H2AG | histone 1, H2ag | 0.644 | 207156_at | NM_021064 | 538 | |
| ZNF403 | unnamed protein product; | 0.644 | 233937_at | AK024883 | 539 | |
| | Homo sapiens cDNA: | | | | | |
| | FLJ21230 fis, clone | | | | | |
| | COL00741. | | | | | |
| PTPN11 | protein tyrosine phosphatase, | 0.644 | 212610_at | U79291 | 540 | |
| | non-receptor type 11 | | | | | |
| | (Noonan syndrome 1) | | | | | |
| OAS2 | 2′-5′-oligoadenylate | 0.644 | 204972_at | NM_016817 | 541 | |
| | synthetase 2, 69/71 kDa | | | | | |
| C6orf166 | chromosome 6 open reading | 0.644 | 223143_s_at | AI742378 | 542 | |
| | frame 166 | | | | | |
| ALS2 | amyotrophic lateral sclerosis | 0.643 | 226291_at | AB046783 | 543 | |
| | 2 (juvenile) | | | | | |
| PRKXP1 | protein kinase, X-linked | 0.643 | 204061_at | NM_005044 | 544 | |
| MYO9B | myosin IXB | 0.643 | 208452_x_at | NM_004145 | 545 | |
| LOC129607 | hypothetical protein | 0.643 | 226702_at | AI742057 | 546 | |
| | LOC129607 | | | | | |
| LOC158402 | Transcribed sequences | 0.643 | 236769_at | BE504242 | 547 | |
| KIAA1856 | KIAA1856 protein | 0.643 | 226691_at | AI936523 | 548 | |
| SIAT8D | sialyltransferase 8D (alpha-2, | 0.643 | 230836_at | AI422986 | 549 | |
| | 8-polysialyltransferase) | | | | | |
| FLJ10525 | hypothetical protein | 0.643 | 218465_at | NM_018126 | 550 | |
| | FLJ10525 | | | | | |
| RAC1 | ras-related C3 botulinum | 0.642 | 1567458_s_at | AJ012502 | 551 | |
| | toxin substrate 1 (rho family, | | | | | |
| | small GTP binding protein | | | | | |
| | Rac1) | | | | | |
| MSI2 | musashi homolog 2 | 0.642 | 226134_s_at | AI978754 | 552 | |
| | (Drosophila) | | | | | |
| MTM1 | myotubular myopathy 1 | 0.642 | 36920_at | U46024 | 553 | |
| SON | SON DNA binding protein | 0.642 | 214988_s_at | X63071 | 554 | |
| | ak38h12.s1 | 0.642 | 244031_at | AA868193 | 555 | |
| | Soares_testis_NHT Homo | | | | | |
| | sapiens cDNA clone | | | | | |
| | IMAGE: 1408295 3′ similar to | | | | | |
| | contains Alu repetitive | | | | | |
| | element;, mRNA sequence. | | | | | |
| HERC4 | DKFZP564G092 protein | 0.642 | 225988_at | AI819938 | 556 | |
| TIMM17A | translocase of inner | 0.642 | 201821_s_at | BC004439 | 557 | |
| | mitochondrial membrane 17 | | | | | |
| | homolog A (yeast) | | | | | |
| EIF1AY | eukaryotic translation | 0.642 | 204410_at | NM_004681 | 558 | |
| | initiation factor 1A, Y-linked | | | | | |
| ASXL2 | additional sex combs like 2 | 0.642 | 226251_at | AW295549 | 559 | |
| | (Drosophila) | | | | | |
| PNPO | pyridoxine-5′-phosphate | 0.642 | 222653_at | AA005137 | 560 | |
| | oxidase | | | | | |
| STAT1 | | 0.642 | AFFX- | AFFX- | 561 | |
| | | | HUMISGF3A/M | HUMISGF3A/ | | |
| | | | 97935_MA_at | M97935_MA | | |
| | yd99a09.s1 Soares fetal liver | 0.641 | 244019_at | T89845 | 562 | |
| | spleen 1NFLS Homo sapiens | | | | | |
| | cDNA clone IMAGE: 116344 | | | | | |
| | 3′ similar to contains Alu | | | | | |
| | repetitive element; contains | | | | | |
| | MSR1 repetitive element;, | | | | | |
| | mRNA sequence. | | | | | |
| | Transcribed sequences | 0.641 | 235646_at | BF515595 | 563 | |
| gm117 | gm117 | 0.641 | 228135_at | AA738437 | 564 | |
| VEZATIN | transmembrane protein | 0.641 | 223675_s_at | AF216644 | 565 | |
| | vezatin | | | | | |
| MS4A6A | membrane-spanning 4- | 0.641 | 232725_s_at | BF338860 | 566 | |
| | domains, subfamily A, | | | | | |
| | member 6A | | | | | |
| TMPO | thymopoietin | 0.641 | 209753_s_at | BG391171 | 567 | |
| PB1 | polybromo 1 | 0.641 | 223400_s_at | AF197569 | 568 | |
| SNX19 | sorting nexin 19 | 0.64 | 202358_s_at | BG434168 | 569 | |
| HSPA4 | heat shock 70 kDa protein 4 | 0.64 | 211016_x_at | BC002526 | 570 | |
| DZIP3 | zinc finger DAZ interacting | 0.64 | 213186_at | BG502305 | 571 | |
| | protein 3 | | | | | |
| ATM | | 0.64 | 212672_at | U82828 | 572 | |
| IKIP | IKK interacting protein | 0.64 | 236249_at | BF057681 | 573 | |
| SF3B3 | splicing factor 3b, subunit 3, | 0.64 | 200687_s_at | NM_012426 | 574 | |
| | 130 kDa | | | | | |
| IGHM | Partial mRNA for IgM | 0.639 | 214916_x_at | BG340548 | 575 | |
| | immunoglobulin heavy chain | | | | | |
| | variable region (IGHV gene), | | | | | |
| | clone LIBPM376 | | | | | |
| SRP9 | signal recognition particle | 0.639 | 201273_s_at | NM_003133 | 576 | |
| | 9 kDa | | | | | |
| ZNF43 | zinc finger protein 43 (HTF6) | 0.639 | 206695_x_at | NM_003423 | 577 | |
| LOC54103 | hypothetical protein | 0.639 | 240061_at | AW664903 | 578 | |
| | LOC54103 | | | | | |
| C6orf130 | Human DNA sequence from | 0.639 | 213322_at | AL031778 | 579 | |
| | clone RP1-34B21 on | | | | | |
| | chromosome 6p12.1-21.1, | | | | | |
| | complete sequence. | | | | | |
| POLK | polymerase (DNA directed) | 0.639 | 223261_at | AF194973 | 580 | |
| | kappa | | | | | |
| | Transcribed sequence with | 0.639 | 228925_at | AW195586 | 581 | |
| | weak similarity to protein | | | | | |
| | ref: NP_003805.2 (H. sapiens) | | | | | |
| | a disintegrin and | | | | | |
| | metalloproteinase domain 20 | | | | | |
| | preproprotein [Homo sapiens ] | | | | | |
| C9orf91 | chromosome 9 open reading | 0.639 | 221865_at | BF969986 | 582 | |
| | frame 91 | | | | | |
| CWF19L2 | CWF19-like 2, cell cycle | 0.639 | 228916_at | BE857467 | 583 | |
| | control (S. pombe) | | | | | |
| | Transcribed sequences | 0.639 | 242983_at | AI806626 | 584 | |
| CDKN1C | cyclin-dependent kinase | 0.639 | 213183_s_at | N95363 | 585 | |
| | inhibitor 1C (p57, Kip2) | | | | | |
| NUMB | numb homolog (Drosophila) | 0.639 | 209073_s_at | AF015040 | 586 | |
| GATA2 | GATA binding protein 2 | 0.639 | 209710_at | AL563460 | 587 | |
| HSPC121 | butyrate-induced transcript 1 | 0.638 | 217777_s_at | NM_016395 | 588 | |
| ZCWCC3 | Homo sapiens genomic DNA, | 0.638 | 213000_at | AP000693 | 589 | |
| | chromosome 21q22.2, BAC | | | | | |
| | clone: KB739C11, CBR1- | | | | | |
| | HLCS region. | | | | | |
| TTF1 | transcription termination | 0.638 | 204772_s_at | NM_007344 | 590 | |
| | factor, RNA polymerase I | | | | | |
| API5 | apoptosis inhibitor 5 | 0.638 | 214959_s_at | AF229253 | 591 | |
| SLC26A2 | solute carrier family 26 | 0.638 | 224959_at | AI718385 | 592 | |
| | (sulfate transporter), member 2 | | | | | |
| TNRC15 | trinucleotide repeat | 0.638 | 212260_at | AL045800 | 593 | |
| | containing 15 | | | | | |
| SMBP | SM-11044 binding protein | 0.638 | 217758_s_at | NM_020123 | 594 | |
| ZNF207 | zinc finger protein 207 | 0.638 | 228157_at | AI125646 | 595 | |
| MAP4K4 | hypothetical protein | 0.638 | 222547_at | AL561281 | 596 | |
| | FLJ20373 | | | | | |
| ABCE1 | ATP-binding cassette, sub- | 0.638 | 201873_s_at | NM_002940 | 597 | |
| | family E (OABP), member 1 | | | | | |
| BTBD11 | BTB (POZ) domain | 0.638 | 238692_at | AL040935 | 598 | |
| | containing 11 | | | | | |
| | cong2.P5.a1 conorm Homo | 0.638 | 242563_at | AI535736 | 599 | |
| | sapiens cDNA 3′, mRNA | | | | | |
| | sequence. | | | | | |
| GTPBP4 | GTP binding protein 4 | 0.637 | 218239_s_at | NM_012341 | 600 | |
| FLJ23047 | AL559474 Homo sapiens T | 0.637 | 235918_x_at | AL559474 | 601 | |
| | CELLS (JURKAT CELL LINE) | | | | | |
| | COT 10-NORMALIZED | | | | | |
| | Homo sapiens cDNA clone | | | | | |
| | CS0DJ013YA17 5-PRIME, | | | | | |
| | mRNA sequence. | | | | | |
| SIN3B | SIN3 homolog B, | 0.637 | 209352_s_at | AB014600 | 602 | |
| | transcriptional regulator | | | | | |
| | (yeast) | | | | | |
| ANKRD27 | ankyrin repeat domain 27 | 0.637 | 221522_at | AL136784 | 603 | |
| | (VPS9 domain) | | | | | |
| FREB | Fc receptor homolog | 0.637 | 235400_at | AL560266 | 604 | |
| | expressed in B cells | | | | | |
| HPS3 | Hermansky-Pudlak syndrome 3 | 0.637 | 238539_at | BG163294 | 605 | |
| CETN3 | centrin, EF-hand protein, 3 | 0.637 | 209662_at | BC005383 | 606 | |
| | (CDC31 homolog, yeast) | | | | | |
| C21orf86 | AV705934 ADB Homo | 0.637 | 226995_at | AV705934 | 607 | |
| | sapiens cDNA clone | | | | | |
| | ADBDCE04 5′, mRNA | | | | | |
| | sequence. | | | | | |
| BRD1 | bromodomain containing 1 | 0.637 | 204520_x_at | NM_014577 | 608 | |
| C20orf19 | chromosome 20 open reading | 0.637 | 228291_s_at | AI806322 | 609 | |
| | frame 19 | | | | | |
| | CDNA FLJ33037 fis, clone | 0.636 | 1557477_at | BQ022900 | 610 | |
| | THYMU2000317 | | | | | |
| | Transcribed sequences | 0.636 | 230383_x_at | AA133285 | 611 | |
| ELMO1 | engulfment and cell motility 1 | 0.636 | 204513_s_at | NM_014800 | 612 | |
| | (ced-12 homolog, C. elegans) | | | | | |
| MOBKL2A | MOB1, Mps One Binder | 0.636 | 235163_at | BE311936 | 613 | |
| | kinase activator-like 2A | | | | | |
| | (yeast) | | | | | |
| CRK7 | Transcribed sequences | 0.636 | 213557_at | AW305119 | 614 | |
| STCH | stress 70 protein chaperone, | 0.636 | 202557_at | AI718418 | 615 | |
| | microsome-associated, | | | | | |
| | 60 kDa | | | | | |
| C6orf68 | Similar to hypothetical | 0.636 | 215207_x_at | BF695847 | 616 | |
| | protein, MGC: 7199 | | | | | |
| | (LOC389850), mRNA | | | | | |
| PNN | pinin, desmosome associated | 0.636 | 1567214_a_at | U59479 | 617 | |
| | protein | | | | | |
| KIAA0436 | putative L-type neutral amino | 0.636 | 212216_at | AW000954 | 618 | |
| | acid transporter | | | | | |
| ZNF21 | zinc finger protein 21 (KOX | 0.636 | 235810_at | AI225224 | 619 | |
| | 14) | | | | | |
| ARL1 | ADP-ribosylation factor-like 1 | 0.636 | 201659_s_at | NM_001177 | 620 | |
| PUM1 | pumilio homolog 1 | 0.636 | 201165_s_at | BE670915 | 621 | |
| | (Drosophila) | | | | | |
| USP15 | ubiquitin specific protease 15 | 0.636 | 210681_s_at | AF153604 | 622 | |
| ATP6V1C1 | ATPase, H+ transporting, | 0.635 | 202872_at | AW024925 | 623 | |
| | lysosomal 42 kDa, V1 subunit | | | | | |
| | C, isoform 1 | | | | | |
| EPB41L3 | erythrocyte membrane | 0.635 | 211776_s_at | BC006141 | 624 | |
| | protein band 4.1-like 3 | | | | | |
| TRIM37 | tripartite motif-containing 37 | 0.635 | 213009_s_at | AK022701 | 625 | |
| TNFRSF6 | tumor necrosis factor receptor | 0.635 | 216252_x_at | Z70519 | 626 | |
| | superfamily, member 6 | | | | | |
| C6orf201 | Similar to RIKEN cDNA | 0.635 | 242739_at | AA121544 | 627 | |
| | 4933417A18 gene, clone | | | | | |
| | IMAGE: 5266746, mRNA | | | | | |
| LOC51136 | PTD016 protein | 0.635 | 227268_at | N51514 | 628 | |
| DCTN6 | dynactin 6 | 0.635 | 203261_at | NM_006571 | 629 | |
| VPS54 | vacuolar protein sorting 54 | 0.635 | 218423_x_at | NM_016516 | 630 | |
| | (yeast) | | | | | |
| DKFZp434L142 | hypothetical protein | 0.635 | 219872_at | NM_016613 | 631 | |
| | DKFZp434L142 | | | | | |
| TRIM4 | tripartite motif-containing 4 | 0.635 | 223384_s_at | BE501464 | 632 | |
| | Clone IMAGE: 5269868, | 0.635 | 1556732_at | BC039354 | 633 | |
| | mRNA | | | | | |
| | Clone IMAGE: 238558, mRNA | 0.635 | 230688_at | AI631029 | 634 | |
| | sequence | | | | | |
| C21orf7 | chromosome 21 open reading | 0.635 | 221211_s_at | NM_020152 | 635 | |
| | frame 7 | | | | | |
| ZNF333 | zinc finger protein 333 | 0.634 | 1569251_a_at | BC040378 | 636 | |
| FLJ23091 | putative NFkB activating | 0.634 | 228949_at | AL534095 | 637 | |
| | protein 373 | | | | | |
| SCARB2 | scavenger receptor class B, | 0.634 | 201646_at | AA885297 | 638 | |
| | member 2 | | | | | |
| DIP13B | DIP13 beta | 0.634 | 218218_at | NM_018171 | 639 | |
| EMR1 | egf-like module containing, | 0.634 | 207111_at | NM_001974 | 640 | |
| | mucin-like, hormone receptor- | | | | | |
| | like 1 | | | | | |
| PIGE | phosphatidylinositol glycan, | 0.634 | 212120_at | BE897886 | 641 | |
| | class F | | | | | |
| STX16 | syntaxin 16 | 0.634 | 221638_s_at | AF008937 | 642 | |
| CNOT7 | CCR4-NOT transcription | 0.634 | 1552344_s_at | NM_054026 | 643 | |
| | complex, subunit 7 | | | | | |
| HNRPR | heterogeneous nuclear | 0.634 | 208766_s_at | BC001449 | 644 | |
| | ribonucleoprotein R | | | | | |
| IGHG1 | Partial mRNA for IgM | 0.633 | 211908_x_at | M87268 | 645 | |
| | immunoglobulin heavy chain | | | | | |
| | variable region (IGHV gene), | | | | | |
| | clone LIBPM376 | | | | | |
| | Transcribed sequences | 0.633 | 242273_at | AA747287 | 646 | |
| RPS6KA2 | ribosomal protein S6 kinase, | 0.633 | 212912_at | AI992251 | 647 | |
| | 90 kDa, polypeptide 2 | | | | | |
| | Transcribed sequence with | 0.633 | 235847_at | BF111312 | 648 | |
| | moderate similarity to protein | | | | | |
| | ref: NP_060265.1 (H. sapiens) | | | | | |
| | hypothetical protein | | | | | |
| | FLJ20378 [Homo sapiens ] | | | | | |
| UNQ1912 | HGS_RE408 | 0.633 | 226752_at | AI816071 | 649 | |
| KIAA1407 | KIAA1407 protein | 0.633 | 228476_at | AW193515 | 650 | |
| SGPP1 | sphingosine-1-phosphate | 0.633 | 223391_at | BE880703 | 651 | |
| | phosphatase 1 | | | | | |
| | Transcribed sequences | 0.633 | 237383_at | AI768144 | 652 | |
| USP16 | ubiquitin specific protease 16 | 0.633 | 218386_x_at | NM_006447 | 653 | |
| MGC4189 | hypothetical protein | 0.633 | 1556088_at | AK098491 | 654 | |
| | MGC4189 | | | | | |
| HNRPU | heterogeneous nuclear | 0.633 | 236244_at | AI458297 | 655 | |
| | ribonucleoprotein U (scaffold | | | | | |
| | attachment factor A) | | | | | |
| CGI-40 | transgelin | 0.633 | 56256_at | AA150165 | 656 | |
| | MRNA; cDNA | 0.633 | 227451_s_at | BF507383 | 657 | |
| | DKFZp686A1197 (from clone | | | | | |
| | DKFZp686A1197) | | | | | |
| OXR1 | oxidation resistance 1 | 0.632 | 223879_s_at | AF309387 | 658 | |
| KIAA1826 | KIAA1826 protein | 0.632 | 227418_at | AI808746 | 659 | |
| LKAP | limkain b1 | 0.632 | 1558697_a_at | BI600341 | 660 | |
| BCOR | BCL6 co-repressor | 0.632 | 219433_at | NM_017745 | 661 | |
| MLL5 | myeloid/lymphoid or mixed- | 0.632 | 226100_at | AI762876 | 662 | |
| | lineage leukemia 5 (trithorax | | | | | |
| | homolog, Drosophila) | | | | | |
| LPIN2 | lipin 2 | 0.632 | 202459_s_at | U55968 | 663 | |
| SRPK2 | SFRS protein kinase 2 | 0.632 | 230091_at | AA046241 | 664 | |
| C5orf3 | chromosome 5 open reading | 0.632 | 222682_s_at | AI307615 | 665 | |
| | frame 3 | | | | | |
| | CDNA: FLJ21350 fis, clone | 0.632 | 215252_at | AW814026 | 666 | |
| | COL02751 | | | | | |
| TXNDC | thioredoxin domain containing | 0.632 | 208097_s_at | NM_030755 | 667 | |
| | 602464219F1 NIH_MGC_75 | 0.631 | 1565689_at | BG400570 | 668 | |
| | Homo sapiens cDNA clone | | | | | |
| | IMAGE: 4592261 5′, mRNA | | | | | |
| | sequence. | | | | | |
| C20orf36 | Homo sapiens mRNA for | 0.631 | 212406_s_at | AB028973 | 669 | |
| | KIAA1050 protein, partial cds. | | | | | |
| LOC158301 | far upstream element (FUSE) | 0.631 | 239193_at | BF060981 | 670 | |
| | binding protein 3 | | | | | |
| NSG-X | staphylococcal nuclease | 0.631 | 210109_at | AF191492 | 671 | |
| | domain containing 1 | | | | | |
| | Clone IMAGE: 5289004, | 0.631 | 224862_at | BF969428 | 672 | |
| | mRNA | | | | | |
| SARA1 | SAR1a gene homolog 1 (S. cerevisiae) | 0.631 | 210790_s_at | BC003658 | 673 | |
| KIAA1374 | KIAA1374 protein | 0.631 | 226098_at | AB037795 | 674 | |
| TJP2 | tight junction protein 2 (zona | 0.631 | 202085_at | NM_004817 | 675 | |
| | occludens 2) | | | | | |
| EIF2C4 | eukaryotic translation | 0.631 | 219190_s_at | NM_017629 | 676 | |
| | initiation factor 2C, 4 | | | | | |
| ZCCHC2 | zinc finger, CCHC domain | 0.631 | 219062_s_at | NM_017742 | 677 | |
| | containing 2 | | | | | |
| FBXL11 | 7f17b04.x1 NCI_CGAP_CLL1 | 0.631 | 208988_at | BE675843 | 678 | |
| | Homo sapiens cDNA clone | | | | | |
| | IMAGE: 3294895 3′, mRNA | | | | | |
| | sequence. | | | | | |
| ATF1 | activating transcription factor 1 | 0.631 | 222103_at | AI434345 | 679 | |
| SFRS8 | splicing factor, | 0.63 | 202773_s_at | AI023864 | 680 | |
| | arginine/serine-rich 8 | | | | | |
| | (suppressor-of-white-apricot | | | | | |
| | homolog, Drosophila) | | | | | |
| CDKN1C | cyclin-dependent kinase | 0.63 | 213348_at | N33167 | 681 | |
| | inhibitor 1C (p57, Kip2) | | | | | |
| SNX10 | sorting nexin 10 | 0.63 | 218404_at | NM_013322 | 682 | |
| TGOLN2 | trans-golgi network protein 2 | 0.63 | 212043_at | W72053 | 683 | |
| SCYE1 | small inducible cytokine | 0.63 | 202541_at | BF589679 | 684 | |
| | subfamily E, member 1 | | | | | |
| | (endothelial monocyte- | | | | | |
| | activating) | | | | | |
| | Transcribed sequence with | 0.63 | 239162_at | H04394 | 685 | |
| | weak similarity to protein | | | | | |
| | ref: NP_060312.1 (H. sapiens) | | | | | |
| | hypothetical protein | | | | | |
| | FLJ20489 [Homo sapiens ] | | | | | |
| BIRC3 | IAP homolog C; interacts with | 0.63 | 210538_s_at | U37546 | 686 | |
| | TRAF1 and TRAF2 in yeast | | | | | |
| | two hybrid system; homolog | | | | | |
| | of Baculovirus IAP genes; | | | | | |
| | Mammalian IAP homolog C; | | | | | |
| | Human IAP homolog C; | | | | | |
| | (MIHC) mRNA, complete cds. | | | | | |
| KIAA1276 | yz86g08.s1 | 0.63 | 227505_at | N64630 | 687 | |
| | Soares_multiple_sclerosis_2 | | | | | |
| | NbHMSP Homo sapiens | | | | | |
| | cDNA clone IMAGE: 289982 | | | | | |
| | 3′, mRNA sequence. | | | | | |
| RELA | v-rel reticuloendotheliosis | 0.629 | 209878_s_at | M62399 | 688 | |
| | viral oncogene homolog A, | | | | | |
| | nuclear factor of kappa light | | | | | |
| | polypeptide gene enhancer in | | | | | |
| | B-cells 3, p65 (avian) | | | | | |
| CDC2L5 | cell division cycle 2-like 5 | 0.629 | 228991_at | BF707423 | 689 | |
| | (cholinesterase-related cell | | | | | |
| | division controller) | | | | | |
| CYP4V2 | hypothetical protein | 0.629 | 228391_at | AI916528 | 690 | |
| | LOC285440 | | | | | |
| ELK3 | ELK3, ETS-domain protein | 0.629 | 221773_at | AW575374 | 691 | |
| | (SRF accessory protein 2) | | | | | |
| ZNF262 | zinc finger protein 262 | 0.629 | 202050_s_at | AI650586 | 692 | |
| | CDNA FLJ33653 fis, clone | 0.629 | 228328_at | AI982758 | 693 | |
| | BRAMY2024715 | | | | | |
| LOC90826 | hypothetical protein | 0.629 | 228536_at | AA574240 | 694 | |
| | BC004337 | | | | | |
| SPAG1 | sperm associated antigen 1 | 0.629 | 210117_at | AF311312 | 695 | |
| FLJ13576 | hypothetical protein | 0.628 | 218962_s_at | NM_022484 | 696 | |
| | FLJ13576 | | | | | |
| KIAA0528 | KIAA0528 gene product | 0.628 | 212943_at | AB011100 | 697 | |
| C14orf103 | chromosome 14 open reading | 0.628 | 219164_s_at | NM_018036 | 698 | |
| | frame 103 | | | | | |
| CDA11 | CDA11 protein | 0.628 | 223231_at | AF212250 | 699 | |
| FLJ39370 | hypothetical protein | 0.628 | 227856_at | AI110850 | 700 | |
| | FLJ39370 | | | | | |
| | Clone IMAGE: 4797534, | 0.628 | 1560271_at | BC030757 | 701 | |
| | mRNA, partial cds | | | | | |
| XTP2 | HBxAg transactivated protein 2 | 0.628 | 230618_s_at | BF110903 | 702 | |
| PPP1R9B | mitochondrial ribosomal | 0.628 | 225124_at | BF508705 | 703 | |
| | protein L39 | | | | | |
| BZW1 | Similar to basic leucine zipper | 0.628 | 200776_s_at | AL518328 | 704 | |
| | and W2 domains 1 | | | | | |
| | (LOC151579), mRNA | | | | | |
| TOB1 | transducer of ERBB2, 1 | 0.628 | 202704_at | AA675892 | 705 | |
| CRI1 | CREBBP/EP300 inhibitory | 0.628 | 208669_s_at | AF109873 | 706 | |
| | protein 1 | | | | | |
| DNAJB6 | DnaJ (Hsp40) homolog, | 0.628 | 208811_s_at | AF080569 | 707 | |
| | subfamily B, member 6 | | | | | |
| SSB | Sjogren syndrome antigen B | 0.627 | 201139_s_at | NM_003142 | 708 | |
| | (autoantigen La) | | | | | |
| RANBP9 | RAN binding protein 9 | 0.627 | 202582_s_at | AF306510 | 709 | |
| C6orf211 | chromosome 6 open reading | 0.627 | 218195_at | NM_024573 | 710 | |
| | frame 211 | | | | | |
| KIAA0492 | MRNA, chromosome 1 | 0.627 | 215109_at | R02172 | 711 | |
| | specific transcript KIAA0492. | | | | | |
| TNFRSF6 | tumor necrosis factor receptor | 0.627 | 215719_x_at | X83493 | 712 | |
| | superfamily, member 6 | | | | | |
| FLJ11151 | hypothetical protein | 0.627 | 239135_at | AI675054 | 713 | |
| | FLJ11151 | | | | | |
| | Transcribed sequence with | 0.627 | 242539_at | AW665509 | 714 | |
| | weak similarity to protein | | | | | |
| | ref: NP_110386.1 (H. sapiens) | | | | | |
| | nuclear receptor binding | | | | | |
| | factor-2 [Homo sapiens ] | | | | | |
| | CDNA FLJ32162 fis, clone | 0.627 | 229342_at | AI708256 | 715 | |
| | PLACE6000325 | | | | | |
| FLJ14753 | hypothetical protein | 0.626 | 211185_s_at | AF130099 | 716 | |
| | FLJ14753 | | | | | |
| PLEKHF2 | pleckstrin homology domain | 0.626 | 218640_s_at | NM_024613 | 717 | |
| | containing, family F (with | | | | | |
| | FYVE domain) member 2 | | | | | |
| MPHOSPH9 | M-phase phosphoprotein 9 | 0.626 | 237158_s_at | AW449069 | 718 | |
| RDH11 | retinol dehydrogenase 11 (all- | 0.626 | 217776_at | AF167438 | 719 | |
| | trans and 9-cis) | | | | | |
| SMBP | SM-11044 binding protein | 0.626 | 222399_s_at | BG104571 | 720 | |
| | EST48728 Fetal spleen | 0.626 | 234997_x_at | AA343057 | 721 | |
| | Homo sapiens cDNA 3′ end, | | | | | |
| | mRNA sequence. | | | | | |
| CBLL1 | Cas-Br-M (murine) ecotropic | 0.626 | 227187_at | AI824009 | 722 | |
| | retroviral transforming | | | | | |
| | sequence-like 1 | | | | | |
| CLK4 | CDC-like kinase 4 | 0.626 | 228751_at | AW975057 | 723 | |
| ABAT | 4-aminobutyrate | 0.626 | 209459_s_at | AF237813 | 724 | |
| | aminotransferase | | | | | |
| TLR10 | toll-like receptor 10 | 0.626 | 223750_s_at | AW665250 | 725 | |
| VEZATIN | transmembrane protein | 0.626 | 223090_x_at | AF225417 | 726 | |
| | vezatin | | | | | |
| FLJ13611 | hypothetical protein | 0.625 | 218674_at | NM_024941 | 727 | |
| | FLJ13611 | | | | | |
| DKFZp434I1610 | hypothetical protein | 0.625 | 223590_at | AL136732 | 728 | |
| | DKFZp434I1610 | | | | | |
| DLEU2 | deleted in lymphocytic | 0.625 | 242854_x_at | AW340891 | 729 | |
| | leukemia, 2 | | | | | |
| PPM2C | protein phosphatase 2C, | 0.625 | 222572_at | BG542521 | 730 | |
| | magnesium-dependent, | | | | | |
| | catalytic subunit | | | | | |
| C9orf102 | RAD26L hypothetical protein, | 0.625 | 232801_at | BG534457 | 731 | |
| | alternatively spliced product; | | | | | |
| | similar to (AF217319) | | | | | |
| | putative repair and | | | | | |
| | recombination helicase | | | | | |
| | RAD26L [Mus musculus] | | | | | |
| BNIP2 | BCL2/adenovirus E1B 19 kDa | 0.625 | 209308_s_at | BC002461 | 732 | |
| | interacting protein 2 | | | | | |
| PRKWNK1 | protein kinase, lysine | 0.624 | 39313_at | AB002342 | 733 | |
| | deficient 1 | | | | | |
| IFI44 | interferon-induced protein 44 | 0.624 | 214453_s_at | NM_006417 | 734 | |
| HSRG1 | HSV-1 stimulation-related | 0.624 | 227116_at | AI934828 | 735 | |
| | gene 1 | | | | | |
| NP220 | NP220 nuclear protein | 0.624 | 231471_at | BF438058 | 736 | |
| KIAA0494 | KIAA0494 gene product | 0.624 | 201775_s_at | AA676790 | 737 | |
| TIF1 | transcriptional intermediary | 0.624 | 213301_x_at | AL538264 | 738 | |
| | factor 1 | | | | | |
| | Transcribed sequence with | 0.624 | 239164_at | BE674896 | 739 | |
| | weak similarity to protein | | | | | |
| | ref: NP_060265.1 (H. sapiens) | | | | | |
| | hypothetical protein | | | | | |
| | FLJ20378 [Homo sapiens ] | | | | | |
| CAPZA2 | capping protein (actin | 0.624 | 201238_s_at | BC005338 | 740 | |
| | filament) muscle Z-line, alpha 2 | | | | | |
| | CDNA FLJ41270 fis, clone | 0.624 | 228528_at | AI927692 | 741 | |
| | BRAMY2036387 | | | | | |
| HELIC1 | helicase, ATP binding 1 | 0.624 | 212815_at | AA156961 | 742 | |
| PFAAP5 | phosphonoformate immuno- | 0.624 | 202258_s_at | U50532 | 743 | |
| | associated protein 5 | | | | | |
| CASP1 | caspase 1, apoptosis-related | 0.624 | 211368_s_at | U13700 | 744 | |
| | cysteine protease (interleukin | | | | | |
| | 1, beta, convertase) | | | | | |
| RBBP6 | retinoblastoma binding | 0.624 | 205178_s_at | NM_006910 | 745 | |
| | protein 6 | | | | | |
| IFIT2 | interferon-induced protein | 0.623 | 226757_at | AA131041 | 746 | |
| | with tetratricopeptide repeats 2 | | | | | |
| BLOC1S2 | 601815191F1 NIH_MGC_56 | 0.623 | 225049_at | BF382281 | 747 | |
| | Homo sapiens cDNA clone | | | | | |
| | IMAGE: 4048918 5′, mRNA | | | | | |
| | sequence. | | | | | |
| C14orf43 | chromosome 14 open reading | 0.623 | 225980_at | AV740879 | 748 | |
| | frame 43 | | | | | |
| YWHAZ | tyrosine 3- | 0.623 | 200641_s_at | U28964 | 749 | |
| | monooxygenase/tryptophan | | | | | |
| | 5-monooxygenase activation | | | | | |
| | protein, zeta polypeptide | | | | | |
| SSR3 | xg42b08.x1 NCI_CGAP_Ut1 | 0.623 | 222412_s_at | AW150923 | 750 | |
| | Homo sapiens cDNA clone | | | | | |
| | IMAGE: 2630199 3′ similar to | | | | | |
| | contains Alu repetitive | | | | | |
| | element;, mRNA sequence. | | | | | |
| SNAPC3 | small nuclear RNA activating | 0.623 | 229712_at | AI066599 | 751 | |
| | complex, polypeptide 3, | | | | | |
| | 50 kDa | | | | | |
| GRCC10 | | 0.623 | 203547_at | U47924 | 752 | |
| CHD2 | chromodomain helicase DNA | 0.623 | 1554015_a_at | BC007347 | 753 | |
| | binding protein 2 | | | | | |
| KIAA0092 | translokin | 0.623 | 203493_s_at | AL525206 | 754 | |
| KIAA0971 | Homo sapiens KIAA0971 | 0.623 | 205976_at | NM_014929 | 755 | |
| | protein (KIAA0971), mRNA. | | | | | |
| GANAB | alpha glucosidase II alpha | 0.623 | 211934_x_at | W87689 | 756 | |
| | subunit | | | | | |
| AFTIPHILIN | aftiphilin protein | 0.623 | 222472_at | BF055271 | 757 | |
| | UI-H-BW0-aiy-a-04-0-UI.s1 | 0.623 | 235157_at | AW297731 | 758 | |
| | NCI_CGAP_Sub6 Homo | | | | | |
| | sapiens cDNA clone | | | | | |
| | IMAGE: 2730894 3′, mRNA | | | | | |
| | sequence. | | | | | |
| | Human mRNA sequence. | 0.622 | 236510_at | N70015 | 759 | |
| RNF146 | ring finger protein 146 | 0.622 | 223886_s_at | AL136829 | 760 | |
| TTC3 | tetratricopeptide repeat | 0.622 | 208661_s_at | AW510696 | 761 | |
| | domain 3 | | | | | |
| PICALM | phosphatidylinositol binding | 0.622 | 215832_x_at | AV722190 | 762 | |
| | clathrin assembly protein | | | | | |
| LRAP | leukocyte-derived arginine | 0.622 | 1554273_a_at | BC017927 | 763 | |
| | aminopeptidase | | | | | |
| C20orf72 | chromosome 20 open reading | 0.622 | 225890_at | AI678096 | 764 | |
| | frame 72 | | | | | |
| | zinc finger, DHHC domain | 0.622 | 1563502_at | AL833080 | 765 | |
| | containing 2 | | | | | |
| | Clone IMAGE: 5550275, | 0.622 | 1562505_at | BC035700 | 766 | |
| | mRNA | | | | | |
| MFN1 | mitofusin 1 | 0.622 | 211801_x_at | AF329637 | 767 | |
| TRIP12 | thyroid hormone receptor | 0.622 | 244659_at | AL120025 | 768 | |
| | interactor 12 | | | | | |
| RNF44 | synonym: KIAA1100; Homo | 0.621 | 203286_at | NM_014901 | 769 | |
| | sapiens ring finger protein 44 | | | | | |
| | (RNF44), mRNA. | | | | | |
| SNRK | Homo sapiens HSNFRK | 0.621 | 209481_at | AF226044 | 770 | |
| | (HSNFRK) mRNA, complete | | | | | |
| | cds. | | | | | |
| | CDNA FLJ42915 fis, clone | 0.621 | 240036_at | AI022082 | 771 | |
| | BRHIP3025702 | | | | | |
| FBXL10 | F-box and leucine-rich repeat | 0.621 | 226215_s_at | AI989996 | 772 | |
| | protein 10 | | | | | |
| HECA | headcase homolog | 0.621 | 218603_at | NM_016217 | 773 | |
| | (Drosophila) | | | | | |
| | Transcribed sequences | 0.621 | 241242_at | BE503118 | 774 | |
| SDCCAG1 | serologically defined colon | 0.62 | 1557950_at | BF984340 | 775 | |
| | cancer antigen 1 | | | | | |
| HSA277841 | ELG protein | 0.62 | 218896_s_at | NM_018553 | 776 | |
| DKFZp434L142 | hypothetical protein | 0.62 | 223204_at | AF260333 | 777 | |
| | DKFZp434L142 | | | | | |
| SMARCC1 | SWI/SNF related, matrix | 0.62 | 201075_s_at | NM_003074 | 778 | |
| | associated, actin dependent | | | | | |
| | regulator of chromatin, | | | | | |
| | subfamily c, member 1 | | | | | |
| FLJ10803 | | 0.62 | 209446_s_at | BC001743 | 779 | |
| PCNP | PEST-containing nuclear | 0.62 | 1554868_s_at | BC013916 | 780 | |
| | protein | | | | | |
| LNK | lymphocyte adaptor protein | 0.619 | 203320_at | NM_005475 | 781 | |
| HIPK2 | xs45a11.x1 | 0.619 | 225116_at | AW300045 | 782 | |
| | NCI_CGAP_Kid11 Homo | | | | | |
| | sapiens cDNA clone | | | | | |
| | IMAGE: 2772572 3′, mRNA | | | | | |
| | sequence. | | | | | |
| UBE4B | ubiquitination factor E4B | 0.619 | 215533_s_at | AF091093 | 783 | |
| | (UFD2 homolog, yeast) | | | | | |
| RAP1A | RAP1A, member of RAS | 0.619 | 202362_at | NM_002884 | 784 | |
| | oncogene family | | | | | |
| BIRC4 | baculoviral IAP repeat- | 0.619 | 228363_at | BF109251 | 785 | |
| | containing 4 | | | | | |
| MYO5A | myosin VA (heavy | 0.619 | 227761_at | AW235548 | 786 | |
| | polypeptide 12, myoxin) | | | | | |
| LOC92017 | similar to RIKEN cDNA | 0.619 | 1558745_at | BC029857 | 787 | |
| | 4933437K13 | | | | | |
| STAM | signal transducing adaptor | 0.619 | 203544_s_at | NM_003473 | 788 | |
| | molecule (SH3 domain and | | | | | |
| | ITAM motif) 1 | | | | | |
| CDC40 | cell division cycle 40 homolog | 0.619 | 203377_s_at | NM_015891 | 789 | |
| | (yeast) | | | | | |
| DENR | density-regulated protein | 0.619 | 221509_at | AB014731 | 790 | |
| MRPL35 | mitochondrial ribosomal | 0.619 | 218890_x_at | NM_016622 | 791 | |
| | protein L35 | | | | | |
| FLJ12969 | hypothetical protein | 0.619 | 219335_at | NM_022838 | 792 | |
| | FLJ12969 | | | | | |
| MEF2A | MADS box transcription | 0.619 | 214684_at | X63381 | 793 | |
| | enhancer factor 2, | | | | | |
| | polypeptide A (myocyte | | | | | |
| | enhancer factor 2A) | | | | | |
| KIAA0423 | KIAA0423 | 0.619 | 213304_at | AI823592 | 794 | |
| ANKRD12 | ankyrin repeat domain 12 | 0.619 | 216550_x_at | X80821 | 795 | |
| CD22 | CD22 antigen | 0.619 | 217422_s_at | X52785 | 796 | |
| KIAA1434 | hypothetical protein | 0.618 | 224826_at | AK001947 | 797 | |
| | KIAA1434 | | | | | |
| HIP14 | huntingtin interacting protein | 0.618 | 217486_s_at | AF161412 | 798 | |
| | 14 | | | | | |
| | CDNA FLJ36584 fis, clone | 0.618 | 226381_at | AW450329 | 799 | |
| | TRACH2013450 | | | | | |
| KIAA0924 | KIAA0924 protein | 0.618 | 243495_s_at | AL036450 | 800 | |
| KIAA1731 | KIAA1731 protein | 0.618 | 215018_at | AB051518 | 801 | |
| EPRS | glutamyl-prolyl-tRNA | 0.617 | 200841_s_at | AI142677 | 802 | |
| | synthetase | | | | | |
| STK4 | serine/threonine kinase 4 | 0.617 | 236259_at | BF433725 | 803 | |
| COL4A3BP | collagen, type IV, alpha 3 | 0.617 | 226277_at | AA889952 | 804 | |
| | (Goodpasture antigen) | | | | | |
| | binding protein | | | | | |
| hIAN7 | immune associated | 0.617 | 228071_at | AA858297 | 805 | |
| | nucleotide | | | | | |
| SLC7A6 | solute carrier family 7 | 0.617 | 203578_s_at | BG230586 | 806 | |
| | (cationic amino acid | | | | | |
| | transporter, y+ system), | | | | | |
| | member 6 | | | | | |
| ABHD3 | AL534702 Homo sapiens | 0.617 | 213017_at | AL534702 | 807 | |
| | FETAL BRAIN Homo sapiens | | | | | |
| | cDNA clone CS0DF006YN17 | | | | | |
| | 3-PRIME, mRNA sequence. | | | | | |
| AHSA2 | AHA1, activator of heat shock | 0.617 | 212980_at | AL050376 | 808 | |
| | 90 kDa protein ATPase | | | | | |
| | homolog 2 (yeast) | | | | | |
| HOZFP | ovarian zinc finger protein | 0.617 | 227220_at | AI743731 | 809 | |
| MTSS1 | metastasis suppressor 1 | 0.617 | 203037_s_at | NM_014751 | 810 | |
| | Transcribed sequences | 0.617 | 239809_at | BF197708 | 811 | |
| | Clone IMAGE: 5300069, | 0.617 | 1562033_at | BC041918 | 812 | |
| | mRNA | | | | | |
| KPNA1 | karyopherin alpha 1 (importin | 0.616 | 202058_s_at | BC002374 | 813 | |
| | alpha 5) | | | | | |
| NY-SAR-41 | Human DNA sequence from | 0.616 | 232362_at | AL139421 | 814 | |
| | clone RP4-717I23 on | | | | | |
| | chromosome 1p21.2-22.3, | | | | | |
| | complete sequence. | | | | | |
| SWAP70 | SWAP-70 protein | 0.616 | 209306_s_at | AI139569 | 815 | |
| ENTH | enthoprotin | 0.616 | 201769_at | NM_014666 | 816 | |
| KIAA0182 | KIAA0182 protein | 0.616 | 212056_at | D80004 | 817 | |
| ANKHD1 | multiple ankyrin repeats, | 0.616 | 208773_s_at | AL136943 | 818 | |
| | single KH-domain (MASK) | | | | | |
| | homolog | | | | | |
| C14orf139 | chromosome 14 open reading | 0.615 | 219563_at | NM_024633 | 819 | |
| | frame 139 | | | | | |
| RASSF5 | Ras association (RaIGDS/AF- | 0.615 | 1554834_a_at | AY062002 | 820 | |
| | 6) domain family 5 | | | | | |
| NP220 | NP220 nuclear protein | 0.615 | 213775_x_at | AI357871 | 821 | |
| ECHDC1 | enoyl Coenzyme A hydratase | 0.615 | 219974_x_at | NM_018479 | 822 | |
| | domain containing 1 | | | | | |
| CG018 | hypothetical gene CG018 | 0.615 | 217197_x_at | AL049785 | 823 | |
| | Transcribed sequences | 0.615 | 229692_at | AW135003 | 824 | |
| TAF1 | TAF1 RNA polymerase II, | 0.615 | 227205_at | AW575233 | 825 | |
| | TATA box binding protein | | | | | |
| | (TBP)-associated factor, | | | | | |
| | 250 kDa | | | | | |
| | 603190322F1 NIH_MGC_95 | 0.615 | 1568983_a_at | BI547087 | 826 | |
| | Homo sapiens cDNA clone | | | | | |
| | IMAGE: 5261717 5′, mRNA | | | | | |
| | sequence. | | | | | |
| SHARP | SMART/HDAC1 associated | 0.615 | 1556059_s_at | BM992098 | 827 | |
| | repressor protein | | | | | |
| USP47 | ubiquitin specific protease 47 | 0.615 | 223117_s_at | AW025093 | 828 | |
| TAX1BP1 | Tax1 (human T-cell leukemia | 0.614 | 213786_at | AI935415 | 829 | |
| | virus type I) binding protein 1 | | | | | |
| AXOT | axotrophin | 0.614 | 202654_x_at | NM_022826 | 830 | |
| SLC6A6 | solute carrier family 6 | 0.614 | 205920_at | NM_003043 | 831 | |
| | (neurotransmitter transporter, | | | | | |
| | taurine), member 6 | | | | | |
| BTBD5 | BTB (POZ) domain | 0.614 | 243982_at | AA455180 | 832 | |
| | containing 5 | | | | | |
| MARCKS | myristoylated alanine-rich | 0.613 | 201669_s_at | NM_002356 | 833 | |
| | protein kinase C substrate | | | | | |
| DKFZp761B128 | 601659695R1 NIH_MGC_70 | 0.613 | 224593_at | BE965646 | 834 | |
| | Homo sapiens cDNA clone | | | | | |
| | IMAGE: 3896279 3′, mRNA | | | | | |
| | sequence. | | | | | |
| EIF2C1 | eukaryotic translation | 0.613 | 222576_s_at | AW071829 | 835 | |
| | initiation factor 2C, 1 | | | | | |
| RFXAP | regulatory factor X-associated | 0.613 | 229431_at | AI742868 | 836 | |
| | protein | | | | | |
| MGC3248 | dynactin 4 | 0.613 | 209231_s_at | AI038068 | 837 | |
| C6orf111 | ok92b01.s1 NCI_CGAP_Lu5 | 0.613 | 212176_at | AA902326 | 838 | |
| | Homo sapiens cDNA clone | | | | | |
| | IMAGE: 1521385 3′, mRNA | | | | | |
| | sequence. | | | | | |
| KLF7 | Kruppel-like factor 7 | 0.613 | 1555420_a_at | BC012919 | 839 | |
| | (ubiquitous) | | | | | |
| LOC129531 | hypothetical protein | 0.612 | 226329_s_at | BF055289 | 840 | |
| | BC018453 | | | | | |
| DKFZp761C169 | vasculin | 0.612 | 223705_s_at | BC000267 | 841 | |
| TMEM23 | mob protein | 0.612 | 212989_at | AI377497 | 842 | |
| C9orf5 | chromosome 9 open reading | 0.611 | 223005_s_at | BG402553 | 843 | |
| | frame 5 | | | | | |
| MGC33371 | hypothetical protein | 0.611 | 1553749_at | NM_144664 | 844 | |
| | MGC33371 | | | | | |
| LOC51315 | hypothetical protein | 0.611 | 233329_s_at | AK025986 | 845 | |
| | LOC51315 | | | | | |
| UPF3A | UPF3 regulator of nonsense | 0.611 | 206958_s_at | AF318575 | 846 | |
| | transcripts homolog A (yeast) | | | | | |
| SPAP1 | SH2 domain containing | 0.611 | 221239_s_at | NM_030764 | 847 | |
| | phosphatase anchor protein 1 | | | | | |
| PTPN22 | protein tyrosine phosphatase, | 0.611 | 236539_at | AW665758 | 848 | |
| | non-receptor type 22 | | | | | |
| | (lymphoid) | | | | | |
| SLC25A24 | calcium-binding transporter | 0.61 | 204342_at | NM_013386 | 849 | |
| RERE | arginine-glutamic acid | 0.61 | 221643_s_at | AF016005 | 850 | |
| | dipeptide (RE) repeats | | | | | |
| ZNF258 | zinc finger protein 258 | 0.61 | 227595_at | AK000927 | 851 | |
| | CDNA FLJ44883 fis, clone | 0.61 | 236196_at | BF939032 | 852 | |
| | BRAMY2036918 | | | | | |
| IRLB | c-myc promoter-binding | 0.61 | 214787_at | BE268538 | 853 | |
| | protein | | | | | |
| | Clone IMAGE: 5260228, | 0.61 | 1568870_at | BC034805 | 854 | |
| | mRNA | | | | | |
| NID | nidogen (enactin) | 0.61 | 202007_at | BF940043 | 855 | |
| PHKB | phosphorylase kinase, beta | 0.61 | 238601_at | AI798207 | 856 | |
| ARRDC4 | arrestin domain containing 4 | 0.61 | 225283_at | AV701177 | 857 | |
| MBNL1 | muscleblind-like (Drosophila) | 0.609 | 201151_s_at | BF512200 | 858 | |
| CREB1 | cAMP responsive element | 0.609 | 204312_x_at | AI655737 | 859 | |
| | binding protein 1 | | | | | |
| TNFRSF6 | tumor necrosis factor receptor | 0.609 | 204780_s_at | AA164751 | 860 | |
| | superfamily, member 6 | | | | | |
| | Transcribed sequences | 0.609 | 230177_at | AI884495 | 861 | |
| | Transcribed sequence with | 0.609 | 242261_at | AW470799 | 862 | |
| | weak similarity to protein | | | | | |
| | ref: NP_055301.1 (H. sapiens) | | | | | |
| | neuronal thread protein | | | | | |
| | [Homo sapiens ] | | | | | |
| FGFR1OP2 | DKFZp564O1863 protein | 0.609 | 243619_at | R91766 | 863 | |
| MLL3 | myeloid/lymphoid or mixed- | 0.608 | 1557158_s_at | AI479224 | 864 | |
| | lineage leukemia 3 | | | | | |
| NNT | nicotinamide nucleotide | 0.608 | 202784_s_at | NM_012343 | 865 | |
| | transhydrogenase | | | | | |
| PIK3C2A | phosphoinositide-3-kinase, | 0.608 | 1553694_a_at | NM_002645 | 866 | |
| | class 2, alpha polypeptide | | | | | |
| FLJ22490 | hypothetical protein | 0.608 | 227105_at | AI400587 | 867 | |
| | FLJ22490 | | | | | |
| LOC51321 | hypothetical protein | 0.608 | 235422_at | AA977218 | 868 | |
| | LOC51321 | | | | | |
| SPEC2 | non-kinase Cdc42 effector | 0.608 | 1552613_s_at | NM_020240 | 869 | |
| | protein SPEC2 | | | | | |
| ATP5C1 | naf35f07.x1 Soares_NPBMC | 0.608 | 214132_at | BG232034 | 870 | |
| | Homo sapiens cDNA clone | | | | | |
| | IMAGE: 4143109 3′, mRNA | | | | | |
| | sequence. | | | | | |
| SCAP2 | Homo sapiens , SKAP55 | 0.608 | 216899_s_at | AC003999 | 871 | |
| | homologue, clone | | | | | |
| | MGC: 10411 | | | | | |
| | IMAGE: 3940826, mRNA, | | | | | |
| | complete cds.; | | | | | |
| | H_DJ1139P01.1 This gene | | | | | |
| | was based on gi(12804080 | | | | | |
| | 16753211) Continues from | | | | | |
| | H_NH0232C20.1; Homo | | | | | |
| | sapiens PAC clone RP5- | | | | | |
| | 1139P1 from 7, complete | | | | | |
| | sequence. | | | | | |
| FUSIP1 | synonyms: NSSR, TASR, | 0.608 | 204299_at | NM_021993 | 872 | |
| | SRp38, TASR1, TASR2, | | | | | |
| | FUSIP2, SRrp40; isoform 2 is | | | | | |
| | encoded by transcript variant | | | | | |
| | 2; TLS-associated serine- | | | | | |
| | arginine protein 1; serine- | | | | | |
| | arginine repressor protein (40 kDa); | | | | | |
| | TLS-associated serine- | | | | | |
| | arginine protein 2; TLS- | | | | | |
| | associated protein TASR-1; | | | | | |
| | go_component: nucleus [goid | | | | | |
| | 0005634] [evidence NR]; | | | | | |
| | go_component: nucleoplasm | | | | | |
| | [goid 0005654] [evidence | | | | | |
| | IDA] [pmid 11684676]; | | | | | |
| | go_component: cytoplasm | | | | | |
| | [goid 0005737] [evidence | | | | | |
| | NAS] [pmid 11684676]; | | | | | |
| | go_function: pre-mRNA | | | | | |
| | splicing factor activity [goid | | | | | |
| | 0008248] [evidence IDA] | | | | | |
| | [pmid 11684676]; | | | | | |
| | go_function: DNA binding | | | | | |
| | [goid 0003677] [evidence | | | | | |
| | IEA]; go_function: nucleic | | | | | |
| | acid binding [goid 0003676] | | | | | |
| | [evidence IEA]; go_function: | | | | | |
| | protein binding [goid | | | | | |
| | 0005515] [evidence IPI] [pmid | | | | | |
| | 11684676]; go_function: | | | | | |
| | chaperone activity [goid | | | | | |
| | 0003754] [evidence NAS] | | | | | |
| | [pmid 11684676]; | | | | | |
| | go_function: RNA binding | | | | | |
| | [goid 0003723] [evidence | | | | | |
| | NAS] [pmid 9774382]; | | | | | |
| | go_function: RS domain | | | | | |
| | binding [goid 0050733] | | | | | |
| | [evidence NAS] [pmid | | | | | |
| | 11684676]; go_process: | | | | | |
| | mRNA processing [goid | | | | | |
| | 0006397] [evidence TAS] | | | | | |
| | [pmid 9774382]; go_process: | | | | | |
| | spliceosome assembly [goid | | | | | |
| | 0000245] [evidence TAS] | | | | | |
| | [pmid 9774382]; go_process: | | | | | |
| | regulation of transcription, | | | | | |
| | DNA-dependent [goid | | | | | |
| | 0006355] [evidence IEA]; | | | | | |
| | go_process: assembly of | | | | | |
| | spliceosomal tri-snRNP [goid | | | | | |
| | 0000244] [evidence NAS] | | | | | |
| | [pmid 11684676]; | | | | | |
| | go_process: mRNA-nucleus | | | | | |
| | export [goid 0006406] | | | | | |
| | [evidence NAS] [pmid | | | | | |
| | 11684676]; go_process: | | | | | |
| | cytoplasmic transport [goid | | | | | |
| | 0016482] [evidence IDA] | | | | | |
| | [pmid 11684676]; | | | | | |
| | go_process: regulation of | | | | | |
| | transcription [goid 0045449] | | | | | |
| | [evidence NAS] [pmid | | | | | |
| | 9774382]; go_process: | | | | | |
| | negative regulation of nuclear | | | | | |
| | mRNA splicing, via | | | | | |
| | spliceosome [goid 0048025] | | | | | |
| | [evidence IDA] [pmid | | | | | |
| | 11684676]; go_process: | | | | | |
| | nuclear mRNA splicing, via | | | | | |
| | spliceosome [goid 0000398] | | | | | |
| | [evidence IDA] [pmid | | | | | |
| | 9774382]; go_process: | | | | | |
| | nuclear mRNA 5′-splice site | | | | | |
| | recognition [goid 0000395] | | | | | |
| | [evidence IDA] [pmid | | | | | |
| | 11684676]; Homo sapiens | | | | | |
| | FUS interacting protein | | | | | |
| | (serine-arginine rich) 1 | | | | | |
| | (FUSIP1), transcript variant 2, | | | | | |
| | mRNA. | | | | | |
| TTC3 | tetratricopeptide repeat | 0.608 | 208663_s_at | AI652848 | 873 | |
| | domain 3 | | | | | |
| LOC400590 | CDNA clone MGC: 70830 | 0.608 | 241910_x_at | AI610396 | 874 | |
| | IMAGE: 5248762, complete | | | | | |
| | cds | | | | | |
| MGEA5 | meningioma expressed | 0.608 | 200898_s_at | AK002091 | 875 | |
| | antigen 5 (hyaluronidase) | | | | | |
| EP400 | E1A binding protein p400 | 0.608 | 212375_at | AL563727 | 876 | |
| SMARCA2 | SWI/SNF related, matrix | 0.607 | 206542_s_at | AV725365 | 877 | |
| | associated, actin dependent | | | | | |
| | regulator of chromatin, | | | | | |
| | subfamily a, member 2 | | | | | |
| MSH2 | mutS homolog 2, colon | 0.607 | 209421_at | U04045 | 878 | |
| | cancer, nonpolyposis type 1 | | | | | |
| | (E. coli) | | | | | |
| KLHL14 | KIAA1384 protein | 0.607 | 228377_at | AB037805 | 879 | |
| GLS | glutaminase | 0.607 | 203158_s_at | AF097493 | 880 | |
| KIAA0701 | KIAA0701 protein | 0.607 | 213118_at | AL136821 | 881 | |
| | chromosome 6 open reading | 0.607 | 229543_at | AV734646 | 882 | |
| | frame 187 | | | | | |
| GPSM1 | G-protein signalling | 0.607 | 226043_at | AI242661 | 883 | |
| | modulator 1 (AGS3-like, C. elegans) | | | | | |
| FLJ13213 | hypothetical protein | 0.607 | 217828_at | NM_024755 | 884 | |
| | FLJ13213 | | | | | |
| ACLY | ATP citrate lyase | 0.606 | 201128_s_at | NM_001096 | 885 | |
| PLEKHA2 | pleckstrin homology domain | 0.606 | 238013_at | BF347859 | 886 | |
| | containing, family A | | | | | |
| | (phosphoinositide binding | | | | | |
| | specific) member 2 | | | | | |
| ZZZ3 | zinc finger, ZZ domain | 0.606 | 212893_at | AL080063 | 887 | |
| | containing 3 | | | | | |
| C20orf158 | chromosome 20 open reading | 0.606 | 227335_at | AW664953 | 888 | |
| | frame 158 | | | | | |
| YT521 | splicing factor YT521-B | 0.606 | 212455_at | N36997 | 889 | |
| FLJ11126 | hypothetical protein | 0.606 | 202578_s_at | NM_018332 | 890 | |
| | FLJ11126 | | | | | |
| FLJ23091 | putative NFkB activating | 0.606 | 221958_s_at | AA775681 | 891 | |
| | protein 373 | | | | | |
| | Transcribed sequences | 0.606 | 242894_at | AA620926 | 892 | |
| F2RL1 | 601659282R1 NIH_MGC_70 | 0.606 | 213506_at | BE965369 | 893 | |
| | Homo sapiens cDNA clone | | | | | |
| | IMAGE: 3895653 3′, mRNA | | | | | |
| | sequence. | | | | | |
| GMIP | Gem-interacting protein | 0.605 | 222782_s_at | BF000144 | 894 | |
| ASAH1 | N-acylsphingosine | 0.605 | 210980_s_at | U47674 | 895 | |
| | amidohydrolase (acid | | | | | |
| | ceramidase) 1 | | | | | |
| CDC16 | CDC16 cell division cycle 16 | 0.605 | 202717_s_at | NM_003903 | 896 | |
| | homolog (S. cerevisiae) | | | | | |
| SENP6 | SUMO1/sentrin specific | 0.605 | 202318_s_at | AF306508 | 897 | |
| | protease 6 | | | | | |
| | Transcribed sequence with | 0.605 | 244803_at | AI335191 | 898 | |
| | moderate similarity to protein | | | | | |
| | ref: NP_060190.1 (H. sapiens) | | | | | |
| | hypothetical protein | | | | | |
| | FLJ20234 [Homo sapiens ] | | | | | |
| FLJ35036 | hypothetical protein | 0.605 | 225512_at | AI005245 | 899 | |
| | FLJ35036 | | | | | |
| | Transcribed sequences | 0.604 | 243505_at | AI937121 | 900 | |
| TPR | translocated promoter region | 0.604 | 1557227_s_at | AW235355 | 901 | |
| | (to activated MET oncogene) | | | | | |
| | Homo sapiens mRNA; cDNA | 0.604 | 1566518_at | AL832405 | 902 | |
| | DKFZp667E1713 (from clone | | | | | |
| | DKFZp667E1713). | | | | | |
| | Transcribed sequences | 0.604 | 229531_at | AW182938 | 903 | |
| IGHG1 | Partial mRNA for | 0.604 | 211634_x_at | M24669 | 904 | |
| | immunoglobulin heavy chain | | | | | |
| | variable region (IGHV gene), | | | | | |
| | isolate B-CLL G026 | | | | | |
| STCH | stress 70 protein chaperone, | 0.604 | 202558_s_at | NM_006948 | 905 | |
| | microsome-associated, | | | | | |
| | 60 kDa | | | | | |
| | MRNA; cDNA | 0.604 | 233224_at | AL137645 | 906 | |
| | DKFZp586D0924 (from clone | | | | | |
| | DKFZp586D0924) | | | | | |
| SNAP23 | synaptosomal-associated | 0.603 | 209131_s_at | U55936 | 907 | |
| | protein, 23 kDa | | | | | |
| WBP11 | WW domain binding protein | 0.603 | 217821_s_at | AF118023 | 908 | |
| | 11 | | | | | |
| RAB2 | RAB2, member RAS | 0.603 | 221960_s_at | AI189609 | 909 | |
| | oncogene family | | | | | |
| NPHP3 | Transcribed sequence with | 0.603 | 235410_at | BG034966 | 910 | |
| | weak similarity to protein | | | | | |
| | ref: NP_060265.1 (H. sapiens) | | | | | |
| | hypothetical protein | | | | | |
| | FLJ20378 [Homo sapiens ] | | | | | |
| CLPX | ClpX caseinolytic protease X | 0.603 | 204809_at | NM_006660 | 911 | |
| | homolog (E. coli) | | | | | |
| USP6 | ubiquitin specific protease 32 | 0.603 | 226505_x_at | AI148567 | 912 | |
| FGFR1OP | FGFR1 oncogene partner | 0.603 | 1568678_s_at | BC037785 | 913 | |
| ATP6AP2 | ATPase, H+ transporting, | 0.603 | 201443_s_at | AF248966 | 914 | |
| | lysosomal accessory protein 2 | | | | | |
| ARFD1 | ADP-ribosylation factor | 0.603 | 210995_s_at | AF230399 | 915 | |
| | domain protein 1, 64 kDa | | | | | |
| UPF3A | UPF3 regulator of nonsense | 0.602 | 217596_at | AA649851 | 916 | |
| | transcripts homolog A (yeast) | | | | | |
| PARN | poly(A)-specific ribonuclease | 0.602 | 203905_at | NM_002582 | 917 | |
| | (deadenylation nuclease) | | | | | |
| M17S2 | membrane component, | 0.602 | 201384_s_at | NM_005899 | 918 | |
| | chromosome 17, surface | | | | | |
| | marker 2 (ovarian carcinoma | | | | | |
| | antigen CA125) | | | | | |
| MRPL42 | mitochondrial ribosomal | 0.602 | 217919_s_at | BE782148 | 919 | |
| | protein L42 | | | | | |
| CLN2 | ceroid-lipofuscinosis, | 0.602 | 200743_s_at | NM_000391 | 920 | |
| | neuronal 2, late infantile | | | | | |
| | (Jansky-Bielschowsky | | | | | |
| | disease) | | | | | |
| | Transcribed sequence with | 0.602 | 236923_x_at | W90039 | 921 | |
| | weak similarity to protein | | | | | |
| | ref: NP_060265.1 (H. sapiens) | | | | | |
| | hypothetical protein | | | | | |
| | FLJ20378 [Homo sapiens ] | | | | | |
| C1orf25 | chromosome 1 open reading | 0.601 | 223404_s_at | AW512122 | 922 | |
| | frame 25 | | | | | |
| | Clone IMAGE: 4250282, | 0.601 | 1569180_at | BC017896 | 923 | |
| | mRNA | | | | | |
| ABHD2 | abhydrolase domain | 0.601 | 87100_at | AI832249 | 924 | |
| | containing 2 | | | | | |
| LACTB | lactamase, beta | 0.601 | 1552485_at | NM_171846 | 925 | |
| NCOA6 | nuclear receptor coactivator 6 | 0.601 | 208979_at | AF128458 | 926 | |
| ZNF207 | zinc finger protein 207 | 0.601 | 1556035_s_at | AI201248 | 927 | |
| BIG1 | brefeldin A-inhibited guanine | 0.601 | 202955_s_at | AF084520 | 928 | |
| | nucleotide-exchange protein 1 | | | | | |
| | Transcribed sequences | 0.6 | 237456_at | AI655806 | 929 | |
| CCNT2 | cyclin T2 | 0.6 | 204645_at | NM_001241 | 930 | |
| MPHOSPH9 | M-phase phosphoprotein 9 | 0.6 | 206205_at | NM_022782 | 931 | |
| FLJ20485 | hypothetical protein | 0.6 | 218984_at | NM_019042 | 932 | |
| | FLJ20485 | | | | | |
| OXR1 | AL541048 Homo sapiens | 0.6 | 222553_x_at | AL541048 | 933 | |
| | PLACENTA Homo sapiens | | | | | |
| | cDNA clone CS0DE005YI17 | | | | | |
| | 3-PRIME, mRNA sequence. | | | | | |
| | Transcribed sequences | 0.6 | 239778_x_at | AA916568 | 934 | |
| FLJ13386 | hypothetical protein | 0.6 | 219242_at | NM_025180 | 935 | |
| | FLJ13386 | | | | | |
| ANKFY1 | ankyrin repeat and FYVE | 0.6 | 219868_s_at | NM_016376 | 936 | |
| | domain containing 1 | | | | | |
| RAB11A | RAB11A, member RAS | 0.6 | 200864_s_at | NM_004663 | 937 | |
| | oncogene family | | | | | |
| LOC55871 | COBW domain containing 2 | 0.599 | 229804_x_at | AW169333 | 938 | |
| TPARL | TPA regulated locus | 0.599 | 226825_s_at | AW665624 | 939 | |
| NUP133 | nucleoporin 133 kDa | 0.599 | 202184_s_at | NM_018230 | 940 | |
| UBE2V2 | ubiquitin-conjugating enzyme | 0.599 | 209096_at | U62136 | 941 | |
| | E2 variant 2 | | | | | |
| | Transcribed sequences | 0.598 | 244868_at | AA001941 | 942 | |
| NYD-SP21 | testes development-related | 0.598 | 229510_at | AL044520 | 943 | |
| | NYD-SP21 | | | | | |
| FLJ13576 | hypothetical protein | 0.598 | 234726_s_at | AK025482 | 944 | |
| | FLJ13576 | | | | | |
| | Transcribed sequences | 0.598 | 1558603_at | AV688060 | 945 | |
| MKLN1 | muskelin 1, intracellular | 0.598 | 204423_at | NM_013255 | 946 | |
| | mediator containing kelch | | | | | |
| | motifs | | | | | |
| | Transcribed sequence with | 0.597 | 235078_at | AI393725 | 947 | |
| | weak similarity to protein | | | | | |
| | ref: NP_060312.1 (H. sapiens) | | | | | |
| | hypothetical protein | | | | | |
| | FLJ20489 [Homo sapiens ] | | | | | |
| LLT1 | lectin-like NK cell receptor | 0.597 | 228426_at | AW268886 | 948 | |
| C9orf99 | chromosome 9 open reading | 0.597 | 209512_at | BC004331 | 949 | |
| | frame 99 | | | | | |
| WASPIP | Wiskott-Aldrich syndrome | 0.597 | 202663_at | AI005043 | 950 | |
| | protein interacting protein | | | | | |
| WHSC1 | Wolf-Hirschhorn syndrome | 0.597 | 209053_s_at | BE793789 | 951 | |
| | candidate 1 | | | | | |
| SEC23A | Sec23 homolog A (S. cerevisiae) | 0.596 | 212887_at | AI753659 | 952 | |
| USP53 | ubiquitin specific protease 53 | 0.596 | 231817_at | H25097 | 953 | |
| USP24 | ubiquitin specific protease 24 | 0.596 | 212381_at | BF444943 | 954 | |
| FLJ11752 | NTKL-binding protein 1 | 0.596 | 226337_at | AK021814 | 955 | |
| KIAA1659 | KIAA1659 protein | 0.596 | 215750_at | AB051446 | 956 | |
| GSPT1 | G1 to S phase transition 1 | 0.596 | 234975_at | BE544748 | 957 | |
| ANKRD17 | ankyrin repeat domain 17 | 0.596 | 212211_at | AI986295 | 958 | |
| | Transcribed sequences | 0.596 | 240733_at | W92005 | 959 | |
| MACF1 | microtubule-actin crosslinking | 0.596 | 207358_x_at | NM_012090 | 960 | |
| | factor 1 | | | | | |
| FOXJ3 | | 0.596 | 206015_s_at | NM_014947 | 961 | |
| HNRPR | heterogeneous nuclear | 0.595 | 232004_at | AK001846 | 962 | |
| | ribonucleoprotein R | | | | | |
| MST4 | Mst3 and SOK1-related | 0.595 | 218499_at | NM_016542 | 963 | |
| | kinase | | | | | |
| | UI-H-BW0-aif-f-02-0-UI.s1 | 0.595 | 236198_at | AW292872 | 964 | |
| | NCI_CGAP_Sub6 Homo | | | | | |
| | sapiens cDNA clone | | | | | |
| | IMAGE: 2729211 3′, mRNA | | | | | |
| | sequence. | | | | | |
| WWP2 | Nedd-4-like ubiquitin-protein | 0.595 | 1552737_s_at | NM_007014 | 965 | |
| | ligase | | | | | |
| ZNF621 | FLJ45246 protein | 0.595 | 1558620_at | AK074366 | 966 | |
| CHD2 | chromodomain helicase DNA | 0.595 | 1554014_at | BC007347 | 967 | |
| | binding protein 2 | | | | | |
| SEC15L1 | SEC15-like 1 (S. cerevisiae) | 0.595 | 232599_at | AF220217 | 968 | |
| TAF4 | TAF4 RNA polymerase II, | 0.595 | 213090_s_at | AI744029 | 969 | |
| | TATA box binding protein | | | | | |
| | (TBP)-associated factor, | | | | | |
| | 135 kDa | | | | | |
| PHKB | phosphorylase kinase, beta | 0.594 | 202738_s_at | BG149218 | 970 | |
| CLECSF2 | C-type (calcium dependent, | 0.594 | 209732_at | BC005254 | 971 | |
| | carbohydrate-recognition | | | | | |
| | domain) lectin, superfamily | | | | | |
| | member 2 (activation- | | | | | |
| | induced) | | | | | |
| FLJ22054 | hypothetical protein | 0.594 | 1555450_a_at | BC032318 | 972 | |
| | FLJ22054 | | | | | |
| | Transcribed sequence with | 0.594 | 214964_at | AA554430 | 973 | |
| | weak similarity to protein | | | | | |
| | ref: NP_060312.1 (H. sapiens) | | | | | |
| | hypothetical protein | | | | | |
| | FLJ20489 [Homo sapiens ] | | | | | |
| PDHX | pyruvate dehydrogenase | 0.593 | 203067_at | NM_003477 | 974 | |
| | complex, component X | | | | | |
| VG5Q | vasculogenesis gene on 5q | 0.593 | 218534_s_at | NM_018046 | 975 | |
| | Clone IMAGE: 5441027, | 0.593 | 1558624_at | BC033250 | 976 | |
| | mRNA, partial cds | | | | | |
| M96 | likely ortholog of mouse metal | 0.593 | 203347_s_at | NM_007358 | 977 | |
| | response element binding | | | | | |
| | transcription factor 2 | | | | | |
| DNAJC10 | ER-resident protein ERdj5 | 0.593 | 225174_at | AW512173 | 978 | |
| FLJ21918 | nuclear factor of activated T- | 0.593 | 225137_at | BF111111 | 979 | |
| | cells, cytoplasmic, | | | | | |
| | calcineurin-dependent 3 | | | | | |
| HNRPH1 | heterogeneous nuclear | 0.593 | 213472_at | AI022387 | 980 | |
| | ribonucleoprotein H1 (H) | | | | | |
| MIB | ubiquitin ligase mind bomb | 0.592 | 224725_at | AB037744 | 981 | |
| TRNT1 | tRNA nucleotidyl transferase, | 0.592 | 223814_at | BC005184 | 982 | |
| | CCA-adding, 1 | | | | | |
| ATP2B1 | | 0.592 | 215716_s_at | L14561 | 983 | |
| UBE2H | ubiquitin-conjugating enzyme | 0.591 | 221962_s_at | AI829920 | 984 | |
| | E2H (UBC8 homolog, yeast) | | | | | |
| | Transcribed sequences | 0.591 | 243919_at | AA747291 | 985 | |
| CIAS1 | cold autoinflammatory | 0.591 | 207075_at | NM_004895 | 986 | |
| | syndrome 1 | | | | | |
| FLJ34588 | Smhs2 homolog (rat) | 0.591 | 212410_at | AI346431 | 987 | |
| ARHGAP6 | Rho GTPase activating | 0.591 | 206167_s_at | NM_001174 | 988 | |
| | protein 6 | | | | | |
| DPYD | dihydropyrimidine | 0.591 | 204646_at | NM_000110 | 989 | |
| | dehydrogenase | | | | | |
| C6orf67 | Human DNA sequence from | 0.59 | 222391_at | AL080250 | 990 | |
| | clone RP1-234P15 on | | | | | |
| | chromosome 6q13-14.3, | | | | | |
| | complete sequence. | | | | | |
| TRRAP | | 0.59 | 214908_s_at | AC004893 | 991 | |
| KIAA1915 | KIAA1915 protein | 0.59 | 225760_at | AI302244 | 992 | |
| RAB7 | RAB7, member RAS | 0.59 | 211960_s_at | BG261416 | 993 | |
| | oncogene family | | | | | |
| G3BP2 | Ras-GTPase activating | 0.59 | 208840_s_at | AU149503 | 994 | |
| | protein SH3 domain-binding | | | | | |
| | protein 2 | | | | | |
| FBXO3 | F-box only protein 3 | 0.589 | 218432_at | NM_012175 | 995 | |
| APAF1 | apoptotic protease activating | 0.589 | 204859_s_at | NM_013229 | 996 | |
| | factor | | | | | |
| DNAJC10 | ER-resident protein ERdj5 | 0.589 | 221781_s_at | BG168666 | 997 | |
| FLJ13373 | | 0.588 | 220577_at | NM_025006 | 998 | |
| | MRNA; cDNA | 0.588 | 243899_at | BE674920 | 999 | |
| | DKFZp686A05147 (from | | | | | |
| | clone DKFZp686A05147) | | | | | |
| HNRPH3 | heterogeneous nuclear | 0.588 | 208990_s_at | AF132362 | 1000 | |
| | ribonucleoprotein H3 (2H9) | | | | | |
| MAK3 | likely ortholog of mouse | 0.588 | 217745_s_at | NM_025146 | 1001 | |
| | Mak3p homolog (S. cerevisiae) | | | | | |
| PTP4A1 | 602135085F1 NIH_MGC_81 | 0.588 | 200730_s_at | BF576710 | 1002 | |
| | Homo sapiens cDNA clone | | | | | |
| | IMAGE: 4290141 5′, mRNA | | | | | |
| | sequence. | | | | | |
| KIAA0779 | KIAA0779 protein | 0.587 | 213352_at | AB018322 | 1003 | |
| GARNL1 | GTPase activating RANGAP | 0.587 | 213049_at | BG436400 | 1004 | |
| | domain-like 1 | | | | | |
| ARTS-1 | type 1 tumor necrosis factor | 0.587 | 210385_s_at | AF106037 | 1005 | |
| | receptor shedding | | | | | |
| | aminopeptidase regulator | | | | | |
| CRK7 | CDC2-related protein kinase 7 | 0.587 | 225694_at | AI823766 | 1006 | |
| DOCK8 | | 0.587 | 225502_at | AL161725 | 1007 | |
| BIG1 | brefeldin A-inhibited guanine | 0.587 | 216266_s_at | AK025637 | 1008 | |
| | nucleotide-exchange protein 1 | | | | | |
| FLJ21908 | hypothetical protein | 0.587 | 218842_at | NM_024604 | 1009 | |
| | FLJ21908 | | | | | |
| C6orf32 | chromosome 6 open reading | 0.586 | 209829_at | AB002384 | 1010 | |
| | frame 32 | | | | | |
| | yq77a11.r1 Soares fetal liver | 0.586 | 239245_at | R99939 | 1011 | |
| | spleen 1NFLS Homo sapiens | | | | | |
| | cDNA clone IMAGE: 201788 | | | | | |
| | 5′, mRNA sequence. | | | | | |
| MTR | 5-methyltetrahydrofolate- | 0.586 | 203774_at | NM_000254 | 1012 | |
| | homocysteine | | | | | |
| | methyltransferase | | | | | |
| IGHM | Partial mRNA for | 0.586 | 211637_x_at | L23516 | 1013 | |
| | immunoglobulin heavy chain | | | | | |
| | variable region (IGHV32-D- | | | | | |
| | JH-Cmu gene), clone ET39 | | | | | |
| | MRNA; cDNA | 0.586 | 215528_at | AL049390 | 1014 | |
| | DKFZp586O1318 (from clone | | | | | |
| | DKFZp586O1318) | | | | | |
| UBE2D3 | ubiquitin-conjugating enzyme | 0.586 | 233303_at | AL110175 | 1015 | |
| | E2D 3 (UBC4/5 homolog, | | | | | |
| | yeast) | | | | | |
| TNRC11 | | 0.585 | 211342_x_at | BC004354 | 1016 | |
| DKFZp547A023 | neural precursor cell | 0.585 | 214731_at | AB037854 | 1017 | |
| | expressed, developmentally | | | | | |
| | down-regulated 5 | | | | | |
| MCP | membrane cofactor protein | 0.585 | 211574_s_at | D84105 | 1018 | |
| | (CD46, trophoblast- | | | | | |
| | lymphocyte cross-reactive | | | | | |
| | antigen) | | | | | |
| KAT3 | RNA binding motif protein, X- | 0.585 | 225310_at | AI928344 | 1019 | |
| | linked | | | | | |
| YY1 | YY1 transcription factor | 0.585 | 200047_s_at | NM_003403 | 1020 | |
| PC4 | activated RNA polymerase II | 0.585 | 224586_x_at | BE784583 | 1021 | |
| | transcription cofactor 4 | | | | | |
| MLLT10 | AL562031 Homo sapiens | 0.584 | 225992_at | AL562031 | 1022 | |
| | NEUROBLASTOMA COT 10- | | | | | |
| | NORMALIZED Homo sapiens | | | | | |
| | cDNA clone CS0DB003YG22 | | | | | |
| | 3-PRIME, mRNA sequence. | | | | | |
| MGAT4A | mannosyl (alpha-1,3-)- | 0.584 | 219797_at | NM_012214 | 1023 | |
| | glycoprotein beta-1,4-N- | | | | | |
| | acetylglucosaminyltransferase, | | | | | |
| | isoenzyme A | | | | | |
| LGALS8 | lectin, galactoside-binding, | 0.584 | 208936_x_at | AF074000 | 1024 | |
| | soluble, 8 (galectin 8) | | | | | |
| FLJ11011 | hypothetical protein | 0.584 | 1562458_at | AL833723 | 1025 | |
| | FLJ11011 | | | | | |
| | Transcribed sequence with | 0.584 | 242191_at | AI701905 | 1026 | |
| | weak similarity to protein | | | | | |
| | ref: NP_060312.1 (H. sapiens) | | | | | |
| | hypothetical protein | | | | | |
| | FLJ20489 [Homo sapiens ] | | | | | |
| ADSS | adenylosuccinate synthase | 0.584 | 221761_at | AA628948 | 1027 | |
| C6orf67 | chromosome 6 open reading | 0.584 | 217743_s_at | NM_018247 | 1028 | |
| | frame 67 | | | | | |
| PSMC6 | proteasome (prosome, | 0.584 | 201699_at | NM_002806 | 1029 | |
| | macropain) 26S subunit, | | | | | |
| | ATPase, 6 | | | | | |
| FAM29A | family with sequence | 0.584 | 233655_s_at | AK022964 | 1030 | |
| | similarity 29, member A | | | | | |
| FLJ30655 | hypothetical protein | 0.583 | 236487_at | AW513286 | 1031 | |
| | FLJ30655 | | | | | |
| FLJ21174 | hypothetical protein | 0.583 | 202371_at | NM_024863 | 1032 | |
| | FLJ21174 | | | | | |
| ITPR1 | inositol 1,4,5-triphosphate | 0.583 | 240052_at | AA648993 | 1033 | |
| | receptor, type 1 | | | | | |
| ZNF439 | zinc finger protein 439 | 0.583 | 236562_at | N29327 | 1034 | |
| IGHG1 | Partial mRNA for | 0.582 | 211635_x_at | M24670 | 1035 | |
| | immunoglobulin heavy chain | | | | | |
| | variable region (IGHV gene), | | | | | |
| | isolate B-CLL G026 | | | | | |
| FLJ12716 | FLJ12716 protein | 0.582 | 218179_s_at | NM_021942 | 1036 | |
| KIAA0310 | KIAA0310 | 0.582 | 215696_s_at | BC001404 | 1037 | |
| ARFIP1 | ADP-ribosylation factor | 0.582 | 214483_s_at | AF124489 | 1038 | |
| | interacting protein 1 (arfaptin | | | | | |
| | 1) | | | | | |
| TCF12 | transcription factor 12 (HTF4, | 0.581 | 208986_at | AL559478 | 1039 | |
| | helix-loop-helix transcription | | | | | |
| | factors 4) | | | | | |
| APBB1IP | amyloid beta (A4) precursor | 0.581 | 219994_at | NM_019043 | 1040 | |
| | protein-binding, family B, | | | | | |
| | member 1 interacting protein | | | | | |
| | Transcribed sequences | 0.581 | 241610_x_at | AI306990 | 1041 | |
| C1orf19 | chromosome 1 open reading | 0.581 | 225400_at | BF111780 | 1042 | |
| | frame 19 | | | | | |
| PRO1073 | PRO1073 protein | 0.581 | 223577_x_at | AA827878 | 1043 | |
| ANKRD12 | ankyrin repeat domain 12 | 0.581 | 212286_at | AW572909 | 1044 | |
| ZNF33A | 602498083F1 NIH_MGC_75 | 0.581 | 215022_x_at | BG429214 | 1045 | |
| | Homo sapiens cDNA clone | | | | | |
| | IMAGE: 4611836 5′, mRNA | | | | | |
| | sequence. | | | | | |
| NOTCH2 | Notch homolog 2 (Drosophila) | 0.581 | 210756_s_at | AF308601 | 1046 | |
| KIAA1219 | KIAA1219 protein | 0.581 | 221736_at | AA156777 | 1047 | |
| LY75 | lymphocyte antigen 75 | 0.581 | 205668_at | NM_002349 | 1048 | |
| GNS | glucosamine (N-acetyl)-6- | 0.58 | 203676_at | NM_002076 | 1049 | |
| | sulfatase (Sanfilippo disease | | | | | |
| | IIID) | | | | | |
| DARS | aspartyl-tRNA synthetase | 0.58 | 201624_at | NM_001349 | 1050 | |
| LOC283824 | Clone IMAGE: 4791553, | 0.58 | 213725_x_at | AI693140 | 1051 | |
| | mRNA | | | | | |
| DKFZp434G0522 | junctophilin 3 | 0.58 | 232171_x_at | AK001742 | 1052 | |
| KIAA0563 | hypothetical protein | 0.58 | 220220_at | NM_018001 | 1053 | |
| | FLJ10120 | | | | | |
| | Transcribed sequences | 0.579 | 241917_at | AA875908 | 1054 | |
| SYNCRIP | synaptotagmin binding, | 0.579 | 209025_s_at | AF037448 | 1055 | |
| | cytoplasmic RNA interacting | | | | | |
| | protein | | | | | |
| FLJ10352 | | 0.579 | 218827_s_at | NM_018069 | 1056 | |
| RAB8B | RAB8B, member RAS | 0.579 | 222846_at | AB038995 | 1057 | |
| | oncogene family | | | | | |
| LOC92017 | similar to RIKEN cDNA | 0.578 | 225624_at | AL044019 | 1058 | |
| | 4933437K13 | | | | | |
| PDE7A | phosphodiesterase 7A | 0.578 | 224046_s_at | U67932 | 1059 | |
| FLI1 | Ewing sarcoma breakpoint | 0.577 | 211825_s_at | AF327066 | 1060 | |
| | region 1 | | | | | |
| ATR | ataxia telangiectasia and | 0.577 | 209903_s_at | U49844 | 1061 | |
| | Rad3 related | | | | | |
| RTN4 | reticulon 4 | 0.577 | 211509_s_at | AB015639 | 1062 | |
| STAT1 | | 0.577 | AFFX- | AFFX- | 1063 | |
| | | | HUMISGF3A/M97935_MB_at | HUMISGF3A/ | | |
| | | | | M97935_MB | | |
| FLJ23556 | | 0.576 | 206548_at | NM_024880 | 1064 | |
| CHERP | calcium homeostasis | 0.576 | 202230_s_at | NM_006387 | 1065 | |
| | endoplasmic reticulum protein | | | | | |
| FLJ11267 | hypothetical protein | 0.576 | 243264_s_at | AI634652 | 1066 | |
| | FLJ11267 | | | | | |
| DEK | DEK oncogene (DNA binding) | 0.576 | 200934_at | NM_003472 | 1067 | |
| PTPRC | protein tyrosine phosphatase, | 0.576 | 207238_s_at | NM_002838 | 1068 | |
| | receptor type, C | | | | | |
| ATP6AP2 | ATPase, H+ transporting, | 0.575 | 201444_s_at | NM_005765 | 1069 | |
| | lysosomal accessory protein 2 | | | | | |
| | ye93f04.r1 Soares fetal liver | 0.575 | 239512_at | R05895 | 1070 | |
| | spleen 1NFLS Homo sapiens | | | | | |
| | cDNA clone IMAGE: 125311 | | | | | |
| | 5′, mRNA sequence. | | | | | |
| CD36 | CD36 antigen (collagen type I | 0.575 | 228766_at | AW299226 | 1071 | |
| | receptor, thrombospondin | | | | | |
| | receptor) | | | | | |
| GMCL1L | germ cell-less homolog | 0.575 | 218458_at | NM_022471 | 1072 | |
| | (Drosophila) | | | | | |
| MGC12916 | Clone IMAGE: 4110919, | 0.575 | 224507_s_at | BC006384 | 1073 | |
| | mRNA | | | | | |
| | Transcribed sequences | 0.575 | 240759_at | AW593931 | 1074 | |
| TPD52 | tumor protein D52 | 0.575 | 201689_s_at | BE974098 | 1075 | |
| | hg91b10.x1 | 0.575 | 1557797_a_at | AW611486 | 1076 | |
| | NCI_CGAP_Kid11 Homo | | | | | |
| | sapiens cDNA clone | | | | | |
| | IMAGE: 2952955 3′, mRNA | | | | | |
| | sequence. | | | | | |
| MGC17943 | hypothetical protein | 0.575 | 224759_s_at | AK001731 | 1077 | |
| | MGC17943 | | | | | |
| | CDNA FLJ33420 fis, clone | 0.575 | 228191_at | AA206016 | 1078 | |
| | BRACE2020028 | | | | | |
| STAT6 | signal transducer and | 0.574 | 201332_s_at | NM_003153 | 1079 | |
| | activator of transcription 6, | | | | | |
| | interleukin-4 induced | | | | | |
| | CDNA: FLJ23243 fis, clone | 0.574 | 232809_s_at | AK026896 | 1080 | |
| | COL01757 | | | | | |
| RDX | radixin | 0.574 | 212398_at | AI057093 | 1081 | |
| FLJ32001 | hypothetical protein | 0.574 | 1554661_s_at | BC036200 | 1082 | |
| | FLJ32001 | | | | | |
| H41 | hypothetical protein H41 | 0.574 | 228746_s_at | AV646549 | 1083 | |
| TLN1 | talin 1 | 0.574 | 203254_s_at | NM_006289 | 1084 | |
| KIAA0500 | MRNA, chromosome 1 | 0.573 | 213839_at | AW028110 | 1085 | |
| | specific transcript KIAA0500. | | | | | |
| MAK3 | likely ortholog of mouse | 0.573 | 222393_s_at | AU149868 | 1086 | |
| | Mak3p homolog (S. cerevisiae) | | | | | |
| C7orf2 | chromosome 7 open reading | 0.573 | 224410_s_at | AF348513 | 1087 | |
| | frame 2 | | | | | |
| LOC115294 | similar to hypothetical protein | 0.573 | 226119_at | AA453163 | 1088 | |
| | FLJ10883 | | | | | |
| ZC3HDC5 | 601660710R1 NIH_MGC_72 | 0.573 | 228357_at | BE966979 | 1089 | |
| | Homo sapiens cDNA clone | | | | | |
| | IMAGE: 3915579 3′, mRNA | | | | | |
| | sequence. | | | | | |
| LOC157697 | hypothetical protein | 0.573 | 227017_at | BE644894 | 1090 | |
| | LOC157697 | | | | | |
| DDX18 | DEAD (Asp-Glu-Ala-Asp) box | 0.573 | 205763_s_at | NM_006773 | 1091 | |
| | polypeptide 18 | | | | | |
| | Transcribed sequences | 0.572 | 243788_at | AA789293 | 1092 | |
| SCOC | short coiled-coil protein | 0.572 | 223341_s_at | AF330205 | 1093 | |
| SEC61A1 | Sec61 alpha 1 subunit (S. cerevisiae) | 0.572 | 222385_x_at | AF346602 | 1094 | |
| INSIG2 | insulin induced gene 2 | 0.571 | 209566_at | AL080184 | 1095 | |
| GOLGIN-67 | golgin-67 | 0.571 | 210424_s_at | AF163441 | 1096 | |
| | MRNA; cDNA | 0.571 | 1562250_at | AF289567 | 1097 | |
| | DKFZp451J0117 (from clone | | | | | |
| | DKFZp451J0117) | | | | | |
| FLJ35934 | Hypothetical gene supported | 0.571 | 1557895_at | BC033201 | 1098 | |
| | by AK093253 (LOC400579), | | | | | |
| | mRNA | | | | | |
| KIDINS220 | likely homolog of rat kinase | 0.571 | 212163_at | AB033076 | 1099 | |
| | D-interacting substance of | | | | | |
| | 220 kDa | | | | | |
| ITGB1 | integrin, beta 1 (fibronectin | 0.57 | 1553678_a_at | NM_133376 | 1100 | |
| | receptor, beta polypeptide, | | | | | |
| | antigen CD29 includes | | | | | |
| | MDF2, MSK12) | | | | | |
| LOC284701 | LOC389222 (LOC389222), | 0.57 | 234664_at | AL137733 | 1101 | |
| | mRNA | | | | | |
| ZBED4 | | 0.57 | 204799_at | NM_014838 | 1102 | |
| RAB18 | RAB18, member RAS | 0.57 | 224787_s_at | AI333232 | 1103 | |
| | oncogene family | | | | | |
| E2F3 | E2F transcription factor 3 | 0.57 | 203693_s_at | NM_001949 | 1104 | |
| ROCK1 | Rho-associated, coiled-coil | 0.57 | 214578_s_at | AV683882 | 1105 | |
| | containing protein kinase 1 | | | | | |
| MTO1 | mitochondrial translation | 0.57 | 222014_x_at | AI249752 | 1106 | |
| | optimization 1 homolog (S. cerevisiae) | | | | | |
| BCL10 | B-cell CLL/lymphoma 10 | 0.569 | 1557257_at | AA994334 | 1107 | |
| MSI2 | musashi homolog 2 | 0.569 | 239232_at | AA521410 | 1108 | |
| | (Drosophila) | | | | | |
| DLG1 | discs, large homolog 1 | 0.569 | 202516_s_at | NM_004087 | 1109 | |
| | (Drosophila) | | | | | |
| IDH3A | isocitrate dehydrogenase 3 | 0.569 | 202069_s_at | AI826060 | 1110 | |
| | (NAD+) alpha | | | | | |
| CUL4B | cullin 4B | 0.569 | 202213_s_at | AI650819 | 1111 | |
| | Full length insert cDNA clone | 0.569 | 1567045_at | AF085866 | 1112 | |
| | YN86A01 | | | | | |
| FLJ10326 | mitochondrial isoleucine | 0.568 | 217900_at | NM_018060 | 1113 | |
| | tRNA synthetase | | | | | |
| | Homo sapiens cDNA: | 0.568 | 216766_at | AK025152 | 1114 | |
| | FLJ21499 fis, clone | | | | | |
| | COL05634. | | | | | |
| | Transcribed sequence with | 0.568 | 244219_at | AI613089 | 1115 | |
| | weak similarity to protein | | | | | |
| | sp: P39194 (H. sapiens) | | | | | |
| | ALU7_HUMAN Alu subfamily | | | | | |
| | SQ sequence contamination | | | | | |
| | warning entry | | | | | |
| GBP3 | guanylate binding protein 3 | 0.568 | 223434_at | AL136680 | 1116 | |
| CPSF5 | wg15b02.x1 | 0.568 | 213461_at | AI800983 | 1117 | |
| | Soares_NSF_F8_9W_OT_PA_P_S1 | | | | | |
| | Homo sapiens cDNA | | | | | |
| | clone IMAGE: 2365131 3′, | | | | | |
| | mRNA sequence. | | | | | |
| LYRIC | LYRIC/3D3 | 0.567 | 212250_at | AV700332 | 1118 | |
| PPARBP | PPAR binding protein | 0.567 | 203497_at | NM_004774 | 1119 | |
| GOSR1 | wj70g04.x1 NCI_CGAP_Lu19 | 0.566 | 213020_at | AI814252 | 1120 | |
| | Homo sapiens cDNA clone | | | | | |
| | IMAGE: 2408214 3′ similar to | | | | | |
| | contains Alu repetitive | | | | | |
| | element;, mRNA sequence. | | | | | |
| KIAA1627 | KIAA1627 protein | 0.566 | 235552_at | AA354181 | 1121 | |
| ARHGDIA | Rho GDP dissociation | 0.566 | 213606_s_at | AI571798 | 1122 | |
| | inhibitor (GDI) alpha | | | | | |
| KIAA0999 | KIAA0999 protein | 0.566 | 204156_at | AA044154 | 1123 | |
| RAB3GAP | RAB3 GTPase-ACTIVATING | 0.566 | 213530_at | AI040009 | 1124 | |
| | PROTEIN | | | | | |
| API5 | apoptosis inhibitor 5 | 0.566 | 201686_x_at | AF229254 | 1125 | |
| | Transcribed sequences | 0.566 | 237663_at | AI681941 | 1126 | |
| MLL5 | myeloid/lymphoid or mixed- | 0.566 | 223189_x_at | AW082219 | 1127 | |
| | lineage leukemia 5 (trithorax | | | | | |
| | homolog, Drosophila) | | | | | |
| HSPC063 | HSPC063 protein | 0.566 | 225845_at | BG253884 | 1128 | |
| CLECSF12 | C-type (calcium dependent, | 0.565 | 1554406_a_at | BC013385 | 1129 | |
| | carbohydrate-recognition | | | | | |
| | domain) lectin, superfamily | | | | | |
| | member 12 | | | | | |
| PAPD1 | hypothetical protein | 0.565 | 229676_at | AA400998 | 1130 | |
| | FLJ10486 | | | | | |
| ZNF216 | zinc finger protein 216 | 0.565 | 210275_s_at | AF062347 | 1131 | |
| PTPN11 | predicted protein of HQ1847; | 0.565 | 209895_at | AF119855 | 1132 | |
| | Homo sapiens PRO1847 | | | | | |
| | mRNA, complete cds. | | | | | |
| PDE4B | phosphodiesterase 4B, | 0.565 | 211302_s_at | L20966 | 1133 | |
| | cAMP-specific | | | | | |
| | (phosphodiesterase E4 dunce | | | | | |
| | homolog, Drosophila) | | | | | |
| AGPS | alkylglycerone phosphate | 0.564 | 205401_at | NM_003659 | 1134 | |
| | synthase | | | | | |
| MGC3121 | hypothetical protein | 0.563 | 1559397_s_at | BE788667 | 1135 | |
| | MGC3121 | | | | | |
| GAS5 | MRNA; cDNA | 0.563 | 227517_s_at | AI056992 | 1136 | |
| | DKFZp564D0164 (from clone | | | | | |
| | DKFZp564D0164) | | | | | |
| | Transcribed sequences | 0.563 | 227565_at | BE501881 | 1137 | |
| PAX5 | paired box gene 5 (B-cell | 0.563 | 221969_at | BF510692 | 1138 | |
| | lineage specific activator | | | | | |
| | protein) | | | | | |
| SEC24B | SEC24 related gene family, | 0.563 | 202798_at | NM_006323 | 1139 | |
| | member B (S. cerevisiae) | | | | | |
| DGKA | diacylglycerol kinase, alpha | 0.563 | 211272_s_at | AF064771 | 1140 | |
| | 80 kDa | | | | | |
| EIF3S10 | eukaryotic translation | 0.563 | 200595_s_at | NM_003750 | 1141 | |
| | initiation factor 3, subunit 10 | | | | | |
| | theta, 150/170 kDa | | | | | |
| RNF6 | ring finger protein (C3H2C3 | 0.563 | 203403_s_at | NM_005977 | 1142 | |
| | type) 6 | | | | | |
| | CDNA FLJ25633 fis, clone | 0.563 | 1559119_at | W01252 | 1143 | |
| | STM04048 | | | | | |
| FLJ20701 | hypothetical protein | 0.562 | 219093_at | NM_017933 | 1144 | |
| | FLJ20701 | | | | | |
| CTNND1 | catenin (cadherin-associated | 0.562 | 208407_s_at | NM_001331 | 1145 | |
| | protein), delta 1 | | | | | |
| PSIP1 | | 0.562 | 205961_s_at | NM_004682 | 1146 | |
| GTF2I | general transcription factor II, i | 0.562 | 210892_s_at | BC004472 | 1147 | |
| MLSTD2 | male sterility domain | 0.562 | 1558014_s_at | BG261090 | 1148 | |
| | containing 2 | | | | | |
| USP38 | ubiquitin specific protease 38 | 0.562 | 223288_at | AW977401 | 1149 | |
| HHL | Homo sapiens IDN4-GGTR7 | 0.562 | 215342_s_at | AB019490 | 1150 | |
| | mRNA, partial cds. | | | | | |
| FLJ23018 | hypothetical protein | 0.562 | 205583_s_at | NM_024810 | 1151 | |
| | FLJ23018 | | | | | |
| ZNF559 | zinc finger protein 559 | 0.561 | 224518_s_at | BC006436 | 1152 | |
| SLC35A3 | solute carrier family 35 (UDP- | 0.561 | 206770_s_at | NM_012243 | 1153 | |
| | N-acetylglucosamine (UDP- | | | | | |
| | GlcNAc) transporter), | | | | | |
| | member A3 | | | | | |
| LENG8 | ty35a06.x1 NCI_CGAP_Ut2 | 0.561 | 224673_at | AI613244 | 1154 | |
| | Homo sapiens cDNA clone | | | | | |
| | IMAGE: 2281042 3′ similar to | | | | | |
| | SW: EXTN_TOBAC P13983 | | | | | |
| | EXTENSIN PRECURSOR;, | | | | | |
| | mRNA sequence. | | | | | |
| CREB1 | cAMP responsive element | 0.561 | 237289_at | AW593801 | 1155 | |
| | binding protein 1 | | | | | |
| ADK | adenosine kinase | 0.561 | 204120_s_at | NM_001123 | 1156 | |
| ARF6 | ADP-ribosylation factor 6 | 0.561 | 224788_at | AV700721 | 1157 | |
| MANEA | mannosidase, endo-alpha | 0.56 | 219003_s_at | NM_024641 | 1158 | |
| THAP5 | THAP domain containing 5 | 0.56 | 227636_at | BG500677 | 1159 | |
| ZNF227 | zinc finger protein 227 | 0.56 | 227689_at | BF739795 | 1160 | |
| MLL3 | myeloid/lymphoid or mixed- | 0.56 | 222413_s_at | AW137099 | 1161 | |
| | lineage leukemia 3 | | | | | |
| C8orf1 | chromosome 8 open reading | 0.56 | 1554414_a_at | BC031054 | 1162 | |
| | frame 1 | | | | | |
| UQCRB | ubiquinol-cytochrome c | 0.56 | 205849_s_at | NM_006294 | 1163 | |
| | reductase binding protein | | | | | |
| MCP | membrane cofactor protein | 0.559 | 207549_x_at | NM_002389 | 1164 | |
| | (CD46, trophoblast- | | | | | |
| | lymphocyte cross-reactive | | | | | |
| | antigen) | | | | | |
| SMARCA4 | SWI/SNF related, matrix | 0.559 | 213720_s_at | AI831675 | 1165 | |
| | associated, actin dependent | | | | | |
| | regulator of chromatin, | | | | | |
| | subfamily a, member 4 | | | | | |
| AGTPBP1 | ATP/GTP binding protein 1 | 0.559 | 204500_s_at | NM_015239 | 1166 | |
| DKFZp762A217 | hypothetical protein | 0.559 | 235775_at | AI765006 | 1167 | |
| | DKFZp762A217 | | | | | |
| LAMP1 | lysosomal-associated | 0.559 | 201551_s_at | J03263 | 1168 | |
| | membrane protein 1 | | | | | |
| TRERF1 | transcriptional regulating | 0.559 | 238520_at | BF724270 | 1169 | |
| | factor 1 | | | | | |
| C13orf23 | hypothetical protein | 0.559 | 225887_at | AL522406 | 1170 | |
| | FLJ12661 | | | | | |
| SIAT8D | sialyltransferase 8D (alpha-2, | 0.559 | 242943_at | AA352113 | 1171 | |
| | 8-polysialyltransferase) | | | | | |
| TNRC6 | trinucleotide repeat | 0.559 | 224705_s_at | AI684626 | 1172 | |
| | containing 6 | | | | | |
| | Clone IMAGE: 5262215, | 0.559 | 1569041_at | BC035102 | 1173 | |
| | mRNA | | | | | |
| EIF1AY | eukaryotic translation | 0.558 | 204409_s_at | BC005248 | 1174 | |
| | initiation factor 1A, Y-linked | | | | | |
| MAP4K5 | mitogen-activated protein | 0.558 | 203552_at | AW298170 | 1175 | |
| | kinase kinase kinase kinase 5 | | | | | |
| FLJ20668 | hypothetical protein | 0.558 | 219574_at | NM_017923 | 1176 | |
| | FLJ20668 | | | | | |
| FNBP4 | formin binding protein 4 | 0.558 | 235101_at | AV683244 | 1177 | |
| SP192 | hypothetical protein SP192 | 0.558 | 1569320_at | CA391618 | 1178 | |
| SLC39A6 | ts65a01.x1 NCI_CGAP_Kid8 | 0.557 | 202088_at | AI635449 | 1179 | |
| | Homo sapiens cDNA clone | | | | | |
| | IMAGE: 2233416 3′, mRNA | | | | | |
| | sequence. | | | | | |
| PXMP3 | peroxisomal membrane | 0.557 | 1561238_at | AL832262 | 1180 | |
| | protein 3, 35 kDa (Zellweger | | | | | |
| | syndrome) | | | | | |
| CYorf15A | chromosome Y open reading | 0.557 | 236694_at | AW468885 | 1181 | |
| | frame 15A | | | | | |
| NRAS | neuroblastoma RAS viral (v- | 0.556 | 202647_s_at | NM_002524 | 1182 | |
| | ras) oncogene homolog | | | | | |
| SPG20 | spastic paraplegia 20, spartin | 0.556 | 212526_at | AK002207 | 1183 | |
| | (Troyer syndrome) | | | | | |
| | Transcribed sequences | 0.556 | 238595_at | AV702101 | 1184 | |
| SRPK2 | SFRS protein kinase 2 | 0.556 | 1558254_s_at | BU155802 | 1185 | |
| FLJ38482 | hypothetical protein | 0.556 | 1555790_a_at | BC036301 | 1186 | |
| | FLJ38482 | | | | | |
| SFRS2 | splicing factor, | 0.556 | 200753_x_at | BE866585 | 1187 | |
| | arginine/serine-rich 2 | | | | | |
| MST4 | Mst3 and SOK1-related | 0.556 | 224407_s_at | AF344882 | 1188 | |
| | kinase | | | | | |
| DNMT2 | DNA (cytosine-5-)- | 0.556 | 206308_at | AJ223333 | 1189 | |
| | methyltransferase 2 | | | | | |
| LIN7C | lin-7 homolog C (C. elegans) | 0.556 | 219399_at | NM_018362 | 1190 | |
| CCNG2 | cyclin G2 | 0.555 | 202769_at | AW134535 | 1191 | |
| RC3 | rabconnectin-3 | 0.555 | 212820_at | AB020663 | 1192 | |
| MFAP3 | microfibrillar-associated | 0.555 | 1552312_a_at | NM_005927 | 1193 | |
| | protein 3 | | | | | |
| TGFBR2 | transforming growth factor, | 0.555 | 207334_s_at | NM_003242 | 1194 | |
| | beta receptor II (70/80 kDa) | | | | | |
| DHX29 | DEAH (Asp-Glu-Ala-His) box | 0.555 | 212648_at | AL079292 | 1195 | |
| | polypeptide 29 | | | | | |
| IREB2 | iron-responsive element | 0.554 | 225892_at | BF438417 | 1196 | |
| | binding protein 2 | | | | | |
| | Transcribed sequences | 0.554 | 235860_at | AA719526 | 1197 | |
| PXK | PX domain containing | 0.554 | 1552275_s_at | BG573647 | 1198 | |
| | serine/threonine kinase | | | | | |
| KIAA0650 | Transcribed sequence with | 0.553 | 212569_at | AV699744 | 1199 | |
| | strong similarity to protein | | | | | |
| | pir: T12523 (H. sapiens) | | | | | |
| | T12523 hypothetical protein | | | | | |
| | DKFZp434K063.1 —human | | | | | |
| | Clone IMAGE: 4106389, | 0.553 | 1560263_at | BC016780 | 1200 | |
| | mRNA | | | | | |
| CTBP2 | C-terminal binding protein 2 | 0.553 | 201218_at | N23018 | 1201 | |
| PTPRC | protein tyrosine phosphatase, | 0.553 | 212588_at | Y00062 | 1202 | |
| | receptor type, C | | | | | |
| C9orf83 | chromosome 9 open reading | 0.553 | 219356_s_at | NM_016410 | 1203 | |
| | frame 83 | | | | | |
| UBA2 | SUMO-1 activating enzyme | 0.553 | 201177_s_at | NM_005499 | 1204 | |
| | subunit 2 | | | | | |
| IGHG1 | Homo sapiens IgH VH gene | 0.552 | 216510_x_at | AB035175 | 1205 | |
| | for immunoglobulin heavy | | | | | |
| | chain, partial cds. | | | | | |
| CYFIP1 | cytoplasmic FMR1 interacting | 0.552 | 208923_at | BC005097 | 1206 | |
| | protein 1 | | | | | |
| PICALM | phosphatidylinositol binding | 0.552 | 212511_at | AI766247 | 1207 | |
| | clathrin assembly protein | | | | | |
| C14orf85 | CDNA: FLJ21234 fis, clone | 0.552 | 234594_at | AK024887 | 1208 | |
| | COL00841 | | | | | |
| | CDNA FLJ31796 fis, clone | 0.552 | 1559067_a_at | AI199398 | 1209 | |
| | NT2RI2008841 | | | | | |
| FLJ14007 | hypothetical protein | 0.552 | 218919_at | NM_024699 | 1210 | |
| | FLJ14007 | | | | | |
| CD36 | CD36 antigen (collagen type I | 0.552 | 206488_s_at | NM_000072 | 1211 | |
| | receptor, thrombospondin | | | | | |
| | receptor) | | | | | |
| MCP | membrane cofactor protein | 0.552 | 208783_s_at | AL570661 | 1212 | |
| | (CD46, trophoblast- | | | | | |
| | lymphocyte cross-reactive | | | | | |
| | antigen) | | | | | |
| MSH6 | mutS homolog 6 (E. coli) | 0.552 | 211450_s_at | D89646 | 1213 | |
| RRN3 | RNA polymerase I | 0.551 | 222204_s_at | AL110238 | 1214 | |
| | transcription factor RRN3 | | | | | |
| | MRNA; cDNA | 0.551 | 226810_at | BE500942 | 1215 | |
| | DKFZp761M0111 (from clone | | | | | |
| | DKFZp761M0111) | | | | | |
| DMTF1 | cyclin D binding myb-like | 0.55 | 203301_s_at | NM_021145 | 1216 | |
| | transcription factor 1 | | | | | |
| ITPR2 | Transcribed sequences | 0.55 | 202660_at | AA834576 | 1217 | |
| CDC2L5 | cell division cycle 2-like 5 | 0.55 | 207319_s_at | NM_003718 | 1218 | |
| | (cholinesterase-related cell | | | | | |
| | division controller) | | | | | |
| SOS2 | son of sevenless homolog 2 | 0.549 | 211665_s_at | L20686 | 1219 | |
| | (Drosophila) | | | | | |
| FLJ11151 | hypothetical protein | 0.549 | 218610_s_at | NM_018340 | 1220 | |
| | FLJ11151 | | | | | |
| BACH1 | BTB and CNC homology 1, | 0.549 | 204194_at | NM_001186 | 1221 | |
| | basic leucine zipper | | | | | |
| | transcription factor 1 | | | | | |
| CR1 | complement component | 0.549 | 208488_s_at | NM_000651 | 1222 | |
| | (3b/4b) receptor 1, including | | | | | |
| | Knops blood group system | | | | | |
| Ufm1 | hypothetical protein BM-002 | 0.549 | 222502_s_at | BC005193 | 1223 | |
| PAFAH1B1 | platelet-activating factor | 0.548 | 200813_s_at | BE256969 | 1224 | |
| | acetylhydrolase, isoform lb, | | | | | |
| | alpha subunit 45 kDa | | | | | |
| | Transcribed sequence with | 0.548 | 1556849_at | AU146310 | 1225 | |
| | weak similarity to protein | | | | | |
| | ref: NP_060265.1 (H. sapiens) | | | | | |
| | hypothetical protein | | | | | |
| | FLJ20378 [Homo sapiens ] | | | | | |
| DC-UbP | 601878594F1 NIH_MGC_55 | 0.547 | 224834_at | BF243404 | 1226 | |
| | Homo sapiens cDNA clone | | | | | |
| | IMAGE: 4107407 5′, mRNA | | | | | |
| | sequence. | | | | | |
| FLJ10074 | hypothetical protein | 0.547 | 221220_s_at | NM_017988 | 1227 | |
| | FLJ10074 | | | | | |
| | Transcribed sequence with | 0.547 | 215392_at | AU148154 | 1228 | |
| | moderate similarity to protein | | | | | |
| | ref: NP_060190.1 (H. sapiens) | | | | | |
| | hypothetical protein | | | | | |
| | FLJ20234 [Homo sapiens ] | | | | | |
| CPSF2 | cleavage and polyadenylation | 0.547 | 225994_at | AK023583 | 1229 | |
| | specific factor 2, 100 kDa | | | | | |
| | CDNA: FLJ21228 fis, clone | 0.546 | 234604_at | AK024881 | 1230 | |
| | COL00739 | | | | | |
| | Transcribed sequences | 0.546 | 241893_at | BE927766 | 1231 | |
| SLMAP | sarcolemma associated | 0.545 | 224149_x_at | AF304450 | 1232 | |
| | protein | | | | | |
| | UI-H-FL1-bfw-c-18-0-UI.s1 | 0.545 | 1555858_at | CA430188 | 1233 | |
| | NCI_CGAP_FL1 Homo | | | | | |
| | sapiens cDNA clone UI-H- | | | | | |
| | FL1-bfw-c-18-0-UI 3′, mRNA | | | | | |
| | sequence. | | | | | |
| DOCK11 | dedicator of cytokinesis 11 | 0.545 | 238356_at | AW968823 | 1234 | |
| | Clone IMAGE: 4798349, | 0.545 | 231576_at | AA829940 | 1235 | |
| | mRNA | | | | | |
| CXorf15 | chromosome X open reading | 0.544 | 1557954_at | BM757251 | 1236 | |
| | frame 15 | | | | | |
| SMARCA5 | SWI/SNF related, matrix | 0.544 | 202303_x_at | NM_003601 | 1237 | |
| | associated, actin dependent | | | | | |
| | regulator of chromatin, | | | | | |
| | subfamily a, member 5 | | | | | |
| | UI-H-FL1-bfw-c-18-0-UI.s1 | 0.544 | 1555860_x_at | CA430188 | 1238 | |
| | NCI_CGAP_FL1 Homo | | | | | |
| | sapiens cDNA clone UI-H- | | | | | |
| | FL1-bfw-c-18-0-UI 3′, mRNA | | | | | |
| | sequence. | | | | | |
| NEDD5 | neural precursor cell | 0.544 | 1554747_a_at | BC033559 | 1239 | |
| | expressed, developmentally | | | | | |
| | down-regulated 5 | | | | | |
| NP220 | NP220 nuclear protein | 0.544 | 1554249_a_at | BC024000 | 1240 | |
| TMOD3 | tropomodulin 3 (ubiquitous) | 0.544 | 220800_s_at | NM_014547 | 1241 | |
| ZNF506 | CDNA FLJ16466 fis, clone | 0.543 | 238493_at | AI559570 | 1242 | |
| | BRHIP2018635, moderately | | | | | |
| | similar to Zinc finger protein | | | | | |
| | 85 | | | | | |
| KIAA1789 | KIAA1789 protein | 0.542 | 226344_at | AI741051 | 1243 | |
| SIAT8D | sialyltransferase 8D (alpha-2, | 0.542 | 230261_at | AA552969 | 1244 | |
| | 8-polysialyltransferase) | | | | | |
| SLC6A6 | solute carrier family 6 | 0.542 | 205921_s_at | U16120 | 1245 | |
| | (neurotransmitter transporter, | | | | | |
| | taurine), member 6 | | | | | |
| MCL1 | 7i06c12.x1 NCI_CGAP_Co16 | 0.542 | 200796_s_at | BF594446 | 1246 | |
| | Homo sapiens cDNA clone | | | | | |
| | IMAGE: 3324694 3′ similar to | | | | | |
| | SW: MCL1_HUMAN Q07820 | | | | | |
| | INDUCED MYELOID | | | | | |
| | LEUKEMIA CELL | | | | | |
| | DIFFERENTIATION | | | | | |
| | PROTEIN MCL-1. [1]; | | | | | |
| | contains MER22.t2 TAR1 | | | | | |
| | TAR1 repetitive element;, | | | | | |
| | mRNA sequence. | | | | | |
| FLJ33860 | hypothetical protein | 0.542 | 1553311_at | NM_173644 | 1247 | |
| | FLJ33860 | | | | | |
| | Transcribed sequences | 0.541 | 239448_at | AI475033 | 1248 | |
| | Similar to dJ132F21.2 | 0.541 | 234987_at | AV715309 | 1249 | |
| | (Contains a novel protein | | | | | |
| | similar to the L82E from | | | | | |
| | Drosophila) (LOC343574), | | | | | |
| | mRNA | | | | | |
| | Transcribed sequence with | 0.541 | 235680_at | AI914925 | 1250 | |
| | moderate similarity to protein | | | | | |
| | sp: P39188 (H. sapiens) | | | | | |
| | ALU1_HUMAN Alu subfamily | | | | | |
| | J sequence contamination | | | | | |
| | warning entry | | | | | |
| | predicted protein of HQ1412; | 0.541 | 210717_at | AF116659 | 1251 | |
| | Homo sapiens PRO1412 | | | | | |
| | mRNA, complete cds. | | | | | |
| MALT1 | mucosa associated lymphoid | 0.541 | 208309_s_at | NM_006785 | 1252 | |
| | tissue lymphoma | | | | | |
| | translocation gene 1 | | | | | |
| GFPT1 | glutamine-fructose-6- | 0.541 | 202722_s_at | NM_002056 | 1253 | |
| | phosphate transaminase 1 | | | | | |
| ITGB2 | integrin, beta 2 (antigen | 0.541 | 236988_x_at | W68403 | 1254 | |
| | CD18 (p95), lymphocyte | | | | | |
| | function-associated antigen 1; | | | | | |
| | macrophage antigen 1 (mac- | | | | | |
| | 1) beta subunit) | | | | | |
| SMARCA2 | SWI/SNF related, matrix | 0.541 | 212257_s_at | AW131754 | 1255 | |
| | associated, actin dependent | | | | | |
| | regulator of chromatin, | | | | | |
| | subfamily a, member 2 | | | | | |
| RNASET2 | ribonuclease T2 | 0.541 | 232722_at | AK001769 | 1256 | |
| MATR3 | matrin 3 | 0.54 | 200626_s_at | NM_018834 | 1257 | |
| FLJ20097 | hypothetical protein | 0.54 | 226031_at | AA523733 | 1258 | |
| | FLJ20097 | | | | | |
| DNAJC13 | RME8 protein | 0.539 | 212467_at | AB014578 | 1259 | |
| LIG4 | ligase IV, DNA, ATP- | 0.539 | 227766_at | AI829314 | 1260 | |
| | dependent | | | | | |
| APP | amyloid beta (A4) precursor | 0.539 | 200602_at | NM_000484 | 1261 | |
| | protein (protease nexin-II, | | | | | |
| | Alzheimer disease) | | | | | |
| LGALS8 | lectin, galactoside-binding, | 0.538 | 210732_s_at | AF342816 | 1262 | |
| | soluble, 8 (galectin 8) | | | | | |
| ARID1A | AT rich interactive domain 1A | 0.538 | 210649_s_at | AF231056 | 1263 | |
| | (SWI-like) | | | | | |
| P2RY10 | purinergic receptor P2Y, G- | 0.538 | 1553856_s_at | NM_014499 | 1264 | |
| | protein coupled, 10 | | | | | |
| | MRNA; cDNA | 0.538 | 238919_at | R49295 | 1265 | |
| | DKFZp686L21117 (from | | | | | |
| | clone DKFZp686L21117) | | | | | |
| MDA5 | melanoma differentiation | 0.536 | 1555464_at | BC046208 | 1266 | |
| | associated protein-5 | | | | | |
| LOC142937 | | 0.536 | 1552986_at | NM_138470 | 1267 | |
| APC | adenomatosis polyposis coli | 0.536 | 203525_s_at | AI375486 | 1268 | |
| RECK | reversion-inducing-cysteine- | 0.536 | 205407_at | NM_021111 | 1269 | |
| | rich protein with kazal motifs | | | | | |
| KIAA1598 | KIAA1598 protein | 0.535 | 221802_s_at | AU157109 | 1270 | |
| PFAAP5 | phosphonoformate immuno- | 0.535 | 235547_at | BG548427 | 1271 | |
| | associated protein 5 | | | | | |
| | aa47c07.s1 | 0.535 | 242176_at | AA491228 | 1272 | |
| | NCI_CGAP_GCB1 Homo | | | | | |
| | sapiens cDNA clone | | | | | |
| | IMAGE: 824076 3′, mRNA | | | | | |
| | sequence. | | | | | |
| CMAH | | 0.534 | 205518_s_at | NM_003570 | 1273 | |
| IBTK | inhibitor of Bruton | 0.534 | 210970_s_at | AF235049 | 1274 | |
| | agammaglobulinemia tyrosine | | | | | |
| | kinase | | | | | |
| RAB18 | NB4 apoptosis related | 0.534 | 224377_s_at | AF274957 | 1275 | |
| | protein; Homo sapiens | | | | | |
| | PNAS-32 mRNA, complete | | | | | |
| | cds. | | | | | |
| RAD21 | RAD21 homolog (S. pombe) | 0.534 | 200608_s_at | NM_006265 | 1276 | |
| TncRNA | MRNA; cDNA | 0.534 | 224565_at | BE675516 | 1277 | |
| | DKFZp686L01105 (from | | | | | |
| | clone DKFZp686L01105) | | | | | |
| PAPOLA | poly(A) polymerase alpha | 0.534 | 212720_at | AI670847 | 1278 | |
| FAM36A | hypothetical protein | 0.534 | 225786_at | AI440495 | 1279 | |
| | LOC116228 | | | | | |
| ZNF430 | zinc finger protein 430 | 0.533 | 238614_x_at | AW954842 | 1280 | |
| TOPORS | topoisomerase I binding, | 0.533 | 204071_s_at | NM_005802 | 1281 | |
| | arginine/serine-rich | | | | | |
| KIAA1618 | oq48f08.s1 NCI_CGAP_Kid5 | 0.532 | 231956_at | AA976354 | 1282 | |
| | Homo sapiens cDNA clone | | | | | |
| | IMAGE: 1589607 3′ similar to | | | | | |
| | contains Alu repetitive | | | | | |
| | element;, mRNA sequence. | | | | | |
| | 601652242F1 NIH_MGC_82 | 0.532 | 235167_at | BE972419 | 1283 | |
| | Homo sapiens cDNA clone | | | | | |
| | IMAGE: 3935554 5′, mRNA | | | | | |
| | sequence. | | | | | |
| C14orf170 | | 0.532 | 214659_x_at | AC007956 | 1284 | |
| | CDNA FLJ38416 fis, clone | 0.532 | 235230_at | AW170015 | 1285 | |
| | FEBRA2009588 | | | | | |
| PLEKHG1 | Human DNA sequence from | 0.532 | 226122_at | AL035086 | 1286 | |
| | clone RP1-44A20 on | | | | | |
| | chromosome 6q23.1-24.3, | | | | | |
| | complete sequence. | | | | | |
| CDC5L | xv38c01.x1 | 0.531 | 209055_s_at | AW268817 | 1287 | |
| | Soares_NFL_T_GBC_S1 | | | | | |
| | Homo sapiens cDNA clone | | | | | |
| | IMAGE: 2815392 3′ similar to | | | | | |
| | TR: O08837 O08837 CDC- | | | | | |
| | LIKE PROTEIN;, mRNA | | | | | |
| | sequence. | | | | | |
| SPG4 | spastic paraplegia 4 | 0.531 | 207724_s_at | NM_014946 | 1288 | |
| | (autosomal dominant; | | | | | |
| | spastin) | | | | | |
| PAI-RBP1 | PAI-1 mRNA-binding protein | 0.531 | 217725_x_at | NM_015640 | 1289 | |
| | CDNA FLJ25345 fis, clone | 0.531 | 239606_at | AA669135 | 1290 | |
| | TST01118 | | | | | |
| | CDNA FLJ11613 fis, clone | 0.531 | 229028_s_at | W73694 | 1291 | |
| | HEMBA1004012 | | | | | |
| ASXL2 | additional sex combs like 2 | 0.531 | 1555266_a_at | BC042999 | 1292 | |
| | (Drosophila) | | | | | |
| | 602503750F1 NIH_MGC_77 | 0.531 | 232744_x_at | BG485129 | 1293 | |
| | Homo sapiens cDNA clone | | | | | |
| | IMAGE: 4617157 5′, mRNA | | | | | |
| | sequence. | | | | | |
| KIAA2026 | KIAA2026 | 0.53 | 238490_at | BG109896 | 1294 | |
| ZNF146 | zinc finger protein 146 | 0.53 | 1554433_a_at | BC005154 | 1295 | |
| CCNG2 | cyclin G2 | 0.53 | 211559_s_at | L49506 | 1296 | |
| DOCK8 | | 0.53 | 232843_s_at | AL161725 | 1297 | |
| IDN3 | IDN3 protein | 0.529 | 213918_s_at | BF221673 | 1298 | |
| ZNFN1A1 | zinc finger protein, subfamily | 0.529 | 216901_s_at | S80876 | 1299 | |
| | 1A, 1 (Ikaros) | | | | | |
| PDGFC | platelet derived growth factor C | 0.528 | 218718_at | NM_016205 | 1300 | |
| SLC39A6 | solute carrier family 39 (zinc | 0.528 | 202089_s_at | NM_012319 | 1301 | |
| | transporter), member 6 | | | | | |
| RANBP2 | RAN binding protein 2 | 0.528 | 201713_s_at | D42063 | 1302 | |
| | Transcribed sequences | 0.528 | 242551_at | AI223854 | 1303 | |
| EKI1 | ethanolamine kinase | 0.527 | 219017_at | NM_018638 | 1304 | |
| ANP32E | acidic (leucine-rich) nuclear | 0.526 | 229128_s_at | AI697657 | 1305 | |
| | phosphoprotein 32 family, | | | | | |
| | member E | | | | | |
| MAP3K7IP2 | mitogen-activated protein | 0.525 | 210284_s_at | AF241230 | 1306 | |
| | kinase kinase kinase 7 | | | | | |
| | interacting protein 2 | | | | | |
| DOCK10 | | 0.525 | 219279_at | NM_017718 | 1307 | |
| KIAA0125 | KIAA0125 | 0.524 | 206478_at | NM_014792 | 1308 | |
| | Clone IMAGE: 4798349, | 0.524 | 226432_at | AI276880 | 1309 | |
| | mRNA | | | | | |
| MAFB | v-maf musculoaponeurotic | 0.523 | 218559_s_at | NM_005461 | 1310 | |
| | fibrosarcoma oncogene | | | | | |
| | homolog B (avian) | | | | | |
| | Transcribed sequence with | 0.522 | 238902_at | T85248 | 1311 | |
| | weak similarity to protein | | | | | |
| | sp: P39188 (H. sapiens) | | | | | |
| | ALU1_HUMAN Alu subfamily | | | | | |
| | J sequence contamination | | | | | |
| | warning entry | | | | | |
| STAG1 | propionyl Coenzyme A | 0.522 | 202293_at | AW168948 | 1312 | |
| | carboxylase, beta polypeptide | | | | | |
| RAI17 | retinoic acid induced 17 | 0.521 | 212124_at | AF070622 | 1313 | |
| | CDNA: FLJ23262 fis, clone | 0.521 | 1566113_at | AA176313 | 1314 | |
| | COL05922 | | | | | |
| PICALM | phosphatidylinositol binding | 0.521 | 215236_s_at | AV721177 | 1315 | |
| | clathrin assembly protein | | | | | |
| KIAA0256 | KIAA0256 gene product | 0.521 | 233406_at | AK022100 | 1316 | |
| EPS15L1 | epidermal growth factor | 0.52 | 222112_at | AV710549 | 1317 | |
| | receptor pathway substrate | | | | | |
| | 15-like 1 | | | | | |
| SUPT16H | suppressor of Ty 16 homolog | 0.519 | 233827_s_at | AK024072 | 1318 | |
| | (S. cerevisiae) | | | | | |
| KIAA0217 | ob72f05.s1 | 0.519 | 208952_s_at | AA811923 | 1319 | |
| | NCI_CGAP_GCB1 Homo | | | | | |
| | sapiens cDNA clone | | | | | |
| | IMAGE: 1336929 3′, mRNA | | | | | |
| | sequence. | | | | | |
| FLJ12687 | hypothetical protein | 0.519 | 205238_at | NM_024917 | 1320 | |
| | FLJ12687 | | | | | |
| CPA3 | carboxypeptidase A3 (mast | 0.519 | 205624_at | NM_001870 | 1321 | |
| | cell) | | | | | |
| STXBP3 | syntaxin binding protein 3 | 0.518 | 203310_at | NM_007269 | 1322 | |
| WSB1 | WD repeat and SOCS box- | 0.518 | 201295_s_at | BF111821 | 1323 | |
| | containing 1 | | | | | |
| JMJD1C | thyroid hormone receptor | 0.515 | 221763_at | AI694023 | 1324 | |
| | interactor 8 | | | | | |
| | Transcribed sequences | 0.515 | 236322_at | AA830854 | 1325 | |
| | CDNA FLJ39417 fis, clone | 0.515 | 229958_at | W93695 | 1326 | |
| | PLACE6016942 | | | | | |
| | HSPC106 mRNA, partial cds | 0.515 | 232551_at | AA521443 | 1327 | |
| CCAR1 | cell division cycle and | 0.515 | 239014_at | W73136 | 1328 | |
| | apoptosis regulator 1 | | | | | |
| USP34 | ubiquitin specific protease 34 | 0.515 | 212065_s_at | AW502434 | 1329 | |
| FLJ20360 | hypothetical protein | 0.514 | 227777_at | BF223040 | 1330 | |
| | FLJ20360 | | | | | |
| PTPN12 | protein tyrosine phosphatase, | 0.512 | 202006_at | NM_002835 | 1331 | |
| | non-receptor type 12 | | | | | |
| SEC10L1 | SEC10-like 1 (S. cerevisiae) | 0.512 | 218748_s_at | NM_006544 | 1332 | |
| NCOA2 | nuclear receptor coactivator 2 | 0.512 | 205732_s_at | NM_006540 | 1333 | |
| PDE3B | phosphodiesterase 3B, | 0.511 | 208591_s_at | NM_000922 | 1334 | |
| | cGMP-inhibited | | | | | |
| XTP2 | HBxAg transactivated protein 2 | 0.511 | 214052_x_at | AW301305 | 1335 | |
| | MRNA; cDNA | 0.51 | 235705_at | BF676361 | 1336 | |
| | DKFZp564O2423 (from clone | | | | | |
| | DKFZp564O2423) | | | | | |
| SEC63 | SEC63-like (S. cerevisiae) | 0.51 | 201916_s_at | NM_007214 | 1337 | |
| | Transcribed sequence with | 0.51 | 244286_at | AI017983 | 1338 | |
| | moderate similarity to protein | | | | | |
| | ref: NP_060265.1 (H. sapiens) | | | | | |
| | hypothetical protein | | | | | |
| | FLJ20378 [Homo sapiens ] | | | | | |
| | CDNA FLJ12399 fis, clone | 0.51 | 232472_at | AK022461 | 1339 | |
| | MAMMA1002780 | | | | | |
| PNN | pinin, desmosome associated | 0.51 | 212036_s_at | AW152664 | 1340 | |
| | protein | | | | | |
| HRH4 | histamine receptor H4 | 0.51 | 221170_at | AF312230 | 1341 | |
| EPPK1 | epiplakin 1 | 0.509 | 232164_s_at | AL137725 | 1342 | |
| PRKWNK1 | | 0.508 | 202940_at | NM_014823 | 1343 | |
| CRSP3 | cofactor required for Sp1 | 0.508 | 242706_s_at | AW959449 | 1344 | |
| | transcriptional activation, | | | | | |
| | subunit 3, 130 kDa | | | | | |
| ZFHX1B | zinc finger homeobox 1b | 0.508 | 203603_s_at | NM_014795 | 1345 | |
| C14orf125 | chromosome 14 open reading | 0.505 | 226510_at | BF435286 | 1346 | |
| | frame 125 | | | | | |
| SNX2 | Homo sapiens sorting nexin 2 | 0.505 | 202113_s_at | AF043453 | 1347 | |
| | (SNX2) mRNA, complete cds. | | | | | |
| SOX4 | | 0.505 | 201417_at | AL136179 | 1348 | |
| LARP | likely ortholog of mouse Ia | 0.504 | 212193_s_at | BE881529 | 1349 | |
| | related protein | | | | | |
| | MRNA; cDNA | 0.504 | 238918_at | AI985021 | 1350 | |
| | DKFZp686K1098 (from clone | | | | | |
| | DKFZp686K1098) | | | | | |
| ADAM10 | a disintegrin and | 0.503 | 214895_s_at | AU135154 | 1351 | |
| | metalloproteinase domain 10 | | | | | |
| CBX3 | chromobox homolog 3 (HP1 | 0.502 | 201091_s_at | BE748755 | 1352 | |
| | gamma homolog, Drosophila) | | | | | |
| NCOA1 | nuclear receptor coactivator 1 | 0.501 | 209105_at | AI672428 | 1353 | |
| ITGA4 | integrin, alpha 4 (antigen | 0.5 | 205885_s_at | L12002 | 1354 | |
| | CD49D, alpha 4 subunit of | | | | | |
| | VLA-4 receptor) | | | | | |
| JAK1 | Janus kinase 1 (a protein | 0.5 | 1552610_a_at | NM_002227 | 1355 | |
| | tyrosine kinase) | | | | | |
| LOC115294 | RC4-HT0276-100300-015- | 0.499 | 232382_s_at | BE150929 | 1356 | |
| | e11 HT0276 Homo sapiens | | | | | |
| | cDNA, mRNA sequence. | | | | | |
| ZNF24 | zinc finger protein 24 (KOX | 0.498 | 203248_at | NM_006965 | 1357 | |
| | 17) | | | | | |
| TIA1 | TIA1 cytotoxic granule- | 0.497 | 1554889_at | BC015944 | 1358 | |
| | associated RNA binding | | | | | |
| | protein | | | | | |
| NDUFA5 | NADH dehydrogenase | 0.497 | 201304_at | NM_005000 | 1359 | |
| | (ubiquinone) 1 alpha | | | | | |
| | subcomplex, 5, 13 kDa | | | | | |
| GOLGIN-67 | MRNA; cDNA | 0.496 | 210425_x_at | AF164622 | 1360 | |
| | DKFZp686O038 (from clone | | | | | |
| | DKFZp686O038) | | | | | |
| RHOQ | ras homolog gene family, | 0.496 | 239258_at | BE551407 | 1361 | |
| | member Q | | | | | |
| CCM1 | Homo sapiens mRNA; cDNA | 0.496 | 216713_at | AL049325 | 1362 | |
| | DKFZp564D036 (from clone | | | | | |
| | DKFZp564D036). | | | | | |
| KLF4 | Kruppel-like factor 4 (gut) | 0.495 | 221841_s_at | BF514079 | 1363 | |
| FLJ31306 | hypothetical protein | 0.495 | 225724_at | AW136120 | 1364 | |
| | FLJ31306 | | | | | |
| TCF8 | transcription factor 8 | 0.495 | 210875_s_at | U12170 | 1365 | |
| | (represses interleukin 2 | | | | | |
| | expression) | | | | | |
| UQCRC2 | ubiquinol-cytochrome c | 0.493 | 241755_at | AI961429 | 1366 | |
| | reductase core protein II | | | | | |
| KLHL5 | Homo sapiens mRNA; cDNA | 0.492 | 232297_at | AL049385 | 1367 | |
| | DKFZp586M1418 (from clone | | | | | |
| | DKFZp586M1418). | | | | | |
| MGC46719 | hypothetical protein | 0.492 | 235036_at | AA923372 | 1368 | |
| | MGC46719 | | | | | |
| ZNF160 | zinc finger protein 160 | 0.492 | 239954_at | AA701249 | 1369 | |
| CASP1 | CARD only protein | 0.49 | 1552703_s_at | NM_052889 | 1370 | |
| RTN1 | reticulon 1 | 0.49 | 203485_at | NM_021136 | 1371 | |
| HSPCA | heat shock 90 kDa protein 1, | 0.489 | 210211_s_at | AF028832 | 1372 | |
| | alpha | | | | | |
| | 7d75g01.x1 | 0.489 | 227391_x_at | BE674143 | 1373 | |
| | NCI_CGAP_Lu24 Homo | | | | | |
| | sapiens cDNA clone | | | | | |
| | IMAGE: 3278832 3′ similar to | | | | | |
| | contains element MER1 | | | | | |
| | repetitive element;, mRNA | | | | | |
| | sequence. | | | | | |
| LOC374443 | 601556050F1 NIH_MGC_58 | 0.489 | 238790_at | BE738988 | 1374 | |
| | Homo sapiens cDNA clone | | | | | |
| | IMAGE: 3825757 5′, mRNA | | | | | |
| | sequence. | | | | | |
| P15RS | hypothetical protein | 0.488 | 228566_at | AA778694 | 1375 | |
| | FLJ10656 | | | | | |
| | Transcribed sequences | 0.488 | 244418_at | AA223929 | 1376 | |
| | CDNA FLJ39805 fis, clone | 0.488 | 1557626_at | CA444630 | 1377 | |
| | SPLEN2007951 | | | | | |
| UTRN | utrophin (homologous to | 0.488 | 213022_s_at | NM_007124 | 1378 | |
| | dystrophin) | | | | | |
| DLAT | dihydrolipoamide S- | 0.487 | 212568_s_at | BF978872 | 1379 | |
| | acetyltransferase (E2 | | | | | |
| | component of pyruvate | | | | | |
| | dehydrogenase complex) | | | | | |
| HSPCB | heat shock 90 kDa protein 1, | 0.486 | 1557910_at | BG612458 | 1380 | |
| | beta | | | | | |
| PPM1B | protein phosphatase 1B | 0.484 | 213225_at | AJ271832 | 1381 | |
| | (formerly 2C), magnesium- | | | | | |
| | dependent, beta isoform | | | | | |
| ANKHD1 | multiple ankyrin repeats, | 0.484 | 233292_s_at | AK024189 | 1382 | |
| | single KH-domain (MASK) | | | | | |
| | homolog | | | | | |
| FLI1 | | 0.484 | 210786_s_at | M93255 | 1383 | |
| ARHGEF7 | Rho guanine nucleotide | 0.483 | 235412_at | AI040887 | 1384 | |
| | exchange factor (GEF) 7 | | | | | |
| | Transcribed sequences | 0.483 | 237746_at | AI168187 | 1385 | |
| GNA13 | guanine nucleotide binding | 0.482 | 206917_at | NM_006572 | 1386 | |
| | protein (G protein), alpha 13 | | | | | |
| C14orf43 | | 0.482 | 220494_s_at | NM_018678 | 1387 | |
| NEK4 | NIMA (never in mitosis gene | 0.481 | 204634_at | NM_003157 | 1388 | |
| | a)-related kinase 4 | | | | | |
| ZF | HCF-binding transcription | 0.481 | 202979_s_at | NM_021212 | 1389 | |
| | factor Zhangfei | | | | | |
| FLJ12716 | FLJ12716 protein | 0.479 | 233558_s_at | AK023390 | 1390 | |
| APG5L | APG5 autophagy 5-like (S. cerevisiae) | 0.479 | 210639_s_at | AF293841 | 1391 | |
| HSPCA | heat shock 90 kDa protein 1, | 0.479 | 211968_s_at | AI962933 | 1392 | |
| | alpha | | | | | |
| | UI-E-EO1-aid-b-20-0-UI.s1 | 0.477 | 1557706_at | BM677619 | 1393 | |
| | UI-E-EO1 Homo sapiens | | | | | |
| | cDNA clone UI-E-EO1-aid-b- | | | | | |
| | 20-0-UI 3′, mRNA sequence. | | | | | |
| DLEU2 | deleted in lymphocytic | 0.477 | 1569600_at | BC006995 | 1394 | |
| | leukemia, 2 | | | | | |
| T3JAM | TRAF3-interacting Jun N- | 0.477 | 215275_at | AW963138 | 1395 | |
| | terminal kinase (JNK)- | | | | | |
| | activating modulator | | | | | |
| PRNP | prion protein (p27-30) | 0.476 | 201300_s_at | NM_000311 | 1396 | |
| | (Creutzfeld-Jakob disease, | | | | | |
| | Gerstmann-Strausler- | | | | | |
| | Scheinker syndrome, fatal | | | | | |
| | familial insomnia) | | | | | |
| CCR2 | chemokine (C-C motif) | 0.475 | 207794_at | NM_000648 | 1397 | |
| | receptor 2 | | | | | |
| TUBGCP3 | tubulin, gamma complex | 0.475 | 203690_at | NM_006322 | 1398 | |
| | associated protein 3 | | | | | |
| OLIG1 | oligodendrocyte transcription | 0.475 | 228170_at | AL355743 | 1399 | |
| | factor 1 | | | | | |
| | Transcribed sequences | 0.474 | 244679_at | AI032730 | 1400 | |
| | AV736963 CB Homo sapiens | 0.473 | 242312_x_at | AV736963 | 1401 | |
| | cDNA clone CBFBOH07 5′, | | | | | |
| | mRNA sequence. | | | | | |
| | MRNA; cDNA | 0.47 | 1566959_at | AL049273 | 1402 | |
| | DKFZp564H023 (from clone | | | | | |
| | DKFZp564H023) | | | | | |
| | MRNA; cDNA | 0.47 | 1566243_at | AL390176 | 1403 | |
| | DKFZp547L144 (from clone | | | | | |
| | DKFZp547L144) | | | | | |
| ZNF146 | zinc finger protein 146 | 0.469 | 200050_at | NM_007145 | 1404 | |
| FER1L3 | fer-1-like 3, myoferlin (C. elegans) | 0.468 | 211864_s_at | AF207990 | 1405 | |
| | UI-H-BI1-acb-b-12-0-UI.s1 | 0.467 | 227265_at | AW135176 | 1406 | |
| | NCI_CGAP_Sub3 Homo | | | | | |
| | sapiens cDNA clone | | | | | |
| | IMAGE: 2713679 3′, mRNA | | | | | |
| | sequence. | | | | | |
| HLA-DRB3 | major histocompatibility | 0.467 | 221491_x_at | AA807056 | 1407 | |
| | complex, class II, DR beta 3 | | | | | |
| EPB41L3 | erythrocyte membrane | 0.467 | 206710_s_at | NM_012307 | 1408 | |
| | protein band 4.1-like 3 | | | | | |
| PLEKHF2 | pleckstrin homology domain | 0.466 | 222699_s_at | BF439250 | 1409 | |
| | containing, family F (with | | | | | |
| | FYVE domain) member 2 | | | | | |
| | Transcribed sequence with | 0.462 | 231152_at | AW452971 | 1410 | |
| | moderate similarity to protein | | | | | |
| | pir: I60307 (E. coli) I60307 | | | | | |
| | beta-galactosidase, alpha | | | | | |
| | peptide - Escherichia coli | | | | | |
| SMC4L1 | SMC4 structural maintenance | 0.461 | 201663_s_at | NM_005496 | 1411 | |
| | of chromosomes 4-like 1 | | | | | |
| | (yeast) | | | | | |
| DDX42 | DEAD (Asp-Glu-Ala-Asp) box | 0.46 | 1559954_s_at | AF147429 | 1412 | |
| | polypeptide 42 | | | | | |
| SMARCA2 | SWI/SNF related, matrix | 0.46 | 206544_x_at | NM_003070 | 1413 | |
| | associated, actin dependent | | | | | |
| | regulator of chromatin, | | | | | |
| | subfamily a, member 2 | | | | | |
| | CDNA clone | 0.459 | 1556527_a_at | BC038203 | 1414 | |
| | IMAGE: 4402168, partial cds | | | | | |
| TAP2 | transporter 2, ATP-binding | 0.459 | 204770_at | NM_000544 | 1415 | |
| | cassette, sub-family B | | | | | |
| | (MDR/TAP) | | | | | |
| HCRP1 | hepatocellular carcinoma- | 0.458 | 216174_at | AK025343 | 1416 | |
| | related HCRP1 | | | | | |
| | Transcribed sequence with | 0.458 | 235274_at | AA740632 | 1417 | |
| | weak similarity to protein | | | | | |
| | ref: NP_060312.1 (H. sapiens) | | | | | |
| | hypothetical protein | | | | | |
| | FLJ20489 [Homo sapiens ] | | | | | |
| TTC3 | tetratricopeptide repeat | 0.458 | 208664_s_at | AU131711 | 1418 | |
| | domain 3 | | | | | |
| SMG1 | | 0.458 | 231989_s_at | AC003007 | 1419 | |
| SGPP1 | sphingosine-1-phosphate | 0.457 | 221268_s_at | NM_030791 | 1420 | |
| | phosphatase 1 | | | | | |
| DDX10 | DEAD (Asp-Glu-Ala-Asp) box | 0.456 | 204977_at | NM_004398 | 1421 | |
| | polypeptide 10 | | | | | |
| NEDD5 | neural precursor cell | 0.456 | 200778_s_at | AI191427 | 1422 | |
| | expressed, developmentally | | | | | |
| | down-regulated 5 | | | | | |
| MEF2C | MADS box transcription | 0.454 | 207968_s_at | NM_002397 | 1423 | |
| | enhancer factor 2, | | | | | |
| | polypeptide C (myocyte | | | | | |
| | enhancer factor 2C) | | | | | |
| LNPEP | leucyl/cystinyl | 0.453 | 207904_s_at | NM_005575 | 1424 | |
| | aminopeptidase | | | | | |
| TGOLN2 | trans-golgi network protein 2 | 0.449 | 203834_s_at | NM_006464 | 1425 | |
| TFCP2 | transcription factor CP2 | 0.446 | 207627_s_at | NM_005653 | 1426 | |
| | Homo sapiens mRNA; cDNA | 0.444 | 216683_at | AL353949 | 1427 | |
| | DKFZp761P1114 (from clone | | | | | |
| | DKFZp761P1114). | | | | | |
| CSPG6 | chondroitin sulfate | 0.44 | 209259_s_at | AF020043 | 1428 | |
| | proteoglycan 6 (bamacan) | | | | | |
| PIK3AP1 | phosphoinositide-3-kinase | 0.44 | 1554508_at | BC029917 | 1429 | |
| | adaptor protein 1 | | | | | |
| JIK | STE20-like kinase | 0.44 | 236002_at | BE178502 | 1430 | |
| FLJ23091 | putative NFkB activating | 0.439 | 228950_s_at | AL534095 | 1431 | |
| | protein 373 | | | | | |
| MSI2 | musashi homolog 2 | 0.439 | 225237_s_at | BF435123 | 1432 | |
| | (Drosophila) | | | | | |
| DSP | desmoplakin | 0.433 | 200606_at | NM_004415 | 1433 | |
| SAMHD1 | SAM domain and HD domain 1 | 0.431 | 1559883_s_at | AF147427 | 1434 | |
| BCOR | BCL6 co-repressor | 0.429 | 223566_s_at | AF317391 | 1435 | |
| ILF3 | interleukin enhancer binding | 0.428 | 211375_s_at | AF141870 | 1436 | |
| | factor 3, 90 kDa | | | | | |
| H41 | hypothetical protein H41 | 0.427 | 213548_s_at | BG257762 | 1437 | |
| LOC284058 | hypothetical protein | 0.426 | 224489_at | BC006271 | 1438 | |
| | LOC284058 | | | | | |
| UPF3B | UPF3 regulator of nonsense | 0.426 | 218757_s_at | NM_023010 | 1439 | |
| | transcripts homolog B (yeast) | | | | | |
| PXN | Paxillin (PXN), mRNA | 0.422 | 211823_s_at | D86862 | 1440 | |
| CTNNA1 | catenin (cadherin-associated | 0.42 | 1558214_s_at | BG330076 | 1441 | |
| | protein), alpha 1, 102 kDa | | | | | |
| C6orf67 | chromosome 6 open reading | 0.42 | 232591_s_at | AK022883 | 1442 | |
| | frame 67 | | | | | |
| ATRX | alpha thalassemia/mental | 0.418 | 236778_at | AA826176 | 1443 | |
| | retardation syndrome X- | | | | | |
| | linked (RAD54 homolog, S. cerevisiae) | | | | | |
| HYPB | huntingtin interacting protein B | 0.418 | 241458_at | AI868267 | 1444 | |
| TTF2 | transcription termination | 0.416 | 204407_at | AF080255 | 1445 | |
| | factor, RNA polymerase II | | | | | |
| | Clone IMAGE: 5267013, | 0.415 | 1559401_a_at | BI052176 | 1446 | |
| | mRNA | | | | | |
| KBTBD2 | kelch repeat and BTB (POZ) | 0.414 | 223585_x_at | AF151831 | 1447 | |
| | domain containing 2 | | | | | |
| PTPRO | protein tyrosine phosphatase, | 0.413 | 208121_s_at | NM_002848 | 1448 | |
| | receptor type, O | | | | | |
| C14orf85 | CDNA: FLJ21234 fis, clone | 0.412 | 1563524_a_at | AK024887 | 1449 | |
| | COL00841 | | | | | |
| | CDNA FLJ35626 fis, clone | 0.411 | 1565599_at | AA700631 | 1450 | |
| | SPLEN2011086 | | | | | |
| ZNF267 | zinc finger protein 267 | 0.411 | 219540_at | AU150728 | 1451 | |
| HTATSF1 | HIV TAT specific factor 1 | 0.41 | 202602_s_at | NM_014500 | 1452 | |
| SP100 | nuclear antigen Sp100 | 0.409 | 210985_s_at | AF056322 | 1453 | |
| FNBP1 | formin binding protein 1 | 0.398 | 230086_at | AA937109 | 1454 | 42 |
| DDX3X | DEAD (Asp-Glu-Ala-Asp) box | 0.398 | 212514_x_at | R60068 | 1455 | 43 |
| | polypeptide 3, X-linked | | | | | |
| UBE4B | ubiquitination factor E4B | 0.396 | 202316_x_at | AW241715 | 1456 | 44 |
| | (UFD2 homolog, yeast) | | | | | |
| C22orf1 | chromosome 22 open reading | 0.392 | 1554948_at | BC034024 | 1457 | 45 |
| | frame 1 | | | | | |
| | CDNA FLJ10256 fis, clone | 0.392 | 215558_at | AK001118 | 1458 | 46 |
| | HEMBB1000870 | | | | | |
| IGHM | Partial mRNA for IgM | 0.39 | 215621_s_at | BG340670 | 1459 | 47 |
| | immunoglobulin heavy chain | | | | | |
| | variable region (IGHV gene), | | | | | |
| | clone LIBPM376 | | | | | |
| | Full length insert cDNA clone | 0.385 | 1557811_a_at | AF085827 | 1460 | 48 |
| | YF47B02 | | | | | |
| MO25 | MO25 protein | 0.363 | 224311_s_at | AF151824 | 1461 | 49 |
| SIAT1 | sialyltransferase 1 (beta- | 0.361 | 214971_s_at | AV695711 | 1462 | 50 |
| | galactoside alpha-2,6- | | | | | |
| | sialyltransferase) | | | | | |
| TncRNA | MRNA; cDNA | 0.338 | 224566_at | AI042152 | 1463 | 51 |
| | DKFZp686L01105 (from | | | | | |
| | clone DKFZp686L01105) | | | | | |
| MTMR1 | myotubularin related protein 1 | 0.32 | 214975_s_at | AK001816 | 1464 | 52 |
| FER1L3 | fer-1-like 3, myoferlin (C. elegans) | 0.32 | 201798_s_at | NM_013451 | 1465 | 53 |
| TncRNA | MRNA; cDNA | 0.302 | 238320_at | AV659198 | 1466 | 54 |
| | DKFZp686L01105 (from | | | | | |
| | clone DKFZp686L01105) | | | | | |
| HIPK3 | homeodomain interacting | 0.248 | 210148_at | AF305239 | 1467 | 55 |
| | protein kinase 3 | | | | | |

TABLE 4
Duchenne muscular dystrophy (DMD) vs. Duchenne muscular dystrophy (DMD)
receiving steroid. Fold changes with parentheses indicate DMD > DMD + steroid;
fold changes without parentheses indicate DMD + steroid > DMD.

| | | | Genbank | | |
|---|---|---|---|---|---|
| | | Fold | Accession | Nucleic | SEQ ID |
| Common Name | Description | Change | Number | Acid No. | NO. |
| TCL1A | T-cell leukemia/lymphoma 1A; | (3.16) | BC003574 | 1468 | |
| | T-cell leukemia/lymphoma 1A | | | | |
| TCL1A | T-cell leukemia/lymphoma 1A | (2.96) | X82240 | 1469 | |
| LAF4 | AF4/FMR2 family, member 3 | (2.53) | AA572675 | 1470 | |
| LAF4 | kelch-like 8 (Drosophila) | (2.40) | AI033582 | 1471 | |
| IGHM | immunoglobulin heavy constant | (2.26) | X17115 | 1472 | |
| | delta | | | | |
| CLC | chemokine-like factor | (2.12) | NM_001828 | 1473 | |
| | superfamily 6 | | | | |
| PCDH9 | protocadherin 9 | (2.10) | AI524125 | 1474 | |
| MBNL1 | Muscleblind-like (Drosophila) | (2.08) | AA744622 | 1475 | |
| BACH2 | UDP-GlcNAc:betaGal beta-1,3- | (2.07) | AA085906 | 1476 | |
| | N- | | | | |
| | acetylglucosaminyltransferase 5 | | | | |
| FCRH3 | FCH domain only 2 | (2.04) | BF514552 | 1477 | |
| CHI3L2 | chondroitin beta 1,4 N- | (1.98) | U58515 | 1478 | |
| | acetylgalactosaminyltransferase | | | | |
| SMA4 | SMA4 | (1.97) | X83300 | 1479 | |
| RALGPS2 | Ral GEF with PH domain and | (1.97) | NM_018037 | 1480 | |
| | SH3 binding motif 2 | | | | |
| FCRL5 | Fc receptor-like 3 | (1.97) | AW241983 | 1481 | |
| IGHM | immunoglobulin heavy constant | (1.95) | BC001872 | 1482 | |
| | mu; immunoglobulin heavy | | | | |
| | constant mu | | | | |
| | Transcribed locus | (1.95) | AW665840 | 28 | 26 |
| HTPAP | HIV TAT specific factor 1 | (1.94) | BF111651 | 1483 | |
| CD79A | CD72 antigen | (1.90) | M74721 | 1484 | |
| HLA-DOB | Homeodomain interacting | (1.89) | NM_002120 | 1485 | |
| | protein kinase 3 | | | | |
| AFF3 | ADAM metallopeptidase | (1.89) | AW085505 | 1486 | |
| | domain 9 (meltrin gamma) | | | | |
| PAPD4 | PAP associated domain | (1.88) | BG542611 | 1487 | |
| | containing 4 | | | | |
| IGHD | interferon-related | (1.88) | AI492643 | 1488 | |
| | developmental regulator 1 | | | | |
| HLA-DQA1; HLA- | major histocompatibility | (1.86) | BG397856 | 1489 | |
| DQA2 | complex, class II, DP beta 1 | | | | |
| VPREB3 | pre-B lymphocyte gene 3 | (1.85) | NM_013378 | 1490 | |
| BCNP1 | BCL2-like 1 | (1.84) | BE646461 | 1491 | |
| JMJD2C | jumonji domain containing 1C | (1.84) | AI341227 | 1492 | |
| CD79B | CD79A antigen | (1.83) | NM_000626 | 1493 | |
| | (immunoglobulin-associated | | | | |
| | alpha); CD79A antigen | | | | |
| | (immunoglobulin-associated | | | | |
| | alpha) | | | | |
| PAX5 | Paired box gene 5 (B-cell | (1.83) | BF510692 | 1138 | |
| | lineage specific activator) | | | | |
| LOC283663 | leukocyte immunoglobulin-like | (1.83) | AI926479 | 1494 | |
| | receptor, subfamily B (with TM | | | | |
| | and ITIM domains), member 2 | | | | |
| CD79A | CD79A antigen | (1.83) | NM_001783 | 1495 | |
| | (immunoglobulin-associated | | | | |
| | alpha) | | | | |
| MRPL43 | mitochondrial ribosomal protein | (1.82) | N74662 | 1496 | |
| | L43 | | | | |
| SPIB | Spi-B transcription factor (Spi- | (1.82) | NM_003121 | 1497 | |
| | 1/PU.1 related); Spi-B | | | | |
| | transcription factor (Spi-1/PU.1 | | | | |
| | related) | | | | |
| BACH2 | BTB and CNC homology 1, | (1.82) | AK027193 | 1498 | |
| | basic leucine zipper | | | | |
| | transcription factor 2 | | | | |
| IGLC2 | Immunoglobulin lambda joining 3 | (1.79) | U96394 | 1499 | |
| BACH2 | BTB and CNC homology 1, | (1.79) | AI052447 | 1500 | |
| | basic leucine zipper | | | | |
| | transcription factor 2 | | | | |
| BCL11A | BTB and CNC homology 1, | (1.78) | BM193618 | 1501 | |
| | basic leucine zipper | | | | |
| | transcription factor 2 | | | | |
| FTO | fucose-1-phosphate | (1.77) | AK022473 | 1502 | |
| | guanylyltransferase | | | | |
| LRRC5 | leucine-rich repeats and | (1.77) | AK025238 | 1503 | |
| | calponin homology (CH) | | | | |
| | domain containing 3 | | | | |
| MEF2C | MADS box transcription | (1.77) | AI523245 | 1504 | |
| | enhancer factor 2, polypeptide | | | | |
| | C (myocyte enhancer factor 2C) | | | | |
| | Immunoglobulin kappa light | (1.77) | AW404894 | 1505 | |
| | chain variable region (IGKV | | | | |
| | gene), clone 25 | | | | |
| | | (1.77) | AI475033 | 1248 | |
| GZMB | guanylate cyclase 1, soluble, | (1.76) | J03189 | 101 | |
| | beta 3 | | | | |
| BLR1 | B-cell linker | (1.76) | AI436580 | 1506 | |
| RUNX1 | Runt-related transcription factor | (1.75) | BU789637 | 1507 | |
| | 1 (acute myeloid leukemia 1; | | | | |
| | aml1 oncogene) | | | | |
| TCF4 | transcription factor 4 | (1.75) | NM_003199 | 1508 | |
| G1P2 | FYN oncogene related to SRC, | (1.74) | NM_005101 | 1509 | |
| | FGR, YES | | | | |
| STRBP | Spermatid perinuclear RNA | (1.74) | AK025613 | 1510 | |
| | binding protein | | | | |
| FGD2 | Fc receptor-like 5; Fc receptor- | (1.73) | AK024456 | 1511 | |
| | like 5 | | | | |
| MAML2 | metastasis associated lung | (1.73) | AU147805 | 1512 | |
| | adenocarcinoma transcript 1 | | | | |
| | (non-coding RNA) | | | | |
| PPP2R5C | Protein phosphatase 2, | (1.72) | AI478850 | 1513 | |
| | regulatory subunit B (B56), | | | | |
| | gamma isoform | | | | |
| MAPRE2 | mitogen-activated protein | (1.71) | BC013931 | 1514 | |
| | kinase 14 | | | | |
| ZCCHC7 | Zinc finger, CCHC domain | (1.71) | AA872187 | 1515 | |
| | containing 7 | | | | |
| FYN | Fatso | (1.71) | BG222258 | 1516 | |
| | CDNA FLJ45435 fis, clone | (1.71) | AA700870 | 1517 | |
| | BRHIP3042817 | | | | |
| MAML2 | Mastermind-like 2 (Drosophila) | (1.71) | AI148006 | 1518 | |
| HLA-DPA1 | major histocompatibility | (1.70) | M27487 | 1519 | |
| | complex, class II, DO beta | | | | |
| ARID5B | AT rich interactive domain 2 | (1.69) | AI289774 | 1520 | |
| | (ARID, RFX-like) | | | | |
| VTI1A | vesicle transport through | (1.68) | NM_145206 | 1521 | |
| | interaction with t-SNAREs | | | | |
| | homolog 1A (yeast) | | | | |
| CCR1 | cell cycle progression 1 | (1.67) | NM_001295 | 1522 | |
| TCF4 | transcription factor 4 | (1.67) | AK026674 | 1523 | |
| FOXO1A | folate receptor 3 (gamma) | (1.66) | AA079839 | 1524 | |
| HLA-DPB1 | major histocompatibility | (1.65) | NM_002121 | 1525 | |
| | complex, class II, DP alpha 1 | | | | |
| CD180 | CD163 antigen | (1.65) | NM_005582 | 1526 | |
| KLF12 | Kruppel-like factor 10 | (1.65) | NM_016285 | 1527 | |
| MAML2 | Mastermind-like 2 (Drosophila) | (1.64) | BF358386 | 1528 | |
| EVL | ecotropic viral integration site | (1.64) | T58044 | 1529 | |
| | 2A | | | | |
| NUP210 | nucleoporin 210 kDa | (1.63) | NM_024923 | 1530 | |
| PRSS33 | protease, serine, 33 | (1.62) | NM_152891 | 1531 | |
| | CDNA FLJ46553 fis, clone | (1.62) | AW575863 | 1532 | |
| | THYMU3038879 | | | | |
| | Transcribed locus | (1.62) | AW291535 | 1533 | |
| BLNK | baculoviral IAP repeat- | (1.61) | AA931562 | 1534 | |
| | containing 1 | | | | |
| RBM6 | RNA binding motif protein 6 | (1.61) | AA936428 | 1535 | |
| RFX3 | Regulatory factor X, 3 | (1.61) | BE466926 | 1536 | |
| | (influences HLA class II | | | | |
| | expression) | | | | |
| | CDNA FLJ43467 fis, clone | (1.60) | AA766296 | 1537 | |
| | OCBBF2036752 | | | | |
| RABGAP1L | RAB GTPase activating protein | (1.60) | AK074233 | 1538 | |
| | 1-like | | | | |
| PPAPDC1B | phosphatidic acid phosphatase | (1.60) | AF212238 | 1539 | |
| | type 2 domain containing 1B | | | | |
| ZCCHC7 | Zinc finger, CCHC domain | (1.60) | AF119847 | 1540 | |
| | containing 7 | | | | |
| | Transcribed locus | (1.60) | AA654772 | 1541 | |
| MOBKL2B | MOB1, Mps One Binder kinase | (1.59) | AI375115 | 1542 | |
| | activator-like 2B (yeast) | | | | |
| KIAA0590 | KIAA0261 | (1.59) | AI651969 | 1543 | |
| CD19 | CD180 antigen | (1.58) | NM_001770 | 1544 | |
| CD72 | CD58 antigen, (lymphocyte | (1.58) | AF283777 | 1545 | |
| | function-associated antigen 3) | | | | |
| ETS1 | ERO1-like (S. cerevisiae) | (1.58) | AA702930 | 1546 | |
| ZBTB20 | zinc finger and BTB domain | (1.58) | AW974823 | 1547 | |
| | containing 20 | | | | |
| BRWD2 | bactericidal/permeability- | (1.58) | BF062828 | 1548 | |
| | increasing protein | | | | |
| P2RX5 | purinergic receptor P2X, ligand- | (1.57) | U49396 | 1549 | |
| | gated ion channel, 5 | | | | |
| MYCPBP | C-myc promoter binding protein | (1.57) | T86874 | 1550 | |
| | LOC441276 | (1.57) | AW974642 | 1551 | |
| LENG10 | lipocalin 2 (oncogene 24p3) | (1.56) | AF211977 | 1552 | |
| LRCH3 | lysophosphatidylglycerol | (1.56) | AL832278 | 1553 | |
| | acyltransferase 1 | | | | |
| FCRL5 | Fc receptor-like 5 | (1.55) | AF343664 | 1554 | |
| RIPX | rap2 interacting protein x | (1.55) | AI871641 | 1555 | |
| IGLC2 | Immunoglobulin kappa variable | (1.55) | AK025231 | 1556 | |
| | 1-5 | | | | |
| | | (1.55) | BF433219 | 1557 | |
| | CDNA FLJ43665 fis, clone | (1.55) | T92265 | 1558 | |
| | SYNOV4006327 | | | | |
| CCR7 | chemokine (C-C motif) receptor | (1.55) | NM_001838 | 1559 | |
| | 2; chemokine (C-C motif) | | | | |
| | receptor 2 | | | | |
| | CDNA FLJ26461 fis, clone | (1.55) | AA968447 | 1560 | |
| | KDN03867 | | | | |
| | Transcribed locus | (1.55) | AA524609 | 1561 | |
| MS4A7 | membrane-spanning 4- | (1.55) | AB026043 | 1562 | |
| | domains, subfamily A, member 7 | | | | |
| THOC2 | THO complex 2 | (1.55) | AI018256 | 1563 | |
| CD22; MAG | CD200 antigen | (1.54) | NM_001771 | 1564 | |
| | IG light chain V-region, VK | (1.54) | AF103529 | 1565 | |
| | gene | | | | |
| SNRPN | Small nuclear ribonucleoprotein | (1.54) | BE783065 | 1566 | |
| | polypeptide N | | | | |
| | RNA, U71A small nucleolar | (1.54) | AA827892 | 1567 | |
| DMXL1 | hypothetical protein | (1.54) | N74444 | 1568 | |
| | DKFZp434L142 | | | | |
| MAD1L1 | lactotransferrin | (1.53) | NM_003550 | 1569 | |
| | Transcribed locus | (1.53) | AA521315 | 1570 | |
| VTI1A | Vesicle transport through | (1.52) | AA744508 | 212 | |
| | interaction with t-SNAREs | | | | |
| | homolog 1A (yeast) | | | | |
| | CDNA FLJ42484 fis, clone | (1.52) | AW293849 | 1571 | |
| | BRACE2032182 | | | | |
| SERPINB9 | serpin peptidase inhibitor, clade | (1.52) | AI986192 | 1572 | |
| | B (ovalbumin), member 9 | | | | |
| AKAP9 | angiogenic factor with G patch | (1.52) | AK000270 | 1573 | |
| | and FHA domains 1 | | | | |
| TARSL2 | Threonyl-tRNA synthetase-like 2 | (1.52) | AA807197 | 1574 | |
| | CDNA FLJ45435 fis, clone | (1.52) | BF725383 | 1575 | |
| | BRHIP3042817 | | | | |
| LOC440253 | hypothetical protein | (1.52) | AL833132 | 1576 | |
| | LOC285550 | | | | |
| CD200 | CD1D antigen, d polypeptide; | (1.51) | AF063591 | 1577 | |
| | CD1D antigen, d polypeptide | | | | |
| ANKRD17 | Ankyrin repeat domain 12 | (1.51) | AU146893 | 1578 | |
| IL24 | interleukin 1 receptor, type II | (1.51) | AI638155 | 1579 | |
| IGKC | immunoglobulin heavy constant | (1.51) | AJ408433 | 1580 | |
| | mu | | | | |
| RHOH | Ras homolog gene family, | (1.51) | BE676335 | 1581 | |
| | member H | | | | |
| THEM4 | thioesterase superfamily | (1.50) | NM_053055 | 1582 | |
| | member 4 | | | | |
| PCSK7 | Proprotein convertase | (1.50) | AK027156 | 1583 | |
| | subtilisin/kexin type 7 | | | | |
| SHPRH | SNF2 histone linker PHD RING | (1.50) | AI631201 | 1584 | |
| | helicase | | | | |
| CHR2SYT | chitinase 3-like 2; chitinase 3- | (1.50) | BE552155 | 1585 | |
| | like 2 | | | | |
| | Similar to 40S ribosomal protein | (1.50) | T83938 | 1586 | |
| | S3 | | | | |
| KIF2 | kinase D-interacting substance | 1.50 | BE872563 | 1587 | |
| | of 220 kDa | | | | |
| RAB33B | RAB33B, member RAS | 1.50 | NM_031296 | 1588 | |
| | oncogene family; RAB33B, | | | | |
| | member RAS oncogene family | | | | |
| HTATSF1 | hydroxysteroid dehydrogenase | 1.50 | NM_014500 | 1452 | |
| | like 2 | | | | |
| ST8SIA4 | ST8 alpha-N-acetyl- | 1.50 | NM_005668 | 1589 | |
| | neuraminide alpha-2,8- | | | | |
| | sialyltransferase 4 | | | | |
| GUCY1B3 | G protein-coupled receptor 27 | 1.50 | W93728 | 1590 | |
| SLC40A1 | solute carrier family 40 (iron- | 1.50 | AL136944 | 1591 | |
| | regulated transporter), member 1 | | | | |
| HMGCR | high-mobility group box 2 | 1.50 | AL518627 | 1592 | |
| ASAH1 | N-acylsphingosine | 1.50 | U47674 | 895 | |
| | amidohydrolase (acid | | | | |
| | ceramidase) 1 | | | | |
| GNAI3 | Guanine nucleotide binding | 1.51 | NM_006496 | 1593 | |
| | protein (G protein), alpha 13 | | | | |
| ZMPSTE24 | zinc metallopeptidase (STE24 | 1.51 | NM_005857 | 1594 | |
| | homolog, yeast) | | | | |
| ANKRD12 | ankyrin repeat domain 12 | 1.51 | X80821 | 795 | |
| PARVB | parvin, beta | 1.51 | NM_013327 | 1595 | |
| ARRDC4 | AT rich interactive domain 5B | 1.51 | AV701177 | 857 | |
| | (MRF1-like) | | | | |
| Ells1 | elastase 2, neutrophil | 1.51 | W73230 | 1596 | |
| PPP1R3D | protein phosphatase 1, | 1.51 | AL109928 | 1597 | |
| | regulatory subunit 3D | | | | |
| H2AFY | granzyme B (granzyme 2, | 1.51 | AF044286 | 1598 | |
| | cytotoxic T-lymphocyte- | | | | |
| | associated serine esterase 1); | | | | |
| | granzyme B (granzyme 2, | | | | |
| | cytotoxic T-lymphocyte- | | | | |
| | associated serine esterase 1) | | | | |
| KCNJ2 | Kelch repeat and BTB (POZ) | 1.51 | BF111326 | 1599 | |
| | domain containing 7 | | | | |
| KIAA0261 | KIAA0232 gene product | 1.51 | AF479418 | 349 | |
| EIF5 | eukaryotic translation initiation | 1.51 | NM_001969 | 492 | |
| | factor 2C, 4 | | | | |
| RNF11 | ring finger protein 11 | 1.51 | AB024703 | 1600 | |
| MGST1 | microsomal glutathione S- | 1.51 | NM_020300 | 1601 | |
| | transferase 1 | | | | |
| CD58 | CD58 antigen, (lymphocyte | 1.51 | NM_001779 | 1602 | |
| | function-associated antigen 3); | | | | |
| | CD58 antigen, (lymphocyte | | | | |
| | function-associated antigen 3) | | | | |
| KLF3 | Kruppel-like factor 12 | 1.51 | BF438116 | 1603 | |
| RAB2 | RAB2, member RAS oncogene | 1.51 | AU158062 | 1604 | |
| | family | | | | |
| MAP3K7IP2 | Mitogen-activated protein | 1.51 | AF241230 | 1306 | |
| | kinase kinase kinase 2 | | | | |
| OSBPL8 | oxysterol binding protein-like 8 | 1.51 | BF970829 | 1605 | |
| KIAA1600 | KIAA1546 protein | 1.51 | AB046820 | 1606 | |
| B3GNT1 | UDP-GlcNAc:betaGal beta-1,3- | 1.51 | AF288208 | 1607 | |
| | N- | | | | |
| | acetylglucosaminyltransferase 1 | | | | |
| FLJ38101 | hypothetical protein FLJ32009 | 1.51 | AI935115 | 1608 | |
| TPM1 | tropomyosin 1 (alpha) | 1.52 | NM_000366 | 1609 | |
| PHKB | Phosphorylase kinase, beta | 1.52 | AI798207 | 856 | |
| TXNDC13 | thioredoxin domain containing | 1.52 | BF572868 | 1610 | |
| | 13 | | | | |
| PRG1 | proteoglycan 1, secretory | 1.52 | J03223 | 1611 | |
| | granule | | | | |
| TncRNA | trophoblast-derived noncoding | 1.52 | AI042152 | 1463 | 51 |
| | RNA | | | | |
| BST1 | bromodomain and WD repeat | 1.52 | NM_004334 | 1612 | |
| | domain containing 2 | | | | |
| UFM1 | ubiquitin-fold modifier 1 | 1.52 | BC005193 | 1223 | |
| MARCH7 | Microtubule-associated protein, | 1.52 | NM_022826 | 830 | |
| | RP/EB family, member 2 | | | | |
| FAM13A1 | coagulation factor II (thrombin) | 1.52 | NM_014883 | 1613 | |
| | receptor-like 1 | | | | |
| MCL1 | myeloid cell leukemia sequence | 1.52 | NM_021960 | 1614 | |
| | 1 (BCL2-related) | | | | |
| STK38L | serine/threonine kinase 38 like | 1.52 | AW779556 | 1615 | |
| CDA08 | CD79B antigen | 1.52 | NM_030790 | 489 | |
| | (immunoglobulin-associated | | | | |
| | beta) | | | | |
| BNIP3L | Burkitt lymphoma receptor 1, | 1.52 | AL132665 | 1616 | |
| | GTP binding protein | | | | |
| | (chemokine (C—X—C motif) | | | | |
| | receptor 5) | | | | |
| VPS54 | vacuolar protein sorting 54 | 1.52 | AL359939 | 1617 | |
| | (yeast) | | | | |
| SSFA2 | sperm specific antigen 2 | 1.52 | NM_006751 | 1618 | |
| SPAST | spastin | 1.52 | AB029006 | 1619 | |
| CTBP2 | colony stimulating factor 2 | 1.52 | N23018 | 1201 | |
| | receptor, alpha, low-affinity | | | | |
| | (granulocyte-macrophage) | | | | |
| EIF2C4 | eukaryotic translation initiation | 1.52 | NM_017629 | 676 | |
| | factor 1A, Y-linked | | | | |
| | MRNA; cDNA DKFZp779L1068 | 1.52 | AW303300 | 1620 | |
| | (from clone DKFZp779L1068) | | | | |
| TTRAP | TRAF and TNF receptor | 1.52 | NM_016614 | 1621 | |
| | associated protein | | | | |
| CSF2RA | cysteine-rich secretory protein 3 | 1.52 | M64445 | 1622 | |
| MS4A6A | membrane-spanning 4- | 1.52 | AF253977 | 1623 | |
| | domains, subfamily A, member | | | | |
| | 6A | | | | |
| KLF10 | Kinesin heavy chain member 2 | 1.53 | NM_005655 | 303 | |
| SNCA | synuclein, alpha (non A4 | 1.53 | L36675 | 1624 | |
| | component of amyloid | | | | |
| | precursor) | | | | |
| C1orf22 | chromosome 15 open reading | 1.53 | NM_025191 | 1625 | |
| | frame 29 | | | | |
| SPTLC2 | Serine palmitoyltransferase, | 1.53 | W81119 | 1626 | |
| | long chain base subunit 2 | | | | |
| B3GNT1 | azurocidin 1 (cationic | 1.53 | NM_006577 | 1627 | |
| | antimicrobial protein 37) | | | | |
| RRM2B | ribonucleotide reductase M2 B | 1.53 | AB036063 | 1628 | |
| | (TP53 inducible) | | | | |
| DHX40 | defensin, alpha 4, corticostatin | 1.53 | BF431360 | 1629 | |
| SLMAP | sarcolemma associated protein | 1.53 | AB046821 | 1630 | |
| RIT1 | Ras-like without CAAX 1 | 1.53 | AF084462 | 1631 | |
| MDM1 | Mdm4, transformed 3T3 cell | 1.53 | AW664850 | 1632 | |
| | double minute 1, p53 binding | | | | |
| | protein (mouse) | | | | |
| LRRFIP1 | leucine rich repeat containing 6 | 1.53 | BC004958 | 1633 | |
| KIAA1546 | hypothetical protein KIAA1434 | 1.53 | AB046766 | 1634 | |
| RAD21 | RAD21 homolog (S. pombe) | 1.53 | NM_006265 | 1635 | |
| RNF146 | ring finger protein 146 | 1.53 | AL136829 | 760 | |
| PPP1CB | protein phosphatase 1, catalytic | 1.53 | AI186712 | 1636 | |
| | subunit, beta isoform | | | | |
| BIRC1 | B-cell novel protein 1 | 1.53 | AI817801 | 1637 | |
| PTEN | phosphatase and tensin | 1.53 | BC005821 | 1638 | |
| | homolog (mutated in multiple | | | | |
| | advanced cancers 1); | | | | |
| | phosphatase and tensin | | | | |
| | homolog (mutated in multiple | | | | |
| | advanced cancers 1) | | | | |
| CKLF | Family with sequence similarity | 1.53 | BG533580 | 1639 | |
| | 62 (C2 domain containing) | | | | |
| | member B | | | | |
| NUMB | numb homolog (Drosophila) | 1.54 | AF015040 | 586 | |
| FAM45A | family with sequence similarity | 1.54 | AI697488 | 1640 | |
| | 13, member A1 | | | | |
| HPGD | haptoglobin; haptoglobin- | 1.54 | NM_000860 | 1641 | |
| | related protein | | | | |
| CPEB2 | Collagen, type IV, alpha 3 | 1.54 | AI202327 | 1642 | |
| | (Goodpasture antigen) binding | | | | |
| | protein | | | | |
| PPP1R12A | protein phosphatase 1, | 1.54 | NM_002480 | 1643 | |
| | regulatory (inhibitor) subunit | | | | |
| | 12A | | | | |
| ARFGEF1 | ADP-ribosylation factor guanine | 1.54 | AK025637 | 1008 | |
| | nucleotide-exchange factor | | | | |
| | 1(brefeldin A-inhibited) | | | | |
| LEPR; LEPROT | leukocyte receptor cluster | 1.54 | AW026535 | 1644 | |
| | (LRC) member 10 | | | | |
| CUL4B | cortactin | 1.54 | AF212995 | 1645 | |
| C6orf55 | chromosome 6 open reading | 1.54 | BF241590 | 1646 | |
| | frame 120 | | | | |
| CCPG1 | cell cycle progression 1 | 1.54 | AB033080 | 1647 | |
| MOBKL1A | MOB1, Mps One Binder kinase | 1.54 | AL162039 | 1648 | |
| | activator-like 1A (yeast) | | | | |
| IKIP | Ig lambda mRNA V-region (V-J) | 1.54 | AW182575 | 1649 | |
| | heterohybridoma; | | | | |
| | Immunoglobulin lambda joining | | | | |
| | 3; Immunoglobulin lambda | | | | |
| | constant 1 (Mcg marker) | | | | |
| GAS7 | UDP-N-acetyl-alpha-D- | 1.54 | BC006454 | 1650 | |
| | galactosamine:polypeptide N- | | | | |
| | acetylgalactosaminyltransferase | | | | |
| | 7 (GalNAc-T7) | | | | |
| IRAK3 | RNA binding motif, single | 1.54 | NM_007199 | 1651 | |
| | stranded interacting protein 1 | | | | |
| GNA13 | GLI pathogenesis-related 1 | 1.54 | AW298099 | 1652 | |
| | (glioma) | | | | |
| YPEL5 | yippee-like 5 (Drosophila) | 1.54 | BC000836 | 1653 | |
| AZIN1 | 5-azacytidine induced 2 | 1.54 | NM_015878 | 1654 | |
| CCPG1 | Cyclin G2 | 1.54 | AU144243 | 1655 | |
| SNRK | SNF related kinase | 1.55 | AF226044 | 770 | |
| HSDL2 | hydroxyprostaglandin | 1.55 | BC004331 | 949 | |
| | dehydrogenase 15-(NAD) | | | | |
| MAN1A1 | Mastermind-like 2 (Drosophila) | 1.55 | BG287153 | 1656 | |
| ARFGEF1 | annexin A3 | 1.55 | AF084520 | 928 | |
| CDC42EP3 | CDC42 effector protein (Rho | 1.55 | AF104857 | 1657 | |
| | GTPase binding) 3 | | | | |
| FLI1 | FK506 binding protein 5 | 1.55 | M93255 | 1383 | |
| PBEF1 | pre-B-cell colony enhancing | 1.55 | NM_005746 | 447 | |
| | factor 1 | | | | |
| TLR4 | toll-like receptor 4; toll-like | 1.55 | NM_003266 | 1658 | |
| | receptor 4 | | | | |
| PELI2 | pellino homolog 2 (Drosophila) | 1.55 | NM_021255 | 1659 | |
| | Transcribed locus | 1.55 | AI916948 | 1660 | |
| ENTPD1 | egf-like module containing, | 1.55 | U87967 | 1661 | |
| | mucin-like, hormone receptor- | | | | |
| | like 2 | | | | |
| SESN3 | sestrin 3 | 1.55 | BF685808 | 1662 | |
| CD1D | CD19 antigen | 1.55 | NM_001766 | 1663 | |
| CDC42EP3 | T-cell immunomodulatory | 1.55 | AL136842 | 1664 | |
| | protein; T-cell | | | | |
| | immunomodulatory protein | | | | |
| SERPINB1 | serpin peptidase inhibitor, clade | 1.55 | AI554300 | 1665 | |
| | B (ovalbumin), member 1 | | | | |
| UBE2H | ubiquitin-conjugating enzyme | 1.55 | AI829920 | 984 | |
| | E2H (UBC8 homolog, yeast) | | | | |
| PTEN | Phosphatase and tensin | 1.55 | BG403361 | 1666 | |
| | homolog (mutated in multiple | | | | |
| | advanced cancers 1) | | | | |
| DPYD | dpy-19-like 3 (C. elegans) | 1.55 | NM_000110 | 989 | |
| PTGS1 | prostaglandin-endoperoxide | 1.55 | S36219 | 1667 | |
| | synthase 1 (prostaglandin G/H | | | | |
| | synthase and cyclooxygenase) | | | | |
| MS4A6A | membrane-spanning 4- | 1.55 | AF237908 | 1668 | |
| | domains, subfamily A, member | | | | |
| | 6A; membrane-spanning 4- | | | | |
| | domains, subfamily A, member | | | | |
| | 6A | | | | |
| SOAT1 | sterol O-acyltransferase (acyl- | 1.55 | L21934 | 1669 | |
| | Coenzyme A: cholesterol | | | | |
| | acyltransferase) 1 | | | | |
| LOC497661 | hypothetical protein | 1.55 | AL572206 | 1670 | |
| | LOC440253 | | | | |
| C14orf106 | Chromosome 10 open reading | 1.55 | BF062175 | 1671 | |
| | frame 112 | | | | |
| SP3 | Sp3 transcription factor | 1.56 | N66313 | 1672 | |
| ERO1L | ectonucleoside triphosphate | 1.56 | AW268365 | 1673 | |
| | diphosphohydrolase 1 | | | | |
| SMARCA5 | SWI/SNF related, matrix | 1.56 | NM_003601 | 1237 | |
| | associated, actin dependent | | | | |
| | regulator of chromatin, | | | | |
| | subfamily a, member 5 | | | | |
| ADAM10 | ADAM metallopeptidase | 1.56 | N51370 | 1674 | |
| | domain 10 | | | | |
| KIDINS220 | KIAA1600 | 1.56 | AB033076 | 1099 | |
| KIAA1434 | KIAA1033 | 1.56 | AK001947 | 797 | |
| KIAA0232 | Potassium inwardly-rectifying | 1.56 | AF143884 | 322 | |
| | channel, subfamily J, member 2 | | | | |
| RIT1 | Ras-like without CAAX 1 | 1.56 | AL037450 | 1675 | |
| ANKRD12 | aldehyde dehydrogenase 2 | 1.56 | X80821 | 795 | |
| | family (mitochondrial) | | | | |
| TLR4 | toll-like receptor 4; toll-like | 1.56 | U93091 | 1676 | |
| | receptor 4 | | | | |
| LPGAT1 | putative NFkB activating protein | 1.56 | NM_014873 | 1677 | |
| IFNGR1 | interferon gamma receptor 1; | 1.56 | NM_000416 | 1678 | |
| | interferon gamma receptor 1 | | | | |
| CALD1 | calcium binding protein 39 | 1.56 | AL583520 | 1679 | |
| KIAA0701 | WD and tetratricopeptide | 1.56 | AL136821 | 881 | |
| | repeats 2 | | | | |
| DCP2 | decay accelerating factor for | 1.56 | AI873425 | 1680 | |
| | complement (CD55, Cromer | | | | |
| | blood group system) | | | | |
| CTSB | chitobiase, di-N-acetyl- | 1.57 | AA020826 | 1681 | |
| CYBRD1 | cullin 4B | 1.57 | NM_024843 | 1682 | |
| NRBF2 | nuclear receptor binding factor 2 | 1.57 | AA883074 | 1683 | |
| FPGT | forkhead box O3A | 1.57 | NM_003838 | 1684 | |
| USP15 | ubiquitin specific peptidase 15 | 1.57 | AF153604 | 622 | |
| SLC8A1 | solute carrier family 8 | 1.57 | Y12885 | 1685 | |
| | (sodium/calcium exchanger), | | | | |
| | member 1 | | | | |
| IL6R | Interleukin 24 | 1.57 | S72848 | 1686 | |
| PYGL | phosphorylase, glycogen; liver | 1.57 | NM_002863 | 1687 | |
| | (Hers disease, glycogen | | | | |
| | storage disease type VI) | | | | |
| PRKAR2B | protein kinase, cAMP- | 1.57 | NM_002736 | 1688 | |
| | dependent, regulatory, type II, | | | | |
| | beta | | | | |
| C15orf29 | chromosome 15 open reading | 1.57 | AI924685 | 1689 | |
| | frame 29 | | | | |
| SLC6A6 | solute carrier family 6 | 1.57 | NM_003043 | 831 | |
| | (neurotransmitter transporter, | | | | |
| | taurine), member 6 | | | | |
| LRRC6 | Leucine rich repeat containing 8 | 1.57 | NM_012472 | 1690 | |
| | family, member D | | | | |
| AGGF1 | AF4/FMR2 family, member 3 | 1.57 | NM_018046 | 975 | |
| KBTBD7 | Jumonji domain containing 2C | 1.57 | AL136782 | 1691 | |
| OACT2 | O-acyltransferase (membrane | 1.57 | W63676 | 1692 | |
| | bound) domain containing 2 | | | | |
| PPP4R1 | protein phosphatase 4, | 1.57 | NM_005134 | 1693 | |
| | regulatory subunit 1 | | | | |
| EGLN1 | E3 ubiquitin protein ligase, | 1.57 | AL117352 | 1694 | |
| | HECT domain containing, 1 | | | | |
| GALNT7 | chondroitin sulfate GalNAcT-2 | 1.58 | NM_017423 | 1695 | |
| SPAG9 | Sperm associated antigen 9 | 1.58 | BG290577 | 1696 | |
| KBTBD7 | kelch repeat and BTB (POZ) | 1.58 | BE672291 | 1697 | |
| | domain containing 7 | | | | |
| | Transcribed locus | 1.58 | AI675054 | 713 | |
| CD24 | CD24 antigen (small cell lung | 1.58 | AA761181 | 1698 | |
| | carcinoma cluster 4 antigen) | | | | |
| MCEMP1 | mast cell-expressed membrane | 1.58 | BF433657 | 1699 | |
| | protein 1 | | | | |
| MICAL2 | microtubule associated | 1.58 | BE965029 | 1700 | |
| | monoxygenase, calponin and | | | | |
| | LIM domain containing 2 | | | | |
| PLXDC2 | plexin domain containing 2 | 1.58 | AI264121 | 1701 | |
| CTTN | cathepsin G | 1.58 | NM_005231 | 1702 | |
| PTPRC | protein tyrosine phosphatase, | 1.58 | NM_002838 | 1068 | |
| | receptor type, C | | | | |
| CD24 | CD24 antigen (small cell lung | 1.58 | L33930 | 1703 | |
| | carcinoma cluster 4 antigen) | | | | |
| FLJ22662 | RNA-binding protein | 1.58 | NM_024829 | 1704 | |
| MAP3K7IP2 | mitogen-activated protein | 1.58 | AL117407 | 318 | |
| | kinase kinase kinase 7 | | | | |
| | interacting protein 2 | | | | |
| ROCK1 | Rho-associated, coiled-coil | 1.58 | N22548 | 340 | |
| | containing protein kinase 1 | | | | |
| | MRNA full length insert cDNA | 1.58 | BE500942 | 1215 | |
| | clone EUROIMAGE 1509279 | | | | |
| MIB1 | mindbomb homolog 1 | 1.59 | AW243097 | 1705 | |
| | (Drosophila) | | | | |
| ALDH2 | activated leukocyte cell | 1.59 | NM_000690 | 1706 | |
| | adhesion molecule | | | | |
| CCNG2 | cyclin G2 | 1.59 | AW134535 | 1191 | |
| LOC285550 | hypothetical protein | 1.59 | BF593037 | 1707 | |
| | LOC283663 | | | | |
| TNFSF8 | tumor necrosis factor (ligand) | 1.59 | NM_001244 | 1708 | |
| | superfamily, member 8 | | | | |
| AZI2 | ATPase, Class I, type 8B, | 1.59 | AI809206 | 1709 | |
| | member 4 | | | | |
| SLC15A4 | Solute carrier family 15, | 1.59 | AI636759 | 1710 | |
| | member 4 | | | | |
| GAS7 | growth arrest-specific 7; growth | 1.59 | BE439987 | 1711 | |
| | arrest-specific 7 | | | | |
| GCA | growth arrest-specific 7 | 1.59 | NM_012198 | 1712 | |
| FOXO3A | Forkhead box O1A | 1.59 | AF041336 | 1713 | |
| | (rhabdomyosarcoma) | | | | |
| DIP13B | DEAH (Asp-Glu-Ala-His) box | 1.59 | NM_018171 | 639 | |
| | polypeptide 40 | | | | |
| NEK4 | NIMA (never in mitosis gene a)- | 1.59 | NM_003157 | 1388 | |
| | related kinase 4 | | | | |
| FAS | Fas (TNF receptor superfamily, | 1.59 | NM_000043 | 1714 | |
| | member 6) | | | | |
| JAK2 | insulin receptor substrate 2 | 1.59 | AF001362 | 1715 | |
| CCNG2 | cyclin G2 | 1.59 | L49506 | 1296 | |
| C14orf94 | chromosome 14 open reading | 1.60 | NM_017815 | 1716 | |
| | frame 106 | | | | |
| MPO | myeloperoxidase | 1.60 | NM_000250 | 72 | |
| PFKFB2 | 6-phosphofructo-2- | 1.60 | AB044805 | 1717 | |
| | kinase/fructose-2,6- | | | | |
| | biphosphatase 2 | | | | |
| VAPA | VAMP (vesicle-associated | 1.60 | AL571942 | 1718 | |
| | membrane protein)-associated | | | | |
| | protein A, 33 kDa | | | | |
| MS4A6A | membrane-spanning 4- | 1.60 | NM_022349 | 1719 | |
| | domains, subfamily A, member | | | | |
| | 6A | | | | |
| LILRB2 | leptin receptor; leptin receptor | 1.60 | AF004231 | 1720 | |
| | overlapping transcript | | | | |
| RRAGD | Ras-related GTP binding D | 1.60 | AL138717 | 1721 | |
| TMOD1 | tropomodulin 1 | 1.60 | BC002660 | 1722 | |
| TGFA | transforming growth factor, | 1.61 | NM_003236 | 1723 | |
| | alpha | | | | |
| CKLFSF6 | Chemokine-like factor | 1.61 | AF116694 | 1724 | |
| IFRD1 | interferon gamma receptor 1 | 1.61 | NM_001550 | 1725 | |
| RAB18 | RAB18, member RAS | 1.61 | AI333232 | 1103 | |
| | oncogene family | | | | |
| C15orf29 | chromosome 14 open reading | 1.61 | NM_024713 | 1726 | |
| | frame 94 | | | | |
| MOSC1 | MOCO sulphurase C-terminal | 1.61 | NM_022746 | 1727 | |
| | domain containing 1 | | | | |
| DDX3X | DCN1, defective in cullin | 1.61 | BG492602 | 1728 | |
| | neddylation 1, domain | | | | |
| | containing 1 (S. cerevisiae) | | | | |
| MAK | MAD1 mitotic arrest deficient- | 1.61 | NM_005906 | 1729 | |
| | like 1 (yeast) | | | | |
| ATP8B4 | ATPase, H+ transporting, | 1.61 | NM_024837 | 1730 | |
| | lysosomal 42 kDa, V1 subunit C, | | | | |
| | isoform 1 | | | | |
| SLC31A2 | solute carrier family 31 (copper | 1.61 | NM_001860 | 1731 | |
| | transporters), member 2 | | | | |
| DCUN1D1 | DCP2 decapping enzyme | 1.61 | NM_020640 | 1732 | |
| | homolog (S. cerevisiae) | | | | |
| PPP1R15B | protein phosphatase 1, | 1.61 | BF796046 | 1733 | |
| | regulatory (inhibitor) subunit | | | | |
| | 15B | | | | |
| PTEN | phosphatase and tensin | 1.62 | NM_000314 | 1734 | |
| | homolog (mutated in multiple | | | | |
| | advanced cancers 1) | | | | |
| SLC18A2 | Solute carrier family 18 | 1.62 | AI890972 | 1735 | |
| | (vesicular monoamine), | | | | |
| | member 2 | | | | |
| SH3BGRL2 | SH3 domain binding glutamic | 1.62 | AL035700 | 1736 | |
| | acid-rich protein like 2 | | | | |
| NUDT4; NUDT4P1 | nudix (nucleoside diphosphate | 1.62 | NM_019094 | 1737 | |
| | linked moiety X)-type motif 4; | | | | |
| | nudix (nucleoside diphosphate | | | | |
| | linked moiety X)-type motif 4 | | | | |
| | pseudogene 1 | | | | |
| NUDT4; NUDT4P1 | nudix (nucleoside diphosphate | 1.62 | AF191653 | 1738 | |
| | linked moiety X)-type motif 4; | | | | |
| | nudix (nucleoside diphosphate | | | | |
| | linked moiety X)-type motif 4 | | | | |
| | pseudogene 1 | | | | |
| TMEM55A | transmembrane protein 55A | 1.62 | AA604382 | 1739 | |
| NRBF2 | nuclear receptor binding factor 2 | 1.62 | AF267866 | 1740 | |
| MFAP3L | microfibrillar-associated protein | 1.62 | NM_021647 | 1741 | |
| | 3-like | | | | |
| TUBB1 | tubulin, beta 1; tubulin, beta 1 | 1.63 | NM_030773 | 1742 | |
| COL4A3BP | C-type lectin domain family 4, | 1.63 | AA889952 | 804 | |
| | member D | | | | |
| IRAK3 | interleukin 6 receptor | 1.63 | BC029493 | 1743 | |
| ADAM10 | membrane-associated ring | 1.63 | AU135154 | 1351 | |
| | finger (C3HC4) 7 | | | | |
| MGST1 | microsomal glutathione S- | 1.63 | AI220117 | 1744 | |
| | transferase 1 | | | | |
| PAM | peptidylglycine alpha-amidating | 1.64 | AI022882 | 1745 | |
| | monooxygenase | | | | |
| C6orf120 | chromosome 1 open reading | 1.64 | BF197222 | 1746 | |
| | frame 22 | | | | |
| GLIPR1 | Glycoprotein, alpha- | 1.64 | AV682252 | 1747 | |
| | galactosyltransferase 1 | | | | |
| MGC26963 | hypothetical protein MGC26963 | 1.64 | AI963083 | 1748 | |
| RC3H1 | ring finger and CCCH-type zinc | 1.64 | AW205418 | 1749 | |
| | finger domains 1 | | | | |
| FGFR1OP2 | FYVE, RhoGEF and PH | 1.64 | W74643 | 1750 | |
| | domain containing 2 | | | | |
| RIN2 | Ras and Rab interactor 2 | 1.64 | AL136924 | 1751 | |
| TPM1 | tropomyosin 1 (alpha) | 1.64 | Z24727 | 1752 | |
| SCAP2 | src family associated | 1.64 | AC003999 | 871 | |
| | phosphoprotein 2 | | | | |
| SNX10 | sorting nexin 10 | 1.64 | NM_013322 | 682 | |
| CCNG2 | caspase recruitment domain | 1.64 | NM_004354 | 1753 | |
| | family, member 12 | | | | |
| BCL2L1 | B-cell CLL/lymphoma 11A (zinc | 1.64 | U72398 | 1754 | |
| | finger protein) | | | | |
| TMCO3 | transmembrane and coiled-coil | 1.64 | AL576117 | 1755 | |
| | domains 3 | | | | |
| SCAP2 | src family associated | 1.64 | AB014486 | 1756 | |
| | phosphoprotein 2 | | | | |
| WDR51B | WD repeat domain 51B | 1.64 | BF131947 | 1757 | |
| EIF1AY | egl nine homolog 1 (C. elegans) | 1.65 | BC005248 | 1174 | |
| E2F3 | dihydropyrimidine | 1.65 | NM_001949 | 1104 | |
| | dehydrogenase | | | | |
| | CDNA FLJ12935 fis, clone | 1.65 | AU151342 | 1758 | |
| | NT2RP2004982 | | | | |
| STXBP3 | syntaxin binding protein 3 | 1.66 | NM_007269 | 1322 | |
| PDCD10 | programmed cell death 10 | 1.66 | BC002506 | 1759 | |
| TPM1 | tropomyosin 1 (alpha) | 1.66 | M19267 | 1760 | |
| SPTLC2 | serine palmitoyltransferase, | 1.66 | U15555 | 1761 | |
| | long chain base subunit 2 | | | | |
| S100A8 | S100 calcium binding protein | 1.66 | NM_002964 | 1762 | |
| | A8 (calgranulin A) | | | | |
| CARD12 | capping protein (actin filament) | 1.66 | NM_021209 | 1763 | |
| | muscle Z-line, alpha 2 | | | | |
| SDPR | serum deprivation response | 1.66 | BF982174 | 1764 | |
| | (phosphatidylserine binding | | | | |
| | protein) | | | | |
| MPO | myeloperoxidase | 1.66 | J02694 | 168 | |
| TGFBR1 | transforming growth factor, beta | 1.66 | AA604375 | 1765 | |
| | receptor I (activin A receptor | | | | |
| | type II-like kinase, 53 kDa) | | | | |
| GGTA1 | grancalcin, EF-hand calcium | 1.66 | AI972498 | 1766 | |
| | binding protein; grancalcin, EF- | | | | |
| | hand calcium binding protein | | | | |
| PPBP | pro-platelet basic protein | 1.66 | R64130 | 1767 | |
| | (chemokine (C—X—C motif) | | | | |
| | ligand 7) | | | | |
| TLR8 | toll-like receptor 8 | 1.66 | NM_016610 | 1768 | |
| MAN2A2 | Mannosidase, alpha, class 1A, | 1.67 | NM_018621 | 1769 | |
| | member 1 | | | | |
| TLR2 | toll-like receptor 2 | 1.67 | NM_003264 | 1770 | |
| CD58 | CD24 antigen (small cell lung | 1.67 | D28586 | 1771 | |
| | carcinoma cluster 4 antigen) | | | | |
| SLC18A2 | Solute carrier family 18 | 1.67 | AI709335 | 1772 | |
| | (vesicular monoamine), | | | | |
| | member 2 | | | | |
| | CDNA FLJ36309 fis, clone | 1.67 | AI683805 | 1773 | |
| | THYMU2004986 | | | | |
| TMEM33 | transmembrane protein 33 | 1.67 | NM_018126 | 550 | |
| CD24 | CD24 antigen (small cell lung | 1.67 | BG327863 | 1774 | |
| | carcinoma cluster 4 antigen) | | | | |
| CD24 | CD22 antigen; myelin | 1.67 | AK000168 | 1775 | |
| | associated glycoprotein | | | | |
| FBXL3 | Fas (TNF receptor superfamily, | 1.67 | AI983021 | 1776 | |
| | member 6) | | | | |
| C9orf72 | chromosome 6 open reading | 1.67 | AI832598 | 1777 | |
| | frame 55 | | | | |
| ASAH1 | arrestin domain containing 4 | 1.68 | BC016828 | 1778 | |
| HIPK3 | histone acetyltransferase 1 | 1.68 | AV693403 | 1779 | |
| PPP1CB | protein phosphatase 1, catalytic | 1.68 | NM_002709 | 1780 | |
| | subunit, beta isoform | | | | |
| TMEM30A | transmembrane protein 30A | 1.68 | NM_018247 | 1028 | |
| DPY19L3 | Dmx-like 1 | 1.68 | BF057717 | 1781 | |
| SEC10L1 | SEC10-like 1 (S. cerevisiae) | 1.68 | NM_006544 | 1332 | |
| CTBS | C-terminal binding protein 2 | 1.68 | AW304174 | 1782 | |
| FLJ32009 | hypothetical protein FLJ22662 | 1.68 | AI862096 | 1783 | |
| RNF13 | ring finger protein 13 | 1.68 | NM_007282 | 1784 | |
| SLC8A1 | solute carrier family 8 | 1.68 | AI741439 | 1785 | |
| | (sodium/calcium exchanger), | | | | |
| | member 1 | | | | |
| | Transcribed locus | 1.68 | BG236273 | 1786 | |
| SERINC1 | serine incorporator 1 | 1.69 | AF164794 | 431 | |
| MAP3K2 | mannosidase, alpha, class 2A, | 1.69 | AA045204 | 1787 | |
| | member 2 | | | | |
| SLC22A4 | solute carrier family 22 (organic | 1.69 | NM_003059 | 1788 | |
| | cation transporter), member 4 | | | | |
| PHF20L1 | PHD finger protein 20-like 1 | 1.69 | AI742668 | 1789 | |
| RIOK3 | RIO kinase 3 (yeast); RIO | 1.69 | AW006290 | 1790 | |
| | kinase 3 (yeast) | | | | |
| ATP6V1C1 | Activating transcription factor 6 | 1.69 | NM_001695 | 1791 | |
| SNAP23 | synaptosomal-associated | 1.69 | U55936 | 907 | |
| | protein, 23 kDa | | | | |
| | Transcribed locus, weakly | 1.69 | BE671123 | 1792 | |
| | similar to XP_517655.1 | | | | |
| | PREDICTED: similar to | | | | |
| | KIAA0825 protein [Pan | | | | |
| | troglodytes] | | | | |
| | CDNA clone IMAGE: 5273245 | 1.69 | AK095236 | 1793 | |
| MCTP1 | Multiple C2-domains with two | 1.69 | BG250585 | 497 | |
| | transmembrane regions 1 | | | | |
| MLSTD1 | Male sterility domain containing 1 | 1.69 | H16791 | 1794 | |
| CAB39 | carbonic anhydrase I | 1.70 | AF151824 | 1461 | 49 |
| SDFR1 | stromal cell derived factor | 1.70 | NM_017455 | 1795 | |
| | receptor 1 | | | | |
| NEK7 | NIMA (never in mitosis gene a)- | 1.70 | AL080111 | 1796 | |
| | related kinase 7 | | | | |
| CD58 | CD58 antigen, (lymphocyte | 1.70 | BC005930 | 1797 | |
| | function-associated antigen 3) | | | | |
| | Similar to Formin binding | 1.70 | BF977829 | 1798 | |
| | protein 2 (srGAP2) | | | | |
| KLHL8 | kelch-like 2, Mayven | 1.70 | BE858995 | 1799 | |
| | (Drosophila) | | | | |
| FLJ20273 | Friend leukemia virus | 1.71 | AW241742 | 1800 | |
| | integration 1 | | | | |
| ADAM9 | ADAM metallopeptidase | 1.71 | NM_003816 | 1801 | |
| | domain 10 | | | | |
| JMJD1C | Janus kinase 2 (a protein | 1.71 | AI694023 | 1324 | |
| | tyrosine kinase) | | | | |
| FCAR | F-box and leucine-rich repeat | 1.72 | NM_002000 | | |
| | protein 3 | | | | |
| GNAQ | guanine nucleotide binding | 1.72 | BF969428 | 672 | |
| | protein (G protein), alpha | | | | |
| | inhibiting activity polypeptide 3 | | | | |
| DAF | dishevelled associated activator | 1.72 | NM_000574 | 1802 | |
| | of morphogenesis 2 | | | | |
| SIPA1L2 | signal-induced proliferation- | 1.72 | AB037810 | 1803 | |
| | associated 1 like 2 | | | | |
| OSBPL11 | oxysterol binding protein-like 11 | 1.72 | NM_022776 | 300 | |
| PTPN12 | protein tyrosine phosphatase, | 1.72 | NM_002835 | 1331 | |
| | non-receptor type 12 | | | | |
| CYP1B1 | cytochrome b reductase 1 | 1.72 | AU154504 | 1804 | |
| FKBP5 | FK506 binding protein 5 | 1.72 | NM_004117 | 1805 | |
| ASPH | N-acylsphingosine | 1.72 | AF289489 | 1806 | |
| | amidohydrolase (acid | | | | |
| | ceramidase) 1 | | | | |
| YWHAH | tyrosine 3- | 1.73 | NM_003405 | 1807 | |
| | monooxygenase/tryptophan 5- | | | | |
| | monooxygenase activation | | | | |
| | protein, eta polypeptide | | | | |
| CLEC4D | Charcot-Leyden crystal protein; | 1.73 | BC032313 | 1808 | |
| | Charcot-Leyden crystal protein | | | | |
| CCR2 | chemokine (C-C motif) receptor 1 | 1.73 | NM_000648 | 1397 | |
| SAMSN1 | SAM domain, SH3 domain and | 1.73 | NM_022136 | 1809 | |
| | nuclear localisation signals, 1 | | | | |
| UBE2J1 | ubiquitin-conjugating enzyme | 1.73 | NM_016021 | 309 | |
| | E2, J1 (UBC6 homolog, yeast) | | | | |
| PBEF1 | pre-B-cell colony enhancing | 1.73 | BF575514 | 1810 | |
| | factor 1 | | | | |
| IFNGR1 | Phosphatidic acid phosphatase | 1.73 | AF056979 | 1811 | |
| | type 2 domain containing 1B | | | | |
| FLJ20273 | RNA-binding protein | 1.73 | NM_019027 | 1812 | |
| SNCA | synuclein, alpha (non A4 | 1.74 | BG260394 | 1813 | |
| | component of amyloid | | | | |
| | precursor); synuclein, alpha | | | | |
| | (non A4 component of amyloid | | | | |
| | precursor) | | | | |
| GPR27 | G protein-coupled receptor 160 | 1.74 | AI703476 | 1814 | |
| PRKAR1A | protein kinase, cAMP- | 1.74 | M18468 | 1815 | |
| | dependent, regulatory, type I, | | | | |
| | alpha (tissue specific | | | | |
| | extinguisher 1) | | | | |
| TSPAN2 | tetraspanin 2 | 1.75 | AI743596 | 1816 | |
| | CDNA FLJ26015 fis, clone | 1.75 | BG499974 | 1817 | |
| | MPC03596 | | | | |
| KIAA1033 | KIAA0701 protein | 1.76 | AL137753 | 1818 | |
| ANKRD12 | ankyrin repeat domain 12 | 1.76 | AB020681 | 1819 | |
| GPR160 | guanine nucleotide binding | 1.76 | BC000181 | 1820 | |
| | protein (G protein), gamma 10; | | | | |
| | hypothetical protein | | | | |
| | LOC552891 | | | | |
| IL13RA1 | IKK interacting protein | 1.76 | U81379 | 1821 | |
| C10orf112 | bone marrow stromal cell | 1.77 | AI420817 | 1822 | |
| | antigen 1 | | | | |
| MAPK14 | mitogen-activated protein | 1.77 | AF100544 | 1823 | |
| | kinase 14 | | | | |
| IL13RA1 | interleukin 13 receptor, alpha 1 | 1.77 | U81380 | 1824 | |
| PAM | peptidylglycine alpha-amidating | 1.77 | NM_000919 | 1825 | |
| | monooxygenase | | | | |
| TLR4 | toll-like receptor 4 | 1.77 | NM_138557 | 1826 | |
| TOB1 | transducer of ERBB2, 1 | 1.77 | AA675892 | 705 | |
| | CDNA clone IMAGE: 5287121 | 1.77 | AI025829 | 1827 | |
| ZBTB16 | zinc finger and BTB domain | 1.78 | NM_006006 | 1828 | |
| | containing 16 | | | | |
| TM6SF1 | transmembrane 6 superfamily | 1.78 | NM_023003 | 1829 | |
| | member 1 | | | | |
| RAB21 | RAB21, member RAS | 1.78 | NM_014999 | 1830 | |
| | oncogene family | | | | |
| MCL1 | myeloid cell leukemia sequence | 1.79 | BF594446 | 1246 | |
| | 1 (BCL2-related) | | | | |
| CAPZA2 | capping protein (actin filament) | 1.80 | BC005338 | 740 | |
| | muscle Z-line, alpha 2 | | | | |
| QKI | quaking homolog, KH domain | 1.81 | AL031781 | 1831 | |
| | RNA binding (mouse) | | | | |
| PARVB | parvin, beta | 1.82 | AA187563 | 1832 | |
| UBE2J1 | ubiquitin-conjugating enzyme | 1.82 | AL562528 | 1833 | |
| | E2, J1 (UBC6 homolog, yeast) | | | | |
| MFSD1 | major facilitator superfamily | 1.82 | NM_022736 | 1834 | |
| | domain containing 1 | | | | |
| PPP4R2 | protein phosphatase 4, | 1.82 | AA206408 | 454 | |
| | regulatory subunit 2 | | | | |
| MAPK14 | mitogen-activated protein | 1.83 | L35253 | 1835 | |
| | kinase kinase kinase 7 | | | | |
| | interacting protein 2 | | | | |
| IRS2 | interleukin-1 receptor- | 1.83 | AF073310 | 1836 | |
| | associated kinase 3 | | | | |
| EDD | enoyl Coenzyme A hydratase | 1.83 | AK095151 | 1837 | |
| | domain containing 3 | | | | |
| MYL9 | myosin, light polypeptide 9, | 1.83 | NM_006097 | 1838 | |
| | regulatory | | | | |
| CAPZA2 | cathelicidin antimicrobial | 1.83 | AV685920 | 1839 | |
| | peptide | | | | |
| | Transcribed locus | 1.84 | AW874263 | 1840 | |
| YOD1 | YOD1 OTU deubiquinating | 1.84 | AI982535 | 1841 | |
| | enzyme 1 homolog (yeast) | | | | |
| CA1 | chromosome 9 open reading | 1.85 | NM_001738 | 1842 | |
| | frame 72 | | | | |
| DKFZp434L142 | DIP13 beta | 1.85 | AF260333 | 777 | |
| ALCAM | A kinase (PRKA) anchor protein | 1.85 | AA156721 | 1843 | |
| | (yotiao) 9 | | | | |
| ZNF267 | zinc finger protein 267 | 1.85 | AU150728 | 1451 | |
| HLA-DRB1 | major histocompatibility | 1.86 | AA807056 | 1407 | |
| | complex, class II, DQ alpha 1; | | | | |
| | major histocompatibility | | | | |
| | complex, class II, DQ alpha 2 | | | | |
| ATF6 | aspartate beta-hydroxylase | 1.86 | R17746 | 1844 | |
| GNG10; | Guanine nucleotide binding | 1.86 | NM_004125 | 1845 | |
| LOC552891 | protein (G protein), q | | | | |
| | polypeptide | | | | |
| XK | Kell blood group precursor | 1.86 | NM_021083 | 1846 | |
| | (McLeod phenotype) | | | | |
| IL13RA1 | interleukin 13 receptor, alpha 1 | 1.86 | U62858 | 1847 | |
| MLSTD1 | male sterility domain containing 1 | 1.87 | NM_018099 | 1848 | |
| MALAT1 | male germ cell-associated | 1.88 | W80468 | 1849 | |
| | kinase | | | | |
| HAT1 | H2A histone family, member Y | 1.89 | NM_003642 | 1850 | |
| EMR2 | hypothetical protein Ells1 | 1.91 | NM_013447 | 1851 | |
| MGC34646 | Hypothetical protein | 1.91 | N30209 | 1852 | |
| | MGC34646 | | | | |
| HP; HPR | haptoglobin | 1.91 | NM_020995 | 42 | 38 |
| RGS18 | regulator of G-protein signalling | 1.91 | AF076642 | 1853 | |
| | 18 | | | | |
| B3GNT5 | UDP-GlcNAc:betaGal beta-1,3- | 1.92 | BE672260 | 1854 | |
| | N- | | | | |
| | acetylglucosaminyltransferase 1 | | | | |
| FCHO2 | Fc fragment of IgA, receptor for | 1.94 | AI627666 | 1855 | |
| FKBP5 | FK506 binding protein 5 | 1.97 | AI753747 | 1856 | |
| PCMTD1 | protein-L-isoaspartate (D- | 1.97 | AA453163 | 1088 | |
| | aspartate) O-methyltransferase | | | | |
| | domain containing 1 | | | | |
| GALNACT-2 | chondroitin sulfate GalNAcT-2 | 1.97 | AL139812 | 1857 | |
| FOLR3 | hypothetical protein FLJ38101 | 1.98 | NM_000804 | 1858 | |
| SLC22A15 | solute carrier family 22 (organic | 1.98 | AI279062 | 1859 | |
| | cation transporter), member 15 | | | | |
| KLHL2 | Kruppel-like factor 3 (basic) | 1.98 | NM_007246 | 1860 | |
| GALNACT-2 | interferon, alpha-inducible | 2.00 | NM_018590 | 1861 | |
| | protein (clone IFI-15K) | | | | |
| EVI2A | V-ets erythroblastosis virus E26 | 2.04 | NM_014210 | 1862 | |
| | oncogene homolog 1 (avian) | | | | |
| MMP8 | matrix metallopeptidase 8 | 2.04 | NM_002424 | 1863 | |
| | (neutrophil collagenase) | | | | |
| TCN1 | transcobalamin I (vitamin B12 | 2.04 | NM_001062 | 1864 | |
| | binding protein, R binder family) | | | | |
| TPST1 | tyrosylprotein sulfotransferase 1 | 2.05 | NM_003596 | 1865 | |
| FAS | family with sequence similarity | 2.05 | AA164751 | 860 | |
| | 45, member A | | | | |
| FAS | Fas (TNF receptor superfamily, | 2.05 | X83493 | 712 | |
| | member 6) | | | | |
| F2RL1 | Enah/Vasp-like | 2.07 | BE965369 | 893 | |
| HP | 3-hydroxy-3-methylglutaryl- | 2.12 | NM_005143 | 47 | |
| | Coenzyme A reductase | | | | |
| IL1R2 | interleukin 1 receptor, type II | 2.13 | NM_004633 | 1866 | |
| HMGB2 | major histocompatibility | 2.20 | BC000903 | 1867 | |
| | complex, class II, DR beta 1; | | | | |
| | major histocompatibility | | | | |
| | complex, class II, DR beta 1 | | | | |
| CD163 | CD163 antigen | 2.24 | NM_004244 | 1868 | |
| CRISP3 | cytoplasmic polyadenylation | 2.24 | NM_006061 | 1869 | |
| | element binding protein 2 | | | | |
| CEACAM6 | carcinoembryonic antigen- | 2.24 | BC005008 | 1870 | |
| | related cell adhesion molecule | | | | |
| | 6 (non-specific cross reacting | | | | |
| | antigen); carcinoembryonic | | | | |
| | antigen-related cell adhesion | | | | |
| | molecule 6 (non-specific cross | | | | |
| | reacting antigen) | | | | |
| TMEM30A | transmembrane protein 30A | 2.28 | AK022883 | 1442 | |
| ChGn | carcinoembryonic antigen- | 2.28 | NM_018371 | 1871 | |
| | related cell adhesion molecule 8 | | | | |
| MS4A3 | membrane-spanning 4- | 2.32 | AY095480 | 1872 | |
| | domains, subfamily A, member | | | | |
| | 3 (hematopoietic cell-specific) | | | | |
| | CDNA clone IMAGE: 5273245 | 2.33 | AI927605 | 1873 | |
| IL1R2 | interleukin 18 receptor 1 | 2.39 | U64094 | 16 | 15 |
| ECHDC3 | E2F transcription factor 3 | 2.44 | NM_024693 | 13 | 13 |
| FKBP5 | FGFR1 oncogene partner 2 | 2.45 | W86302 | 1874 | |
| | MRNA; cDNA DKFZp586B0220 | 2.54 | AL049435 | 1875 | |
| | (from clone DKFZp586B0220) | | | | |
| CD163 | chemokine (C-C motif) receptor | 2.65 | Z22969 | 1876 | |
| | 7; chemokine (C-C motif) | | | | |
| | receptor 7 | | | | |
| ANXA3 | Ankyrin repeat domain 17 | 2.68 | M63310 | 1877 | |
| CTSG | cathepsin B | 2.72 | NM_001911 | 20 | 19 |
| SLPI | secretory leukocyte peptidase | 2.77 | NM_003064 | 1878 | |
| | inhibitor | | | | |
| IL18R1 | interleukin 13 receptor, alpha 1; | 2.84 | NM_003855 | 1879 | |
| | interleukin 13 receptor, alpha 1 | | | | |
| OLFM4 | olfactomedin 4 | 3.06 | AL390736 | 1880 | |
| RNASE3 | ribonuclease, RNase A family, | 3.24 | NM_002935 | 1881 | |
| | 3 (eosinophil cationic protein) | | | | |
| DAAM2 | cytochrome P450, family 1, | 3.26 | BF513244 | 1882 | |
| | subfamily B, polypeptide 1 | | | | |
| CAMP | caldesmon 1 | 3.29 | U19970 | 1883 | |
| | Transcribed locus | 3.42 | AW337833 | 19 | 18 |
| LCN2 | AF4/FMR2 family, member 3 | 3.53 | NM_005564 | 1884 | |
| CEACAM6 | CDC42 effector protein (Rho | 3.55 | M18728 | 1885 | |
| | GTPase binding) 3 | | | | |
| BPI | BCL2/adenovirus E1B 19 kDa | 3.55 | NM_001725 | 1886 | |
| | interacting protein 3-like; | | | | |
| | BCL2/adenovirus E1B 19 kDa | | | | |
| | interacting protein 3-like | | | | |
| ARG1 | ADP-ribosylation factor guanine | 3.71 | NM_000045 | 1887 | |
| | nucleotide-exchange factor | | | | |
| | 1(brefeldin A-inhibited) | | | | |
| ELA2 | eukaryotic translation initiation | 4.27 | NM_001972 | 9 | 9 |
| | factor 5 | | | | |
| AZU1 | antizyme inhibitor 1 | 4.51 | NM_001700 | 8 | 8 |
| MS4A3 | membrane-spanning 4- | 4.78 | L35848 | 1888 | |
| | domains, subfamily A, member | | | | |
| | 3 (hematopoietic cell-specific) | | | | |
| TUBB2 | tubulin, beta 2 | 5.70 | NM_001069 | 1889 | |
| DEFA1; DEFA3 | DEAD (Asp-Glu-Ala-Asp) box | 5.91 | NM_004084 | 1890 | |
| | polypeptide 3, X-linked | | | | |
| CEACAM8 | carcinoembryonic antigen- | 7.19 | M33326 | 5 | 5 |
| | related cell adhesion molecule | | | | |
| | 6 (non-specific cross reacting | | | | |
| | antigen) | | | | |
| LTF | leucine rich repeat (in FLII) | 8.00 | NM_002343 | 1891 | |
| | interacting protein 1 | | | | |
| DEFA4 | defensin, alpha 1; defensin, | 14.00 | NM_001925 | 1 | 1 |
| | alpha 3, neutrophil-specific | | | | |

TABLE 5
Duchenne muscular dystrophy (DMD) receiving deflazacort vs.
Duchenne muscular dystrophy (DMD) receiving prednisone

| | | | Genbank | |
|---|---|---|---|---|
| | | Fold | Accession | Nucleic |
| Common Name | Description | Change | Number | Acid No. |
| AATF | apoptosis antagonizing transcription factor | 0.00348 | AF083208 | 1892 |
| ABCA1 | ATP-binding cassette, sub-family A (ABC1), | 0.00709 | NM_005502 | 1893 |
| | member 1 | | | |
| ABCA1 | ATP-binding cassette, sub-family A (ABC1), | 0.0117 | AF285167 | 1894 |
| | member 1 | | | |
| ABHD3 | Abhydrolase domain containing 3 | 0.0499 | BF477544 | 1895 |
| ACSL4 | Acyl-CoA synthetase long-chain family | 0.0111 | W95007 | 1896 |
| | member 4 | | | |
| ACTN4 | Actinin, alpha 4 | 0.0325 | AU158358 | 1897 |
| ACTN4 | actinin, alpha 4 | 0.0349 | U48734 | 1898 |
| ADAM8 | ADAM metallopeptidase domain 8; ADAM | 0.0468 | NM_001109 | 1899 |
| | metallopeptidase domain 8 | | | |
| AFF4 | AF4/FMR2 family, member 4 | 0.044 | N59653 | 1900 |
| ANKRD44 | ankyrin repeat domain 44 | 0.0389 | AI985590 | 1901 |
| ANXA5 | annexin A5 | 0.0116 | NM_001154 | 1902 |
| AOAH | acyloxyacyl hydrolase (neutrophil) | 0.0269 | NM_001637 | 1903 |
| AOF1 | amine oxidase (flavin containing) domain 1 | 0.0249 | BE348688 | 1904 |
| AP3B1 | Adaptor-related protein complex 3, beta 1 | 0.048 | AI342146 | 1905 |
| | subunit | | | |
| APBB1IP | amyloid beta (A4) precursor protein-binding, | 0.0085 | AI093231 | 1906 |
| | family B, member 1 interacting protein | | | |
| APBB1IP | amyloid beta (A4) precursor protein-binding, | 0.0288 | NM_019043 | 1040 |
| | family B, member 1 interacting protein | | | |
| APOB48R | apolipoprotein B48 receptor | 0.00658 | NM_018690 | 1907 |
| APP | amyloid beta (A4) precursor protein | 0.00856 | NM_000484 | 1261 |
| | (peptidase nexin-II, Alzheimer disease) | | | |
| ARF3 | ADP-ribosylation factor 3; ADP-ribosylation | 0.00749 | NM_001659 | 1908 |
| | factor 3 | | | |
| ARFRP2 | ADP-ribosylation factor related protein 2 | 0.046 | AU145206 | 1909 |
| ARID2 | AT rich interactive domain 2 (ARID, RFX-like) | 0.0457 | NM_152641 | 1576 |
| ARNT | aryl hydrocarbon receptor nuclear translocator | 0.0137 | AI768497 | 1910 |
| ARNT | Aryl hydrocarbon receptor nuclear | 0.0346 | BE465380 | 1911 |
| | translocator | | | |
| ARPC1B | actin related protein ⅔ complex, subunit 1B, | 0.0485 | NM_005720 | 1912 |
| | 41 kDa | | | |
| ARRB1 | arrestin, beta 1 | 0.00136 | BE207758 | 1913 |
| ARRB1 | arrestin, beta 1 | 0.0324 | NM_004041 | 1914 |
| ASAHL | N-acylsphingosine amidohydrolase (acid | 0.00386 | AK024677 | 1915 |
| | ceramidase)-like | | | |
| ASAHL | N-acylsphingosine amidohydrolase (acid | 0.00828 | AI436803 | 1916 |
| | ceramidase)-like | | | |
| ATP6V1A | ATPase, H+ transporting, lysosomal 70 kDa, | 0.021 | NM_001690 | 1917 |
| | V1 subunit A | | | |
| AXUD1 | AXIN1 up-regulated 1 | 0.00592 | AI091372 | 1918 |
| BCL2L11 | BCL2-like 11 (apoptosis facilitator) | 0.0496 | AF455755 | 1919 |
| BCL3 | B-cell CLL/lymphoma 3 | 0.0489 | NM_005178 | 218 |
| BHLHB2 | basic helix-loop-helix domain containing, class | 0.0244 | NM_003670 | 1920 |
| | B, 2 | | | |
| BIRC4 | baculoviral IAP repeat-containing 4 | 0.0212 | AW675725 | 1921 |
| BIRC4 | baculoviral IAP repeat-containing 4 | 0.0309 | BE380045 | 1922 |
| BMP2K | BMP2 inducible kinase | 0.00192 | AI034356 | 1923 |
| C1orf24 | chromosome 1 open reading frame 24 | 0.0078 | AF288391 | 1924 |
| C1orf24 | chromosome 1 open reading frame 24 | 0.031 | NM_022083 | 1925 |
| C22orf9 | chromosome 22 open reading frame 9 | 0.0279 | AB023147 | 1926 |
| C3AR1 | complement component 3a receptor 1 | 0.0384 | U62027 | 1927 |
| CA5BL | Carbonic anhydrase VB-like | 0.0459 | AI801563 | 1928 |
| CAMK1 | Calcium/calmodulin-dependent protein kinase I | 0.0184 | AL041824 | 1929 |
| CAMK2G | calcium/calmodulin-dependent protein kinase | 0.0219 | AI093569 | 1930 |
| | (CaM kinase) II gamma | | | |
| CAPNS1 | calpain, small subunit 1; calpain, small | 0.00326 | NM_001749 | 1931 |
| | subunit 1 | | | |
| CARD15 | caspase recruitment domain family, member | 0.0111 | NM_022162 | 1932 |
| | 15 | | | |
| CASP5 | caspase 5, apoptosis-related cysteine | 0.00661 | NM_004347 | 1933 |
| | peptidase | | | |
| CBARA1 | calcium binding atopy-related autoantigen 1 | 0.0215 | AK022697 | 1934 |
| CBL | Cas-Br-M (murine) ecotropic retroviral | 0.00555 | AI828967 | 1935 |
| | transforming sequence | | | |
| CBL | Cas-Br-M (murine) ecotropic retroviral | 0.0204 | AV710415 | 1936 |
| | transforming sequence | | | |
| CCR1 | chemokine (C-C motif) receptor 1 | 0.00422 | AI421071 | 1937 |
| CD14 | CD14 antigen; CD14 antigen | 0.0071 | NM_000591 | 1938 |
| CD36 | CD36 antigen (collagen type I receptor, | 0.0451 | M98399 | 1939 |
| | thrombospondin receptor) | | | |
| CD58 | CD58 antigen, (lymphocyte function- | 0.0151 | R64696 | 1940 |
| | associated antigen 3) | | | |
| CDC14B | CDC14 cell division cycle 14 homolog B (S. cerevisiae); | 0.0115 | NM_003671 | 1941 |
| | CDC14 cell division cycle 14 | | | |
| | homolog B (S. cerevisiae) | | | |
| CDC42EP3 | CDC42 effector protein (Rho GTPase binding) 3 | 0.0142 | AL136842 | 1664 |
| CENTA2 | centaurin, alpha 2 | 0.0273 | NM_018404 | 1942 |
| CFLAR | CASP8 and FADD-like apoptosis regulator | 0.0064 | AF009619 | 1943 |
| CFLAR | CASP8 and FADD-like apoptosis regulator | 0.00822 | AF041459 | 1944 |
| CFLAR | CASP8 and FADD-like apoptosis regulator; | 0.0104 | AF005774 | 1945 |
| | CASP8 and FADD-like apoptosis regulator | | | |
| CFLAR | CASP8 and FADD-like apoptosis regulator | 0.0107 | AF015451 | 1946 |
| CFLAR | CASP8 and FADD-like apoptosis regulator | 0.013 | AF041461 | 1947 |
| CFLAR | CASP8 and FADD-like apoptosis regulator | 0.0159 | NM_003879 | 1948 |
| CFLAR | CASP8 and FADD-like apoptosis regulator | 0.0264 | U97075 | 1949 |
| CFLAR | CASP8 and FADD-like apoptosis regulator | 0.0293 | AF009616 | 1950 |
| CHC1L | Regulator of chromosome condensation | 0.0127 | AA868809 | 1951 |
| | (RCC1) and BTB (POZ) domain containing | | | |
| | protein 2 | | | |
| CHST11 | Carbohydrate (chondroitin 4) sulfotransferase | 0.0306 | BC027983 | 1952 |
| | 11 | | | |
| CHST13 | carbohydrate (chondroitin 4) sulfotransferase | 0.0188 | AA677272 | 1953 |
| | 13 | | | |
| CLEC7A | C-type lectin domain family 7, member A | 0.0148 | AF400602 | 1954 |
| CLEC7A | C-type lectin domain family 7, member A | 0.0263 | AF400600 | 1955 |
| CLEC7A | C-type lectin domain family 7, member A; C- | 0.0435 | AF313468 | 1956 |
| | type lectin domain family 7, member A | | | |
| CNOT2 | CCR4-NOT transcription complex, subunit 2 | 0.0474 | AI859990 | 1957 |
| COMMD1 | Copper metabolism (Murr1) domain | 0.024 | AF086372 | 1958 |
| | containing 1 | | | |
| CORO1C | coronin, actin binding protein, 1C | 0.0121 | AL162070 | 1959 |
| CORO1C | coronin, actin binding protein, 1C | 0.0178 | BC002342 | 1960 |
| CRTAP | cartilage associated protein | 0.0473 | BQ876971 | 1961 |
| CSF1R | colony stimulating factor 1 receptor, formerly | 0.00104 | NM_005211 | 1962 |
| | McDonough feline sarcoma viral (v-fms) | | | |
| | oncogene homolog; colony stimulating factor | | | |
| | 1 receptor, formerly McDonough feline | | | |
| | sarcoma viral (v-fms) oncogene homolog | | | |
| CSF2RB | colony stimulating factor 2 receptor, beta, low- | 0.0184 | AV756141 | 1963 |
| | affinity (granulocyte-macrophage); colony | | | |
| | stimulating factor 2 receptor, beta, low-affinity | | | |
| | (granulocyte-macrophage) | | | |
| CSNK1G1 | casein kinase 1, gamma 1 | 0.0415 | AK025179 | 1964 |
| CST3 | cystatin C (amyloid angiopathy and cerebral | 0.0263 | NM_000099 | 1965 |
| | hemorrhage) | | | |
| CTDSPL | CTD (carboxy-terminal domain, RNA | 0.0379 | BF031714 | 1966 |
| | polymerase II, polypeptide A) small | | | |
| | phosphatase-like | | | |
| CTSC | cathepsin C | 0.000945 | AI246687 | 1967 |
| CTSS | Cathepsin S | 0.0203 | BE675415 | 1968 |
| CTSZ | cathepsin Z | 0.0317 | AF073890 | 1969 |
| CTTN | cortactin | 0.0232 | NM_005231 | 1702 |
| CUTL1 | cut-like 1, CCAAT displacement protein | 0.00418 | BE046521 | 1970 |
| | (Drosophila) | | | |
| CXorf20 | chromosome X open reading frame 20 | 0.00457 | NM_153346 | 1971 |
| DAB2 | disabled homolog 2, mitogen-responsive | 0.00966 | NM_001343 | 1972 |
| | phosphoprotein (Drosophila) | | | |
| DCPS | Decapping enzyme, scavenger | 0.0254 | AI743903 | 1973 |
| DCPS | Decapping enzyme, scavenger | 0.0287 | BF343672 | 1974 |
| DDEF1 | DDEF1 intronic transcript 1 | 0.0169 | AK021983 | 1975 |
| DIAPH2 | diaphanous homolog 2 (Drosophila) | 0.0243 | NM_006729 | 1976 |
| DISC1 | disrupted in schizophrenia 1 | 0.0359 | NM_018662 | 1977 |
| DKFZP434H132 | DKFZP434H132 protein | 0.0011 | NM_015492 | 1978 |
| DKFZP434H132 | DKFZP434H132 protein | 0.00161 | AW516789 | 1979 |
| DKFZp547K1113 | Hypothetical protein DKFZp547K1113 | 0.0279 | AV711227 | 1980 |
| DKFZp564K142 | implantation-associated protein | 0.0289 | AL136636 | 1981 |
| DKFZP564O0463 | WD repeats and SOF1 domain containing | 0.0399 | AI308176 | 1982 |
| DKFZp761E198 | hypothetical protein DKFZp761E198 | 0.00661 | BC004895 | 1983 |
| DMXL2 | Dmx-like 2 | 0.00407 | AK000156 | 1984 |
| DOCK10 | dedicator of cytokinesis 10 | 0.0468 | AB014594 | 1985 |
| DOCK8 | Dedicator of cytokinesis 8 | 0.0264 | T92908 | 1986 |
| DTX3L | deltex 3-like (Drosophila) | 0.0253 | AA577672 | 1987 |
| DUSP6 | dual specificity phosphatase 6 | 0.0341 | BC003143 | 333 |
| DVL3 | dishevelled, dsh homolog 3 (Drosophila) | 0.0497 | NM_004423 | 1988 |
| ECGF1 | endothelial cell growth factor 1 (platelet- | 0.0213 | NM_001953 | 1989 |
| | derived) | | | |
| EIF4G3 | Eukaryotic translation initiation factor 4 | 0.00379 | AA704163 | 1990 |
| | gamma, 3 | | | |
| ELF4 | E74-like factor 4 (ets domain transcription | 0.000539 | U32645 | 1991 |
| | factor) | | | |
| ELL | Elongation factor RNA polymerase II | 0.0476 | AL050168 | 1992 |
| Ells1 | hypothetical protein Ells1 | 0.0185 | W73230 | 1596 |
| ELOVL7 | ELOVL family member 7, elongation of long | 0.00768 | AW138767 | 1993 |
| | chain fatty acids (yeast) | | | |
| EML4 | Echinoderm microtubule associated protein | 0.0399 | BC039354 | 633 |
| | like 4 | | | |
| EPB41L3 | erythrocyte membrane protein band 4.1-like 3 | 0.001 | NM_012307 | 1408 |
| ETV6 | ets variant gene 6 (TEL oncogene) | 0.000689 | AV722693 | 1994 |
| ETV6 | Ets variant gene 6 (TEL oncogene) | 0.0271 | AF147300 | 1995 |
| ETV6 | ets variant gene 6 (TEL oncogene) | 0.032 | AI762695 | 1996 |
| ETV6 | Ets variant gene 6 (TEL oncogene) | 0.0404 | AK025217 | 1997 |
| F11R | F11 receptor | 0.00251 | AF172398 | 1998 |
| F13A1 | coagulation factor XIII, A1 polypeptide | 0.0277 | NM_000129 | 1999 |
| FAM49A | family with sequence similarity 49, member A; | 0.042 | NM_030797 | 2000 |
| | family with sequence similarity 49, member A | | | |
| FBP1 | fructose-1,6-bisphosphatase 1 | 0.0196 | D26054 | 2001 |
| FCER1G | Fc fragment of IgE, high affinity I, receptor for; | 0.00291 | NM_004106 | 2002 |
| | gamma polypeptide | | | |
| FCER1G | Fc fragment of IgE, high affinity I, receptor for; | 0.0033 | BC020763 | 2003 |
| | gamma polypeptide | | | |
| FCGR2A | Fc fragment of IgG, low affinity IIa, receptor | 0.0262 | NM_021642 | 2004 |
| | (CD32) | | | |
| FCGR2C | Fc fragment of IgG, low affinity IIc, receptor | 0.00806 | U90940 | 2005 |
| | for (CD32) | | | |
| FCGR2C | Fc fragment of IgG, low affinity IIc, receptor | 0.0276 | U90939 | 2006 |
| | for (CD32) | | | |
| FGD2 | FYVE, RhoGEF and PH domain containing 2 | 0.0479 | NM_173558 | 2007 |
| FHL1 | four and a half LIM domains 1 | 0.0256 | NM_001449 | 2008 |
| FLJ11151 | hypothetical protein FLJ11151 | 0.00206 | NM_018340 | 1220 |
| FLJ11151 | hypothetical protein FLJ11151 | 0.0378 | AL572407 | 2009 |
| FLJ20481 | hypothetical protein FLJ20481 | 0.0291 | AA789296 | 2010 |
| FLJ20719; AE01 | hypothetical protein FLJ20719; AE01 mRNA | 0.0311 | BC039454 | 2011 |
| FLJ25006 | Hypothetical protein FLJ25006 | 0.0484 | AW081561 | 2012 |
| FLJ31951 | Hypothetical protein FLJ31951 | 0.0452 | AA830854 | 1325 |
| FLJ35390 | hypothetical protein FLJ35390 | 0.0392 | AK092709 | 2013 |
| FLJ44635 | TPT1-like protein | 0.0029 | AI250910 | 2014 |
| FLNA | filamin A, alpha (actin binding protein 280) | 0.0172 | NM_001456 | 2015 |
| FN5 | FN5 protein | 0.0196 | NM_020179 | 2016 |
| FNBP1 | Formin binding protein 1 | 0.0323 | AI017983 | 1338 |
| FOSL2 | FOS-like antigen 2 | 0.0109 | AI670862 | 2017 |
| FOSL2 | FOS-like antigen 2 | 0.026 | AI860150 | 2018 |
| FOSL2 | FOS-like antigen 2 | 0.0384 | N36408 | 2019 |
| FPR1 | formyl peptide receptor 1; formyl peptide | 0.0333 | M60626 | 2020 |
| | receptor 1 | | | |
| FPRL1 | formyl peptide receptor-like 1; formyl peptide | 0.0425 | U81501 | 2021 |
| | receptor-like 1 | | | |
| FYB | FYN binding protein (FYB-120/130) | 0.0334 | AL832727 | 2022 |
| GAB2 | GRB2-associated binding protein 2 | 0.0448 | AL049273 | 1402 |
| GAS7 | growth arrest-specific 7 | 0.00498 | NM_003644 | 2023 |
| GAS7 | growth arrest-specific 7; growth arrest- | 0.0101 | BC006454 | 1650 |
| | specific 7 | | | |
| GAS7 | growth arrest-specific 7 | 0.0106 | BE439987 | 1711 |
| GAS7 | growth arrest-specific 7 | 0.0113 | NM_005890 | 2024 |
| GAS7 | growth arrest-specific 7 | 0.0219 | BC001152 | 2025 |
| GIT2 | G protein-coupled receptor kinase interactor 2 | 0.0133 | R38084 | 2026 |
| GIT2 | G protein-coupled receptor kinase interactor 2 | 0.0388 | AW978350 | 2027 |
| GK | glycerol kinase | 0.0146 | AI830490 | 2028 |
| GLB1 | galactosidase, beta 1 | 0.000522 | NM_000404 | 2029 |
| GNA15 | guanine nucleotide binding protein (G | 0.00544 | NM_002068 | 2030 |
| | protein), alpha 15 (Gq class) | | | |
| GNB1 | Guanine nucleotide binding protein (G | 0.043 | AI056689 | 2031 |
| | protein), beta polypeptide 1 | | | |
| GNB4 | guanine nucleotide binding protein (G | 0.0109 | AW504458 | 2032 |
| | protein), beta polypeptide 4 | | | |
| GNS | glucosamine (N-acetyl)-6-sulfatase (Sanfilippo | 0.00733 | BE880245 | 2033 |
| | disease IIID) | | | |
| GPD2 | Glycerol-3-phosphate dehydrogenase 2 | 0.0345 | AI393727 | 2034 |
| | (mitochondrial) | | | |
| GPR109B | G protein-coupled receptor 109B; G protein- | 0.000507 | NM_006018 | 2035 |
| | coupled receptor 109B | | | |
| GPR43 | G protein-coupled receptor 43 | 0.00696 | NM_005306 | 2036 |
| GRN | granulin | 0.0274 | AK023348 | 2037 |
| GRN | granulin | 0.0294 | NM_002087 | 2038 |
| GUCY1A3 | guanylate cyclase 1, soluble, alpha 3 | 0.00281 | AI719730 | 2039 |
| GUCY1B3 | guanylate cyclase 1, soluble, beta 3 | 0.0103 | W93728 | 1590 |
| HCK | hemopoietic cell kinase | 0.0352 | NM_002110 | 2040 |
| HINT3 | histidine triad nucleotide binding protein 3 | 0.0262 | BF694956 | 2041 |
| HM13 | histocompatibility (minor) 13 | 0.0406 | AK074686 | 2042 |
| HNRPA3P1; HNRPA3; | heterogeneous nuclear ribonucleoprotein A3 | 0.0289 | BC012090 | 2043 |
| LOC387933 | pseudogene 1; heterogeneous nuclear | | | |
| | ribonucleoprotein A3; similar to | | | |
| | heterogeneous nuclear ribonucleoprotein A3 | | | |
| HNRPM | Heterogeneous nuclear ribonucleoprotein M | 0.0287 | AI906424 | 2044 |
| HNRPM | heterogeneous nuclear ribonucleoprotein M | 0.0429 | AK024911 | 2045 |
| HRASLS | HRAS-like suppressor | 0.0129 | NM_020386 | 2046 |
| HSH2D | hematopoietic SH2 domain containing | 0.0274 | BC025237 | 2047 |
| HSMPP8 | M-phase phosphoprotein, mpp8 | 0.00709 | BC003542 | 2048 |
| HSPA1A | heat shock 70 kDa protein 1A | 0.0354 | NM_005345 | 275 |
| HSPA6 | heat shock 70 kDa protein 6 (HSP70B′) | 0.0206 | NM_002155 | 2049 |
| HSPA6 | heat shock 70 kDa protein 6 (HSP70B′) | 0.0352 | X51757 | 2050 |
| HSPC157 | HSPC157 protein | 0.0407 | NM_014179 | 2051 |
| HYPB | Huntingtin interacting protein B | 0.0285 | AI868267 | 1444 |
| IBRDC3 | IBR domain containing 3 | 0.0306 | W27419 | 2052 |
| ICAM1 | intercellular adhesion molecule 1 (CD54), | 6.16E−05 | AI608725 | 2053 |
| | human rhinovirus receptor | | | |
| IDS | iduronate 2-sulfatase (Hunter syndrome) | 0.0136 | AI926544 | 2054 |
| IFI30 | interferon, gamma-inducible protein 30 | 0.0414 | NM_006332 | 2055 |
| IFITM3 | interferon induced transmembrane protein 3 | 0.0148 | BF338947 | 2056 |
| | (1-8U) | | | |
| IGFBP7 | insulin-like growth factor binding protein 7 | 0.0297 | NM_001553 | 2057 |
| IL17R | interleukin 17 receptor | 0.0463 | NM_014339 | 2058 |
| IL1B | interleukin 1, beta | 0.00127 | NM_000576 | 2059 |
| IL1B | interleukin 1, beta | 0.0137 | M15330 | 2060 |
| IL1RN | interleukin 1 receptor antagonist | 0.00419 | U65590 | 2061 |
| IL1RN | interleukin 1 receptor antagonist | 0.00559 | BE563442 | 2062 |
| IL6R | interleukin 6 receptor | 0.0226 | S72848 | 1686 |
| IL6R | interleukin 6 receptor | 0.0254 | AV700030 | 2063 |
| IL6R | interleukin 6 receptor; interleukin 6 receptor | 0.0489 | NM_000565 | 2064 |
| IL8RB | interleukin 8 receptor, beta | 0.0421 | NM_001557 | 2065 |
| ILK | integrin-linked kinase | 0.0161 | NM_004517 | 2066 |
| IQGAP2 | IQ motif containing GTPase activating protein 2 | 0.0185 | AA679303 | 2067 |
| IRF2 | interferon regulatory factor 2 | 0.0118 | NM_002199 | 2068 |
| ITGA2B | integrin, alpha 2b (platelet glycoprotein IIb of | 0.0202 | AF098114 | 2069 |
| | IIb/IIIa complex, antigen CD41B) | | | |
| ITGA2B | integrin, alpha 2b (platelet glycoprotein IIb of | 0.0421 | NM_000419 | 2070 |
| | IIb/IIIa complex, antigen CD41B) | | | |
| ITGB3 | integrin, beta 3 (platelet glycoprotein IIIa, | 0.014 | M35999 | 2071 |
| | antigen CD61) | | | |
| ITSN2 | Intersectin 2 | 0.0422 | AI814925 | 2072 |
| JAK2 | Janus kinase 2 (a protein tyrosine kinase) | 0.0446 | BC043187 | 2073 |
| JARID2 | Jumonji, AT rich interactive domain 2 | 0.000753 | BG029530 | 2074 |
| JMJD3 | jumonji domain containing 3 | 0.0149 | AI830331 | 2075 |
| JUNB | jun B proto-oncogene | 0.0299 | NM_002229 | 2076 |
| KCNJ15 | potassium inwardly-rectifying channel, | 0.0322 | D87291 | 2077 |
| | subfamily J, member 15 | | | |
| KDELR2 | KDEL (Lys-Asp-Glu-Leu) endoplasmic | 0.0129 | NM_006854 | 2078 |
| | reticulum protein retention receptor 2 | | | |
| KIAA0040 | KIAA0040 | 0.00329 | NM_014656 | 2079 |
| KIAA0493 | Family with sequence similarity 91, member | 0.0288 | AK090660 | 2080 |
| | A2 | | | |
| KIAA0999 | KIAA0999 protein | 0.00521 | AB023216 | 2081 |
| KIAA0999 | KIAA0999 protein | 0.0481 | AW590838 | 2082 |
| KIAA1280 | KIAA1280 protein | 0.0446 | AB033106 | 2083 |
| KIAA1632 | KIAA1632 | 0.0326 | AL833448 | 2084 |
| KIAA1919 | KIAA1919 | 0.0484 | BE964989 | 2085 |
| KLF4 | Kruppel-like factor 4 (gut) | 0.0432 | BF514079 | 1363 |
| KPNA3 | Karyopherin alpha 3 (importin alpha 4) | 0.0416 | AL137645 | 906 |
| KPNA4 | karyopherin alpha 4 (importin alpha 3) | 0.0114 | U93240 | 2086 |
| KPNB1 | karyopherin (importin) beta 1 | 0.00518 | BG249565 | 2087 |
| LATS2 | LATS, large tumor suppressor, homolog 2 | 0.00744 | AF207547 | 2088 |
| | (Drosophila) | | | |
| LATS2 | LATS, large tumor suppressor, homolog 2 | 0.0426 | AI535735 | 2089 |
| | (Drosophila) | | | |
| LHFPL2 | lipoma HMGIC fusion partner-like 2 | 0.0434 | N66633 | 2090 |
| LILRA5 | leukocyte immunoglobulin-like receptor, | 0.0369 | AF499918 | 2091 |
| | subfamily A (with TM domain), member 5 | | | |
| LILRB1; LILRA1 | leukocyte immunoglobulin-like receptor, | 0.00957 | NM_006863 | 2092 |
| | subfamily B (with TM and ITIM domains), | | | |
| | member 1; leukocyte immunoglobulin-like | | | |
| | receptor, subfamily A (with TM domain), | | | |
| | member 1 | | | |
| LIMK2 | LIM domain kinase 2 | 0.0261 | NM_005569 | 2093 |
| LITAF | lipopolysaccharide-induced TNF factor | 0.0123 | NM_004862 | 2094 |
| LMO2 | LIM domain only 2 (rhombotin-like 1) | 0.0476 | NM_005574 | 2095 |
| LOC115294 | Protein-L-isoaspartate (D-aspartate) O- | 0.0383 | T85248 | 1311 |
| | methyltransferase domain containing 1 | | | |
| LOC115648; | similar to hypothetical protein FLJ13659; | 0.0423 | AI122756 | 2096 |
| LOC148203; ZNF493; | hypothetical protein LOC148203; zinc finger | | | |
| LOC401905 | protein 493; similar to zinc finger protein 91 | | | |
| | (HPF7, HTF10) | | | |
| LOC23117; LOC440345; | KIAA0220-like protein; hypothetical protein | 0.0463 | AC003007 | 206 |
| LOC641298 | LOC440345; PI-3-kinase-related kinase | | | |
| | SMG-1-like locus | | | |
| LOC283901 | hypothetical protein LOC283901 | 0.0021 | AK095480 | 2097 |
| LOC286272 | Hypothetical protein LOC286272 | 0.038 | H12280 | 2098 |
| LOC286437 | hypothetical protein LOC286437 | 0.0412 | AI080505 | 2099 |
| LOC340061 | hypothetical protein LOC340061 | 0.0498 | AA100891 | 2100 |
| LOC348162 | hypothetical protein 348162 | 0.0486 | AL049250 | 2101 |
| LOC51326 | ADP-ribosylation factor-like | 0.0133 | BC017896 | 487 |
| LOC57168 | similar to aspartate beta hydroxylase (ASPH) | 0.0423 | BE550881 | 2102 |
| LOC641298 | PI-3-kinase-related kinase SMG-1-like locus | 0.0214 | BC042832 | 2103 |
| LRRC25 | leucine rich repeat containing 25 | 0.026 | AJ422148 | 2104 |
| LRRC33 | leucine rich repeat containing 33 | 0.0123 | AA534416 | 2105 |
| LRRFIP1 | leucine rich repeat (in FLII) interacting protein 1 | 0.0293 | NM_004735 | 2106 |
| LYN | v-yes-1 Yamaguchi sarcoma viral related | 0.0105 | AI356412 | 2107 |
| | oncogene homolog; v-yes-1 Yamaguchi | | | |
| | sarcoma viral related oncogene homolog | | | |
| LYN | v-yes-1 Yamaguchi sarcoma viral related | 0.0172 | M79321 | 2108 |
| | oncogene homolog | | | |
| LYST | lysosomal trafficking regulator | 0.0255 | U70064 | 414 |
| MAFB | v-maf musculoaponeurotic fibrosarcoma | 0.0166 | AW135013 | 2109 |
| | oncogene homolog B (avian) | | | |
| MAFB | v-maf musculoaponeurotic fibrosarcoma | 0.0317 | NM_005461 | 1310 |
| | oncogene homolog B (avian) | | | |
| MAFF | v-maf musculoaponeurotic fibrosarcoma | 0.0121 | AL021977 | 2110 |
| | oncogene homolog F (avian) | | | |
| MAP3K5 | mitogen-activated protein kinase kinase | 0.0371 | NM_005923 | 369 |
| | kinase 5 | | | |
| MAP3K5 | mitogen-activated protein kinase kinase | 0.0424 | D84476 | 2111 |
| | kinase 5 | | | |
| MAP3K7IP2 | Mitogen-activated protein kinase kinase | 0.0376 | AF075067 | 2112 |
| | kinase 7 interacting protein 2 | | | |
| MBP | myelin basic protein | 0.0198 | AW070431 | 2113 |
| MBP | myelin basic protein | 0.034 | L18865 | 2114 |
| MBTD1 | mbt domain containing 1 | 0.0451 | NM_017643 | 2115 |
| MCTP2 | Multiple C2-domains with two transmembrane | 0.0453 | AA702409 | 2116 |
| | regions 2 | | | |
| MGC14126 | hypothetical protein MGC14126 | 0.0434 | AY099509 | 2117 |
| MGC21874 | transcriptional adaptor 2 (ADA2 homolog, | 0.0333 | AI862537 | 2118 |
| | yeast)-beta | | | |
| MGC2752 | hypothetical protein MGC2752 | 0.0107 | AI651726 | 2119 |
| MGC3260 | Hypothetical protein MGC3260 | 0.0383 | BE727883 | 2120 |
| MGC40405 | Zinc finger, SWIM-type containing 6 | 0.0375 | AK000119 | 2121 |
| MGC5618 | hypothetical protein MGC5618 | 0.00608 | BF575213 | 2122 |
| MGLL | monoglyceride lipase; monoglyceride lipase | 0.0131 | BC006230 | 2123 |
| MKLN1 | muskelin 1, intracellular mediator containing | 0.0328 | D81987 | 2124 |
| | kelch motifs | | | |
| MLKL | mixed lineage kinase domain-like | 0.031 | AA706818 | 2125 |
| MLL3 | Myeloid/lymphoid or mixed-lineage leukemia 3 | 0.0474 | AI057455 | 2126 |
| MLLT2 | AF4/FMR2 family, member 1 | 0.0149 | AA703523 | 2127 |
| MLSTD1 | male sterility domain containing 1 | 0.0331 | NM_018099 | 1848 |
| M-RIP | Myosin phosphatase-Rho interacting protein | 0.0466 | BF515158 | 2128 |
| MS4A7 | membrane-spanning 4-domains, subfamily A, | 0.0291 | AF237916 | 2129 |
| | member 7; membrane-spanning 4-domains, | | | |
| | subfamily A, member 7 | | | |
| MTF1 | metal-regulatory transcription factor 1 | 5.84E−05 | NM_005955 | 2130 |
| MX2 | myxovirus (influenza virus) resistance 2 | 0.0283 | NM_002463 | 2131 |
| | (mouse) | | | |
| MYADM | myeloid-associated differentiation marker | 0.0206 | BE908995 | 2132 |
| MYLIP | myosin regulatory light chain interacting | 0.00321 | AW292746 | 2133 |
| | protein | | | |
| MYO1F | myosin IF | 0.0266 | BF740152 | 2134 |
| N4BP1 | Nedd4 binding protein 1 | 0.0291 | AB014515 | 2135 |
| NCF2 | neutrophil cytosolic factor 2 (65 kDa, chronic | 0.0348 | BC001606 | 2136 |
| | granulomatous disease, autosomal 2) | | | |
| NCOA1 | nuclear receptor coactivator 1 | 0.00399 | BF576458 | 2137 |
| NCOA6 | nuclear receptor coactivator 6 | 0.0221 | AF128458 | 926 |
| NDE1 | nudE nuclear distribution gene E homolog 1 | 0.024 | BF973292 | 2138 |
| | (A. nidulans) | | | |
| NDE1 | nudE nuclear distribution gene E homolog 1 | 0.0358 | NM_017668 | 2139 |
| | (A. nidulans) | | | |
| NFIC | Nuclear factor I/C (CCAAT-binding | 0.0141 | AW134798 | 2140 |
| | transcription factor) | | | |
| NKTR | natural killer-tumor recognition sequence | 0.0222 | AI880383 | 2141 |
| NOTCH2 | Notch homolog 2 (Drosophila) | 0.00739 | AA291203 | 2142 |
| NOTCH2 | Notch homolog 2 (Drosophila); Notch | 0.0265 | AF308601 | 1046 |
| | homolog 2 (Drosophila) | | | |
| NOTCH2NL | Notch homolog 2 (Drosophila) N-terminal like | 0.0362 | AW516297 | 2143 |
| NSF | Ribosomal protein S7 | 0.0411 | AK025172 | 2144 |
| OLIG1 | oligodendrocyte transcription factor 1 | 0.00583 | AL355743 | 1399 |
| OSTF1 | osteoclast stimulating factor 1 | 0.0203 | NM_012383 | 2145 |
| PACSIN2 | protein kinase C and casein kinase substrate | 0.0409 | NM_007229 | 2146 |
| | in neurons 2 | | | |
| PAPD4 | PAP associated domain containing 4 | 0.00878 | AW104325 | 2147 |
| PARP9 | poly (ADP-ribose) polymerase family, member 9 | 0.0394 | AI738416 | 2148 |
| PARVG | parvin, gamma | 0.0293 | AL355092 | 2149 |
| PB1 | Polybromo 1 | 0.0371 | H79538 | 2150 |
| PBX2 | pre-B-cell leukemia transcription factor 2 | 0.0221 | BE397715 | 2151 |
| PCBP2 | Poly(rC) binding protein 2 | 0.0394 | AW025150 | 2152 |
| POLIM5 | PDZ and LIM domain 5 | 0.0238 | NM_006457 | 2153 |
| PECAM1 | platelet/endothelial cell adhesion molecule | 0.00421 | M37780 | 2154 |
| | (CD31 antigen) | | | |
| PECAM1 | platelet/endothelial cell adhesion molecule | 0.00909 | AA702701 | 2155 |
| | (CD31 antigen) | | | |
| PF4V1 | platelet factor 4 variant 1 | 0.0164 | NM_002620 | 2156 |
| PFKFB3 | 6-phosphofructo-2-kinase/fructose-2,6- | 0.0338 | NM_004566 | 2157 |
| | biphosphatase 3 | | | |
| PHACTR2 | phosphatase and actin regulator 2 | 0.039 | AA551142 | 2158 |
| PHACTR2 | phosphatase and actin regulator 2 | 0.0451 | NM_014721 | 2159 |
| PHCA | phytoceramidase, alkaline | 0.0221 | R12678 | 2160 |
| PHF21A | PHD finger protein 21A | 0.0231 | BC015714 | 2161 |
| PHGDHL1 | Phosphoglycerate dehydrogenase like 1 | 0.0444 | AF339764 | 2162 |
| PIAS1 | Protein inhibitor of activated STAT, 1 | 0.0349 | AI732802 | 2163 |
| PICALM | phosphatidylinositol binding clathrin assembly | 0.0165 | AL135735 | 2164 |
| | protein | | | |
| PICALM | phosphatidylinositol binding clathrin assembly | 0.0363 | AV722190 | 762 |
| | protein | | | |
| PLEK | pleckstrin | 0.000252 | NM_002664 | 2165 |
| PLEKHA2 | pleckstrin homology domain containing, family | 0.0183 | BF347859 | 886 |
| | A (phosphoinositide binding specific) member 2 | | | |
| PLEKHM1 | pleckstrin homology domain containing, family | 0.0134 | AB002354 | 2166 |
| | M (with RUN domain) member 1 | | | |
| PLSCR1 | phospholipid scramblase 1 | 0.0487 | AI825926 | 2167 |
| PLXNB2 | plexin B2 | 0.0177 | BC004542 | 2168 |
| pp9099 | PH domain-containing protein | 0.0311 | NM_025201 | 2169 |
| PPGB | protective protein for beta-galactosidase | 0.0193 | NM_000308 | 2170 |
| | (galactosialidosis) | | | |
| PPIG | Peptidyl-prolyl isomerase G (cyclophilin G) | 0.011 | AW970944 | 2171 |
| PPM1F | Protein phosphatase 1F (PP2C domain | 0.0208 | BC013798 | 2172 |
| | containing) | | | |
| PPP3CA | Protein phosphatase 3 (formerly 2B), catalytic | 0.0165 | AA532718 | 2173 |
| | subunit, alpha isoform (calcineurin A alpha) | | | |
| PRKACA | protein kinase, cAMP-dependent, catalytic, | 0.00184 | NM_002730 | 2174 |
| | alpha | | | |
| PRKAR1A | protein kinase, cAMP-dependent, regulatory, | 0.0262 | AI682905 | 2175 |
| | type I, alpha (tissue specific extinguisher 1) | | | |
| PRKAR2B | protein kinase, cAMP-dependent, regulatory, | 0.0441 | NM_002736 | 1688 |
| | type II, beta | | | |
| PRKCD | protein kinase C, delta | 0.0077 | NM_006254 | 2176 |
| PROK2 | prokineticin 2 | 0.0338 | AF182069 | 2177 |
| PSENEN | presenilin enhancer 2 homolog (C. elegans) | 0.0201 | NM_018468 | 2178 |
| PTGS1 | prostaglandin-endoperoxide synthase 1 | 0.0419 | NM_000962 | 329 |
| | (prostaglandin G/H synthase and | | | |
| | cyclooxygenase) | | | |
| PTGS1 | prostaglandin-endoperoxide synthase 1 | 0.0474 | S36219 | 1667 |
| | (prostaglandin G/H synthase and | | | |
| | cyclooxygenase) | | | |
| PTGS1 | prostaglandin-endoperoxide synthase 1 | 0.0226 | NM_000962 | 329 |
| | (prostaglandin G/H synthase and | | | |
| | cyclooxygenase) | | | |
| PTGS2 | prostaglandin-endoperoxide synthase 2 | 0.0187 | AY151286 | 2179 |
| | (prostaglandin G/H synthase and | | | |
| | cyclooxygenase) | | | |
| PTPN12 | Protein tyrosine phosphatase, non-receptor | 0.00163 | AL079909 | 2180 |
| | type 12 | | | |
| PTPRE | protein tyrosine phosphatase, receptor type, E | 0.0161 | AA775177 | 2181 |
| RAB11FIP1 | RAB11 family interacting protein 1 (class I) | 0.0397 | NM_025151 | 2182 |
| RAB11FIP4 | RAB11 family interacting protein 4 (class II) | 0.047 | BG248313 | 465 |
| RAB18 | RAB18, member RAS oncogene family | 0.0414 | AU145607 | 2183 |
| RAB31 | RAB31, member RAS oncogene family | 0.0378 | NM_006868 | 2184 |
| RAB8A | RAB8A, member RAS oncogene family | 0.00561 | BC002977 | 2185 |
| RAC1 | ras-related C3 botulinum toxin substrate 1 | 0.0328 | AJ012502 | 551 |
| | (rho family, small GTP binding protein Rac1) | | | |
| RAI17 | retinoic acid induced 17 | 0.0318 | AF070622 | 1313 |
| RAP1A | RAP1A, member of RAS oncogene family | 0.0194 | AW793677 | 2186 |
| RAP80 | Receptor associated protein 80 | 0.00717 | AF113675 | 2187 |
| RARA | retinoic acid receptor, alpha | 0.00262 | AJ417079 | 2188 |
| RASA1 | RAS p21 protein activator (GTPase activating | 0.0372 | AK021812 | 2189 |
| | protein) 1 | | | |
| RBBP5 | retinoblastoma binding protein 5 | 0.0383 | NM_005057 | 2190 |
| REL | v-rel reticuloendotheliosis viral oncogene | 0.0166 | NM_002908 | 2191 |
| | homolog (avian) | | | |
| RERE | Arginine-glutamic acid dipeptide (RE) repeats | 0.0354 | H71242 | 2192 |
| RFWD2 | Ring finger and WD repeat domain 2 | 0.0334 | BF725688 | 2193 |
| RFX2 | regulatory factor X, 2 (influences HLA class II | 0.0472 | AK024288 | 2194 |
| | expression) | | | |
| RG9MTD3 | RNA (guanine-9-) methyltransferase domain | 0.0449 | AI214944 | 2195 |
| | containing 3 | | | |
| RHOQ | ras homolog gene family, member Q | 0.0402 | BC033251 | 2196 |
| RIN3 | Ras and Rab interactor 3 | 0.0322 | AA625133 | 2197 |
| RIT1 | Ras-like without CAAX 1 | 0.0214 | AL037450 | 1675 |
| RNF24 | ring finger protein 24 | 0.0415 | BC000213 | 2198 |
| ROCK1 | Rho-associated, coiled-coil containing protein | 0.0356 | AL050032 | 2199 |
| | kinase 1 | | | |
| RSU1 | Ras suppressor protein 1 | 0.0152 | AW515913 | 2200 |
| RUFY1 | RUN and FYVE domain containing 1 | 0.0258 | NM_025158 | 2201 |
| RUFY2 | RUN and FYVE domain containing 2 | 0.00853 | NM_017987 | 2202 |
| SEC14L1 | SEC14-like 1 (S. cerevisiae) | 0.0303 | NM_003003 | 2203 |
| SEC23B | Sec23 homolog B (S. cerevisiae) | 0.0301 | BC005032 | 2204 |
| SECTM1 | secreted and transmembrane 1 | 0.0499 | BF939675 | 2205 |
| SERPINA1 | serpin peptidase inhibitor, clade A (alpha-1 | 0.022 | NM_000295 | 2206 |
| | antiproteinase, antitrypsin), member 1 | | | |
| SERPINA1 | serpin peptidase inhibitor, clade A (alpha-1 | 0.0459 | AF119873 | 2207 |
| | antiproteinase, antitrypsin), member 1 | | | |
| SF3A2 | splicing factor 3a, subunit 2, 66 kDa | 0.0469 | L21990 | 2208 |
| SGK | serum/glucocorticoid regulated kinase | 0.028 | NM_005627 | 2209 |
| SH3BGRL2 | SH3 domain binding glutamic acid-rich protein | 0.0258 | AL035700 | 1736 |
| | like 2 | | | |
| SIGLEC7 | sialic acid binding Ig-like lectin 7 | 0.0193 | NM_016543 | 2210 |
| SIGLEC9 | sialic acid binding Ig-like lectin 9 | 0.0364 | AF247180 | 2211 |
| SIRPB1 | signal-regulatory protein beta 1 | 0.0432 | NM_006065 | 2212 |
| SLC33A1 | Solute carrier family 33 (acetyl-CoA | 0.0407 | BC029450 | 2213 |
| | transporter), member 1 | | | |
| SLC7A7 | solute carrier family 7 (cationic amino acid | 0.0242 | NM_003982 | 2214 |
| | transporter, y+ system), member 7 | | | |
| SLIC1 | Selectin ligand interactor cytoplasmic-1 | 0.0159 | AI081246 | 2215 |
| SMARCA4 | SWI/SNF related, matrix associated, actin | 0.0291 | D26156 | 2216 |
| | dependent regulator of chromatin, subfamily | | | |
| | a, member 4 | | | |
| SMC5L1 | SMC5 structural maintenance of | 0.0179 | BC035661 | 2217 |
| | chromosomes 5-like 1 (yeast) | | | |
| SND1 | Staphylococcal nuclease domain containing 1 | 0.0162 | AI374686 | 2218 |
| SNX1 | Sorting nexin 1 | 0.0426 | AK098001 | 2219 |
| SOD2 | superoxide dismutase 2, mitochondrial | 0.0239 | W46388 | 2220 |
| SP110 | SP110 nuclear body protein | 0.0401 | AF280094 | 2221 |
| SP2 | Sp2 transcription factor | 0.0386 | D28588 | 2222 |
| SP3 | Sp3 transcription factor | 0.0499 | N23846 | 2223 |
| SPAG9 | sperm associated antigen 9 | 0.0493 | BC007524 | 2224 |
| ST8SIA4 | ST8 alpha-N-acetyl-neuraminide alpha-2,8- | 0.0441 | AA352113 | 1171 |
| | sialyltransferase 4 | | | |
| STAT3 | signal transducer and activator of transcription | 0.025 | BC000627 | 2225 |
| | 3 (acute-phase response factor) | | | |
| STAT3 | signal transducer and activator of transcription | 0.0328 | AA634272 | 2226 |
| | 3 (acute-phase response factor) | | | |
| STAT3 | signal transducer and activator of transcription | 0.0405 | AI139252 | 2227 |
| | 3 (acute-phase response factor) | | | |
| STEAP4 | STEAP family member 4 | 0.0195 | AA650281 | 2228 |
| SULT1A1 | sulfotransferase family, cytosolic, 1A, phenol- | 0.0233 | NM_001055 | 2229 |
| | preferring, member 1 | | | |
| SULT1A3; SULT1A4 | sulfotransferase family, cytosolic, 1A, phenol- | 0.0234 | U08032 | 2230 |
| | preferring, member 3; sulfotransferase family, | | | |
| | cytosolic, 1A, phenol-preferring, member 4 | | | |
| SVIL | Supervillin | 0.0273 | BC022883 | 2231 |
| SYK | Spleen tyrosine kinase | 0.0336 | AW467357 | 2232 |
| TAX1BP3 | Tax1 (human T-cell leukemia virus type I) | 0.027 | AF234997 | 2233 |
| | binding protein 3 | | | |
| TBC1D22A | TBC1 domain family, member 22A | 0.0353 | AI597838 | 2234 |
| TGFB1 | transforming growth factor, beta 1 (Camurati- | 0.0114 | BC000125 | 2235 |
| | Engelmann disease) | | | |
| TGFBR3 | transforming growth factor, beta receptor III | 0.0469 | NM_003243 | 2236 |
| | (betaglycan, 300 kDa) | | | |
| TGOLN2 | trans-golgi network protein 2 | 0.00127 | NM_006464 | 1425 |
| THRAP2 | Thyroid hormone receptor associated protein 2 | 0.04 | AK025348 | 2237 |
| TIAF1; MYO18A | TGFB1-induced anti-apoptotic factor 1; | 0.0229 | NM_004740 | 2238 |
| | myosin XVIIIA | | | |
| TJP2 | tight junction protein 2 (zona occludens 2) | 0.0455 | NM_004817 | 675 |
| TLE3 | transducin-like enhancer of split 3 (E(sp1) | 0.0127 | AI567426 | 2239 |
| | homolog, Drosophila) | | | |
| TLE3 | transducin-like enhancer of split 3 (E(sp1) | 0.0216 | NM_005078 | 2240 |
| | homolog, Drosophila) | | | |
| TLR1 | toll-like receptor 1 | 0.0328 | AL050262 | 2241 |
| TMCC1 | transmembrane and coiled-coil domain family 1 | 0.0328 | AB018322 | 1003 |
| TMCC1 | transmembrane and coiled-coil domain family 1 | 0.0345 | AI934469 | 2242 |
| TMEM16F | transmembrane protein 16F | 0.0268 | AL041280 | 2243 |
| TNFSF10 | tumor necrosis factor (ligand) superfamily, | 0.0148 | NM_003810 | 2244 |
| | member 10; tumor necrosis factor (ligand) | | | |
| | superfamily, member 10 | | | |
| TNFSF10 | tumor necrosis factor (ligand) superfamily, | 0.0367 | U57059 | 2245 |
| | member 10; tumor necrosis factor (ligand) | | | |
| | superfamily, member 10 | | | |
| TNFSF13; TNFSF12- | tumor necrosis factor (ligand) superfamily, | 0.012 | AF114013 | 2246 |
| TNFSF13 | member 13; tumor necrosis factor (ligand) | | | |
| | superfamily, member 12-member 13 | | | |
| TNFSF13; TNFSF12- | tumor necrosis factor (ligand) superfamily, | 0.0208 | AF114011 | 2247 |
| TNFSF13 | member 13; tumor necrosis factor (ligand) | | | |
| | superfamily, member 12-member 13 | | | |
| TNFSF13B | tumor necrosis factor (ligand) superfamily, | 0.0196 | AF134715 | 2248 |
| | member 13b | | | |
| TNRC5 | trinucleotide repeat containing 5 | 0.00226 | AF161347 | 2249 |
| TPARL | TPA regulated locus | 0.0263 | AW665624 | 939 |
| TPP1 | tripeptidyl peptidase I | 0.000155 | NM_000391 | 920 |
| TPP1 | tripeptidyl peptidase I | 0.00621 | BG231932 | 2250 |
| TRIM38 | Tripartite motif-containing 38 | 0.018 | AI809831 | 2251 |
| TRIM8 | tripartite motif-containing 8; tripartite motif- | 0.00322 | NM_030912 | 2252 |
| | containing 8 | | | |
| UBE2D3 | ubiquitin-conjugating enzyme E2D 3 (UBC4/5 | 0.0322 | AI239832 | 2253 |
| | homolog, yeast) | | | |
| UBE2J1 | Ubiquitin-conjugating enzyme E2, J1 (UBC6 | 0.0247 | N64079 | 2254 |
| | homolog, yeast) | | | |
| UBE3B | ubiquitin protein ligase E3B | 0.0134 | AK022019 | 2255 |
| USP15 | Ubiquitin specific peptidase 15 | 0.0357 | AU156202 | 2256 |
| VCL | vinculin | 0.000443 | NM_014000 | 2257 |
| VPS37C | vacuolar protein sorting 37C (yeast) | 0.00954 | BM452172 | 2258 |
| VPS41 | vacuolar protein sorting 41 (yeast) | 0.0126 | AF135593 | 342 |
| WARS | tryptophanyl-tRNA synthetase | 0.0139 | NM_004184 | 2259 |
| WASL | Wiskott-Aldrich syndrome-like | 0.0268 | AK025323 | 2260 |
| WIRE | WIRE protein | 0.00508 | AK026913 | 2261 |
| WNK1 | WNK lysine deficient protein kinase 1 | 0.0169 | AL049278 | 2262 |
| XPO1 | Exportin 1 (CRM1 homolog, yeast) | 0.0377 | BC041658 | 2263 |
| YWHAH | tyrosine 3-monooxygenase/tryptophan 5- | 0.018 | NM_003405 | 1807 |
| | monooxygenase activation protein, eta | | | |
| | polypeptide | | | |
| ZAK | sterile alpha motif and leucine zipper | 0.00126 | AW069181 | 2264 |
| | containing kinase AZK | | | |
| ZCCHC2 | zinc finger, CCHC domain containing 2 | 0.0109 | BE676543 | 2265 |
| ZCCHC2 | zinc finger, CCHC domain containing 2 | 0.022 | NM_017742 | 677 |
| ZCCHC2 | zinc finger, CCHC domain containing 2 | 0.0424 | AU147903 | 2266 |
| ZCCHC6 | zinc finger, CCHC domain containing 6 | 0.0477 | AA584428 | 2267 |
| ZFHX1B | Zinc finger homeobox 1b | 0.0124 | AF086037 | 2268 |
| ZFHX1B | Zinc finger homeobox 1b | 0.0233 | AI623184 | 2269 |
| ZFP36L1 | zinc finger protein 36, C3H type-like 1 | 0.0409 | BE620915 | 2270 |
| ZNF160 | zinc finger protein 160 | 0.028 | AA701249 | 1369 |
| ZNF264 | zinc finger protein 264 | 0.00262 | NM_003417 | 2271 |
| ZNF652 | zinc finger protein 652 | 0.0417 | NM_014897 | 2272 |
| ZYG11B | zyg-11 homolog B (C. elegans) | 0.00211 | AV701229 | 2273 |
| ZYG11B | zyg-11 homolog B (C. elegans) | 0.0262 | N21008 | 2274 |
| ZYX | zyxin | 0.0212 | BC002323 | 2275 |
| | MRNA; cDNA DKFZp564O0862 (from clone | 0.00258 | AW130600 | 2276 |
| | DKFZp564O0862) | | | |
| | CDNA clone IMAGE: 4769453 | 0.00433 | AI350995 | 2277 |
| | | 0.00555 | BF055351 | 334 |
| | CDNA FLJ12367 fis, clone MAMMA1002413 | 0.00572 | AL119001 | 2278 |
| | Homo sapiens, clone IMAGE: 4695648, mRNA | 0.00609 | BC016013 | 2279 |
| | Similar to hypothetical protein FLJ36144 | 0.00719 | AI090764 | 2280 |
| | | 0.0108 | U20489 | 2281 |
| | Transcribed locus, weakly similar to | 0.0115 | AA838727 | 2282 |
| | NP_060312.1 hypothetical protein FLJ20489 | | | |
| | [Homo sapiens] | | | |
| | Transcribed locus | 0.0133 | AW451107 | 2283 |
| | | 0.0147 | NM_173704 | 2284 |
| | Full length insert cDNA YR33G07 | 0.0165 | H48516 | 2285 |
| | CDNA FLJ41375 fis, clone BRCAN2007700 | 0.017 | BE739287 | 2286 |
| | CDNA clone IMAGE: 4769453 | 0.017 | AA806989 | 2287 |
| | | 0.0176 | AK026267 | 2288 |
| | Homo sapiens, clone IMAGE: 5745627, mRNA | 0.0182 | BC040914 | 2289 |
| | CDNA FLJ39805 fis, clone SPLEN2007951 | 0.0205 | CA444630 | 1377 |
| | | 0.0206 | BC035170 | 2290 |
| | Clone 25194 mRNA sequence | 0.0209 | BF438386 | 2291 |
| | Transcribed locus | 0.0211 | AI718964 | 2292 |
| | CDNA FLJ14081 fis, clone HEMBB1002280 | 0.0214 | BM728567 | 2293 |
| | Homo sapiens, clone IMAGE: 4272847, mRNA | 0.0226 | BC008462 | 2294 |
| | CDNA FLJ43285 fis, clone MESAN2000067 | 0.0243 | AI302271 | 2295 |
| | CDNA FLJ33139 fis clone UTERU1000109 | 0.0246 | AK057701 | 2296 |
| | CDNA clone IMAGE: 6254031 | 0.0299 | AI800713 | 2297 |
| | Transcribed locus | 0.0303 | AI916948 | 1660 |
| | | 0.0304 | N35922 | 2298 |
| | CDNA FLJ46440 fis, clone THYMU3016022 | 0.0309 | BG288330 | 2299 |
| | Full length insert cDNA YR33G07 | 0.0312 | AW340891 | 729 |
| | Transcribed locus | 0.0326 | AW293956 | 2300 |
| | | 0.0334 | AI371550 | 2301 |
| | | 0.0336 | BG389789 | 2302 |
| | | 0.0338 | AF116653 | 2303 |
| | | 0.0344 | AA207241 | 2304 |
| | | 0.0353 | BC035958 | 2305 |
| | | 0.04 | H43637 | 2306 |
| | | | | 2307 |
| | | | | 2308 |
| | | | | 2309 |
| BGR | Acyl-CoA synthetase bubblegum family | 0.00516 | AW341515 | 2310 |
| | member 2 | | | |
| C1orf128 | chromosome 1 open reading frame 128 | 0.031 | AF271784 | 2311 |
| CDC27 | Cell division cycle 27 | 0.0359 | AA166684 | 2312 |
| FGFR1OP2 | FGFR1 oncogene partner 2 | 0.0301 | W74643 | 1750 |
| FLJ37562 | hypothetical protein FLJ37562 | 0.0328 | BF436799 | 2313 |
| HBLD2 | HESB like domain containing 2; HESB like | 0.0311 | NM_030940 | 2314 |
| | domain containing 2 | | | |
| KLF9 | Kruppel-like factor 9 | 0.0319 | NM_001206 | 2315 |
| LOC51326 | ADP-ribosylation factor-like | 0.0429 | AF119889 | 2316 |
| MLC1SA | myosin light chain 1 slow a | 0.0202 | NM_002475 | 2317 |
| MTP18 | mitochondrial protein 18 kDa | 0.0129 | AF060924 | 2318 |
| NUDT4; NUDT4P1 | nudix (nucleoside diphosphate linked moiety | 0.0352 | AF191653 | 1738 |
| | X)-type motif 4; nudix (nucleoside | | | |
| | diphosphate linked moiety X)-type motif 4 | | | |
| | pseudogene 1 | | | |
| OSBP2 | oxysterol binding protein 2 | 0.0334 | BE501253 | 2319 |
| RAB2B | RAB2B, member RAS oncogene family | 0.0428 | AA531016 | 2320 |
| RARA | retinoic acid receptor, alpha; retinoic acid | 0.0198 | U41742 | 2321 |
| | receptor, alpha | | | |
| SLC25A36 | solute carrier family 25, member 36 | 0.0384 | AL136803 | 56 |
| TAL1 | T-cell acute lymphocytic leukemia 1 | 0.0248 | AW300901 | 2322 |
| TMPRSS6 | transmembrane protease, serine 6 | 0.0304 | AL022314 | 2323 |
| WDR40A | WD repeat domain 40A | 0.0469 | AL555107 | 2324 |
| YOD1 | YOD1 OTU deubiquinating enzyme 1 | 0.0189 | AF090896 | 2325 |
| | homolog (yeast) | | | |
| ZNF595 | Zinc finger protein 595 | 0.00776 | AI580142 | 2326 |
| ZNF626 | zinc finger protein 626 | 0.0179 | NM_145297 | 2327 |
| | CDNA FLJ43311 fis, clone NT2RI2009855 | 0.0424 | AI985021 | 1350 |
| | Hypothetical gene supported by AK024248; | 0.043 | AF086554 | 2328 |
| | AL137733 | | | |
| | Transcribed locus | 0.0435 | H94876 | 2329 |
| | Transcribed locus | 0.0439 | BF514098 | 2330 |
| | Similar to transcript increased in | 0.044 | AK026679 | 2331 |
| | spermiogenesis 78 | | | |
| | Full-length cDNA clone CS0DJ002YF02 of T | 0.0449 | AI300571 | 2332 |
| | cells (Jurkat cell line) Cot 10-normalized of | | | |
| | Homo sapiens (human) | | | |
| | Transcribed locus | 0.0452 | AW954199 | 2333 |
| | | 0.047 | AU120130 | 2334 |
| | | 0.0472 | AL117451 | 2335 |

TABLE 6
PAM Analysis of Duchenne muscular dystrophy (DMD) vs.
Duchenne muscular dystrophy (DMD) receiving steroid gene set

| | | | SEQ |
|---|---|---|---|
| | | Nucleic | ID |
| Affy ID | Gene Symbol | Acid No. | NO. |
| 209369_at | ANXA3 | 1877 | |
| 206177_s_at | ARG1 | 1887 | |
| 209135_at | ASPH | 1806 | |
| 239825_at | ATF6 | 1844 | |
| 214575_s_at | AZU1 | 8 | 8 |
| 205557_at | BPI | 1886 | |
| 210244_at | CAMP | 1883 | |
| 215049_x_at | CD163 | 1876 | |
| 203645_s_at | CD163 | 1868 | |
| 211657_at | CEACAM6 | 1885 | |
| 206676_at | CEACAM8 | 5 | 5 |
| 219049_at | ChGn | 1871 | |
| 207802_at | CRISP3 | 1869 | |
| 205653_at | CTSG | 20 | 19 |
| 212793_at | DAAM2 | 1882 | |
| 205033_s_at | DEFA1; DEFA3 | 1890 | |
| 207269_at | DEFA4 | 1 | 1 |
| 219298_at | ECHDC3 | 13 | 13 |
| 206871_at | ELA2 | 9 | 9 |
| 213506_at | F2RL1 | 893 | |
| 224856_at | FKBP5 | 1874 | |
| 224840_at | FKBP5 | 1856 | |
| 204560_at | FKBP5 | 1805 | |
| 218871_x_at | GALNACT-2 | 1861 | |
| 222235_s_at | GALNACT-2 | 1857 | |
| 203138_at | HAT1 | 1850 | |
| 208808_s_at | HMGB2 | 1867 | |
| 206697_s_at | HP | 47 | |
| 208470_s_at | HP; HPR | 42 | 38 |
| 211612_s_at | IL13RA1 | 1847 | |
| 201888_s_at | IL13RA1 | 1821 | |
| 206618_at | IL18R1 | 1879 | |
| 211372_s_at | IL1R2 | 16 | 15 |
| 205403_at | IL1R2 | 1866 | |
| 209185_s_at | IRS2 | 1836 | |
| 219157_at | KLHL2 | 1860 | |
| 232286_at | LAF4 | 1470 | |
| 244696_at | LAF4 | 1471 | |
| 212531_at | LCN2 | 1884 | |
| 202018_s_at | LTF | 1891 | |
| 226675_s_at | MALAT1 | 1849 | |
| 230026_at | MRPL43 | 1496 | |
| 210254_at | MS4A3 | 1888 | |
| 1554892_a_at | MS4A3 | 1872 | |
| 212768_s_at | OLFM4 | 1880 | |
| 206851_at | RNASE3 | 1881 | |
| 228497_at | SLC22A15 | 1859 | |
| 203021_at | SLPI | 1878 | |
| 39318_at | TCL1A | 1469 | |
| 209995_s_at | TCL1A | 1468 | |
| 205513_at | TCN1 | 1864 | |
| 204924_at | TLR2 | 1770 | |
| 232591_s_at | TMEM30A | 1442 | |
| 204140_at | TPST1 | 1865 | |
| 205883_at | ZBTB16 | 1828 | |
| 231688_at | | 19 | 18 |
| 213817_at | | 1875 | |
| 229460_at | | 1873 | |
| 230354_at | | 1786 | |

TABLE 7
PAM Analysis of Duchenne muscular dystrophy (DMD) receiving deflazacort vs.
Duchenne muscular dystrophy (DMD) receiving prednisone gene set

| | | | Genbank | |
|---|---|---|---|---|
| | | Fold | Accession | Nucleic |
| Common Name | Description | Change | Number | Acid No. |
| AATF | apoptosis antagonizing transcription factor | 0.00348 | AF083208 | 1892 |
| ABCA1 | ATP-binding cassette, sub-family A (ABC1), | 0.00709 | NM_005502 | 1893 |
| | member 1 | | | |
| ABCA1 | ATP-binding cassette, sub-family A (ABC1), | 0.0117 | AF285167 | 1894 |
| | member 1 | | | |
| ABHD3 | Abhydrolase domain containing 3 | 0.0499 | BF477544 | 1895 |
| ACSL4 | Acyl-CoA synthetase long-chain family | 0.0111 | W95007 | 1896 |
| | member 4 | | | |
| ACTN4 | Actinin, alpha 4 | 0.0325 | AU158358 | 1897 |
| ACTN4 | actinin, alpha 4 | 0.0349 | U48734 | 1898 |
| AFF4 | AF4/FMR2 family, member 4 | 0.044 | N59653 | 1900 |
| ANKRD44 | ankyrin repeat domain 44 | 0.0389 | AI985590 | 1901 |
| ANXA5 | annexin A5 | 0.0116 | NM_001154 | 1902 |
| AOAH | acyloxyacyl hydrolase (neutrophil) | 0.0269 | NM_001637 | 1903 |
| AOF1 | amine oxidase (flavin containing) domain 1 | 0.0249 | BE348688 | 1904 |
| AP3B1 | Adaptor-related protein complex 3, beta 1 | 0.048 | AI342146 | 1905 |
| | subunit | | | |
| APBB1IP | amyloid beta (A4) precursor protein-binding, | 0.0085 | AI093231 | 1906 |
| | family B, member 1 interacting protein | | | |
| APBB1IP | amyloid beta (A4) precursor protein-binding, | 0.0288 | NM_019043 | 1040 |
| | family B, member 1 interacting protein | | | |
| APOB48R | apolipoprotein B48 receptor | 0.00658 | NM_018690 | 1907 |
| APP | amyloid beta (A4) precursor protein | 0.00856 | NM_000484 | 1261 |
| | (peptidase nexin-II, Alzheimer disease) | | | |
| ARF3 | ADP-ribosylation factor 3; ADP-ribosylation | 0.00749 | NM_001659 | 1908 |
| | factor 3 | | | |
| ARFRP2 | ADP-ribosylation factor related protein 2 | 0.046 | AU145206 | 1909 |
| ARID2 | AT rich interactive domain 2 (ARID, RFX-like) | 0.0457 | NM_152641 | 1576 |
| ARNT | aryl hydrocarbon receptor nuclear translocator | 0.0137 | AI768497 | 1910 |
| ARNT | Aryl hydrocarbon receptor nuclear | 0.0346 | BE465380 | 1911 |
| | translocator | | | |
| ARRB1 | arrestin, beta 1 | 0.00136 | BE207758 | 1913 |
| ARRB1 | arrestin, beta 1 | 0.0324 | NM_004041 | 1914 |
| ASAHL | N-acylsphingosine amidohydrolase (acid | 0.00386 | AK024677 | 1915 |
| | ceramidase)-like | | | |
| ASAHL | N-acylsphingosine amidohydrolase (acid | 0.00828 | AI436803 | 1916 |
| | ceramidase)-like | | | |
| ATP6V1A | ATPase, H+ transporting, lysosomal 70 kDa, | 0.021 | NM_001690 | 1917 |
| | V1 subunit A | | | |
| AXUD1 | AXIN1 up-regulated 1 | 0.00592 | AI091372 | 1918 |
| BCL2L11 | BCL2-like 11 (apoptosis facilitator) | 0.0496 | AF455755 | 1919 |
| BHLHB2 | basic helix-loop-helix domain containing, class | 0.0244 | NM_003670 | 1920 |
| | B, 2 | | | |
| BIRC4 | baculoviral IAP repeat-containing 4 | 0.0212 | AW675725 | 1921 |
| BIRC4 | baculoviral IAP repeat-containing 4 | 0.0309 | BE380045 | 1922 |
| BMP2K | BMP2 inducible kinase | 0.00192 | AI034356 | 1923 |
| C1orf24 | chromosome 1 open reading frame 24 | 0.0078 | AF288391 | 1924 |
| C1orf24 | chromosome 1 open reading frame 24 | 0.031 | NM_022083 | 1925 |
| C22orf9 | chromosome 22 open reading frame 9 | 0.0279 | AB023147 | 1926 |
| C3AR1 | complement component 3a receptor 1 | 0.0384 | U62027 | 1927 |
| CA5BL | Carbonic anhydrase VB-like | 0.0459 | AI801563 | 1928 |
| CAMK1 | Calcium/calmodulin-dependent protein kinase I | 0.0184 | AL041824 | 1929 |
| CAMK2G | calcium/calmodulin-dependent protein kinase | 0.0219 | AI093569 | 1930 |
| | (CaM kinase) II gamma | | | |
| CAPNS1 | calpain, small subunit 1; calpain, small | 0.00326 | NM_001749 | 1931 |
| | subunit 1 | | | |
| CARD15 | caspase recruitment domain family, member | 0.0111 | NM_022162 | 1932 |
| | 15 | | | |
| CASP5 | caspase 5, apoptosis-related cysteine | 0.00661 | NM_004347 | 1933 |
| | peptidase | | | |
| CBARA1 | calcium binding atopy-related autoantigen 1 | 0.0215 | AK022697 | 1934 |
| CBL | Cas-Br-M (murine) ecotropic retroviral | 0.00555 | AI828967 | 1935 |
| | transforming sequence | | | |
| CBL | Cas-Br-M (murine) ecotropic retroviral | 0.0204 | AV710415 | 1936 |
| | transforming sequence | | | |
| CCR1 | chemokine (C-C motif) receptor 1 | 0.00422 | AI421071 | 1937 |
| CD14 | CD14 antigen; CD14 antigen | 0.0071 | NM_000591 | 1938 |
| CD36 | CD36 antigen (collagen type I receptor, | 0.0451 | M98399 | 1939 |
| | thrombospondin receptor) | | | |
| CD58 | CD58 antigen, (lymphocyte function- | 0.0151 | R64696 | 1940 |
| | associated antigen 3) | | | |
| CDC14B | CDC14 cell division cycle 14 homolog B (S. cerevisiae); | 0.0115 | NM_003671 | 1941 |
| | CDC14 cell division cycle 14 | | | |
| | homolog B (S. cerevisiae) | | | |
| CDC42EP3 | CDC42 effector protein (Rho GTPase binding) 3 | 0.0142 | AL136842 | 1664 |
| CENTA2 | centaurin, alpha 2 | 0.0273 | NM_018404 | 1942 |
| CFLAR | CASP8 and FADD-like apoptosis regulator | 0.0064 | AF009619 | 1943 |
| CFLAR | CASP8 and FADD-like apoptosis regulator | 0.00822 | AF041459 | 1944 |
| CFLAR | CASP8 and FADD-like apoptosis regulator; | 0.0104 | AF005774 | 1945 |
| | CASP8 and FADD-like apoptosis regulator | | | |
| CFLAR | CASP8 and FADD-like apoptosis regulator | 0.0107 | AF015451 | 1946 |
| CFLAR | CASP8 and FADD-like apoptosis regulator | 0.013 | AF041461 | 1947 |
| CFLAR | CASP8 and FADD-like apoptosis regulator | 0.0159 | NM_003879 | 1948 |
| CFLAR | CASP8 and FADD-like apoptosis regulator | 0.0264 | U97075 | 1949 |
| CFLAR | CASP8 and FADD-like apoptosis regulator | 0.0293 | AF009616 | 1950 |
| CHC1L | Regulator of chromosome condensation | 0.0127 | AA868809 | 1951 |
| | (RCC1) and BTB (POZ) domain containing | | | |
| | protein 2 | | | |
| CHST11 | Carbohydrate (chondroitin 4) sulfotransferase | 0.0306 | BC027983 | 1952 |
| | 11 | | | |
| CHST13 | carbohydrate (chondroitin 4) sulfotransferase | 0.0188 | AA677272 | 1953 |
| | 13 | | | |
| CLEC7A | C-type lectin domain family 7, member A | 0.0148 | AF400602 | 1954 |
| CLEC7A | C-type lectin domain family 7, member A | 0.0263 | AF400600 | 1955 |
| CLEC7A | C-type lectin domain family 7, member A; C- | 0.0435 | AF313468 | 1956 |
| | type lectin domain family 7, member A | | | |
| CNOT2 | CCR4-NOT transcription complex, subunit 2 | 0.0474 | AI859990 | 1957 |
| COMMD1 | Copper metabolism (Murr1) domain | 0.024 | AF086372 | 1958 |
| | containing 1 | | | |
| CORO1C | coronin, actin binding protein, 1C | 0.0121 | AL162070 | 1959 |
| CORO1C | coronin, actin binding protein, 1C | 0.0178 | BC002342 | 1960 |
| CRTAP | cartilage associated protein | 0.0473 | BQ876971 | 1961 |
| CSF1R | colony stimulating factor 1 receptor, formerly | 0.00104 | NM_005211 | 1962 |
| | McDonough feline sarcoma viral (v-fms) | | | |
| | oncogene homolog; colony stimulating factor | | | |
| | 1 receptor, formerly McDonough feline | | | |
| | sarcoma viral (v-fms) oncogene homolog | | | |
| CSF2RB | colony stimulating factor 2 receptor, beta, low- | 0.0184 | AV756141 | 1963 |
| | affinity (granulocyte-macrophage); colony | | | |
| | stimulating factor 2 receptor, beta, low-affinity | | | |
| | (granulocyte-macrophage) | | | |
| CSNK1G1 | casein kinase 1, gamma 1 | 0.0415 | AK025179 | 1964 |
| CST3 | cystatin C (amyloid angiopathy and cerebral | 0.0263 | NM_000099 | 1965 |
| | hemorrhage) | | | |
| CTDSPL | CTD (carboxy-terminal domain, RNA | 0.0379 | BF031714 | 1966 |
| | polymerase II, polypeptide A) small | | | |
| | phosphatase-like | | | |
| CTSC | cathepsin C | 0.000945 | AI246687 | 1967 |
| CTSS | Cathepsin S | 0.0203 | BE675415 | 1968 |
| CTSZ | cathepsin Z | 0.0317 | AF073890 | 1969 |
| CTTN | cortactin | 0.0232 | NM_005231 | 1702 |
| CUTL1 | cut-like 1, CCAAT displacement protein | 0.00418 | BE046521 | 1970 |
| | (Drosophila) | | | |
| CXorf20 | chromosome X open reading frame 20 | 0.00457 | NM_153346 | 1971 |
| DAB2 | disabled homolog 2, mitogen-responsive | 0.00966 | NM_001343 | 1972 |
| | phosphoprotein (Drosophila) | | | |
| DCPS | Decapping enzyme, scavenger | 0.0254 | AI743903 | 1973 |
| DCPS | Decapping enzyme, scavenger | 0.0287 | BF343672 | 1974 |
| DDEF1 | DDEF1 intronic transcript 1 | 0.0169 | AK021983 | 1975 |
| DIAPH2 | diaphanous homolog 2 (Drosophila) | 0.0243 | NM_006729 | 1976 |
| DISC1 | disrupted in schizophrenia 1 | 0.0359 | NM_018662 | 1977 |
| DKFZP434H132 | DKFZP434H132 protein | 0.0011 | NM_015492 | 1978 |
| DKFZP434H132 | DKFZP434H132 protein | 0.00161 | AW516789 | 1979 |
| DKFZp547K1113 | Hypothetical protein DKFZp547K1113 | 0.0279 | AV711227 | 1980 |
| DKFZp564K142 | implantation-associated protein | 0.0289 | AL136636 | 1981 |
| DKFZP564O0463 | WD repeats and SOF1 domain containing | 0.0399 | AI308176 | 1982 |
| DKFZp761E198 | hypothetical protein DKFZp761E198 | 0.00661 | BC004895 | 1983 |
| DMXL2 | Dmx-like 2 | 0.00407 | AK000156 | 1984 |
| DOCK10 | dedicator of cytokinesis 10 | 0.0468 | AB014594 | 1985 |
| DOCK8 | Dedicator of cytokinesis 8 | 0.0264 | T92908 | 1986 |
| DTX3L | deltex 3-like (Drosophila) | 0.0253 | AA577672 | 1987 |
| DUSP6 | dual specificity phosphatase 6 | 0.0341 | BC003143 | 333 |
| DVL3 | dishevelled, dsh homolog 3 (Drosophila) | 0.0497 | NM_004423 | 1988 |
| ECGF1 | endothelial cell growth factor 1 (platelet- | 0.0213 | NM_001953 | 1989 |
| | derived) | | | |
| EIF4G3 | Eukaryotic translation initiation factor 4 | 0.00379 | AA704163 | 1990 |
| | gamma, 3 | | | |
| ELF4 | E74-like factor 4 (ets domain transcription | 0.000539 | U32645 | 1991 |
| | factor) | | | |
| ELL | Elongation factor RNA polymerase II | 0.0476 | AL050168 | 1992 |
| Ells1 | hypothetical protein Ells1 | 0.0185 | W73230 | 1596 |
| ELOVL7 | ELOVL family member 7, elongation of long | 0.00768 | AW138767 | 1993 |
| | chain fatty acids (yeast) | | | |
| EML4 | Echinoderm microtubule associated protein | 0.0399 | BC039354 | 633 |
| | like 4 | | | |
| EPB41L3 | erythrocyte membrane protein band 4.1-like 3 | 0.001 | NM_012307 | 1408 |
| ETV6 | ets variant gene 6 (TEL oncogene) | 0.000689 | AV722693 | 1994 |
| ETV6 | Ets variant gene 6 (TEL oncogene) | 0.0271 | AF147300 | 1995 |
| ETV6 | ets variant gene 6 (TEL oncogene) | 0.032 | AI762695 | 1996 |
| ETV6 | Ets variant gene 6 (TEL oncogene) | 0.0404 | AK025217 | 1997 |
| F11R | F11 receptor | 0.00251 | AF172398 | 1998 |
| F13A1 | coagulation factor XIII, A1 polypeptide | 0.0277 | NM_000129 | 1999 |
| FAM49A | family with sequence similarity 49, member A; | 0.042 | NM_030797 | 2000 |
| | family with sequence similarity 49, member A | | | |
| FBP1 | fructose-1,6-bisphosphatase 1 | 0.0196 | D26054 | 2001 |
| FCER1G | Fc fragment of IgE, high affinity I, receptor for; | 0.00291 | NM_004106 | 2002 |
| | gamma polypeptide | | | |
| FCER1G | Fc fragment of IgE, high affinity I, receptor for; | 0.0033 | BC020763 | 2003 |
| | gamma polypeptide | | | |
| FCGR2A | Fc fragment of IgG, low affinity IIa, receptor | 0.0262 | NM_021642 | 2004 |
| | (CD32) | | | |
| FCGR2C | Fc fragment of IgG, low affinity IIc, receptor | 0.00806 | U90940 | 2005 |
| | for (CD32) | | | |
| FCGR2C | Fc fragment of IgG, low affinity IIc, receptor | 0.0276 | U90939 | 2006 |
| | for (CD32) | | | |
| FHL1 | four and a half LIM domains 1 | 0.0256 | NM_001449 | 2008 |
| FLJ11151 | hypothetical protein FLJ11151 | 0.00206 | NM_018340 | 1220 |
| FLJ11151 | hypothetical protein FLJ11151 | 0.0378 | AL572407 | 2009 |
| FLJ20481 | hypothetical protein FLJ20481 | 0.0291 | AA789296 | 2010 |
| FLJ20719; AE01 | hypothetical protein FLJ20719; AE01 mRNA | 0.0311 | BC039454 | 2011 |
| FLJ25006 | Hypothetical protein FLJ25006 | 0.0484 | AW081561 | 2012 |
| FLJ31951 | Hypothetical protein FLJ31951 | 0.0452 | AA830854 | 1325 |
| FLJ35390 | hypothetical protein FLJ35390 | 0.0392 | AK092709 | 2013 |
| FLJ44635 | TPT1-like protein | 0.0029 | AI250910 | 2014 |
| FLNA | filamin A, alpha (actin binding protein 280) | 0.0172 | NM_001456 | 2015 |
| FN5 | FN5 protein | 0.0196 | NM_020179 | 2016 |
| FNBP1 | Formin binding protein 1 | 0.0323 | AI017983 | 1338 |
| FOSL2 | FOS-like antigen 2 | 0.0109 | AI670862 | 2017 |
| FOSL2 | FOS-like antigen 2 | 0.026 | AI860150 | 2018 |
| FOSL2 | FOS-like antigen 2 | 0.0384 | N36408 | 2019 |
| FPR1 | formyl peptide receptor 1; formyl peptide | 0.0333 | M60626 | 2020 |
| | receptor 1 | | | |
| FPRL1 | formyl peptide receptor-like 1; formyl peptide | 0.0425 | U81501 | 2021 |
| | receptor-like 1 | | | |
| GAB2 | GRB2-associated binding protein 2 | 0.0448 | AL049273 | 1402 |
| GAS7 | growth arrest-specific 7 | 0.00498 | NM_003644 | 2023 |
| GAS7 | growth arrest-specific 7; growth arrest- | 0.0101 | BC006454 | 1650 |
| | specific 7 | | | |
| GAS7 | growth arrest-specific 7 | 0.0106 | BE439987 | 1711 |
| GAS7 | growth arrest-specific 7 | 0.0113 | NM_005890 | 2024 |
| GAS7 | growth arrest-specific 7 | 0.0219 | BC001152 | 2025 |
| GIT2 | G protein-coupled receptor kinase interactor 2 | 0.0133 | R38084 | 2026 |
| GIT2 | G protein-coupled receptor kinase interactor 2 | 0.0388 | AW978350 | 2027 |
| GK | glycerol kinase | 0.0146 | AI830490 | 2028 |
| GLB1 | galactosidase, beta 1 | 0.000522 | NM_000404 | 2029 |
| GNA15 | guanine nucleotide binding protein (G | 0.00544 | NM_002068 | 2030 |
| | protein), alpha 15 (Gq class) | | | |
| GNB1 | Guanine nucleotide binding protein (G | 0.043 | AI056689 | 2031 |
| | protein), beta polypeptide 1 | | | |
| GNB4 | guanine nucleotide binding protein (G | 0.0109 | AW504458 | 2032 |
| | protein), beta polypeptide 4 | | | |
| GNS | glucosamine (N-acetyl)-6-sulfatase (Sanfilippo | 0.00733 | BE880245 | 2033 |
| | disease IIID) | | | |
| GPD2 | Glycerol-3-phosphate dehydrogenase 2 | 0.0345 | AI393727 | 2034 |
| | (mitochondrial) | | | |
| GPR109B | G protein-coupled receptor 109B; G protein- | 0.000507 | NM_006018 | 2035 |
| | coupled receptor 109B | | | |
| GPR43 | G protein-coupled receptor 43 | 0.00696 | NM_005306 | 2036 |
| GRN | granulin | 0.0274 | AK023348 | 2037 |
| GRN | granulin | 0.0294 | NM_002087 | 2038 |
| GUCY1A3 | guanylate cyclase 1, soluble, alpha 3 | 0.00281 | AI719730 | 2039 |
| GUCY1B3 | guanylate cyclase 1, soluble, beta 3 | 0.0103 | W93728 | 1590 |
| HINT3 | histidine triad nucleotide binding protein 3 | 0.0262 | BF694956 | 2041 |
| HM13 | histocompatibility (minor) 13 | 0.0406 | AK074686 | 2042 |
| HNRPA3P1; HNRPA3; | heterogeneous nuclear ribonucleoprotein A3 | 0.0289 | BC012090 | 2043 |
| LOC387933 | pseudogene 1; heterogeneous nuclear | | | |
| | ribonucleoprotein A3; similar to | | | |
| | heterogeneous nuclear ribonucleoprotein A3 | | | |
| HNRPM | Heterogeneous nuclear ribonucleoprotein M | 0.0287 | AI906424 | 2044 |
| HNRPM | heterogeneous nuclear ribonucleoprotein M | 0.0429 | AK024911 | 2045 |
| HRASLS | HRAS-like suppressor | 0.0129 | NM_020386 | 2046 |
| HSMPP8 | M-phase phosphoprotein, mpp8 | 0.00709 | BC003542 | 2048 |
| HSPA1A | heat shock 70 kDa protein 1A | 0.0354 | NM_005345 | 275 |
| HSPA6 | heat shock 70 kDa protein 6 (HSP70B′) | 0.0206 | NM_002155 | 2049 |
| HSPA6 | heat shock 70 kDa protein 6 (HSP70B′) | 0.0352 | X51757 | 2050 |
| HSPC157 | HSPC157 protein | 0.0407 | NM_014179 | 2051 |
| HYPB | Huntingtin interacting protein B | 0.0285 | AI868267 | 1444 |
| IBRDC3 | IBR domain containing 3 | 0.0306 | W27419 | 2052 |
| ICAM1 | intercellular adhesion molecule 1 (CD54), | 6.16E−05 | AI608725 | 2053 |
| | human rhinovirus receptor | | | |
| IDS | iduronate 2-sulfatase (Hunter syndrome) | 0.0136 | AI926544 | 2054 |
| IFI30 | interferon, gamma-inducible protein 30 | 0.0414 | NM_006332 | 2055 |
| IFITM3 | interferon induced transmembrane protein 3 | 0.0148 | BF338947 | 2056 |
| | (1-8U) | | | |
| IGFBP7 | insulin-like growth factor binding protein 7 | 0.0297 | NM_001553 | 2057 |
| IL17R | interleukin 17 receptor | 0.0463 | NM_014339 | 2058 |
| IL1B | interleukin 1, beta | 0.00127 | NM_000576 | 2059 |
| IL1B | interleukin 1, beta | 0.0137 | M15330 | 2060 |
| IL1RN | interleukin 1 receptor antagonist | 0.00419 | U65590 | 2061 |
| IL1RN | interleukin 1 receptor antagonist | 0.00559 | BE563442 | 2062 |
| IL6R | interleukin 6 receptor | 0.0226 | S72848 | 1686 |
| IL6R | interleukin 6 receptor | 0.0254 | AV700030 | 2063 |
| IL6R | interleukin 6 receptor; interleukin 6 receptor | 0.0489 | NM_000565 | 2064 |
| IL8RB | interleukin 8 receptor, beta | 0.0421 | NM_001557 | 2065 |
| ILK | integrin-linked kinase | 0.0161 | NM_004517 | 2066 |
| IQGAP2 | IQ motif containing GTPase activating protein 2 | 0.0185 | AA679303 | 2067 |
| IRF2 | interferon regulatory factor 2 | 0.0118 | NM_002199 | 2068 |
| ITGA2B | integrin, alpha 2b (platelet glycoprotein IIb of | 0.0202 | AF098114 | 2069 |
| | IIb/IIIa complex, antigen CD41B) | | | |
| ITGA2B | integrin, alpha 2b (platelet glycoprotein IIb of | 0.0421 | NM_000419 | 2070 |
| | IIb/IIIa complex, antigen CD41B) | | | |
| ITGB3 | integrin, beta 3 (platelet glycoprotein IIIa, | 0.014 | M35999 | 2071 |
| | antigen CD61) | | | |
| ITSN2 | Intersectin 2 | 0.0422 | AI814925 | 2072 |
| JAK2 | Janus kinase 2 (a protein tyrosine kinase) | 0.0446 | BC043187 | 2073 |
| JARID2 | Jumonji, AT rich interactive domain 2 | 0.000753 | BG029530 | 2074 |
| JMJD3 | jumonji domain containing 3 | 0.0149 | AI830331 | 2075 |
| JUNB | jun B proto-oncogene | 0.0299 | NM_002229 | 2076 |
| KCNJ15 | potassium inwardly-rectifying channel, | 0.0322 | D87291 | 2077 |
| | subfamily J, member 15 | | | |
| KDELR2 | KDEL (Lys-Asp-Glu-Leu) endoplasmic | 0.0129 | NM_006854 | 2078 |
| | reticulum protein retention receptor 2 | | | |
| KIAA0040 | KIAA0040 | 0.00329 | NM_014656 | 2079 |
| KIAA0493 | Family with sequence similarity 91, member | 0.0288 | AK090660 | 2080 |
| | A2 | | | |
| KIAA0999 | KIAA0999 protein | 0.00521 | AB023216 | 2081 |
| KIAA0999 | KIAA0999 protein | 0.0481 | AW590838 | 2082 |
| KIAA1280 | KIAA1280 protein | 0.0446 | AB033106 | 2083 |
| KIAA1632 | KIAA1632 | 0.0326 | AL833448 | 2084 |
| KIAA1919 | KIAA1919 | 0.0484 | BE964989 | 2085 |
| KLF4 | Kruppel-like factor 4 (gut) | 0.0432 | BF514079 | 1363 |
| KPNA3 | Karyopherin alpha 3 (importin alpha 4) | 0.0416 | AL137645 | 906 |
| KPNA4 | karyopherin alpha 4 (importin alpha 3) | 0.0114 | U93240 | 2086 |
| KPNB1 | karyopherin (importin) beta 1 | 0.00518 | BG249565 | 2087 |
| LATS2 | LATS, large tumor suppressor, homolog 2 | 0.00744 | AF207547 | 2088 |
| | (Drosophila) | | | |
| LATS2 | LATS, large tumor suppressor, homolog 2 | 0.0426 | AI535735 | 2089 |
| | (Drosophila) | | | |
| LHFPL2 | lipoma HMGIC fusion partner-like 2 | 0.0434 | N66633 | 2090 |
| LILRA5 | leukocyte immunoglobulin-like receptor, | 0.0369 | AF499918 | 2091 |
| | subfamily A (with TM domain), member 5 | | | |
| LILRB1; LILRA1 | leukocyte immunoglobulin-like receptor, | 0.00957 | NM_006863 | 2092 |
| | subfamily B (with TM and ITIM domains), | | | |
| | member 1; leukocyte immunoglobulin-like | | | |
| | receptor, subfamily A (with TM domain), | | | |
| | member 1 | | | |
| LIMK2 | LIM domain kinase 2 | 0.0261 | NM_005569 | 2093 |
| LITAF | lipopolysaccharide-induced TNF factor | 0.0123 | NM_004862 | 2094 |
| LMO2 | LIM domain only 2 (rhombotin-like 1) | 0.0476 | NM_005574 | 2095 |
| LOC115294 | Protein-L-isoaspartate (D-aspartate) O- | 0.0383 | T85248 | 1311 |
| | methyltransferase domain containing 1 | | | |
| LOC115648; | similar to hypothetical protein FLJ13659; | 0.0423 | AI122756 | 2096 |
| LOC148203; ZNF493; | hypothetical protein LOC148203; zinc finger | | | |
| LOC401905 | protein 493; similar to zinc finger protein 91 | | | |
| | (HPF7, HTF10) | | | |
| LOC23117; LOC440345; | KIAA0220-like protein; hypothetical protein | 0.0463 | AC003007 | 206 |
| LOC641298 | LOC440345; PI-3-kinase-related kinase | | | |
| | SMG-1-like locus | | | |
| LOC283901 | hypothetical protein LOC283901 | 0.0021 | AK095480 | 2097 |
| LOC286437 | hypothetical protein LOC286437 | 0.0412 | AI080505 | 2099 |
| LOC348162 | hypothetical protein 348162 | 0.0486 | AL049250 | 2101 |
| LOC51326 | ADP-ribosylation factor-like | 0.0133 | BC017896 | 487 |
| LOC57168 | similar to aspartate beta hydroxylase (ASPH) | 0.0423 | BE550881 | 2102 |
| LOC641298 | PI-3-kinase-related kinase SMG-1-like locus | 0.0214 | BC042832 | 2103 |
| LRRC25 | leucine rich repeat containing 25 | 0.026 | AJ422148 | 2104 |
| LRRC33 | leucine rich repeat containing 33 | 0.0123 | AA534416 | 2105 |
| LRRFIP1 | leucine rich repeat (in FLII) interacting protein 1 | 0.0293 | NM_004735 | 2106 |
| LYN | v-yes-1 Yamaguchi sarcoma viral related | 0.0105 | AI356412 | 2107 |
| | oncogene homolog; v-yes-1 Yamaguchi | | | |
| | sarcoma viral related oncogene homolog | | | |
| LYN | v-yes-1 Yamaguchi sarcoma viral related | 0.0172 | M79321 | 2108 |
| | oncogene homolog | | | |
| LYST | lysosomal trafficking regulator | 0.0255 | U70064 | 414 |
| MAFB | v-maf musculoaponeurotic fibrosarcoma | 0.0166 | AW135013 | 2109 |
| | oncogene homolog B (avian) | | | |
| MAFB | v-maf musculoaponeurotic fibrosarcoma | 0.0317 | NM_005461 | 1310 |
| | oncogene homolog B (avian) | | | |
| MAFF | v-maf musculoaponeurotic fibrosarcoma | 0.0121 | AL021977 | 2110 |
| | oncogene homolog F (avian) | | | |
| MAP3K5 | mitogen-activated protein kinase kinase | 0.0371 | NM_005923 | 369 |
| | kinase 5 | | | |
| MAP3K5 | mitogen-activated protein kinase kinase | 0.0424 | D84476 | 2111 |
| | kinase 5 | | | |
| MAP3K7IP2 | Mitogen-activated protein kinase kinase | 0.0376 | AF075067 | 2112 |
| | kinase 7 interacting protein 2 | | | |
| MBP | myelin basic protein | 0.0198 | AW070431 | 2113 |
| MBP | myelin basic protein | 0.034 | L18865 | 2114 |
| MBTD1 | mbt domain containing 1 | 0.0451 | NM_017643 | 2115 |
| MCTP2 | Multiple C2-domains with two transmembrane | 0.0453 | AA702409 | 2116 |
| | regions 2 | | | |
| MGC14126 | hypothetical protein MGC14126 | 0.0434 | AY099509 | 2117 |
| MGC21874 | transcriptional adaptor 2 (ADA2 homolog, | 0.0333 | AI862537 | 2118 |
| | yeast)-beta | | | |
| MGC2752 | hypothetical protein MGC2752 | 0.0107 | AI651726 | 2119 |
| MGC3260 | Hypothetical protein MGC3260 | 0.0383 | BE727883 | 2120 |
| MGC40405 | Zinc finger, SWIM-type containing 6 | 0.0375 | AK000119 | 2121 |
| MGC5618 | hypothetical protein MGC5618 | 0.00608 | BF575213 | 2122 |
| MGLL | monoglyceride lipase; monoglyceride lipase | 0.0131 | BC006230 | 2123 |
| MKLN1 | muskelin 1, intracellular mediator containing | 0.0328 | D81987 | 2124 |
| | kelch motifs | | | |
| MLKL | mixed lineage kinase domain-like | 0.031 | AA706818 | 2125 |
| MLL3 | Myeloid/lymphoid or mixed-lineage leukemia 3 | 0.0474 | AI057455 | 2126 |
| MLLT2 | AF4/FMR2 family, member 1 | 0.0149 | AA703523 | 2127 |
| MLSTD1 | male sterility domain containing 1 | 0.0331 | NM_018099 | 1848 |
| M-RIP | Myosin phosphatase-Rho interacting protein | 0.0466 | BF515158 | 2128 |
| MS4A7 | membrane-spanning 4-domains, subfamily A, | 0.0291 | AF237916 | 2129 |
| | member 7; membrane-spanning 4-domains, | | | |
| | subfamily A, member 7 | | | |
| MTF1 | metal-regulatory transcription factor 1 | 5.84E−05 | NM_005955 | 2130 |
| MYADM | myeloid-associated differentiation marker | 0.0206 | BE908995 | 2132 |
| MYLIP | myosin regulatory light chain interacting | 0.00321 | AW292746 | 2133 |
| | protein | | | |
| MYO1F | myosin IF | 0.0266 | BF740152 | 2134 |
| N4BP1 | Nedd4 binding protein 1 | 0.0291 | AB014515 | 2135 |
| NCF2 | neutrophil cytosolic factor 2 (65 kDa, chronic | 0.0348 | BC001606 | 2136 |
| | granulomatous disease, autosomal 2) | | | |
| NCOA1 | nuclear receptor coactivator 1 | 0.00399 | BF576458 | 2137 |
| NCOA6 | nuclear receptor coactivator 6 | 0.0221 | AF128458 | 926 |
| NDE1 | nudE nuclear distribution gene E homolog 1 | 0.024 | BF973292 | 2138 |
| | (A. nidulans) | | | |
| NDE1 | nudE nuclear distribution gene E homolog 1 | 0.0358 | NM_017668 | 2139 |
| | (A. nidulans) | | | |
| NFIC | Nuclear factor I/C (CCAAT-binding | 0.0141 | AW134798 | 2140 |
| | transcription factor) | | | |
| NKTR | natural killer-tumor recognition sequence | 0.0222 | AI880383 | 2141 |
| NOTCH2 | Notch homolog 2 (Drosophila) | 0.00739 | AA291203 | 2142 |
| NOTCH2 | Notch homolog 2 (Drosophila); Notch | 0.0265 | AF308601 | 1046 |
| | homolog 2 (Drosophila) | | | |
| NOTCH2NL | Notch homolog 2 (Drosophila) N-terminal like | 0.0362 | AW516297 | 2143 |
| NSF | Ribosomal protein S7 | 0.0411 | AK025172 | 2144 |
| OLIG1 | oligodendrocyte transcription factor 1 | 0.00583 | AL355743 | 1399 |
| OSTF1 | osteoclast stimulating factor 1 | 0.0203 | NM_012383 | 2145 |
| PACSIN2 | protein kinase C and casein kinase substrate | 0.0409 | NM_007229 | 2146 |
| | in neurons 2 | | | |
| PAPD4 | PAP associated domain containing 4 | 0.00878 | AW104325 | 2147 |
| PARP9 | poly (ADP-ribose) polymerase family, member 9 | 0.0394 | AI738416 | 2148 |
| PARVG | parvin, gamma | 0.0293 | AL355092 | 2149 |
| PB1 | Polybromo 1 | 0.0371 | H79538 | 2150 |
| PBX2 | pre-B-cell leukemia transcription factor 2 | 0.0221 | BE397715 | 2151 |
| PCBP2 | Poly(rC) binding protein 2 | 0.0394 | AW025150 | 2152 |
| PDLIM5 | PDZ and LIM domain 5 | 0.0238 | NM_006457 | 2153 |
| PECAM1 | platelet/endothelial cell adhesion molecule | 0.00421 | M37780 | 2154 |
| | (CD31 antigen) | | | |
| PECAM1 | platelet/endothelial cell adhesion molecule | 0.00909 | AA702701 | 2155 |
| | (CD31 antigen) | | | |
| PF4V1 | platelet factor 4 variant 1 | 0.0164 | NM_002620 | 2156 |
| PFKFB3 | 6-phosphofructo-2-kinase/fructose-2,6- | 0.0338 | NM_004566 | 2157 |
| | biphosphatase 3 | | | |
| PHACTR2 | phosphatase and actin regulator 2 | 0.039 | AA551142 | 2158 |
| PHACTR2 | phosphatase and actin regulator 2 | 0.0451 | NM_014721 | 2159 |
| PHCA | phytoceramidase, alkaline | 0.0221 | R12678 | 2160 |
| PHF21A | PHD finger protein 21A | 0.0231 | BC015714 | 2161 |
| PHGDHL1 | Phosphoglycerate dehydrogenase like 1 | 0.0444 | AF339764 | 2162 |
| PICALM | phosphatidylinositol binding clathrin assembly | 0.0165 | AL135735 | 2164 |
| | protein | | | |
| PICALM | phosphatidylinositol binding clathrin assembly | 0.0363 | AV722190 | 762 |
| | protein | | | |
| PLEK | pleckstrin | 0.000252 | NM_002664 | 2165 |
| PLEKHA2 | pleckstrin homology domain containing, family | 0.0183 | BF347859 | 886 |
| | A (phosphoinositide binding specific) member 2 | | | |
| PLEKHM1 | pleckstrin homology domain containing, family | 0.0134 | AB002354 | 2166 |
| | M (with RUN domain) member 1 | | | |
| PLSCR1 | phospholipid scramblase 1 | 0.0487 | AI825926 | 2167 |
| PLXNB2 | plexin B2 | 0.0177 | BC004542 | 2168 |
| pp9099 | PH domain-containing protein | 0.0311 | NM_025201 | 2169 |
| PPGB | protective protein for beta-galactosidase | 0.0193 | NM_000308 | 2170 |
| | (galactosialidosis) | | | |
| PPIG | Peptidyl-prolyl isomerase G (cyclophilin G) | 0.011 | AW970944 | 2171 |
| PPM1F | Protein phosphatase 1F (PP2C domain | 0.0208 | BC013798 | 2172 |
| | containing) | | | |
| PPP3CA | Protein phosphatase 3 (formerly 2B), catalytic | 0.0165 | AA532718 | 2173 |
| | subunit, alpha isoform (calcineurin A alpha) | | | |
| PRKACA | protein kinase, cAMP-dependent, catalytic, | 0.00184 | NM_002730 | 2174 |
| | alpha | | | |
| PRKAR1A | protein kinase, cAMP-dependent, regulatory, | 0.0262 | AI682905 | 2175 |
| | type I, alpha (tissue specific extinguisher 1) | | | |
| PRKAR2B | protein kinase, cAMP-dependent, regulatory, | 0.0441 | NM_002736 | 1688 |
| | type II, beta | | | |
| PRKCD | protein kinase C, delta | 0.0077 | NM_006254 | 2176 |
| PROK2 | prokineticin 2 | 0.0338 | AF182069 | 2177 |
| PSENEN | presenilin enhancer 2 homolog (C. elegans) | 0.0201 | NM_018468 | 2178 |
| PTGS1 | prostaglandin-endoperoxide synthase 1 | 0.0419 | NM_000962 | 329 |
| | (prostaglandin G/H synthase and | | | |
| | cyclooxygenase) | | | |
| PTGS1 | prostaglandin-endoperoxide synthase 1 | 0.0474 | S36219 | 1667 |
| | (prostaglandin G/H synthase and | | | |
| | cyclooxygenase) | | | |
| PTGS1 | prostaglandin-endoperoxide synthase 1 | 0.0226 | NM_000962 | 329 |
| | (prostaglandin G/H synthase and | | | |
| | cyclooxygenase) | | | |
| PTGS2 | prostaglandin-endoperoxide synthase 2 | 0.0187 | AY151286 | 2179 |
| | (prostaglandin G/H synthase and | | | |
| | cyclooxygenase) | | | |
| PTPN12 | Protein tyrosine phosphatase, non-receptor | 0.00163 | AL079909 | 2180 |
| | type 12 | | | |
| PTPRE | protein tyrosine phosphatase, receptor type, E | 0.0161 | AA775177 | 2181 |
| RAB11FIP1 | RAB11 family interacting protein 1 (class I) | 0.0397 | NM_025151 | 2182 |
| RAB11FIP4 | RAB11 family interacting protein 4 (class II) | 0.047 | BG248313 | 465 |
| RAB18 | RAB18, member RAS oncogene family | 0.0414 | AU145607 | 2183 |
| RAB31 | RAB31, member RAS oncogene family | 0.0378 | NM_006868 | 2184 |
| RAB8A | RAB8A, member RAS oncogene family | 0.00561 | BC002977 | 2185 |
| RAC1 | ras-related C3 botulinum toxin substrate 1 | 0.0328 | AJ012502 | 551 |
| | (rho family, small GTP binding protein Rac1) | | | |
| RAI17 | retinoic acid induced 17 | 0.0318 | AF070622 | 1313 |
| RAP1A | RAP1A, member of RAS oncogene family | 0.0194 | AW793677 | 2186 |
| RAP80 | Receptor associated protein 80 | 0.00717 | AF113675 | 2187 |
| RARA | retinoic acid receptor, alpha | 0.00262 | AJ417079 | 2188 |
| RASA1 | RAS p21 protein activator (GTPase activating | 0.0372 | AK021812 | 2189 |
| | protein) 1 | | | |
| RBBP5 | retinoblastoma binding protein 5 | 0.0383 | NM_005057 | 2190 |
| REL | v-rel reticuloendotheliosis viral oncogene | 0.0166 | NM_002908 | 2191 |
| | homolog (avian) | | | |
| RERE | Arginine-glutamic acid dipeptide (RE) repeats | 0.0354 | H71242 | 2192 |
| RFWD2 | Ring finger and WD repeat domain 2 | 0.0334 | BF725688 | 2193 |
| RFX2 | regulatory factor X, 2 (influences HLA class II | 0.0472 | AK024288 | 2194 |
| | expression) | | | |
| RG9MTD3 | RNA (guanine-9-) methyltransferase domain | 0.0449 | AI214944 | 2195 |
| | containing 3 | | | |
| RHOQ | ras homolog gene family, member Q | 0.0402 | BC033251 | 2196 |
| RIN3 | Ras and Rab interactor 3 | 0.0322 | AA625133 | 2197 |
| RIT1 | Ras-like without CAAX 1 | 0.0214 | AL037450 | 1675 |
| RNF24 | ring finger protein 24 | 0.0415 | BC000213 | 2198 |
| ROCK1 | Rho-associated, coiled-coil containing protein | 0.0356 | AL050032 | 2199 |
| | kinase 1 | | | |
| RSU1 | Ras suppressor protein 1 | 0.0152 | AW515913 | 2200 |
| RUFY1 | RUN and FYVE domain containing 1 | 0.0258 | NM_025158 | 2201 |
| RUFY2 | RUN and FYVE domain containing 2 | 0.00853 | NM_017987 | 2202 |
| SEC14L1 | SEC14-like 1 (S. cerevisiae) | 0.0303 | NM_003003 | 2203 |
| SEC23B | Sec23 homolog B (S. cerevisiae) | 0.0301 | BC005032 | 2204 |
| SECTM1 | secreted and transmembrane 1 | 0.0499 | BF939675 | 2205 |
| SERPINA1 | serpin peptidase inhibitor, clade A (alpha-1 | 0.022 | NM_000295 | 2206 |
| | antiproteinase, antitrypsin), member 1 | | | |
| SERPINA1 | serpin peptidase inhibitor, clade A (alpha-1 | 0.0459 | AF119873 | 2207 |
| | antiproteinase, antitrypsin), member 1 | | | |
| SF3A2 | splicing factor 3a, subunit 2, 66 kDa | 0.0469 | L21990 | 2208 |
| SGK | serum/glucocorticoid regulated kinase | 0.028 | NM_005627 | 2209 |
| SH3BGRL2 | SH3 domain binding glutamic acid-rich protein | 0.0258 | AL035700 | 1736 |
| | like 2 | | | |
| SIGLEC7 | sialic acid binding Ig-like lectin 7 | 0.0193 | NM_016543 | 2210 |
| SIRPB1 | signal-regulatory protein beta 1 | 0.0432 | NM_006065 | 2212 |
| SLC33A1 | Solute carrier family 33 (acetyl-CoA | 0.0407 | BC029450 | 2213 |
| | transporter), member 1 | | | |
| SLC7A7 | solute carrier family 7 (cationic amino acid | 0.0242 | NM_003982 | 2214 |
| | transporter, y+ system), member 7 | | | |
| SLIC1 | Selectin ligand interactor cytoplasmic-1 | 0.0159 | AI081246 | 2215 |
| SMARCA4 | SWI/SNF related, matrix associated, actin | 0.0291 | D26156 | 2216 |
| | dependent regulator of chromatin, subfamily | | | |
| | a, member 4 | | | |
| SMC5L1 | SMC5 structural maintenance of | 0.0179 | BC035661 | 2217 |
| | chromosomes 5-like 1 (yeast) | | | |
| SND1 | Staphylococcal nuclease domain containing 1 | 0.0162 | AI374686 | 2218 |
| SNX1 | Sorting nexin 1 | 0.0426 | AK098001 | 2219 |
| SOD2 | superoxide dismutase 2, mitochondrial | 0.0239 | W46388 | 2220 |
| SP110 | SP110 nuclear body protein | 0.0401 | AF280094 | 2221 |
| SP2 | Sp2 transcription factor | 0.0386 | D28588 | 2222 |
| SP3 | Sp3 transcription factor | 0.0499 | N23846 | 2223 |
| SPAG9 | sperm associated antigen 9 | 0.0493 | BC007524 | 2224 |
| ST8SIA4 | ST8 alpha-N-acetyl-neuraminide alpha-2,8- | 0.0441 | AA352113 | 1171 |
| | sialyltransferase 4 | | | |
| STAT3 | signal transducer and activator of transcription | 0.025 | BC000627 | 2225 |
| | 3 (acute-phase response factor) | | | |
| STAT3 | signal transducer and activator of transcription | 0.0328 | AA634272 | 2226 |
| | 3 (acute-phase response factor) | | | |
| STAT3 | signal transducer and activator of transcription | 0.0405 | AI139252 | 2227 |
| | 3 (acute-phase response factor) | | | |
| STEAP4 | STEAP family member 4 | 0.0195 | AA650281 | 2228 |
| SULT1A1 | sulfotransferase family, cytosolic, 1A, phenol- | 0.0233 | NM_001055 | 2229 |
| | preferring, member 1 | | | |
| SULT1A3; SULT1A4 | sulfotransferase family, cytosolic, 1A, phenol- | 0.0234 | U08032 | 2230 |
| | preferring, member 3; sulfotransferase family, | | | |
| | cytosolic, 1A, phenol-preferring, member 4 | | | |
| SVIL | Supervillin | 0.0273 | BC022883 | 2231 |
| SYK | Spleen tyrosine kinase | 0.0336 | AW467357 | 2232 |
| TAX1BP3 | Tax1 (human T-cell leukemia virus type I) | 0.027 | AF234997 | 2233 |
| | binding protein 3 | | | |
| TBC1D22A | TBC1 domain family, member 22A | 0.0353 | AI597838 | 2234 |
| TGFB1 | transforming growth factor, beta 1 (Camurati- | 0.0114 | BC000125 | 2235 |
| | Engelmann disease) | | | |
| TGFBR3 | transforming growth factor, beta receptor III | 0.0469 | NM_003243 | 2236 |
| | (betaglycan, 300 kDa) | | | |
| TGOLN2 | trans-golgi network protein 2 | 0.00127 | NM_006464 | 1425 |
| THRAP2 | Thyroid hormone receptor associated protein 2 | 0.04 | AK025348 | 2237 |
| TIAF1; MYO18A | TGFB1-induced anti-apoptotic factor 1; | 0.0229 | NM_004740 | 2238 |
| | myosin XVIIIA | | | |
| TJP2 | tight junction protein 2 (zona occludens 2) | 0.0455 | NM_004817 | 675 |
| TLE3 | transducin-like enhancer of split 3 (E(sp1) | 0.0127 | AI567426 | 2239 |
| | homolog, Drosophila) | | | |
| TLE3 | transducin-like enhancer of split 3 (E(sp1) | 0.0216 | NM_005078 | 2240 |
| | homolog, Drosophila) | | | |
| TLR1 | toll-like receptor 1 | 0.0328 | AL050262 | 2241 |
| TMCC1 | transmembrane and coiled-coil domain family 1 | 0.0328 | AB018322 | 1003 |
| TMCC1 | transmembrane and coiled-coil domain family 1 | 0.0345 | AI934469 | 2242 |
| TMEM16F | transmembrane protein 16F | 0.0268 | AL041280 | 2243 |
| TNFSF10 | tumor necrosis factor (ligand) superfamily, | 0.0148 | NM_003810 | 2244 |
| | member 10; tumor necrosis factor (ligand) | | | |
| | superfamily, member 10 | | | |
| TNFSF10 | tumor necrosis factor (ligand) superfamily, | 0.0367 | U57059 | 2245 |
| | member 10; tumor necrosis factor (ligand) | | | |
| | superfamily, member 10 | | | |
| TNFSF13; TNFSF12- | tumor necrosis factor (ligand) superfamily, | 0.012 | AF114013 | 2246 |
| TNFSF13 | member 13; tumor necrosis factor (ligand) | | | |
| | superfamily, member 12-member 13 | | | |
| TNFSF13; TNFSF12- | tumor necrosis factor (ligand) superfamily, | 0.0208 | AF114011 | 2247 |
| TNFSF13 | member 13; tumor necrosis factor (ligand) | | | |
| | superfamily, member 12-member 13 | | | |
| TNFSF13B | tumor necrosis factor (ligand) superfamily, | 0.0196 | AF134715 | 2248 |
| | member 13b | | | |
| TNRC5 | trinucleotide repeat containing 5 | 0.00226 | AF161347 | 2249 |
| TPARL | TPA regulated locus | 0.0263 | AW665624 | 939 |
| TPP1 | tripeptidyl peptidase I | 0.000155 | NM_000391 | 920 |
| TPP1 | tripeptidyl peptidase I | 0.00621 | BG231932 | 2250 |
| TRIM38 | Tripartite motif-containing 38 | 0.018 | AI809831 | 2251 |
| TRIM8 | tripartite motif-containing 8; tripartite motif- | 0.00322 | NM_030912 | 2252 |
| | containing 8 | | | |
| UBE2D3 | ubiquitin-conjugating enzyme E2D 3 (UBC4/5 | 0.0322 | AI239832 | 2253 |
| | homolog, yeast) | | | |
| UBE2J1 | Ubiquitin-conjugating enzyme E2, J1 (UBC6 | 0.0247 | N64079 | 2254 |
| | homolog, yeast) | | | |
| UBE3B | ubiquitin protein ligase E3B | 0.0134 | AK022019 | 2255 |
| USP15 | Ubiquitin specific peptidase 15 | 0.0357 | AU156202 | 2256 |
| VCL | vinculin | 0.000443 | NM_014000 | 2257 |
| VPS37C | vacuolar protein sorting 37C (yeast) | 0.00954 | BM452172 | 2258 |
| VPS41 | vacuolar protein sorting 41 (yeast) | 0.0126 | AF135593 | 342 |
| WARS | tryptophanyl-tRNA synthetase | 0.0139 | NM_004184 | 2259 |
| WASL | Wiskott-Aldrich syndrome-like | 0.0268 | AK025323 | 2260 |
| WIRE | WIRE protein | 0.00508 | AK026913 | 2261 |
| WNK1 | WNK lysine deficient protein kinase 1 | 0.0169 | AL049278 | 2262 |
| XPO1 | Exportin 1 (CRM1 homolog, yeast) | 0.0377 | BC041658 | 2263 |
| YWHAH | tyrosine 3-monooxygenase/tryptophan 5- | 0.018 | NM_003405 | 1807 |
| | monooxygenase activation protein, eta | | | |
| | polypeptide | | | |
| ZAK | sterile alpha motif and leucine zipper | 0.00126 | AW069181 | 2264 |
| | containing kinase AZK | | | |
| ZCCHC2 | zinc finger, CCHC domain containing 2 | 0.0109 | BE676543 | 2265 |
| ZCCHC2 | zinc finger, CCHC domain containing 2 | 0.022 | NM_017742 | 677 |
| ZCCHC2 | zinc finger, CCHC domain containing 2 | 0.0424 | AU147903 | 2266 |
| ZCCHC6 | zinc finger, CCHC domain containing 6 | 0.0477 | AA584428 | 2267 |
| ZFHX1B | Zinc finger homeobox 1b | 0.0124 | AF086037 | 2268 |
| ZFHX1B | Zinc finger homeobox 1b | 0.0233 | AI623184 | 2269 |
| ZFP36L1 | zinc finger protein 36, C3H type-like 1 | 0.0409 | BE620915 | 2270 |
| ZNF160 | zinc finger protein 160 | 0.028 | AA701249 | 1369 |
| ZNF264 | zinc finger protein 264 | 0.00262 | NM_003417 | 2271 |
| ZNF652 | zinc finger protein 652 | 0.0417 | NM_014897 | 2272 |
| ZYG11B | zyg-11 homolog B (C. elegans) | 0.00211 | AV701229 | 2273 |
| ZYG11B | zyg-11 homolog B (C. elegans) | 0.0262 | N21008 | 2274 |
| ZYX | zyxin | 0.0212 | BC002323 | 2275 |
| | MRNA; cDNA DKFZp564O0862 (from clone | 0.00258 | AW130600 | 2276 |
| | DKFZp564O0862) | | | |
| | CDNA clone IMAGE: 4769453 | 0.00433 | AI350995 | 2277 |
| | | 0.00555 | BF055351 | 334 |
| | CDNA FLJ12367 fis, clone MAMMA1002413 | 0.00572 | AL119001 | 2278 |
| | Homo sapiens, clone IMAGE: 4695648, mRNA | 0.00609 | BC016013 | 2279 |
| | Similar to hypothetical protein FLJ36144 | 0.00719 | AI090764 | 2280 |
| | | 0.0108 | U20489 | 2281 |
| | Transcribed locus, weakly similar to | 0.0115 | AA838727 | 2282 |
| | NP_060312.1 hypothetical protein FLJ20489 | | | |
| | [Homo sapiens] | | | |
| | Transcribed locus | 0.0133 | AW451107 | 2283 |
| | | 0.0147 | NM_173704 | 2284 |
| | Full length insert cDNA YR33G07 | 0.0165 | H48516 | 2285 |
| | CDNA FLJ41375 fis, clone BRCAN2007700 | 0.017 | BE739287 | 2286 |
| | CDNA clone IMAGE: 4769453 | 0.017 | AA806989 | 2287 |
| | | 0.0176 | AK026267 | 2288 |
| | Homo sapiens, clone IMAGE: 5745627, mRNA | 0.0182 | BC040914 | 2289 |
| | CDNA FLJ39805 fis, clone SPLEN2007951 | 0.0205 | CA444630 | 1377 |
| | | 0.0206 | BC035170 | 2290 |
| | Clone 25194 mRNA sequence | 0.0209 | BF438386 | 2291 |
| | Transcribed locus | 0.0211 | AI718964 | 2292 |
| | CDNA FLJ14081 fis, clone HEMBB1002280 | 0.0214 | BM728567 | 2293 |
| | Homo sapiens, clone IMAGE: 4272847, mRNA | 0.0226 | BC008462 | 2294 |
| | CDNA FLJ43285 fis, clone MESAN2000067 | 0.0243 | AI302271 | 2295 |
| | CDNA FLJ33139 fis, clone UTERU1000109 | 0.0246 | AK057701 | 2296 |
| | CDNA clone IMAGE: 6254031 | 0.0299 | AI800713 | 2297 |
| | Transcribed locus | 0.0303 | AI916948 | 1660 |
| | | 0.0304 | N35922 | 2298 |
| | CDNA FLJ46440 fis, clone THYMU3016022 | 0.0309 | BG288330 | 2299 |
| | Full length insert cDNA YR33G07 | 0.0312 | AW340891 | 729 |
| | Transcribed locus | 0.0326 | AW293956 | 2300 |
| | | 0.0334 | AI371550 | 2301 |
| | | 0.0336 | BG389789 | 2302 |
| | | 0.0338 | AF116653 | 2303 |
| | | 0.0344 | AA207241 | 2304 |
| | | 0.0353 | BC035958 | 2305 |
| | | 0.04 | H43637 | 2306 |
| | | | | 2307 |
| | | | | 2308 |
| | | | | 2309 |
| BGR | Acyl-CoA synthetase bubblegum family | 0.00516 | AW341515 | 2310 |
| | member 2 | | | |
| C1orf128 | chromosome 1 open reading frame 128 | 0.031 | AF271784 | 2311 |
| CDC27 | Cell division cycle 27 | 0.0359 | AA166684 | 2312 |
| FGFR1OP2 | FGFR1 oncogene partner 2 | 0.0301 | W74643 | 1750 |
| FLJ37562 | hypothetical protein FLJ37562 | 0.0328 | BF436799 | 2313 |
| HBLD2 | HESB like domain containing 2; HESB like | 0.0311 | NM_030940 | 2314 |
| | domain containing 2 | | | |
| KLF9 | Kruppel-like factor 9 | 0.0319 | NM_001206 | 2315 |
| LOC51326 | ADP-ribosylation factor-like | 0.0429 | AF119889 | 2316 |
| MLC1SA | myosin light chain 1 slow a | 0.0202 | NM_002475 | 2317 |
| MTP18 | mitochondrial protein 18 kDa | 0.0129 | AF060924 | 2318 |
| NUDT4; NUDT4P1 | nudix (nucleoside diphosphate linked moiety | 0.0352 | AF191653 | 1738 |
| | X)-type motif 4; nudix (nucleoside | | | |
| | diphosphate linked moiety X)-type motif 4 | | | |
| | pseudogene 1 | | | |
| OSBP2 | oxysterol binding protein 2 | 0.0334 | BE501253 | 2319 |
| RAB2B | RAB2B, member RAS oncogene family | 0.0428 | AA531016 | 2320 |
| RARA | retinoic acid receptor, alpha; retinoic acid | 0.0198 | U41742 | 2321 |
| | receptor, alpha | | | |
| SLC25A36 | solute carrier family 25, member 36 | 0.0384 | AL136803 | 56 |
| TAL1 | T-cell acute lymphocytic leukemia 1 | 0.0248 | AW300901 | 2322 |
| TMPRSS6 | transmembrane protease, serine 6 | 0.0304 | AL022314 | 2323 |
| WDR40A | WD repeat domain 40A | 0.0469 | AL555107 | 2324 |
| YOD1 | YOD1 OTU deubiquinating enzyme 1 | 0.0189 | AF090896 | 2325 |
| | homolog (yeast) | | | |
| ZNF595 | Zinc finger protein 595 | 0.00776 | AI580142 | 2326 |
| ZNF626 | zinc finger protein 626 | 0.0179 | NM_145297 | 2327 |
| | CDNA FLJ43311 fis, clone NT2RI2009855 | 0.0424 | AI985021 | 1350 |
| | Hypothetical gene supported by AK024248; | 0.043 | AF086554 | 2328 |
| | AL137733 | | | |
| | Transcribed locus | 0.0435 | H94876 | 2329 |
| | Transcribed locus | 0.0439 | BF514098 | 2330 |
| | Similar to transcript increased in | 0.044 | AK026679 | 2331 |
| | spermiogenesis 78 | | | |
| | Full-length cDNA clone CS0DJ002YF02 of T | 0.0449 | AI300571 | 2332 |
| | cells (Jurkat cell line) Cot 10-normalized of | | | |
| | Homo sapiens (human) | | | |
| | Transcribed locus | 0.0452 | AW954199 | 2333 |
| | | 0.047 | AU120130 | 2334 |
| | | 0.0472 | AL117451 | 2335 |

TABLE 8
Age, Race, Gender of Individuals in Study

| | | | Duchenne muscular |
|---|---|---|---|
| | Description | Control | dystrophy |
| Number | Subjects | 21 | 34 |
| Age | Years | 11 ± 3 | 9 ± 3 |
| Gender | Male | 100% | 100% |
| | Female | 0% | 0% |
| Race | White | 95% | 91% |
| | Black | 5% | 0% |
| | Other | 0% | 9% |

TABLE 9
PAM Analysis of Control vs. Duchenne muscular dystrophy (DMD)
gene set using ≧2.2 Fold Cut-off

| Gene | | Nucleic | SEQ | | Fold |
|---|---|---|---|---|---|
| Symbol | Gene Name | Acid No. | ID NO. | p-value | Change |
| DEFA4 | defensin, alpha 4, corticostatin | 1 | 1 | 2.04E−04 | 26.06 |
| SH3GL3 | SH3-domain GRB2-like 3 | 2 | 2 | 4.77E−06 | 11.96 |
| CEACAM8 | carcinoembryonic antigen-related cell adhesion molecule 8 | 5 | 5 | 6.41E−03 | 7.12 |
| AZU1 | azurocidin 1 (cationic antimicrobial protein 37) | 8 | 8 | 6.91E−04 | 6.27 |
| ELA2 | elastase 2, neutrophil | 9 | 9 | 7.86E−03 | 5.80 |
| RASEF | RAS and EF-hand domain containing | 12 | 12 | 1.63E−06 | 3.96 |
| ECHDC3 | enoyl Coenzyme A hydratase domain containing 3 | 13 | 13 | 3.95E−02 | 3.84 |
| RAB45 | hypothetical protein FLJ31614 | 15 | | 1.59E−06 | 3.65 |
| IL1R2 | interleukin 1 receptor, type II | 16 | 15 | 3.85E−02 | 3.62 |
| TRIM4 | tripartite motif-containing 4 | 17 | 16 | 1.21E−03 | 3.57 |
| CTSG | cathepsin G | 20 | 19 | 3.52E−02 | 3.44 |
| SLC4A4 | solute carrier family 4, sodium bicarbonate transporter | 24 | 22 | 2.12E−03 | 3.08 |
| SLC16A3 | solute carrier family 16, member 3 | 25 | 23 | 5.14E−05 | 2.98 |
| FNDC3B | Fibronectin type III domain containing 3B | 33 | 30 | 1.99E−02 | 2.66 |
| ZNF33A | zinc finger protein 33A | 38 | 34 | 9.48E−04 | 2.56 |
| CCDC144C | coiled-coil domain containing 144C | 41 | 37 | 2.48E−04 | 2.54 |
| MIDN | midnolin | 46 | | 1.56E−07 | 2.47 |
| HP | haptoglobin | 47 | | 1.79E−05 | 2.47 |
| AP2B1 | adaptor-related protein complex 2, beta 1 subunit | 48 | | 1.38E−04 | 2.47 |
| GOLGA1 | Golgi autoantigen, golgin subfamily a, 1 | 49 | | 1.41E−06 | 2.46 |
| ERGIC1 | endoplasmic reticulum-golgi intermediate compartment 1 | 52 | | 8.75E−05 | 2.38 |
| CA4 | carbonic anhydrase IV | 54 | | 6.92E−04 | 2.35 |
| OPHN1 | oligophrenin 1 | 55 | | 1.82E−02 | 2.34 |
| SLC25A36 | solute carrier family 25, member 36 | 56 | | 1.64E−03 | 2.34 |
| POLK | polymerase (DNA directed) kappa | 57 | | 1.71E−02 | 2.32 |
| UBA6 | ubiquitin-like modifier activating enzyme 6 | 60 | | 8.66E−03 | 2.26 |
| TIMM8A | translocase of inner mitochondrial membrane 8 homolog A | 65 | | 1.38E−05 | 2.22 |

TABLE 10
PAM Analysis of Control vs. Duchenne muscular dystrophy (DMD)
gene set using ≦2.2 Fold Cut-off

| Gene | | Nucleic | SEQ | | Fold |
|---|---|---|---|---|---|
| Symbol | Gene Name - | Acid No. | ID NO | p-value | Change |
| HIPK3 | homeodomain interacting protein kinase 3 | 1467 | 55 | 9.74E−09 | 0.25 |
| FER1L3 | fer-I-like 3, myoferlin (C. elegans) | 1465 | 53 | 4.47E−10 | 0.32 |
| MTMR1 | myotubularin related protein 1 | 1464 | 52 | 4.24E−09 | 0.32 |
| TncRNA | trophoblast-derived noncoding RNA | 1463 | 51 | 4.96E−09 | 0.34 |
| CAB39 | calcium binding protein 39 | 1462 | 50 | 3.58E−07 | 0.36 |
| ST6GAL1 | ST6 beta-galactosamide alpha-2,6-sialyltranferase 1 | 1461 | 49 | 4.77E−07 | 0.36 |
| IGHD | immunoglobulin heavy constant delta | 1459 | 47 | 6.53E−05 | 0.39 |
| UBR2 | Ubiquitin protein ligase E3 component n-recognin 2 | 1456 | 44 | 4.73E−05 | 0.39 |
| DDX3X | DEAD (Asp-Glu-Ala-Asp) box polypeptide 3, X-linked | 1455 | 43 | 7.23E−10 | 0.40 |
| FNBP1 | formin binding protein 1 | 1454 | 42 | 5.17E−11 | 0.40 |
| UBE4B | ubiquitination factor E4B (UFD2 homolog, yeast) | 1453 | | 2.17E−09 | 0.40 |
| HTATSFI | HIV-1 Tat specific factor 1 | 1452 | | 6.00E−08 | 0.41 |
| KBTBD2 | kelch repeat and BTB (POZ) domain containing 2 | 1451 | | 3.30E−08 | 0.41 |
| PTPRO | protein tyrosine phosphatase, receptor type, O | 1450 | | 5.69E−11 | 0.41 |
| SP100 | SP100 nuclear antigen | 1449 | | 9.78E−11 | 0.41 |
| ZNF267 | zinc finger protein 267 | 1448 | | 2.03E−06 | 0.41 |
| CTNNA1 | catenin (cadherin-associated protein), alpha 1, 102 kDa | 1441 | | 1.51E−06 | 0.42 |
| PXN | Paxillin | 1440 | | 2.05E−07 | 0.42 |
| TMEM30A | transmembrane protein 30A | 1439 | | 5.27E−07 | 0.42 |
| TTF2 | transcription termination factor, RNA polymerase II | 1438 | | 3.82E−09 | 0.42 |
| BCOR | BCL6 co-repressor | 1435 | | 8.33E−08 | 0.43 |
| CDV3 | CDV3 homolog (mouse) | 1434 | | 1.40E−07 | 0.43 |
| DSP | Desmoplakin | 1433 | | 4.12E−04 | 0.43 |
| ILF3 | interleukin enhancer binding factor 3, 90 kDa | 1432 | | 2.43E−09 | 0.43 |
| SAMHD1 | SAM domain and HD domain 1 | 1431 | | 1.44E−08 | 0.43 |
| UPF3B | UPF3 regulator of nonsense transcripts homolog B (yeast) | 1430 | | 3.23E−06 | 0.43 |
| GPR177 | G protein-coupled receptor 177 | 1429 | | 7.09E−06 | 0.44 |
| MSI2 | musashi homolog 2 (Drosophila) | 1432 | | 6.37E−08 | 0.44 |
| PIK3AP1 | phosphoinositide-3-kinase adaptor protein 1 | 1429 | | 6.19E−08 | 0.44 |
| SMC3 | structural maintenance of chromosomes 3 | 1428 | | 6.67E−07 | 0.44 |
| LNPEP | leucyl/cystinyl aminopeptidase | 1424 | | 9.83E−06 | 0.45 |
| MEF2C | myocyte enhancer factor 2C | 1423 | | 6.37E−08 | 0.45 |
| TFCP2 | transcription factor CP2 | 1422 | | 7.08E−08 | 0.45 |
| TGOLN2 | trans-golgi network protein 2 | 1421 | | 3.27E−11 | 0.45 |

TABLE 11
KEGG Analysis of Control vs. Duchenne muscular dystrophy (DMD)
gene set with ≧|2.0| Fold change

| | Number of | | Differentially |
|---|---|---|---|
| | Differentially | | Expressed |
| | Expressed Genes | | Genes and Probe Sets |
| Pathway Name | in Pathway | P-value | in Pathway |
| Leukocyte | 9 | 0.01 | LOC651633 |
| Transendothelial | | | ITGB1 |
| Migration | | | PXN |
| | | | ITGA4 |
| | | | PKC |
| | | | CTNNA1 |
| | | | ROCK |
| | | | a-CATENIN |
| | | | PAXILLIN |
| Regulation of Actin | 7 | 0.03 | APC |
| Cytoskeleton | | | ARHGEF7 |
| | | | LOC651633 |
| | | | ITGB1 |
| | | | PXN |
| | | | ITGA4 |
| | | | GNA13 |
| Antigen Processing | 4 | 0.06 | HLA-DRB1 |
| and Presentation | | | HSP90AA1 |
| | | | HSP90AB1 |
| | | | TAP2 |
| Neurodegenerative | 3 | 0.06 | APP |
| Disorders | | | CASP1 |
| | | | PRNPc |

TABLE 12
Transcription Factor Binding Motif Analysis

| | | Fold | |
|---|---|---|---|
| Transcription factor | TBFM | difference* | p value |
| Overrepresented motifs | | | |
| myogenic enhancer factor 2 | V$MEF2_01 | 85.7 | 1.00E−10 |
| CP2 | V$CP2_01 | 3.8 | 4.02E−05 |
| Octamer binding site | V$OCT_C | 5.5 | 0.0016 |
| transcriptional repressor CDP | V$CDP_02 | 5.1 | 0.002 |
| POU-factor Tst-1/Oct-6 | V$TST1_01 | 3.3 | 0.0046 |
| CCAAT/enhancer binding | V$CEBPA_01 | 1.7 | 0.0057 |
| protein alpha | | | |
| octamer-binding factor 1 | V$OCT1_05 | 4.2 | 0.0087 |
| cap signal for transcription | V$CAP_01 | 1.1 | 0.0092 |
| initiation | | | |
| POU factor Brn-2 | V$BRN2_01 | 2.1 | 0.0123 |
| activator protein 4 | V$AP4_01 | 4.7 | 0.0161 |
| S8 | V$S8_01 | 1.6 | 0.0222 |
| Clox | V$CLOX_01 | 4.2 | 0.0266 |
| c-Myb | V$CMYB_01 | 2 | 0.0375 |
| Underrepresented motifs | | | |
| Ikaros 1 | V$IK1_01 | −2.5 | 0.0467 |

*Fold difference of frequency of TFBMs in 45 regulated genes compared with the frequency observed in genes not differentially expressed, using Affymetrix U133 Plus 2.0 probes as background.

TABLE 13
DAVID Analysis of Duchenne muscular dystrophy (DMD) vs. Duchenne
muscular dystrophy (DMD) receiving steroid gene set.

| | | # | | |
|---|---|---|---|---|
| Annotation | Description | genes | P_Value | FDR |
| SP_PIR_KEYWORDS | antibiotic | 7 | 4.30E−09 | 4.60E−06 |
| SP_PIR_KEYWORDS | antimicrobial | 7 | 4.90E−09 | 2.60E−06 |
| GOTERM_BP_ALL | defense response | 14 | 1.10E−08 | 5.80E−05 |
| GOTERM_BP_ALL | defense response to bacterium | 7 | 1.90E−07 | 4.90E−04 |
| GOTERM_BP_ALL | response to bacterium | 7 | 4.10E−07 | 7.10E−04 |
| GOTERM_BP_ALL | response to other organism | 8 | 1.80E−06 | 2.40E−03 |
| GOTERM_BP_ALL | response to biotic stimulus | 9 | 2.10E−06 | 2.20E−03 |
| GOTERM_BP_ALL | multi-organism process | 8 | 4.10E−05 | 3.00E−02 |
| GOTERM_BP_ALL | response to fungus | 3 | 1.50E−03 | 5.50E−01 |
| SP_PIR_KEYWORDS | Direct protein sequencing | 15 | 2.20E−03 | 2.80E−01 |

TABLE 14
DAVID Analysis of Duchenne muscular dystrophy (DMD) receiving
deflazacort vs. Duchenne muscular dystrophy (DMD)
receiving prednisone gene set

| | | # | | |
|---|---|---|---|---|
| Annotation | Description | genes | P_Value | FDR |
| GOTERM_BP_ALL | intracellular | 75 | 1.70E−10 | 9.00E−07 |
| | signaling cascade | | | |
| GOTERM_BP_ALL | signal | 120 | 1.40E−07 | 3.70E−04 |
| | transduction | | | |
| GOTERM_BP_ALL | cell | 126 | 1.40E−06 | 1.30E−03 |
| | communication | | | |

TABLE 15
Set of Genes Common to both the Duchenne muscular dystrophy
(DMD) vs. Duchenne muscular dystrophy (DMD) receiving steroid
gene set, and the Duchenne muscular dystrophy (DMD) receiving
deflazacort vs. Duchenne muscular dystrophy (DMD)
receiving prednisone gene set

| | | | Nucleic |
|---|---|---|---|
| Systematic | Gene Symbol | Description | Acid No. |
| 201059_at | CTTN | cortactin | 1702 |
| 220615_s_at | MLSTD1 | male sterility domain | 1848 |
| | | containing 1 | |
| 1556283_s_at | FGFR1OP2 | FGFR1 oncogene | 1750 |
| | | partner 2 | |
| 1553349_at | ARID2 | AT rich interactive | 1576 |
| | | domain 2 (ARID, | |
| | | RFX-like) | |
| 206303_s_at | NUDT4; | nudix (nucleoside | 1738 |
| | NUDT4P1 | diphosphate linked | |
| | | moiety X)-type motif 4; | |
| | | nudix (nucleoside | |
| | | diphosphate linked | |
| | | moiety X)-type motif 4 | |
| | | pseudogene 1 | |
| 202191_s_at | GAS7 | growth arrest-specific 7 | 1711 |
| 211067_s_at | GAS7 | growth arrest-specific 7; | 1650 |
| | | growth arrest-specific 7 | |
| 217489_s_at | IL6R | interleukin 6 receptor | 1686 |
| 236224_at | RIT1 | Ras-like without CAAX 1 | 1675 |
| 236223_s_at | RIT1 | Ras-like without CAAX 1 | 1675 |
| 201020_at | YWHAH | tyrosine 3- | 1807 |
| | | monooxygenase/tryptophan | |
| | | 5-monooxygenase | |
| | | activation protein, eta | |
| | | polypeptide | |
| 209288_s_at | CDC42EP3 | CDC42 effector protein | 1664 |
| | | (Rho GTPase binding) 3 | |
| 203817_at | GUCY1B3 | guanylate cyclase 1, | 1590 |
| | | soluble, beta 3 | |
| 225354_s_at | SH3BGRL2 | SH3 domain binding | 1736 |
| | | glutamic acid-rich | |
| | | protein like 2 | |
| 226018_at | Ells1 | hypothetical protein Ells1 | 1596 |
| 203680_at | PRKAR2B | protein kinase, cAMP- | 1688 |
| | | dependent, regulatory, | |
| | | type II, beta | |
| 215813_s_at | PTGS1 | prostaglandin- | 1667 |
| | | endoperoxide synthase | |
| | | 1 (prostaglandin G/H | |
| | | synthase and | |
| | | cyclooxygenase) | |
| 235670_at | | Transcribed locus | 1660 |

TABLE 16
Classify Individuals based on Duchenne muscular dystrophy (DMD)
vs. Duchenne muscular dystrophy (DMD) receiving steroid gene set

| Cross Validation Result | DMD-Steroid | DMD-No Steroid |
|---|---|---|
| Total number of samples | 14 | 20 |
| Correct/incorrect prediction | 9/5 | 20/0 |
| Prediction Sensitivity | 64.29% | 100% |
| Prediction Specificity | 100% | 64.29% |

TABLE 17
Classify Individuals based on Duchenne muscular dystrophy
(DMD) receiving deflazacort vs. Duchenne muscular
dystrophy (DMD) receiving prednisone gene set

| Cross Validation Result | DEFL | PRED |
|---|---|---|
| Total number of samples | 6 | 6 |
| Correct/incorrect prediction | 6/0 | 6/0 |
| Prediction Sensitivity | 100% | 100% |
| Prediction Specificity | 100% | 100% |

Other variations or embodiments of the invention will also be apparent to one of ordinary skill in the art from the above figures and descriptions. Thus, the forgoing embodiments are not to be construed as limiting the claim scope.

Applicants incorporate by reference the material contained in the accompanying computer readable Sequence Listing identified as Sequence_Listing.txt, having a file creation date of Oct. 13, 2009 3:54:51 P.M. and file size of 115 KB.

What is claimed is:

1. A method to evaluate an individual's propensity for Duchenne muscular dystrophy (DMD), the method comprising
determining from blood of a test individual expression of at least one gene selected from the group consisting of SEQ ID NOS. 1-55,
comparing the expression of the at least one gene from the test individual with an expression profile of the same gene(s) from a healthy control individual,
evaluating the test individual's propensity for DMD by determining if the at least one gene in the test individual is either over-expressed ≧1.5 times or is under-expressed ≧1.5 times compared to expression of the same gene(s) from the healthy control individual, and
ranking the test individual's propensity for DMD based on at least one of
(a) the extent that over-expression or under-expression exceeds 1.5, or
(b) the number of genes that are over-expressed or under-expressed,
where the test individual's propensity for DMD is ranked higher when (a) is farther from 1.5, and (b) is farther from 1 compared to the healthy control individual.

2. The method of claim 1 where the at least one gene is selected from the group consisting of SEQ ID NOS. 1-5, 6-11, 51-55, 46-50, and combinations thereof.

3. The method of claim 1 further comprising ranking the test individual's propensity higher if at least one of the genes over-expressed or under-expressed is selected from at least one of SEQ ID NOS. 1-5 or SEQ ID NOS. 51-55.

4. The method of claim 1 further comprising ranking the test individual's propensity higher if at least one of the genes over-expressed or under-expressed encodes defensin.

5. The method of claim 1 where in (a) over-expression or under-expression exceeds 2.

6. The method of claim 1 where in (a) over-expression or under-expression exceeds 2.5.

7. A method to diagnose Duchenne muscular dystrophy (DMD) in a test individual, the method comprising
determining from blood of a test individual expression of at least one gene selected from the group consisting of SEQ ID NOS. 1-55,
comparing expression of the at least one gene from the test individual with expression of the same gene(s) from a healthy control individual,
diagnosing DMD by determining if the at least one gene in the test individual is either over-expressed ≧2.5 times or is under-expressed ≧2.5 times compared to expression of the same gene(s) from the healthy control individual, and
determining a confidence level for the diagnosis of DMD based on at least one of
(a) the extent that over-expression or under-expression exceeds 2.5, or
(b) the number of genes that are over-expressed or under-expressed,
where the diagnosis for DMD in the test individual has a higher confidence level when (a) is farther from 2.5, and (b) is farther from 1.

8. The method of claim 7 further comprising ranking the confidence level for the diagnosis higher if at least one of the gene over-expressed or under-expressed is selected from at least one of SEQ ID NOS. 1-5 or SEQ ID NOS. 51-55.

9. The method of claim 7 further comprising ranking the confidence level for the diagnosis higher if at least one of the gene over-expressed or under-expressed encodes defensin.

10. A method of ranking a gene as a target for ameliorating Duchenne muscular dystrophy (DMD), the method comprising
(a) determining from blood of a test individual with DMD expression of at least one gene selected from the group consisting of SEQ ID NOS. 1-55,
(b) comparing the expression of the at least one gene from the test individual with an expression from the same gene(s) from a healthy control individual, and
(c) ranking the gene as a target by determining if the at least one gene in the test individual is either over-expressed ≧1.5 times or is under-expressed ≧1.5 times compared to the same gene(s) in the expression profile from the healthy control individual,
where the gene is a better target the farther (c) is from 1.5.

11. The method of claim 10 where in (c) over-expression or under-expression ≧2 and where the gene is a better target the farther (c) is from 2.

12. The method of claim 10 where in (c) over-expression or under-expression ≧2.5 and where the gene is a better target the farther (c) is from 2.5.

13. The method of claim 10 wherein in (a) the gene is selected from at least one of SEQ ID NO. 1-5 or SEQ ID NOS. 51-55.

14. The method of claim 10 further comprising administering at least one agent specific for the highest ranked gene.

15. The method of claim 14 where the agent modulates the immune system of the individual with DMD and/or where the agent modulates iron utilization of the individual with DMD.

16. A method of monitoring therapy in an individual with Duchenne muscular dystrophy (DMD), the method comprising
determining from blood of a test individual expression of at least one gene selected from the group consisting of SEQ ID NOS. 1-55,
comparing the expression of the at least one gene from the test individual with an expression profile of the same gene(s) from either
(i) a healthy control individual, or
(ii) a previous result from the test individual;
evaluating the test individual's response to therapy by determining if the at least one gene in the test individual is either over-expressed or is under-expressed compared to expression of the same gene(s) from either (i) or (ii), and
ranking the test individual's response to therapy based on at least one of
(a) the extent of over-expression or under-expression, or
(b) the number of genes that are over-expressed or under-expressed,
where the test individual's response to therapy is ranked higher when (a) and (b) are closer to either (i) or (ii).

17. The method of claim 16 where over-expression or under-expression is ≧1.5.

18. The method of claim 16 where over-expression or under-expression ≧2.

19. The method of claim 16 where over-expression or under-expression ≧2.5.

20. A kit for diagnosing Duchenne's muscular dystrophy from peripheral blood, the kit comprising
at least one primer, the primer comprising a forward oligonucleotide primer and/or a reverse oligonucleotide primer, the primer annealing to at least one of SEQ ID NO. 1-55, and
instructions for using the primer to quantitate SEQ ID NO. 1-55 in peripheral blood, where diagnosis of DMD is made by
determining from blood of a test individual expression of at least one gene selected from the group consisting of SEQ ID NOS. 1-55,
comparing expression of the at least one gene from the test individual with expression of the same gene(s) from a healthy control individual,
diagnosing DMD by determining if the at least one gene in the test individual is either over-expressed ≧2.5 times or is under-expressed ≧2.5 times compared to expression of the same gene(s) from the control individual, and
determining a confidence level for the diagnosis of DMD based on at least one of
(a) the extent that over-expression or under-expression exceeds 2.5, or
(b) the number of genes that are over-expressed or under-expressed,
where the diagnosis for DMD in the test individual has a higher confidence level when (a) is farther from 2.5, and (b) is farther from 1.

21. The kit of claim 20 where the primer anneals to the gene encoding defensin.

22. The kit of claim 20 wherein the primer anneals to the gene encoding T cell leukemia/lymphoma 1a.

23. The kit of claim 20 where the primer is used in the polymerase chain reaction.


SEQUENCE LISTING (55 sequences)

SEQ ID NO: 1 - DNA, 542 bp, Homo sapiens
gtctgccctctctgctcgccctgcctagcttgaggatctgtcaccccagccatgaggattatcgccctcctcgctgctattctcttggtagccctccaggtccgggcaggcccactccaggcaagaggtgatgaggctccaggccaggagcagcgtgggccagaagaccaggacatatctatttcctttgcatgggataaaagctctgctcttcaggtttcaggctcaacaaggggcatggtctgctcttgcagattagtattctgccggcgaacagaacttcgtgttgggaactgcctcattggtggtgtgagtttcacatactgctgcacgcgtgtcgattaacgttctgctgtccaagagaatgtcatgctgggaacgccatcatcggtggtgttagcttcacatgcttctgcagctgagcttgcagaatagagaaaaatgagctcataatttgctttgagagctacaggaaatggttgtttctcctatactttgtccttaacatctttcttgatcctaaatatatatctcgtaacaag

SEQ ID NO: 2 - DNA, 2738 bp, Homo sapiens
gcgatgtcggtggccgggctgaagaagcagttccacaaagccagccagctatttagtgaaaaaataagtggtgctgaaggaactaaactagacgatgaatttcttgacatggaaaggaaaatagatgttaccaataaagttgttgcagaaattctttcaaaaaccactgaatatcttcagccaaatccagcatacagagctaagctaggaatgctgaacactgtgtcgaagatccgagggcaggtgaagaccacaggatacccgcagacggaaggcttgctgggggactgtatgctgaaatacgggaaggagctcggggaagactccacctttggcaatgcattgatagaagttggtgaatccatgaagctaatggctgaggtgaaagactctcttgatattaatgtaaagcaaacttttattgatccacttcagttactacaagataaagatttaaaagagatcgggcatcacctgaaaaagctggaaggccgccgcctggattacgattataaaaagaaacgagtaggtaagataccagacgaagaagtcagacaagcggtagaaaaatttgaagagtcaaaggagttggctgaaagaagcatgtttaactttttagaaaatgatgtagaacaagtcagccagttggctgtgttcatagaggcagcattagactatcacagacagtccacagagattctgcaggagctgcagagcaagctacagatgcgaatatcagctgcatccagtgtccccagacgagaatacaagccaaggcctgtgaaaaggagttctagtgagctcaatggagtttccaccacctctgtagtgaagacgacaggttctaacattcccatggaccagccctgctgtcgtggtctctatgactttgagccagaaaaccaaggagaattaggatttaaagaaggggacggcaccttcagaaaaatcaaaagggaaactaagattaaaatgtgcagaaagaaaattgtcaatatttacaaattaaaagatcagcaacattgattttctgtgtcgttgttatgaaagccttactagtggatgcaaagtggcatctcatttgggttttgccttgcattttattaatgaataatggtgtttagcatcttttcttttccttcttagacatttgtgtaacttctttgggagaaatgtctgttcaagtcctttgcctatttttttaattgggatcttagaaattctgttgttgagttgtgggatattaagcttttatcagatacacattttgattttatcagatacatattttctcacatattatgggttgccttttcactcccttgatagtatcctttgatttttattttgattaaatctaattttcgtgtattttcttttgttatctgtgcttttctgtcatatttcaaaatacacttaaaactcaaaggtcataaaggtttaccgtgtgttttcttctaagagttacatatttttagtccttacatttaagtcttttattaatttagaattaatttttgtatatactgcaaggtaggggtctaacttctctcttgtacactgacatccagctgttgaagagactgttctttcctcccttgactagacttggccaccttgttgaacagtcattgaccatatatgtgaggactaacttgtaggatctcaaatctgtcctattgtatttgtctgaaagtctattggtcttattccagtaccacactctcttgattactgtagatttgtagtagtctgtgaaactgaaaaatgtgagttttccaatgttctttttcaagactgttttgtctgtcagatcctttgaatttttgtatgattttagaatgagtttctttgtttctgcaaaaatgcctttgggattttgatggtattgcattgaatctgtagattactttagatggtattgtcatcttaacaatattgtcttacaacccgtgaacacagaatgtctttccacttatttccactctctttagttttttgcagcaatgttttgtgtataccaccatggttagatttatgcctgaataacgtattctttgatgtcattataaatggaatttttaaaatgttttcatagttctttacaactatatagaaatatagctcatttgcctatgtttgtttgcatcctgcctcctttattagttataatcggttttgtgttttgtttggagctttatacctataagatcatgtgtagatataattttacacctattttttatttctaatttagatgccttttatttctttgtcttgcctaattgctctggctagaactgccagtgctacgttgaatacaagtggcaaatgcaccatccttttcttctagatgttaggaaaacagctttcagtgtttcatcattgatcatgatattaactgttgggtttttgtacatcccattgtcatgttgcagaagatcccttctatgcctagtttatagagtatttttattatagaagggtgttgtatttcatcaatgttttctctgcagcaattgaaataatcacgtgcttattcattttactgttacagcatattacactgattgattttttatatgttgaaccacccatgcattttggggataaatctcaaagggtgatagtttacaatcctttgattatacagtattgctgctagtattttgctagtattgctagtattttgctgagatttttgcttatatattcataggggatatagtgctgtattt

SEQ ID NO: 3 - DNA, 3527 bp, Homo sapiens
gggggatttggggctgggtcggccggggtcggggaggggggtggtgaaaaggtgacagggagctgccctcgctcaagagccggtggttgggggtctgagaagaagtcaccaatatgaagttattcggcttcgggagccgcaggggccagacggcccagggctccatagaccacgtctacacgggttccggataccgaatccgggactccgaactgcagaagatccacagggcagctgtcaaaggcgacgccgcggaggtggagcgctgcttggcgcgcaggagcggagacctggacgccctggacaagcagcacagaactgctctacacttggcctgtgccagtggccatgtgcaagtggtcactctcctggttaacagaaaatgccagattgatgtctgtgacaaagaaaacagaacgcctttgatacaggctgtccattgccaggaagaggcttgtgccgttattctgctggaacatggcgccaatccaaactttaaggatatctacggcaacactgctctccattatgccgtgtatagtgagagcacctcactggcagaaaaactgctttcccatggtgcacatattgaagcactggacaaggacaataataccccacttttatttgctataatttgcaagaaagagaaaatggtggaatttttattgaaaaagaaagcaagttcacatgccgttgataggctgagacggtcagctctcatgcttgctgtatactatgactcaccaggtattgtcaatatccttcttaagcaaaatattgatgtcttcgctcaagacatgtgtggacgagatgcagaagattatgctatttctcatcatttgacaaaaattcaacaacaaattttggaacataaaaagaagatacttaaaaaggagaaatcagatgttggaagttctgatgaatctgcagtcagcattttccatgaactgcgtgtggattcattgcctgcatcggatgacaaagacttgaatgttgctactaagcagtgtgtccccgagaaagtgtcagagcctttacctggatcttcgcatgaaaaaggaaacagaatagtcaatggacaaggagaagggcctcctgcaaaacatccttccttgaagcctagcactgaagtggaagatcctgctgtgaaaggagcagtacaaagaaagaatgtacagacattgagagcagaacaagccttaccagtggcttcagaggaagagcaagaaaggcatgaaagaagtgaaaagaagcaaccacaggtcaaagaaggaaataatacaaacaaaagtgaaaaaatacaactttcagaaaatatatgtgatagtacatcttctgctgctgctggcagattaacccaacaaagaaagattgggaaaacgtatcctcagcaatttcccaagaagctgaaggaagagcatgatagatgcaccttaaaacaagaaaatgaagaaaaaacaaatgttaatatgctgtacaaaaaaaatagagaagaattagaaaggaaagagaaacaatataagaaagaagttgaagcaaaacaacttgaaccaactgttcagtcactagagatgaaatcaaagactgcaagaaatactccaaatcgggattttcataatcatgaagaaatgaaaggtctgatggatgaaaattgcattttgaaggcagatattgctatactcagacaggaaatatgtacaatgaaaaatgacaacttggaaaaagaaaataaatatcttaaggacattaaaattgttaaagaaacaaatgctgcccttgaaaagtatataaaactcaatgaggaaatgataacagaaacagcattccggtatcaacaagagcttaatgatctcaaggctgagaatacaaggctcaatgccgaactgttgaaggaaaaagaaagcaagaaaagactggaagctgacattgaatcttatcagtctagactggctgctgctataagcaaacacagtgaaagtgtgaaaacagaaagaaacctaaaacttgctttagagagaacacgagatgtttctgtacaagtagaaatgagttctgctatttccaaagtaaaagctgagaatgagtttcttactgaacaactttctgaaacacaaattaaattcaatgccttaaaagataagttctgtaagacaagagatagtctcagaaaaaagtcattggctttagaaactgtacaaaacgacctaagccaaacacagcagcaaacacaggaaatgaaagagatgtatcaaaatgcagaagctaaagtgaataattccactggaaagtggaactgtgtagaagagaggatatgtcacctccaacgtgaaaatgcgtggcttgtacagcaactagatgacgttcatcagaaagaggatcataaagagatagtaactaatatccaaagaggctttattgagagtgaaaagaaagacctcgtgctagaagagaaaagtaagaagctaatgaatgaatgtgatcatttaaaagaaagtctctttcagtatgagagagagaaaacaggaggagtagtaagtatcaaggaagataaatattttcaaacttctagaaagacaatttaaacatttggttctggatacatgttgaacttagttgaatataaaaatctagattaaaagtgtgtttaccatactgtataattccatttacatgaagcatccagaaaagataaatgtatagggacaaaaagtagattcatgtttgcaaggggctggggctggaagctggtagtgactgctaatgggcatgaggaatcttacagtgatggaaatgctctaaagttggattgtagagatggctgcacaactcagtaaatgtactaaaaatcttttaactttaagttaaaacagatacattctatagtatgtaaattatatttcaacaaagctgttttaataaaaaaaggaaaaatgtgtttactatatcggcttagaaacatgcctcatttctaggaaataaaagatagaggtgagagatgatttactttgagaaaagacattgtgtcacctatgaaattttattaggcacagagtcatattttaaggtagatagttctgtattgctgaaatagtaattttaatgtctttatgttgccacatgttaagaccataatgtagttataaatggaaatgtttacacctgaagtgagtattttcaaattaaaatttaattaagtgattttcttcgacacttaattctagattccccagatgaattgaagtgtattgctgtgtcttgtaataccttgctttaactagctttttatgtattttagttggtatagctttgttattattcatattaacaaatctgaaaatatgtcaaattacatgtttttatgaccatgtaatgttttaaaggcacctacttgttataaaatcataatttaggataaatgtggtaaaacttagcaaaactatatttggtttagttttcccattggtatttatagtttactttgaatatttatattaataattagctcataatttttaaaaaaaaaa

SEQ ID NO: 4 - DNA, 1998 bp, Homo sapiens
aaagtatacaattttgagtaataagaacagaaatcaaagtaactattgtatgctgtattttagtagagcaacactgaagaagaaagaaaacaagaaataatattaaatatgaacatatggagaacagaataatatttttaaaatttttagtttctaagcttatctgaaattttaattttcttttcttatgtaataccagagttattaggaaggtattatctaataacactattttcagtgatattttaagtagttgtcctagaaaaatttctattttttaaaaatgtatatttaaaaatacattaaatgtgtatatacatcaatcatatgtatcgatttctgtttttcttgaattgcaaatgaaatttgtatttttgtgttcctggaataaaataaactcgaatgggtcgtaatatattattcatgctgtaattcaacgtatttgaatactttaagaatgttacatttatagttaacagatactgacctataaattttctgtcatataatgatgctgtgagacaatctgagaagaattaaaatttaaattcatgtattcctacttttttctctattctctaactgtaaaatattttcattacagatggaggaacaaatagatgttagataaatagatatataatagatcatccaaaattcttattcttatggttttatgtagtcagtatttacctctatttttctacatgtttatccttccaatttagttcattacttcctgcacctttgatgtcatatacataaacaggaaataacacatggtggctgggatgtagagagagccacaggacttgtgaatgaaatccacaggcaaggatgtggcgattccttttgcaatattggagggaatgccaaaccctatgtttgctgtggaaaagagtatggtagttcctcaaaacatcaaaatggtattgccttatgattcagcagacccacatcccaagacagcaaaagaattgaaagcagagtcttgaaaaaatatttgcacatccatgtttgcagcagcattattggcaatagctaaaacgtagaagcaattgaactgtccaacaacagatgaatggatgagcaaaacatgatatatacatacaatggaaaattattcagccttaaacgagggaaatattctgacatatgttgcaacttggatgaaacttgaggatattatgccaagtgaaataagttagtcagtgaaggacaaatacagtataattccatatgtataagggacttgaagtggacagaatcatagagatagtacaatgatggttgccagaagctggggggaggaagaaatggggaagtattgtttaatgggtatagtttcagttttacaagatgaaacgaattatggagatggatggtagggatggctgcacaatgttatgactatatttagtaccactgaactgtacacttaaaatgtttaacagagtacattttatgttttatgtatcttaccacaataaaaaaataaaataccttaggaacattttcctgaaagagtccacataaaattcattttaatgcatgtgtttatgcatagctttctatttttctcttttctctttacattccaaattagaatataatgctaatcaagtatagtggctgcgtttctttcttcctctagtctgcaggtagcatacaaatgtaataaagtactcattcatgtcacatctatttattttctgccttataccaagcttgtgggattctcttaaatacaacatttttatacttacacctatgcaatacccattagcatcgccttcctaaatcaggggaaattgagtctctgtaaggtgcagtaacttactaagatacaaaactcagcattaaagtctgtatacttcaatatcctgccctcttctcatttgtctttactgccttttatgtatgtgttagatgttcaataaattctcttttttaaactgaaaaaaaaaaaaaaaaaaaaaaaaaaaaaaaaaaaaaaaa

SEQ ID NO: 5 - DNA, 2287 bp, Homo sapiens
ggacagcacagctgacagccgtgctcagaaagtttctggatcccaggctcatctccacagaggagaacacgcaggcagcagagaccatggggcccatctcagccccttcctgcagatggcgcatcccctggcaggggctcctgctcacagcctcacttttcaccttctggaacccgcccaccactgctcagctcactattgaagctgtgccatccaatgctgcagaggggaaggaggttcttctacttgtccacaatctgccccaggaccctcgtggctacaactggtacaaaggggaaacagtggatgccaaccgtcgaattataggatatgtaatatcaaatcaacagattaccccagggcctgcatacagcaatcgagagacaatataccccaatgcatccctgctgatgcggaacgtcaccagaaatgacacaggatcctacaccctacaagtcataaagctaaatcttatgagtgaagaagtaactggccagttcagcgtacatccggagactcccaagccctccatctccagcaacaactccaaccccgtggaggacaaggatgctgtggccttcacctgtgaacctgagactcagaacacaacctacctgtggtgggtaaatggtcagagtctcccggtcagtcccaggctgcagctgtccaatggcaacaggaccctcactctactcagtgtcacaaggaatgacgtaggaccctatgaatgtgaaatacagaacccagcgagtgcaaacttcagtgacccagtcaccctgaatgtcctctatggcccagatgcccccaccatttccccttcagacacctattaccatgcaggggtaaatctcaacctctcctgccatgcggcctctaatccaccctcacagtattcttggtctgtcaatggcacattccagcaatacacacaaaagctctttatccccaacatcactacaaagaacagcggatcctatgcctgccacaccactaactcagccactggccgcaacaggaccacagtcaggatgatcacagtctctgatgctgtagtacaaggaagttctcctggcctctcagctagagccactgtcagcatcatgattggagtactggccagggtggctctgatatagtagctctggtgtagtttctgcatttcaagaagactggcagacagttgtttttattcttcctcaaagcatttgcaatcagctaccattcaaaattgcttcttcttcaagatttatggaaaatactctgacgagtactcttgaacacaagttcctgataactttaagatcacgccactggactgtctatgaacttgcaaacaggctgatacctttgtgaagttgcccaccaaaacacagaaggaaaaaaacatgaatttcattgaactaaataataatgaggataatgtttttaagattttttttttttttttttttgagatggaatctcgctctgtcgcccaggctggagtgcagtggcacgatctcaactcactgcaacgtccgcctcctgggttcacaccattctcctgcctcagcctcctgagtagctgggactacaggcgcctgccacaacgcccggctaattttttgtatttttagtagagacggggtttcactgtggtctcaatctcctgacttcatggtccgcctgcctcagcctcccaaagttctgggattacaggtgtgagccaccgcgcccagcccgtttttaagattttttatttgaaaaattgccaattctttaagtgttttctttttcagatttatgaatttctttatcttttaagctatctataccttactgcaatttggtaaagcagacttttgtgaacaaaaattataacatttacttttgctccctacctgactgccacagaactgggcaactattcatgagtattcatatgtttatggtaattcagttatttgcacaagttcagtgagaatctgctgtctttataatgggatatagtttaaaacattggttatattaccaaggctttgattgggatgttatatttgagaaaatacagagaatgatagattaacggagtgtctaatctatcgtgtcaaccccaaatttttacgtatgagatcctttagtccacccaatggctgacagtaacagcatctttaacacaactctttgttcaaatgtactatggtctcttttagagtcagactcctagactcacttgttctcactgtctgttttaatttaacccaggcatgcaatgctagataataaaattgctccctattggctgatc

SEQ ID NO: 6 - DNA, 2593 bp, Homo sapiens
acagtgaccgccaccgttgcccggggatgggtccctgagacttggcgaagtaggagccctttgtgattgtgcgtcagagtcggggctgagaccagtcctggccagggcagttaccaggacggtctccggaggccgggattcgcggagggtccgccaggaggaagaaacctcagacagatcgccggggacgcagcacgggatcccagcctcaggcgtgcacggacggtgtgcggaatcctccatctcaggaaggccccagtattgccaggctgggagaaacccaagccatgaggatagaccatgaataggagttctggttgacaatcccagttcactcccagcttcaaatacacaaggccaagtgccagatacgggcataaaaagcaattttggaagtggacactccagtcccagctgtcctagccctcagctatttgagttgcatccagtcatgtaaatcttcccagctgaggcctcatcattgaggtgcagaaaaggggcatgcccattgtgcctgtttctaaattcctgacccacagaacctctaatcagcaatacaactgggatttgatatcagattcatgcgattccgaagaccctgaccaccaaatgcaaaagtgaaatggtctcatcctacctgcaactgaggctgaaaaatacagcatttccgtatggtccaaaggaggaaataatgttatcatctcaccagtctctgtcaagacaagttctgaagtcttcagccaatcatgagtatctgtccctagcttactcatcttaagaaggagccaaaaacatcagacaggtgtgcttactgctcatttattaaagatttaagtttcctttacttggagttcttcatctgtgacacaaactgtgtgtagcatcaacttgaactccttgtcatcatctccagtaaacttgagggaaaacaggaaccagagcaaacatgaagctcgtgcaactctgtgctgtgctgcaaataatggaagtctttctgtctctgaccaaacagttatgtgtcttctgccagcatccataaaactgaagcaggctaacttgtaagctcagatccttcacagttcttgacaatagtcccttaaaactgtctgcagatgcacatctcttggaaaaattttgcatccttttaagaataattaatatcctagttaatgacaactatataacaggatagatcactttaagcttctacccagattagtataacttttaggtaaataatgtatatgccattttttctaaggttatctagccatttttgatgtggaaaacaatattgtgtaagataagctgagcatatttttcacttcgtttttgttttgcaaaacaattttattgcttttgttaatgccccaatcatcaatactgcttggcataagagccaagtttgaagaattatcatgttgcaaatgtattttgctcatttatttcaaattagtagacagaattgagaggtggaaaattttagaaaaatcttggttaatcatacttggaatttggttatgtaatatctctttcccttttggggaatacaaaggttcataaactagtgataatcttcactcttggaacctttagatgctatcatgatgttagcacagaagataaatttataatgttgcagtgaggtaaggaaataaaacatgtaagtctttgaaaacaacactatgagagcccttgcaggctaaaaattagacaccaaagcattttttgttacttttggagaataaaagaagtgtgcccctcaatgtcttgtgggagaaaatatatagtttgatacacttgtcaatatctctaactcaaaaatgatgagtataaggagtgacccttattgtagtcttatatttctctccaacaattaagagagttggtggggatgttttttccatgagttcatcccgggccccaggagattttcatcttgtattctctttataatcctatcttagtgtttcatcctcatggctaagttcagcaatacttctgcattccagccagtaaaatgtgtaagaagagatgtaaaaacaaccggctttgtctttatggagattaacttaaatttatgctcataatataattcatgttatacttgatcaaaattaatcactgataagagactatagaaggtaatctcctgcttgtggtcatatgtacttaggattctattactaaacaaaatgaaagaatgcattatatggacaattagtggtctctagtacaaagaaattaccaggtcatttccaattctcaatattaaaaaaaaagtttatttcttactgcactatcaaattggaaaggtcagtacaggggcattcaataatagtaaaaatatgaatagcaatcatatgtatcacaacagaatcatttaaaaagatgtatatttaaaatatgattattgcattttatttcctgtggcagaaaattataaattggagaaaaatcacatttgattgatgtctgttacatggatttttaaaatttgtatcaatgaatgctttttaaaggtaaactaattgaggcctaccaaagataaataaatctttagttattttggtaaataaaaaaaaaaaaaaaa

SEQ ID NO: 7 - DNA, 1093 bp, Homo sapiens
ctgttgaaggaagaagaaagcaacaaaagactggaagctgaaattgaatcatcagtctagactgactgctgctataagtaaacacagtgaaagtgtgaaaacagaaagaaacctaaaacttgcattagagtgaacacaagacgtttccgtacaagtaaaaatgagttctgatatttccgaagtagaagataagaatgagtttcttactgaacaactttctaaaacgcaaattaaactcaataccttaaaagataagttccgtaagaaaagagatactctcagaaaaaagtcatcggctttagaaactctccaaacgacctaagccaaacacagcagcaaataaaggaaatgaaagagatgtatgaaaatgcagaagataaagtgaataattccactggaaagtggagctgtgtagaagagaggatatgtcatctccaacatgaaaatccgtgccttgaacagcaactagatgatgttcatcagaaagaggatcataaagagatagtaactaatatccaaagaggctttattgagagtggaaagaaagacctcatgctagaagagaaaaataagaagctaatgaatgaatgtgatcatttaaaagaaagtctctttcaatatgagagagagaaagcagaaagagtagtaagtatcaaggaagataaatattttcaaacttttagaaagaaaatttaaacatttggttctggatacatgttgaacttagttgaatataaaaatcaatggataaaaagtgtgtttaccatactgtataattccatttacatgaagcatccagaaaagataaacgtatagggacaaaaagtagactaatgtttgcaaagggctggggctgaaagctggtagtgactgctaatgggagtgagggatcttgcagtgatggaaatgctgtaaagttggattgtagagatggctgcacaactcagtaaatggactaaaaaatcttttaactttaagttaaaacagatacattctatagtatgtaaattatatttgaacaaagctgttttaataaaaaaaaggaaaaccgtgtttactatagaaaaaaaaaaaaaaaaaaaaaaaaaaaaaa

SEQ ID NO: 8 - DNA, 912 bp, Homo sapiens
acagacctgccccgccatgacccggctgacagtcctggccctgctggctggtctgctggcgtcctcgagggccggctccagcccccttttggacatcgttggcggccggaaggcgaggccccgccagttcccgttcctggcctccattcagaatcaaggcaggcacttctgcgggggtgccctgatccatgcccgcttcgtgatgaccgcggccagctgcttccaaagccagaaccccggggttagcaccgtggtgctgggtgcctatgacctgaggcggcgggagaggcagtcccgccagacgttttccatcagcagcatgagcgagaatggctacgacccccagcagaacctgaacgacctgatgctgcttcagctggaccgtgaggccaacctcaccagcagcgtgacgatactgccactgcctctgcagaacgccacggtggaagccggcaccagatgccaggtggccggctgggggagccagcgcagtggggggcgtctctcccgttttcccaggtttgtcaacgtgactgtgacccccgaggaccagtgtcgccccaacaacgtgtgcaccggtgtgctcacccgccgcggtggcatctgcaatggggacgggggcacccccctcgtctgcgagggcctggcccacggcgtggcctccttttccctggggccctgtggccgaggccctgacttcttcacccgagtggcgctcttccgagactggatcgatggtgttctcaacaacccgggaccggggccagcctaggggggcctgtgacctcccatggagcccagccccgccctccacacctccggcgctccgcacccacctcccacggccccgcccctgcccccgctccggccagaggggccctggctgtaataaagaagccgatctctcctctg

SEQ ID NO: 9 - DNA, 938 bp, Homo sapiens
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgtagaaaaaaaaaaaaaaaa

SEQ ID NO: 10 - DNA, 255 bp, Homo sapiens
tatacggctgcgagaagacgacagaagggaataaggctctgtgcatacgaatggggctttgaaaggcgttgccgcggcttccaaagcgatgcgctatgcctcgcctcgcctcgccccgcccagagcgagactccgtctgaaaataagtacacaaataaatcataagataattcagcaagaaagaaagaaagaaagaaagaaagaaagaaagaaagaaaaaaaaaaaaaaaaaaaaaaaaaaaaaagtcgtatcga

SEQ ID NO: 11 - DNA, 547 bp, Homo sapiens
ggaggagaagaaatagaagaaagttctgcaacaaccatgtctgagaacttccagtattaatgtcagacaccaaaccaaagatccaggaagctccgagaacaccaggcagaataaatgccaacaacctacacttggacatataattttcaaactatatgaaataaaagataaaggaaaactctgaaagaaaccagaggtggggcagaaaacaccttacctacagagacacaaagataagaactgcattcaacattgcagaaactgtgaaagcaagaagacagtgaaatgaaaaattcaaaatgttgacagaaaaaaacccaccaacctaagtttctgtacccactgaaaacacccttcaaaagtgaaggagaattaaggccttcctcagaaaaataaaaattcaagaaacttgttgccaggagacctgtcttgcaagaaattttaaatgaaattctttagggaaacaaaagatatgtaactgaaacctggatcaacatttttttaaaaagagcattaaagaaagaattgtggtacaataaaaaaaaaa

SEQ ID NO: 12 - DNA, 2770 bp, Homo sapiens
ctccttggcggtgcctggagcccgggcgcaccccaccgctcccgggacctgttgggggctggcccgaaccgtcgtcgaagggagccgctcggccacccccgacgttcctcgccccgcccgacgttccctcaagtggccgaaccagccggacgagccaaactcgccgggcctcccggcggcagcaggtggccccgtccttccagggagggccctgcgccccgcggcgctccggagccctctcggccgcccccgccaggcgggatggaggcggatggggacggagaggagctggcccggctgcgctcagtcttcgccgcctgcgacgcgaaccgctcggggcgcctggagcgcgaggagttccgggcactgtgcacggagctgcgggtgcggccggccgacgccgaggcagtattccagcggctggacgccgaccgtgacggcgccatcaccttccaggagttcgcgcgtggcttcctcgggtccctccgcggggggcggcgccgggactggggtcctctggatcccgcgcccgccgtgtctgaggcggggccggagacacacgacagcgaggaggacgaaggcgacgaggacgcggcggcggcgctggccacctcgtgcggcccggcgagtcccggccgggcttggcaggatttccaggcgcgacttggggacgaagccaagttcattcccagagaagagcaagttagtaccttgtaccaaaacatcaaccttgtggagccaagattaattcagccatatgaacatgttataaagaacttcatccgtgagatcagacttcaaagcacagaaatggaaaatttggccattgcggtgaagagagcccaggacaaggcagctatgcagttgagtgagttggaagaggaaatggatcagaggattcaggctgcagaacataagacacggaaagacgaaaaacgcaaagctgaggaagccctcagtgacctcagacgtcagtatgaaactgaagtaggagatctgcaggtgaccattaaaaagctaagaaagctcgaagaacaatcaaaacgcgtaagtcaaaaggaagatgtggctgcattgaaaaaacaaatttatgatttatcaatggaaaaccagaaagttaagaaagaccttttagaagcacagacaaacatagcctttcttcagagtgagttagatgctttgaaaagtgattatgctgatcagagtctgaatactgaaagggatctggaaataatccgagcatacacagaagatcgaaatagtcttgagaggcaaattgaaatactccaaacagctaaccggaagctacatgacagtaatgatggccttagaagtgcccttgaaaacagttatagcaagttcaacagatctttgcatataaataatatctcaccagggaatacaatttctagaagcagtcccaaattcattggtcattcccctcaacctctaggctatgacaggtcatcccgctcttcctatgtggatgaggactgtgactccctggccctctgtgatcctctgcagaggacaaattgtgaagttgacagcctgcctgaaagctgcttcgacagcggcttgtctaccttgagagatcccaatgagtatgactcagaagtggaatacaagcaccagaggggatttcagaggtcacacggggtgcaggagagctttggaggtgatgcttcagacacagatgttcctgacataagggatgaagagacatttggtttagaagatgtggcttccgtcttagactggaagccccaagggtctgttagtgaaggcagcattgttagttcatcaagaaagcccatctcagcactctcgccccagacagacctggtagatgacaacgctaaatcttttagctcacagaaggcttacaagattgtacttgctggggacgctgcagtggggaagtctagtttcctcatgagactttgcaagaatgaatttcgagaaaatataagcgccaccctgggagttgatttccaaatgaaaaccctcattgtggatggagaacgaacagttctgcagctctgggatacagctggtcaggagagattcagaagtattgccaagtcttacttcagaaaggcagatggtgttttgctgctgtatgatgttacatgtgagaaaagctttcttaacatacgagaatgggtagatatgattgaggatgcagcccatgagactgttcccattatgctggtaggaaacaaggctgacattcgtgacactgctgctacagagggacaaaaatgtgtcccagggcactttggagagaaactggccatgacgtatggggcattattctgtgaaacaagtgccaaagatggttctaacatagtggaggctgttctgcaccttgctcgagaagtgaaaaagagaactgacaaggatgacagcagatccattaccaatctaaccgggaccaattccaaaaagtcaccacagatgaagaattgttgcaatggctaaatcccaaacatccttggcctgtgaagtcttcatttccagaatactgaatttgtgtgacttatttggctcttaacagagtggcacatcctactgacactgtcctatggagagttacagtgcaggaaacctgaacccagctctcaggtccctctggaactttggctcttctttgttttgtctcagtgagtgatttgggccctctggctaaatagactagtcatgtccttacaggtcttaaaagataacatgtaaatgtttttaaaatggtaaaaaaaaaaaaaaaaaa

SEQ ID NO: 13 - DNA, 1674 bp, Homo sapiens
ggggcggggcgtgccggggcggggcgtagtacggactgggcctggcctggggcgtccccgcgaagcctgggcctgtcaggcggttccgtccgggtctcggccaccgtcgagttccgtcgagttccgtcccggccctgctcacagcagcgccctcggagcgcccagcacctgcggccggccaggcagcgcgatcctgcggcgtctggccatcccgaatgctatggccgccgtcgccgtcttgcgggccttcggggcaagtgggcccatgtgtctccggcgcggcccctgggcccagctccccgcccgcttctgcagccgggacccggccggggcggggcggcgggagtcggagccgcggcccaccagcgcgcggcagctggacggcataaggaacatcgtcttgagcaatcccaagaagaggaacacgttgtcacttgcaatgctgaaatctctccaaagtgacattcttcatgacgctgacagcaacgatctgaaagtcattatcatctcggctgaggggcctgtgttttcttctgggcatgacttaaaggagctgacagaggagcaaggccgtgattaccatgccgaagtatttcagacctgttccaaggtcatgatgcacatccggaaccaccccgtccccgtcattgccatggtcaatggcctggccacggctgccggctgtcaactggttgccagctgcgacattgccgtggcgagcgacaagtcctcttttgccactcctggggtgaacgtcgggctcttctgttctacccctggggttgccttggcaagagcagtgcctagaaaggtggccttggagatgctctttactggtgagcccatttctgcccaggaggccctgctccacgggctgcttagcaaggtggtgccagaggcggagctgcaggaggagaccatgcggatcgctaggaagatcgcatcgctgagccgtccggtggtgtccctgggcaaagccaccttctacaagcagctgccccaggacctggggacggcttactacctcacctcccaggccatggtggacaacctggccctgcgggacgggcaggagggcatcacggccttcctccagaagagaaaacctgtctggtcacacgagccagtgtgagtggaggcagaggagtgaggcccacgggcagcgcccaggagcccaccttcccctctggcccagccaccactgcctctcagcttcaacaggtgacaggctgctttcgtgacttgatattggtgtcatagcatttggcctacattaaaagccacaatttcatggggaaaggacaaaatggagagtgactgaggtgctgacctcagtgcaaggctggtgaaccctgcagcgggccagctatggtgggaagcctggcatttggggtgctccttgcaacgtcttaagcaagcgacccccctgacatagcaaaaggtggcaacccatggaggcagaaagaaggacgccagcctgacccttatctgaaacgtcctaagcagagttaatcctggctgctcaggagaggcgacacatttcaaatctccacgagatattctccacacagaaaatcttcttgattctatagagacttaatcatgcctatggctttgaataatcttatgtgatttaaataaattaaatctttataaaaaaaaaaaaaaaaaaaa

SEQ ID NO: 14 - DNA, 513 bp, Homo sapiens
tttttttttttataaaagtactctgatttttattgttatacaacatatatatataattgtttccccaaaatatgcacaattacatgtgtcaattttaaaaaatgaatgaagactataatgtaaaacctatagctgtaaaattcctagcacaatacagaagggtgaagcttcatgacaactggtcgtggcaataatttgggggacgtaacatcaacggatgagacaacaaaagcaagggaatacacatggtactgaatcagtgtatgaaaaatatcccaaacagacaaagcagaacatggaatagatatatgcacattgtagtattactcacaaacatgttacctggaagcaaatgtacccttaaggatgagtagattcagcaaacagggcacgtacaatcactgggatagcattcagccttaaaaataaggaaatcttgaaaagtactacaataaggacaaatattcaaaacattctgttaagtaaaataagacagtcaaaaaggaaagctgt

SEQ ID NO: 15 - DNA, 1037 bp, Homo sapiens
gccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccagtaagtgggccagggtcttctgttgagaactctgtgggtttcgctcttccttttggagacagttatcactatgacccacataccacatg

SEQ ID NO: 16 - DNA, 3407 bp, Homo sapiens
tgcaggtgcgttgtacccttttacgtgaggcggtgacggcggttcggaagtcgtctggcctccccgcggccgctcgcagcttgctggcctctcccgcgcctcacgtcggactccgtctccgcggcagggaagcagcatggaagctgaggacatccaggaggagttgacctgccccatctgcctggactatttccaggacccggtgtccatcgagtgcggccacaacttctgccgcggctgcctgcaccgcaactgggcgccgggcggcggcccgttcccctgccccgaatgtcggcacccatcggcgcccgccgcgctgcgacccaactgggccctggccaggctgactgagaagacgcagcgccggcgcctgggccccgtgcccccgggcctgtgcggccgccactgggagccgctgcggctcttctgcgaggacgaccagcggccagtgtgcctggtgtgcagggagtcccaggagcaccagactcacgccatggcacccatcgacgaggccttcgagagctaccggacaggtaactttgacatccacgtggatgaatggaagagaagactaattaggctgctcttgtaccattctaagcaggaggagaaacttcttaagtctcagcgtaatctcgtggccaagatgaagaaagtcatgcatttacaggatgtagaagtgaagaacgccacacagtggaaggataagataaagagtcagcgaatgagaatcagcacggagttttcaaagctgcacaacttcctggttgaagaagaggacctgtttcttcagagattgaacaaagaagaagaagagacgaagaagaagctgaatgagaacacgttaaaactcaatcaaactatcgcttcattgaagaagctcatcttagaggtgggggagaagagccaggctcccaccctggagctgcttcagaatccaaaagaagtgttgaccaggagtgagatccaggatgtgaactattcccttgaagctgtgaaggtgaagacagtgtgccagataccattgatgaaggaaatgctaaagcgattccaagtggctgtaaacctagctgaagacacagctcatcccaaactcgtcttctcccaggaagggagatacgtgaaaaatacagcatcagccagttcttggccagtgttttcttcagcatggaactactttgctggatggaggaatcctcagaagactgcttttgtagagagatttcagcacttaccctgtgttctgggaaaaaacgttttcacctcagggaaacattactgggaagttgagagtagagatagtctggaggttgctgttggggtgtgtcgggaggacgtcatgggaattactgatcgttcaaaaatgtccccagatgtgggcatctgggcgatttattggagtgctgctggctattggcccttgataggcttccctggaactcccacccagcaagagccagctctccaccgagtgggggtttacctggatcgtgggactgggaatgtctccttctacagcgctgtggacggagtgcacctgcacaccttttcttgttcttctgtctcacgcctccggccatttttttggttgagtccattagcatctttagtcattccaccagtgactgataggaaatgaggcttttcttcccctgaccaaaactccttccctgtagtccagctgagggacacacatccctgggccctcttctgcccttcatgtctctatcctggatggtccatcttctgggtctccctaacggtaccgtttggtatctgccctttgtgtgcttcacaagaggcagtcccatgggaggtgggtctggccaatggagatgggacaggaaattttccaagacgcttttgggaaatctttttgtagcttttaaagagatgtgcggggaagacatatggatgttagcagccatattggaactgagaacacaggaatggtggaaaggtagaagaaacagagtttttgttgccgttgtgaaattgttgaaatttccttcatgccaagcttcttgttatatgagataattacgcccttattgtataagacaattttagttgtatttggttacttgcagcctgaagtaccgtaactgcactaaagggacgtagtgtgaacatcccgcagtataggcttaagtcacttttgtgaaatttgacaaaggcatagaatctttttctatccagtcaggcattgcctattctttccagtaactactgattcccccacttttctgtcttagaaaattgtgggaatcccccctcactctgcctatgttgcactctctctcttcccaaccataactctgccctcagctattaactgtgctgtgtatttatcaagttggtatgttgtatgtacagtgttattcatgtcatcatgagaatgttgaacgcttgttaaataccttttgctagcctcttcatatgctgttgcatatgactctcatcacaactcagtgagatggaaagacaaatcctatttgtacaaatgagaaaactgaactctttagagtaactagctcagtattggccagctggtaaatggcagtgttgggattaaaatccagttcttatctactctccctttattcagaagcatttattggatgttgatctttgtttcaggttttgattttgttacttttttatactgtgtatattttcctcagtctacccttctgctctagattgtctggactcaggagattgtggcagttactggatagttatttttaagataatgattgcttttctctgtttatataagtcatgtgtacttattgtagaaagtttgtaagatgcaaaaagtataaaaattaaagttatgcactactaacatttcaatatattttctcccagattttcaataaagactttcaggcagtgatttaacgaggattttattttaacttatattttagtttaaggggaacacgtgcaggtttgttctatagatcaattacatgtcacaggggtttggtgtacatattatttcattacccaggtaataagcatagtacccaatagatacttttttgataccccaccctcctccaccctctaccctcaagtagtccctggtgtctgttgttcccttttttgtgtccatgggtactcaatgtttagctctcccttatacgtgagaacatgtgatgtttggttttctgttcatgtgttggtttgcttagggtaatggcctccagctccatccatgttgctgcaaagacatgatctcattctttttttatggctgcatagtattccatggtgtatatgtactatgttttctttatccagtctactgttgatgggcatttaggttgattccatgtctttgctattgtgaatagtgctgcaatgaatatatacatgcaaaaaaaaaaaaaaaaaaa

SEQ ID NO: 17 - DNA, 778 bp, Homo sapiens
tggctataaacacatctgctgcacagctgacatttatcttctacaataagcaatgacaattttattttttaataatcagtatggactaatcctgatgattttttttttaacattttcaaatagggctgcatatggcttaaaattaatatatacatgtgtacctatataatattcttatttattaatggacttcctacatagctcatatgacgttagatttaaatgaaattccagaagggttttctataggtaagtcatacattggatttccatattacctatgattatgaagtatttatttctgtttttaagacttcagagcaatttgctggtcattgttttctgtgtttttatttggaaatgttcttgaggcatgtcctattacatttttaaggtatgttaataaaataatatttttaagaaaaaaaaaaaaaaaaaaaaaaaaaaaaaatttttggggggctggcgccggaaaggtttttaaacccatggtggggggggggcaccccagaagtgtaatagaaccagcgggccacacgggtcgtccaaagaaaatccggggtcacacagcaaggtgtggtggtctcccaccatataaaaaacggccggcggcaccggactgtgtttggaaccatcccaagagggtgtcagggacaaggggtctcacacagcggtccaggagtcagataataccccggcggggaaaaacacgaacgagagacgacaaactcacgacgggcagagacgcagcacagcagagcagaaacagaagacacaa

SEQ ID NO: 18 - DNA, 482 bp, Homo sapiens
tttttgaagtaaaaatctttaatttgaacttatttaatagattattttaatttttgaacaagcataaaggtgccaatttaattatctgtgggatttaaaaacttcaacagagaaagaaaataggaggtactagtgagaaatatattttacagtctacatatttattgtataataagctatattatattctaataacagcaaatataacattataatgattttgatatctggactcgctagacttggtcccataaatcctatagttcttaacaaaaaaacccatccgttagcaaacttacacatcaaagctcagtttaatgcaacttaatgagacattgaattttatgcagctaacccaagttatctatagtgtgtgccctcctgagtaccccaggaaatagaagtaaagaactgacgaacatcagatccaactggcccatttaggtttggactctaggagagagcaaggaggaggcaggaga

SEQ ID NO: 19 - DNA, 924 bp, Homo sapiens
gcacagcagcaactgactgggcagcctttcaggaaagatgcagccactcctgcttctgctggcctttctcctacccactggggctgaggcaggggagatcatcggaggccgggagagcaggccccactcccgcccctacatggcgtatcttcagatccagagtccagcaggtcagagcagatgtggagggttcctggtgcgagaagactttgtgctgacagcagctcattgctggggaagcaatataaatgtcaccctgggcgcccacaatatccagagacgggaaaacacccagcaacacatcactgcgcgcagagccatccgccaccctcaatataatcagcggaccatccagaatgacatcatgttattgcagctgagcagaagagtcagacggaatcgaaacgtgaacccagtggctctgcctagagcccaggagggactgagacccgggacgctgtgcactgtggccggctggggcagggtcagcatgaggaggggaacagatacactccgagaggtgcagctgagagtgcagagggataggcagtgcctccgcatcttcggttcctacgacccccgaaggcagatttgtgtgggggaccggcgggaacggaaggctgccttcaagggggattccggaggccccctgctgtgtaacaatgtggcccacggcatcgtctcctatggaaagtcgtcaggggttcctccagaagtcttcaccagggtctcaagtttcctgccctggataaggacaacaatgagaagcttcaaactgctggatcagatggagacccccctgtgactgactcttcttctcggggacacaggccagctccacagtgttgccagagccttaataaacgtccacagagtataaataaccaattcctcatttgttcattaaacgtcattcagtactta

SEQ ID NO: 20 - DNA, 2532 bp, Homo sapiens
ccacgcgtccgctttctaatgagtagaatgtagtgaaagggataccatgtggcttccctatctcagactgttttcccttggaacccagccccaattgtgagagcctgaaagtgcctgtgtcagtgttcatgctgcctgtcccaaccaagtttacagccgatggccagcatcaaccatcaaacaagtgggtgaccaaagcttcagaggattccttttccccaactgatcagctcttcctagggaagctgaagggagcagagacaagctgttctggccaagtttttcccaaaccacaggttcatgaacaaaataaatgtttttctttcaagccacaagactctgggtaattgttagaaaaataacttttaaaaagagacaacaggaaacataacttatgcagcagaaaagtctcctttaaagtaggatctaataaatgttgagatttatttattgatgtcaaacattattgagaagcagcagataaccaggagagagacataagctgctgaggaggaattttcctaaaactccttcaattatgaactctgataacaaggcaaggatgtctccttacaatttcccctcaagttaggaaataagactgggaagcaagaagatgtatgatttgaaaaacaactagaaatacttggtgacaaaggcaaaatcagacatttacctgatttcaattaactaaaattctaaaagaagaagctttgagtgtttattaatcaacctagtattcaattttcattttcttttctaaatgagcaaataaggagattatggaatgatttttagtcttcacagaagtaaaataagcatagtgtgttttgagtgttaagacatcaaatgcaatttctcctttaccttactccaagcttgtttgtatggagaagttaagaccatcccatctctatgttatgccacaatgcttctctatagcacacaacttggctccgaaattttgaaagtcaaaatactaacctactatgtgtctctgataaattgcctgaacattacctgattttgaagtgctgcactcctaagactttttcttggaatgagttaaacgttttattccaagaatcctctactgagctagaaagcagagctgtgcatctctgtttcagtaaaaggaggtcaatacagggaactgtggtctctgagaatgcaagatctgcactaagaaaaggatgagctacagtactacccaagagaaccagctaccaggaggaaagagggtctgtaaactgcaagatgatgacttcacttgatttccactgaggaaagctggtggttcagacttaaacttctccttcctcgatggtaaacatctacggaaggttctatgaattataatgagttagtaaaacataatgcactaaatattagactatgtcagcagatcctgcgaccaaaacttactgaaaatataactatagcgggaggcaatggaaaagagactaaaggcttgaatggagaaaaaaagaaattaaatgtgtcttgtaagcctggcgtctgatcatgtcttagacgaagtaaggtataagctggccagagactcctttgtgacacaaaaggtgaagttacagatattccactaaatttaatttttattatgacataagacaactggtaatatgcaacatgattgaaaaaaacttctcattcaattcgattgggctttgacataagaatagacataaagaagctaagaattgacaatctaaaaataagcttgcacttttacagtcaattgattttatacaaacttaacgaaagaacacaatgggaaaagaatagtcttttcaacaaatggtgctgggacaactgggtatccataagaaaaaataaataaagttcgaccaaatatcttatttaaaaattaactcaaaataaaatagttaactgtaaaagctaaaactataaaaccctcagaagaaaacactggcataaatctttgtgacagcatttgccagtgttttcttagctatgactccaaaggaaaaatggattcaatggacttcaaaattgaaaactgctgtgcctcagaagacagtatgaagaagtgaaaaggtaagacgccaagtagaagaaagtatttgaaaagcgtgtatatgataagggacttacatatataggaaatataaataactcttgcaattaataaataacaagataagccaattttaaaaaatggtcaaagattttgaatagatatatctgcaaagcagatataaagatggataagcacattaatagatgcttaatgtaattagtcattaggaaaatgtaaatcaaaaccacatgtggtatcacttcacaccacaggataaaatctttgttcaagaaaaaagagggttagaaaaaaaatgtaaagaaattaaaacccttatctaatgctgctgggaatgtaaagtgatgcagccactttggaaaacaaactggcagctccttaaagggttaagcataaagttaccatatgacacagaaattccagtcataagtatatactctaaaaaaaaaaaaaaa

SEQ ID NO: 21 - DNA, 352 bp, Homo sapiens
  modified_base (346)..(346): a, c, t, g, unknown or other
ttttttttttgctaacaataccatttttactcaatctcataggctttggctaggatgacttgtatactgcagttcacttgtagataccaaattttaataaatttattcttctttgcatctaataaacacagagggaagagttcttactgcattaattacctaccaatacgtataatgaatgttaattctaataagatcccaggcatgctcccaaaggaatgctttgtaacaaagcgtcagtcttatgctttaaaaaaccaaaccaaaccaaaacaacaacaacaacaacaacaacaacaacaacaacaaacaggatctaaagcatacacacaagtgtgcacaattnttttat

SEQ ID NO: 22 - DNA, 4220 bp, Homo sapiens
aggagaagtctgagcattctttttttcttttttattactataggatggaggatgaagctgtcctggacagaggggcttccttcctcaagcatgtgtgtgatgaagaagaagtagaaggccaccataccatttacatcggagtccatgtgccgaagagttacaggagaaggagacgtcacaagagaaagacagggcacaaagaaaagaaggaaaaggagagaatctctgagaactactctgacaaatcagatattgaaaatgctgatgaatccagcagcagcatcctaaaacctctcatctctcctgctgcagaacgcatccgattcatcttgggagaggaggatgacagcccagctccccctcagctcttcacggaactggatgagctgctggccgtggatgggcaggagatggagtggaaggaaacagccaggtggatcaagtttgaagaaaaagtggaacagggtggggaaagatggagcaagccccatgtggccacattgtcccttcatagtttatttgagctgaggacatgtatggagaaaggatccatcatgcttgatcgggaggcttcttctctcccacagttggtggagatgattgttgaccatcagattgagacaggcctattgaaacctgaacttaaggataaggtgacctatactttgctccggaagcaccggcatcaaaccaagaaatccaaccttcggtccctggctgacattgggaagacagtctccagtgcaagtaggatgtttaccaaccctgataatggtagcccagccatgacccataggaatctgacttccttcagtctgaatgacatttctgataaaccggagaaggaccagctgaagaataagttcatgaaaaaattgccacgtgatgcagaagcttccaacgtgcttgttggggaggttgactttttggatactcctttcattgcctttgttaggctacagcaggctgtcatgctgggtgccctgactgaagttcctgtgcccacaaggttcttgttcattctcttaggtcctaaggggaaagccaagtcctaccacgagattggcagagccattgccaccctgatgtctgatgaggtgttccatgacattgcttataaagcaaaagacaggcacgacctgattgctggtattgatgagttcctagatgaagtcatcgtccttccacctggggaatgggatccagcaattaggatagagcctcctaagagtcttccatcctctgacaaaagaaagaatatgtactcaggtggagagaatgttcagatgaatggggatacgccccatgatggaggtcacggaggaggaggacatggggattgtgaagaattgcagcgaactggacggttctgtggtggactaattaaagacataaagaggaaagcgccattttttgccagtgatttttatgatgctttaaatattcaagctctttcggcaattctcttcatttatctggcaactgtaactaatgctatcacttttggaggactgcttggggatgccactgacaacatgcagggcgtgttggagagtttcctgggcactgctgtctctggagccatcttttgcctttttgctggtcaaccactcactattctgagcagcaccggacctgtcctagtttttgagaggcttctatttaatttcagcaaggacaataattttgactatttggaatttcgcctttggattggcctgtggtccgccttcctatgtctcattttggtagccactgatgccagcttcttggttcaatacttcacacgtttcacggaggagggcttttcctctctgattagcttcatctttatctatgatgctttcaagaagatgatcaagcttgcagattactaccccatcaactccaacttcaaagtgggctacaacactctcttttcctgtacctgtgtgccacctgacccagctaatatctcaatatctaatgacaccacactggccccagagtatttgccaactatgtcttctactgacatgtaccataatactacctttgactgggcatttttgtcgaagaaggagtgttcaaaatacggaggaaacctcgtcgggaacaactgtaattttgttcctgatatcacactcatgtcttttatcctcttcttgggaacctacacctcttccatggctctgaaaaaattcaaaactagtccttattttccaaccacagcaagaaaactgatcagtgattttgccattatcttgtccattctcatcttttgtgtaatagatgccctagtaggcgtggacaccccaaaactaattgtgccaagtgagttcaagccaacaagtccaaaccgaggttggttcgttccaccgtttggagaaaacccctggtgggtgtgccttgctgctgctatcccggctttgttggtcactatactgattttcatggaccaacaaattacagctgtgattgtaaacaggaaagaacataaactcaagaaaggagcagggtatcacttggatctcttttgggtggccatcctcatggttatatgctccctcatggctcttccgtggtatgtagctgctacggtcatctccattgctcacatcgacagtttgaagatggagacagagacttctgcacctggagaacaaccaaagtttctaggagtgagggaacaaagagtcactggaacccttgtgtttattctgactggtctgtcagtctttatggctcccatcttgaagtttatacccatgcctgtactctatggtgtgttcctgtatatgggagtagcatcccttaatggtgtgcagttcatggatcgtctgaagctgcttctgatgcctctgaagcatcagcctgacttcatctacctgcgtcatgttcctctgcgcagagtccacctgttcactttcctgcaggtgttgtgtctggccctgctttggatcctcaagtcaacggtggctgctatcatttttccagtaatgatcttggcacttgtagctgtcagaaaaggcatggactacctcttctcccagcatgacctcagcttcctggatgatgtcattccagaaaaggacaagaaaaagaaggaggatgagaagaaaaagaaaaagaagaagggaagtctggacagtgacaatgatgattctgactgcccatactcagaaaaagttccaagtattaaaattccaatggacatcatggaacagcaacctttcctaagcgatagcaaaccttctgacagagaaagatcaccaacattccttgaacgccacacatcatgctgaaattcctttccttcagtcactcggtatgccaagtcctcctagaactccagtaaaagttgtgcctcaaattagaatagaacttgaacctgaagacaatgattatttctggaggagcaagggaacagaaactacattgtaacctgtttgtctttcttaaaactgacatttgttgttaatgtcatttgtttttgtttggctgtttgtttattttttaacttttatttcgtctcagtttttggtcacaggccaaataatacagcgctctctctgcttctctcttgcatagatacaatcaagacaatagtgcaccgttccttaaaaacagcatctgaggaatcccccttttgttcttaaactttcagatgtgtcctttgataaccaaattctgtcactcaagacacagacacccacagaccctgtcctttgcctctattaagcagaggatggaagtattaaggattttgtaacaccttttatgaaaatgttgaaggaacttaaaactttagctttggagctgtgcttactggcttgtctttgtctggtagaacaaaccttgacctccagacagagtcccttctcacttatagagctctccaggactggaaaaagtgctgctattttaacttgctcttgcttgtaaatcctaatcttagagttatcaaaagaagaaaaaactgaaggtactttactccctatagagaaaccattgccatcattgtagcaagtgctggaatgtcccttttttcctatgcaactttttttaaccctttaatgaacttatctgttgagtacattgaagaatatttttcttcctagattttgttgtttaaattatggggcctaacctgccacttattttttgtcaatttttaaaacttttttttaattactgtaaagaaaatgaattttttcctgcagcaggaaacataattttga

SEQ ID NO: 23 - DNA, 2035 bp, Homo sapiens
ttaaagtcgcccccggcgcggagcggaccggcagaggcgggcagaggcggcgagaggcggcgagaggcgggctgaggcggcccagcggcggcaggtgaggcggaaccaaccctcctggccatgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctgagtggctgggcggggccggcaggcacagggaggaggtacagaagccggcaacgcttgctatttattttacaaactggactggctcaggcagggccacggctgggctccagctgccggcccagcggatcgtcgcccgatcagtgttttgagggggaaggtggcggggtgggaaccgtgtcattccagagtggatctgcggtgaagccaagccgcaaggttacaaggcatcctcaccaggggccccgcctgctgctcccaggtggcctgcggccactgctatgctcaaggacctggaaacccatgcttcgagacaacgtgactttaatgggagggtgggtgggccgcagacaggctggcagggcaggtgctgcgtggggccctctccagcccgtcctaccctgggctcacatggggcctgtgcccacccctcttgagtgtcttggggacagctctttccacccctggaagatggaaataaacctgcgtgtgggtggagtgttaggaccaacggtttccta

SEQ ID NO: 24 - DNA, 2459 bp, Homo sapiens
cataaatgtctaaggggcttagaaccttacctaacatattgtatgtgctaagtaccaactactctaatgcgtcaaattgtctttctataccattcttaaaatagaatattttttatttatttttattaaaaatttttttgcctaatctaattatttacgaaaattatgaggtctactcagtttgtcctcttgataaaagccaaagtttttttcttctctcttatttttttgagacggagtctccctctgttccccagactggagtgcagtgacacaatctcggcccaccacaacctccacttcccgggttcaagtgattctcctgcttcaacctcccgagtaactgggactactatgctccaccatgccaggctaatttttgtatttttagtagagacggggtttcactatattggccaggctggtcttcaactcctgaccacgttaactgcccgcattagcctcccaaaatgctgggattataggcttgagccactgcaccaggcctttttttttctctatattaatacgttagagagtacaaatgcagctccctcacagaagcatgttgcataatgatgaagtatgggcttttggtgtgacaacattatctgaatgctctttattgtcccaattagttattttctcattcctaaaccctctgccaacctcccaactttctgagtctccagtgtctatttttccagtctctgtatccaagtgtatgcataattgagttcacacttacaagtgagaaaatgaagaatttgattttctgtttctgaggtttttcacttacaataatggcctctggttttattcatgttgtgcaaaagacgtgatgttattcttcttcatggctgagtagcattccatggtataaacgtatagcacactttctttattcgattattgactgataaatttaaattgatttcatatattggttattgtgaatagtgctgcaataaacatatgagtgtgggtattttctttgtgtaataaattattttcctttaggtagatacaaagtagtgggactgcagaatcaaatggaagttctacttttagtcttttttgaaatctccatacagttttccatagaggttgtagaaagttacattcccacaagcaatgtctaagtgttctcttttctctgtatccttgccaatatttcatttttctgcttttcagtaatagcaattctgaatggtgtaagttggtatctcattgtggtttttaattggcatttctctgatcattgacaacgttgagcatcttttacgtgcttgttgaccatcgttgtctttttttatttaaatgttcatgttctttgcttgctcattgtctttttttttaaaatgttcatgttctttgcttgctttttaaggttacttgttttatttttattttattgagctgtttgagttctttgtatattctggatgttagatctttgtcacatgcaaaacttgtaaacattatctcgttccataggttttctgttcactgtgttaattaaaaagcatttttaggagatttttagtttaattaagttccttttgtctatttttgttattgttacatctgcttttgagatcttagtcataaattctttgtccaagtcaatctctagaagaatttatcctagcatttctcctagcatttttatagtttcaggtcttacattttagtcttttaatccatatttagttgacttttgcatatggtgggacataggggtcccattccttacttctgcatacggaaatataatttttccagcacaatttgttgactaggatgtcatttctccagtgtatgtttttgctgactttgtaaaatataagttgtttgtaggtatgtggctttatttctgggttctctattctgtaccattgatctatgtgtcgatttatatcagtaccatgctgttttggttactaaagccttctagtataattttaagtcagataatgtaatatctccagctttgctctctttgcttagatttgctttggctattcaggctctttttgtggttccatgtgaattttagatttttttttctaattttataagaaataacattggcattttggtaggaattgcatttaacatgtagattgctttgggtagtagagtcattttaataatcataattcttccaatccatgagcatgggatgtttttctcatttgtgtcatgtacaatttctttcatcagtgttttgtagttttccttgtagggatctgtcatctctttgactaattgtatttctaagcattttaccttttatgtagctattgtaaaaggaagtgacattttaattcggttctcagcttgatcaccattagtgtataaaaatgctactaatttctgtaagttgatttttgcattctgaaacattattaaatttatttatcaaatctaaaaaaaaaaaaaaaaaagaaaaagag

SEQ ID NO: 25 - DNA, 811 bp, Homo sapiens
gtatagagccgggttgcgtttatatcggcaaaacctatgtcagagttgaagaagtagtcaggaaaaagcgtctggtcgcaggccggggaacatcttaaaagcaaacttctagcctgctgactcttggcaatgagtgtggatcctggctaaagtgccttgaatgcagcatgaggccaatccatgaatccaacttctcatggagaaatgttaatattttttcagttgaatcaatcagggtgaaactaccatgctatggttgcttactttttattatttcatgtaaagtctaagacaaaatacattaaatgcttattgacatatgtatttattcttcaccaggctgataatatctgcctaattttaaactttcttccatttgtaggtttcaacttattctattgtaagatactgttaaatctaatagaggcattgtcacttttacgtataattttattttatttcatatatttcctattggctttttacatttaaattatggagcacttcatcatataaaaaacttcaattatatttaaacagtagtcttcggatttctttgcttgttaatttccatatttacattataatgagattaaaccatttaatgtttttccgggctactttaaattcttagataattactcatgtttccatgtgcacattggtctactggtgtgtggggggggggagggtacctctggatacatttaaatattcaaagaagctaactcgtggggggggagggaatatcaaagggggccaacaaaagagtggggggagggaaaaaaaagggggtgttaaaaaaatgggg

SEQ ID NO: 26 - DNA, 496 bp, Homo sapiens
gtctctctcctgctaatcttccagacctcaaggacctttccagaccaaggtcctggaggtgaagtgaagagagcacaagctttgaactcatcagacaacaaaattgaagatgaggatgtagataaaaatgccattattacggcttctccttagtctgtcggaggcattcatttgtctggaggaagttagaattttttccaaaaatataagactgaggtaatattagtcttttgacatccaaatggattatgccatgcatcaactttaggattcattcacagaaatgtagcacagcatggtcagaaagaagaaggcttaattttacaaaggtgccttgagtttttatgcagtttggatactgaattgaaacccagtggattgtcataggcctacaatagcagaatgagttgactcaacaataaggtagcgtagactgctggaccaatttgcaggctggaggatgtggcaatcaactcagggttctgcagacagcaaa

SEQ ID NO: 27 - DNA, 506 bp, Homo sapiens
aatttttttttttttttttttttttgcattcatagatactttatttaaaaagtacactaattgaagttttacaccattttatgattcatcaaacacagcaactgaatactggcgtatagcaacattacagatgatgttagtctaagataaaactcataacacattttaaaaagggtaaaaggaaggaaaggaaaaaggatccaaggctgtagaaagattttaaaaacaaacatcgtatgtctgaacctttgcctggagtttcttattttgtgaaaagcagtttgtaagtagaggactcgtaagtgcacctagtacactgcaatggcacagacacggcagatagtcaataaaatgatctttaacttctgatctagattatttttgttgttgttcatgtattaacctagtgaatctcagccaatagatggagtggcattgacctgcacaacttccaaaatcccatccaaccttagtgtcctctgaagttctccataacttattgtctg

SEQ ID NO: 28 - DNA, 2042 bp, Homo sapiens
aaatctctgtttcagtcagaaaccagaggccttgcttagaaatttcacaaaggacctgttctgatatagagacatcggtgataccatctgttgattgcaaagaaaaatatacccgactaataagaagaaaatgaatggtgcataatgacccaatctcctttggatataataaaactatgcctgatttgtggtcttcatttacatttaggggcaagaatcattgctctctttgaatgactctagttcccagcatagaggtttgtatttactagaccaccaataaacattggttaggttcaggctctgagcctctgactgtctggtccagatagcaagtgtctgcaggatttctagaccccatgctcttacaccagcttgtctatgaagatggaagacaaagcttttctttccaagaaaaaactgaaaacatcaacccaagtcttgactttggttgggaaagagtaactgcagtaattaggacccaattgatggcaaagggatatgatctagaatacctgaaacttggtaatttatgaagaaaaggaattcatttcttacagttatggaggctgagaagtccaaggtccaggggcataacagatctgattgttcgcaatttttctactggggtcttcagtgctatgaattctatacaagatacatatgaagagtaagaactcactgcctattaagattgttgcaaatattttctcatttgtcagttttatttataatccttttttgtttataattgtaaagcagtttaaaactattgagtttttcttcctctgcttttattctttttcaccctacttatcactttcaaagaaagtatagaaataatcatcttaatgtgattttttaaaattatgatttcttttaccttaccaagaatctcctcggatgccagaattgacttttactcccttatacattaatgattatataacagaaatcattatcatgttgatgtaaccaattactaaaatatgtaaattcactttcagtatcttttacccaaagaatcattctatacttctgcacaaggtgagaataaaaaaggttactttataaaatgactgtaaaaatagtgagtacaaatattcttttggtcgttatgatgctgtaacattctctgctggtttcaacaatattccttttttttagtcttcctgtttgtctttggacttccaaacagtgagtttaaatatcatagcaacagtgaaccaggttttgtactatttgatttattttttaatctatcttatttgccgtataaaattattaatcttcatcttttaacttacatatcttttctccagcctagcattatatattgagaggaaatccactaaaagtagatcacaaaatctacttttcaaaaaagctatttcgttttttatatcaaaattaccatgggcttaagacagatactaaaatttttaatgaatacaattaaatttttaaaataactggttactaattatattacaatataagttcacctggaatcagataatttgacagccataaactgctctggaggactagggccctcatcaactattggagaaaaaacatttgaaaataaatttgacatatgctataaatataaagacattattttgctttaaaaaatgtggctattttcttctgcaattaaatgtaagaatattcagatatactgatgtcactgtaatactgtatctttggaatcaagatctattttaccttcttttaactatagtgctaattttatacactgagtaagacagggtgatataatgcttatttaataactttcgagatagcttctctttgttttaaaatacagtcataaataagcacttatttaaaaaagctaaatgctttcatttattcaatggatggccttgctgacgaaatgatactgctttttatcttctaattacttcgtatctcattagtgcttcctctaatgggctaaagaaaatgtggaaacttcaaactgttaaatgcacccaggttagttttggtaataggtctgaataaaaaagaaattc

SEQ ID NO: 29 - DNA, 539 bp, Homo sapiens
tttttcatgtttttcgtttattaaaacaaaaggatgcagattcttatcaacaaaggaaaaagattctcagggcaaattccaggagactccaggcacaaatttacaagtgtccatcacagtagaatcacatgcagatacacttcaatctcccagtaatgctgcatacaaccacatgtgaattcttggcaattaaagcagcttatttattttttgatgtcccaggtttttattggtggttaatcacataggcatgcaatgcccacgtggcaaaactcagcaaattcagcttcctttccatgatccctctagaacaaaagcaggctttcagcataaatcatatcttagaataaacgtatctggttaaattgataccacatggttcagataaatacagcatgactcagggcctcaggcatataaagtcagtcattcaccataaacccaaggcctcagatatacaaaaacactcttcacggtaagactgtttccagaactcagaagctagctagtcctaaaaacaaaccttacctttgaatgtg

SEQ ID NO: 30 - DNA, 588 bp, Homo sapiens
  modified_base (80)..(80): a, c, t, g, unknown or other
gtatttttagtagagacggtgtttcatcgtgttagccaggatggtcttgatctcctgacctcgtgttccacctgccttgnctcccaaagtgctgggattacaggcgtgagtcatcgcgccgacctacagttgctttttagaaatgaaaggcttctaggtcaagtgtttcctgcctgagattctgtgtgtagagttagctaggccagaaatgcactgggtccaccagttggcacacagtggctttgagaataaggacactccaccgtagataccttccaagggcctctggtgacatgaacagcaaggggggcatctgatacaggacgttctaacccacagggcactcgcacatgggtaggatgtgacttctatgtactgctctgctctcacagaggccactcaacagtgtaacaccaaagtacatacatggcattcaaaaaggaaaagcaaattatgctttccaagcggcagtgtgttttaccttatcaaaatcatctgagcagtcaagatgaccaagcagtccatttttaatacaaattantggctggctagacactggagatcccaaatagataagaccccnctttt

SEQ ID NO: 31 - DNA, 220 bp, Homo sapiens
tggaagttgatccgccagtccccaggaaggggctggagtggattggcgaactcaatcatagtggctgcacctactgcatttcgtccctcaacagtcgagtcaccatttcactagacacgtccaagaactagtcctccctgaagctgagctctgtgaccgccgcggacacggctgtgtttaactctgcgacatgcgggggactatggttcgggggaactgg

SEQ ID NO: 32 - DNA, 502 bp, Homo sapiens
ttaaaaccacacacaccttgcaaagaaaaagggaaactggcagtctctgtagaggaagccggtggcatcgctcagagccacaaactgtatttctaaacagccctttccctggttccctctctcctgccccactttttttaaaatccagactgtaaaaaacacatctactgacactcactttactttaaaaaaagaagagaaaaagtaaagcgttacaagactttcctcctggaaactataaactgaaaaaaaaatccataaaagattaaatcctggcgggttgtggggtggcgggggccggcggggagggggcgcggagtggagattggctctctgaggtggtcaggggccctgtgacagcttgggactttcagcacctggtttggggtcatttatctgctcaactgtcagaccccccacccccaaaccccagccaccaacacaaccatcgtagaagggaacacaacacagagggtcttttttcattttttttaaaaatcgg

SEQ ID NO: 33 - DNA, 2141 bp, Homo sapiens
gagaaaatttgaaagaatagtacagtgatcacctgaaaatacgtgttctttaaagtccaagaatgcaaaatagcaaggagtgattaggaagttgtcttattaacctttcttttttcagaaaaaaagttctaaagaagtgataaaaataaatattttaaagttttaattacattgacaatatgtgccttttttttttttaaaaaagggactttattattcttacttcaagaccattattgaagcaccttcatttttgggaggactgtggatgattatgaatgacaggcttactgaatatcctcttgtaattaatgcagtaaaacgcttccatatttatccagaggtgagtcataatttgagacaaataatatttgtagagtgatcctctttgtcagataaaaatagtaagcacaatcttgttaagaaagggaggggacgtttcaggtttattaattgtatgtatccttatagttgagtagaatatcataagttcctcttaaaatttaaagaccccagaaacagttctctcatgaactcgaagaatgaaccttagtgtgtaaataatggccagggtaatagacaagccaaatggaaaagagggtcatgggagccaaaaagttctaaatagggatgggcttttggccttggacacctcgtgggtgagcacgatgaccacgtgaagggacagtggagtgatgagcttgaatagtgatcttggagttatgttagtaaagtaagatttatttagtcacacagccagaaatctattactattttccaacacttagcctaaaagagtatggttgttatagtagtctataaatgctagaaatttgaaaaacggagacaaaattagtgaactcagtttgtgaactcaagataaatgcttaatttgacaccatggtaaatactactgttgagtttaaagttgtctcagaatccagcatattaatgaaaagttactgctttctagtcctttattcatatttttatagcttttaaaaattattattctttagattttgaaatgcttaaaactacagacttagaaaataatattcaaatatgtgtgatcaaccatctaaaataaataaatgctaacctttgtcatttttgcatcagctattttaaaataattaaaatattataaatacagttaaaatcacctttgttctgctcctagtccaacttatacctttattaaaatttggtgtgcgtatccatccaatctctgttttcagagattgttttattatatgctttaagtccacataaatgatctcatactgaatgtgtcactgtaggattttttcttcaaaattgtatttttgaactttctgtatatatatatttctttaattttaaccattttatgaattgtaaagtttatgtatttccccatcttaatatattatttccagttttttgattacaagcaatgtaatctaaaatgtacatttttgtatggttttcttctgtatatgtgcaatattttgtctaggatatatacctagaagtggaattgttgttatagagtgtgtgctttttcagctttaccagatattggtaaattgctgtctgaagcaaatgtagcagttcacactgctgtctagaattttctcatatccttattagattttgaattgtcagaccctgcgatttttactcttatgatggctgtgatatgctttttgttttaatttgtacttcctttattatttatggggataaatatatttccatgtttaataagcatttggatttgttcttctgtcaacaacatatataaatctttgtcaccaaatcagcttttttgaggtatgacttacatgtaataaaatgtgccaattttgattgtaccattctctgagttttgacaaatgtatgaaactatatatccatcaccacaattgagttacaaaacattttcagaatgccaaaaaatgctccattgtggtaactgtcttttcccccattgaattaattgtcttggtacaatcgttgaaaattggttgaccacattatatgtgaatgtgtttctggattctattattctcttgatctataattatgccaataccacactgtcttgattactgtaactttagaataaatcttgaaagtaaaaaaaaaaaaaaaaa

SEQ ID NO: 34 - DNA, 1356 bp, Homo sapiens
gaggagtggcagcacctggaccctagtcagagggctctgtatagagatgtgatgctggagaactacagcaaccttgtctcagtggggtattgtgttcacaaaccagaggtgattttcaggctgcaacaaggagaagagccatggaaacaggaggaagaattcccaagccaaagctttccagagatggggtttcaccatgttcctgtggctggtcttaaactcctgagttcaagtgatccacctgccttggcctcccaaagtggtaggattacaggcatgagccacagcatcccacctgtaatggggagttttaaatttacaacttcaatcttttttacttgtgtaggtcttttcagatttttctttttctacttgagttggttttagttgtttatgttttatggattttcccattttgttgaagttaaataatttgttggcatacattgttcataccattctttgtaattatttttatttctgtaagagtactagtaatacatctttaatccctacttttagtaatttgaaccttctctctcttttttttctttttcaatgtcagtctagctagaggtttgtatattttgttgatcttttccaggaatcagctattgtttttctgttctctagctcattaatttttactctataatctttctgctttcctgccttctgcttgatttggttttgtattgctgctcctgtccaagtgttttaaggtggaaaattagtttactgatttgagatctttttaaatatggttattacagctacaaatctctaaggattgctttatctgtgttccataagtttgagtatgtcttcatttttatctcaaagcatttttttctaattttccttcttattgcttctttaactttttttttttaactccggaatatgttatataatttgtgcatgtttgtgcattctaaaaattcctttgtcttactgatttgtaattcgactgtggtcagagaatcattttaaatttattctggggaatgtttcatgtgtacttttgaagcatacctgctccactgttcgagtggaatgttctgtctatatctcttagatctggttgctttagagtgtcatctatatcttctgtttccatgtttatattgtgcccagttgtcctaaccattattgaaaatggggtattacagtttgcaataagtattgtagacttgtatatttttttcttttaattctgtcagtttttgctacacatactttgaggctctgttagtagtacatacgcttagaattgttatgtctcctttattgattgacccttttattgttatattaataaaattcccctctttatccttagtaaaaaaaaaaaaa

SEQ ID NO: 35 - DNA, 2048 bp, Homo sapiens
ctcacagtacgttcacatcagacctttctgtgcagacaacctccagtaccagcccagagcctggtagactcctctggttgctagatccagaagagagataacaatctttacagcttggctctcaggaagccgatcccctaggaaaaggggctccgtactacatcaagggactactccatgggacggcacaatttgaacaacagcctttggccttagacctctgtgacaaatgagaagaaaccagagaactttcaagttccatgggacaaaacaatgttcttcaccacccaagacctgatcacactagcggatctgcaatggattccaagaatcaagaagtccctgaggtacctgaaaaagaattcaggaggtcctgtgttaagctaatcagggaggcaacagagaaaggtgaagcccaatctaaggaaaggaaaaaaaaagatacaagaagtgatgggagaaatactcaatgaaatagatagcataaataaaaaatcaaaatttcaggaaacaatagacacacttatagaaatgctaaatgttctggaaagtctcagcaatagaattcaacaagcagaagaaagaacttcagagcttgaagacaagttctttgaattaaccaaatccaacaaagacaaagaagaaagaataagaaaaattgaacaaagcctccatgaggtctgggttcacgttaaactaccaaccctaagaataattggcattcctgaggaagaagaggaacctagaagtttggaaaacatatttgggagaaaaattgaggaaaacttccctagccttgctagagacctaggcatccaaatacaagaagcacaaagaacacctgggaaattcattgcacaaagatcatcacctagacacattgtcatcagattacctaaagttaagatgaaggaaagaatcttaagaactgtgagtcaaaagcgccaggaaacctataaaggaaaacctatcagattaacagcagatttctcagcagaaatcctacaagctagaagacattggggacctatcttcagcctcctcaaacaaaacaattatcagccaagaatttgtatccagtgaaactaatcttcataaatgaaggaaaaatggtattttttagacaaacaaatgctgaaaggtctcgccactaccaggtaagcactacccctgatcttaaaaggagctctggatcttgaaacaaatccggatgacacatgaaaagataacccctttagagcataaatctcttaggacctataaaacaaggatcgaatataaaaaaaaaaccggatccaaacaaaaccgtgaaggtatacagggaacaaatagtatgatgaatgaaatggtaactcccatctcaatactaatgttgaatgtaaatggcctaaatgctccccttaaaagatacagaattgcagaatggataagaactcaccaaccatctgctgccttcaagagactcacctaacacataaggactcacataaacttaaagggatggaaaaagacattccatgcaaatggacaccaaaagtgagcaagagtagctattcctatatcagacagaacaaactttaaagcaacagcagttaaaaaagacaaagagggacattatataaatgataaaatgtcttgttcaacaggaaaatattacaatcctaaatatatatgcacctaacactggaagtccctaatttataaaacaattactaatagacttaagaaatgagatagcaacacaataatagttggggacttcagtactccactgacagcactagacaggtcatcaagacagaaattcaacaaagaaacaatcagtgtaaactataccctggaacaaatggatttgacagatttatacagaacattctacccaacaaccacagaatatacattttattcaacagcacatggaactttctccaagatagaccatataataggccataaaatgagtctcaataaatttaaaataatcactctctcagaccatagtggaccgatactagaaatcacctacaaaaggaaccaccttcaaaacc

SEQ ID NO: 36 - DNA, 490 bp, Homo sapiens
ttggatgcattcatagatactttatttaaaaagtacactaattgaagttttacaccattttatgattcatcaaacacagcaactgaatactggcgtatagcaacattacagatgatgttagtctaagataaaactcataacacattttaaaaagggtgaaaggaaggaaaggaaaaaggatacaaggctgtagaaagattttaaaaacaaacgtcgtatgtctgaacctttgcctggagtttcatattttgtgaaaagcagtttgtaagtagaggactcgtaagtgcacctagtacactgcaatggcacagacacggcagatagtcaataaaatgatctttaacttctgatctaggttatttttgttgttgttcatgtattaacctagtgaaactcagccaatagatggagtggcattgacctgttacaacttccaaaatcccatccaaccttagtgtcctctgaagttctccataacttattgtctgtt

SEQ ID NO: 37 - DNA, 2844 bp, Homo sapiens
tggcagtggctgcttggcttggcagtggtcgcttggcttggcgtggcagtgatcgcttggcttggcatttctggcttagcggtcctcctttcgcagattggaaaccgcgggctatcctgctgggaggttgtggccgaggcagtagctcgctactgatggtctcctggggtggagaaaagcggggaggggctgaggggtctccgaagccggcagtctacgccacgaggaaaaccggtagcgtcaggagccaggaggaccagtggtacttgggctacccgggggaccagtggtcctcgggcttctcctacagctggtggaaaaacagcgtcggcagcgagagcaagcacggtgagggcgccttagaccagccccagcacgatgtccgcctggaagatcttggcgagctccacagagctgcccggtcgggcgacgtccctggggtggagcacgtcttggttcctggagacactggcgtggacaagagggataggaagaagagcattcagcaactagttcctgaatataaggaaaaacagacacctgaaagtcttcctcaaaataacaatccagattggcatcctactaatttgacccttagtgatgagacttgtcagagatccaagaatctgaaagttgatgataaatgtccatctgtatcaccatcaatgcctgaaaatcagtcagcaaccaaagaactgggacagatgaacttaacagaacgagaaaagatggacactggggttgtacttctctcagggaatgatactctccatgacctgtgccaatcacagctaccagaaaacaaagagagcaaagaaggtaacaaagaagcatagagaattcagtcctagagtgcctctgcttggctgcatagagctatagttctgacttaaaaaaaactttttctggtaaaatgtgctttgtgtttactcaccttttgcactgaacagaataccttttggcaggatatttgtgttcttcctttaaccaaagcaacatctagataacagagaattaggggaaagcaggattttctttaggaagtaggattatatttagaatatagggactaagtatgtttagggactaacataggattatatttaggataacttagtagagacctaaagattgctgactcaaacacaatgtggtttctttgctttatttttacagctctgaattcacagctgttagttataactatatgcactataatttaaaagacaccttcccacacctgcaatctcagcactttaggaggccgaggtgggtggatcacgaggtcagtagttcgagaccagcctggccaatatggtgaaaccccatctctactaaaaatacaaaaaaaactagccaggcatggtggtgaatgcctgtagtcccagctactcaggaggctgaagcaggagaatcgctggaacccagaaggtggaggttgcagtgagccgagattgcgccactgcactccaacccgggcaatagaggaagactccgtctcaaaaaacaaaaacaaaaacaaaaaaacaccttccaaaagcaaatattaagtgacttcctatcgtttctatgactttatgtattatagaattgactttccaagtcagtaattaatgagacttattgagaatttgctgcatagtatctccttggctgtgtccacatgagctaccaccttgcgttaataaatgtgttcgctagggatgttggttttggcattgacagtgttctatcaccaatgcaaataagaccagggaccactcttgagaacattaatgtccaagcatcttaaagttatacgtaaggctttcataatgtgattctgctcagctctccatctttagccctttcccccgtgtgccccttctctggccttactgaactgctggtcctgtcctgctgacccatccttcgactgcaggcaaacacatacactctttcaggcctctttctgccttttgttgctgcctgtcatgctctcactctttcctgtcccctaccccttctaattaggctagcctcactctttaagcctcagctcgggcaggatctctagaaaagtcatctctgacatgctttcttttcactctttaaatcctagtgcctagcacatagcaagactcaatacataattgctgagtaaaatagataatgactctttgtacaaagatgattttcaagattgttccctacagtcaaggagcagagggaatagacatgtaaagcaataattgacaattcgggtggtggagatgcagtagaaaaatcagtaaagttctagggcagccctaaggaaggagatacctagatacctgtttacctggggaaagatgaggatgagggtcaacttcacctagcagtctgtttttttattttatgtattttatttatttatttttagcagtctgttttaaaagatgaaaatattaatacatttgtcagaataatacagggaaccatttcggatgttaggaagaggttgtacaccaataaaggtgtccacagtcattttggaaatcattaataaggtactattatgaaatggagaacagtatgcagcattgcctgataatattgctactgcattccaaaattttgttttgtttgtgatggctttgttcattgatgttgggtggatgaatctgtgagtgaatcttcaacatgattgtatttctttaacctggtggcatctagtgtctcccagaaagtggtttgttgaagttttggagaattagaattatttcttaaagaagtaagtattccactaaagatcaagcttcatttaaactctcaatttatgaaataaaatgaaatgtaatttaaaatccaaaaaaaaaaaaaaaa

SEQ ID NO: 38 - DNA, 1245 bp, Homo sapiens
actgctcttccagaggcaagaccaaccaagatgagtgacctgggagctgtcatttccctcctgctctggggacgacagctttttgcactgtactcaggcaatgatgtcacggatatttcagatgaccgcttcccgaagccccctgagattgcaaatggctatgtggagcacttgtttcgctaccagtgtaagaactactacagactgcgcacagaaggagatggagtatacaccttaaatgataagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactaccaccaggtagatattgggctcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgtatgtgaaggtgacttccatccagcactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccaaaa

SEQ ID NO: 39 - DNA, 1599 bp, Homo sapiens
gtccaaaggctatggatttgtttcgtttgttaatgacgtggatgtccagaagatagtaggatcacagatacatttccatggtaaaaagctgaagctgggccctgcaatcaggaaacaaaagttatgtgctcgtcatgtgcagccacgtcctttggtagttaatcctcctcctccaccacagtttcagaacgtctggcggaatccaaacactgaaacctacctgcagccccaaatcacgccgaatcctgtaactcagcacgttcagtctgctgcaaatcctgagactccaaactcaaccatctccagagaggccagcacccagtcttcatcagctgcagctagccaaggctgggtgttaccagaaggcaaaatcgtgccaaacactgtttttgttggtggaattgatgctaggatggatgaaactgagattggaagctgctttggtagatacggttcagtgaaagaagtgaagataatcacgaatcgaactggtgtgtccaaaggctatggatttgtttcgtttgttaatgacgtggatgtccagaagatagtaggatcacagatacatttccatggtaaaaagctgaagctgggccctgcaatcaggaaacaaaagttatgtgctcgtcatgtgcagccacgtcctttggtagttaatcctcctcctccaccacagtttcagaacgtctggcggaatccaaacactgaaacctacctgcagccccaaatcacgccgaatcctgtaactcagcacgttcaggcttactctgcttatccacattcaccaggtcaggtcatcactggatgtcagttgcttgtatataattatcaggaatatcctacttatcccgattcagcatttcaggtcaccactggatatcagttgcctgtatataattatcagccatttcctgcttatccaagatcaccatttcaggtcactgctggatatcagttgcctgtatataattatcaggcatttcctgcttatccaaattcaccatttcaagtcgccactggatatcagttccctgtatacaattatcagccatttcctgcttatccaagttcaccatttcaggtcactgctggatatcagttgcctgtatataattatcaggcatttcctgcttatccaaattcaccatttcaagtcgccactggatatcagttccctgtatacaattatcaggcatttcctgcttatccaaattcaccagttcaggtcaccactggatatcagttgcctgtatacaattatcaggcatttcctgcttatccaagttcaccatttcaggtcaccactggatatcagttgcctgtatataattatcaggcatttcctgcttatccaaattcagcagttcaggtcaccactggatatcagttccatgtatacaattaccagatgccaccgcagtgccctgttggggagcaaaggagaaatctgtggaccgaagcatacaaatggtggtatcttgtctgtttaatccagagaagagactgataaattccgttgttactcaagatgactgcttcaagggtaaaagagtgcatcgctttagaagaagtttggcagtatttaaatctgttggatcc

SEQ ID NO: 40 - DNA, 1543 bp, Homo sapiens
cccacgcgtccgagttgagctctatgattattctgatgaagtggattggagaccatcacctttctcttagtaaatatttgtccttcacataccaatcgtcaagtctaaaaattggaccactacaggctctgaccacatgtatgagaagccaaaggacctgtgatctcacctgcctcaatcatgtgccattatttactccacttgcctcttctgaatgggtatgatcccaaccaaacatagtgaaggggaaagttaaacaatatttggaaagccatgtatttggaaagccaatgtaaaagtgaaaagaaggccacccacgagagagagagagagagagagagagacagagagagagatctttcatttgtgaattgcaaagcttcagaggtccgaatttggccagtatgtttgctgcaaagccttggaatgctgttttgcagtttcaaaagtgatcatactcagcttcccagaattttttatgcctggaggatgccacaagaacaagggcagccttgttaacaagttccaatctctcaaaaacaagataggattcggatataaatcatcatttcagctcacaaagccatgttctccaccccactgaagggcttcatttacatctaaaatggttaatactgatctgcaccagaactcctaagtggaggaatgcagctgccatgtgcttagtgactctggggttagggaagaatgacatcaccaaatgtaagcatattatcatgctgacatagaggcaccttgttttctcctctgtggatagagcaaggattgtgttctgtctcttgtttttttccgctcgaagaatacctttccccctcccttcttagcagctgcaatatgaagggctgaaaatgtcacttcaactaatgatgaatgatgcttgacctctggatgggttactcagaattttatgcaattctaaattaatatgaaagatcccaactcaaaggacttcggccgttaagatttgcttcaaggatagatttgtgtaaagcagttagtagctttggggattttgttgctgttgttattgtacttggtgggggactgggagactcgtgctctgtaagcaaggtaaaaggcagaatgtgctaacatttttatgtgttatttttttcctgtgagattaatatctttttctactagaaatattgtcagtaagcagaaccagtttgtgaaaaaaacattctcaaaagggcacaggcccacacgtctttgttgtttttatttgaatataattaattatgtaaagcaatagttagtgtaagattaacaattagtagattctgtcaagtagtcacacggaaattactaactaattgaactactggtcgtctattgtgtcagaaagcagtactgagtgtcattctttttatctttttactttgttgttttcacagatcctcttgtttctaagatgtatgtttcacagccgactaataaataaataaccctggttgtaatttcataacatgcaaaaaggtttgaaataaaaacacctgttttaattaaaaaaaaaaaaaaa

SEQ ID NO: 41 - DNA, 3771 bp, Homo sapiens
ggcagtggcagtggccgctgcagccccacactccgccgccaaactggaggagcgacggaagccagaccccaggaggatggaggatgaagctgtcctggacagaggggcttccttcctcaagcatgtgtgtgatgaagaagaagtagaaggccaccataccatttacatcggagtccatgtgccgaagagttacaggagaaggagacgtcacaagagaaagacagggcacaaagaaaagaaggaaaaggagagaatctctgagaactactctgacaaatcagatattgaaaatgctgatgaatccagcagcagcatcctaaaacctctcatctctcctgctgcagaacgcatccgattcatcttgggagaggaggatgacagcccagctccccctcagctcttcacggaactggatgagctgctggccgtggatgggcaggagatggagtggaaggaaacagccaggtggatcaagtttgaagaaaaagtggaacagggtggggaaagatggagcaagccccatgtggccacattgtcccttcatagtttatttgagctgaggacatgtatggagaaaggatccatcatgcttgatcgggaggcttcttctctcccacagttggtggagatgattgttgaccatcagattgagacaggcctattgaaacctgaacttaaggataaggtgacctatactttgctccggaagcaccggcatcaaaccaagaaatccaaccttcggtccctggctgacattgggaagacagtctccagtgcaagtaggatgtttaccaaccctgataatggtagcccagccatgacccataggaatctgacttcctccagtctgaatgacatttctgataaaccggagaaggaccagctgaagaataagttcatgaaaaaattgccacgtgatgcagaagcttccaacgtgcttgttggggaggttgactttttggatactcctttcattgcctttgttaggctacagcaggctgtcatgctgggtgccctgactgaagttcctgtgcccacaaggttcttgttcattctcttaggtcctaaggggaaagccaagtcctaccacgagattggcagagccattgccaccctgatgtctgatgaggtgttccatgacattgcttataaagcaaaagacaggcacgacctgattgctggtattgatgagttcctagatgaagtcatcgtccttccacctggggaatgggatccagcaattaggatagagcctcctaagagtcttccatcctctgacaaaagaaagaatatgtactcaggtggagagaatgttcagatgaatggggatacgccccatgatggaggtcacggaggaggaggacatggggattgtgaagaattgcagcgaactggacggttctgtggtggactaattaaagacataaagaggaaagcgccattttttgccagtgatttttatgatgctttaaatattcaagctctttcggcaattctcttcatttatctggcaactgtaactaatgctatcacttttggaggactgcttggggatgccactgacaacatgcagggcgtgttggagagtttcctgggcactgctgtctctggagccatcttttgcctttttgctggtcaaccactcactattctgagcagcaccggacctgtcctagtttttgagaggcttctatttaatttcagcaaggacaataattttgactatttggagtttcgcctttggattggcctgtggtccgccttcctatgtctcattttggtagccactgatgccagcttcttggttcaatacttcacacgtttcacggaggagggcttttcctctctgattagcttcatctttatctatgatgctttcaagaagatgatcaagcttgcagattactaccccatcaactccaacttcaaagtgggctacaacactctcttttcctgtacctgtgtgccacctgacccagctaatatctcaatatctaatgacaccacactggccccagagtatttgccaactatgtcttctactgacatgtaccataatactacctttgactgggcatttttgtcgaagaaggagtgttcaaaatacggaggaaacctcgtcgggaacaactgtaattttgttcctgatatcacactcatgtcttttatcctcttcttgggaacctacacctcttccatggctctgaaaaaattcaaaactagtccttattttccaaccacagcaagaaaactgatcagtgattttgccattatcttgtccattctcatcttttgtgtaatagatgccctagtaggcgtggacaccccaaaactaattgtgccaagtgagttcaagccaacaagtccaaaccgaggttggttcgttccaccgtttggagaaaacccctggtgggtgtgccttgctgctgctatcccggctttgttggtcactatactgattttcatggaccaacaaattacagctgtgattgtaaacaggaaagaacataaactcaagtttatacccatgcctgtactctatggtgtgttcctgtatatgggagtagcatcccttaatggtgtgcagttcatggatcgtctgaagctgcttctgatgcctctgaagcatcagcctgacttcatctacctgcgtcatgttcctctgcgcagagtccacctgttcactttcctgcaggtgttgtgtctggccctgctttggatcctcaagtcaacggtggctgctatcatttttccagtaatgatcttggcacttgtagctgtcagaaaaggcatggactacctcttctcccagcatgacctcagcttcctggatgatgtcattccagaaaaggacaagaaaaagaaggaggatgagaagaaaaagaaaaagaagaagggaagtctggacagtgacaatgatgattctgactgcccatactcagaaaaagttccaagtattaaaattccaatggacatcatggaacagcaacctttcctaagcgatagcaaaccttctgacagagaaagatcaccaacattccttgaacgccacacatcatgctgataaaattcctttccttcagtcactcggtatgccaagtcctcctagaactccagtaaaagttgtgcctcaaattagaatagaacttgaacctgaagacaatgattatttctggaggagcaagggaacagaaactacattgtaacctgtttgtctttcttaaaactgacatttgttgttaatgtcatttgtttttgtttggctgtttgtttattttttaacttttatttcgtctcagtttttggtcacaggccaaataatacagcgctctctctgcttctctcttgcatagatacaatcaagacaatagtgcaccgttccttaaaaacagcatctgaggaatcccccttttgttcttaaactttcagatgtgtcctttgataaccaaattctgtcactcaagacacagacacccacagaccctgtcctttgcctctattaagcagaggatggaagtattaaggattttgtaacaccttttatgaaaatgttgaaggaacttaaaactttagctttggagctgtgcttactggcttgtctttgtctggtagaacaaaccttgacctccagacagagtcccttctcacttatagagctctccaggactggaaaaagtgctgctattttaacttgctcttgcttgtaaatcctaatcttagagttatcaaaagaagaaaaaactgaaggtactttactccctatagagaaac

SEQ ID NO: 42 - DNA, 511 bp, Homo sapiens
tttttttttaacttttttatatgttatgggaaaacacaatggattcattcacaaacaaaattagttatgcagaaccaaacatgcaaccaaagcacgccagagagacaggattccggagcagaacttaccccacgctttaggctcctgaagcagtggattttgggtttggaggtcatgaactcgttgaagcggtgggagaggggttccttttgctgcttgggagactgggggccgttgggaacagcagagggtgaggcagaggcaggagggggagggggaggctgatggggggatgttaaaagggacataagctacatgtaagagatggagccgtgacagagtaaaaatcagaaacgagaagacacaaaacaaaaataagcatcaaataatatttgaaatccaacgcaagtaaaacacaaacaattaaattttaggccatggtaccaaagaaaacatgtaggaggtggttaaatgaagaatatgatgagagttgggaggtttagtaagtaat

SEQ ID NO: 43 - DNA, 524 bp, Homo sapiens
  modified_base (11)..(11): a, c, t, g, unknown or other
ttttttttttntttttgattttagcccaagttcagtaaatttattttgtcaaaacaattgatatcttgagcattactgagccaacaggacatcaaaataaaaagttgtctaaagttaaaggcacagtacattaacaaacaaatgancctcagtcacaaanttataaatgcagtttgggatgggggtaggcgggaggggagttaatccaaactacagcantgaagtcttcaaaccaccttctgancagggctgctgattgttcctttcatttcttcttgtgaccttgagctcccttaaanaaaaaatttcagtggagantcacagctggtaatgtactgcgagttcttggaagctacacaaggtatagtctgggctaagttacatgtggtttccatattagcttgtttgggcagggtgacctactggcaaagngggcttcagttaccccaccagttcaaccccnggggagtttntaatttcccccttatcctcatgttgtaaaaggctcctaggcacctcaccaa

SEQ ID NO: 44 - DNA, 651 bp, Homo sapiens
  modified_base (57)..(57): a, c, t, g, unknown or other
atcgctgttctgtttctctctcatccacgcctgaatctgctctttcagttctggcanctggttccagcatgnctctctgtcagcgtctgccggttgaaggggtccgtgggggagttgagcaggtgccgcaggatgatggagcggtccatgatggtgccagagggcagccgcacggggtctgtcatgagggtgtccatcagagggtctctgaactcatcaggagcgtcgctgtagtcgatttctgcgcgtgcgttcttggccactatctcctccactttctcggcgagcagcttaaatttttctattgctattgtggatttgatccctggccttcgcatctttgaaataacttcttcaaacaattgcctacctggaggacctccaaccctagatgggcgggcctacaccacgtcgaggctctatggtatagcctattgctcctaggctacaaaactgtacagtgtgggactgtactgaatcctgtcggcagttgtaacacaatggtgagtattctggtcgtcagcaatggctttcgcgaaccgagcacagtccagctgtaagtaaatatgcgtcagttggtccaacagcttcttttgttcaagccgtatttctcatggctgtcaactgtcagtcacggcacttgggcccaca

SEQ ID NO: 45 - DNA, 1183 bp, Homo sapiens
atccctttttattgctgagcagtattctatggtatgaatgtatgatggttcggacatatgggttgtttcctattttaggctgttatgaatgaagctgctctgaacattcatgtatagatttttgtgtatacaggtattttttagaaaatagagataagccaggcgtggtgatgtgtacctgtagtcccagctactctggaggctgaggcaggagaatctcttgagcccagaggttcaagatgagcctgggcaacattttggagatcctgtctccaaaaaaaaaagatggaggtagttttttgtaatggcaagattgtacattagaagatagaaaagtaggttttgcatcttactagggaagatacatgtatatttctctccctttggactgatcatttaagttgtctcaatttccttatcaagaaactggcataatatatgttctgcctacatcccagggtggttgtatagataaactgagataatatatggcctcctcccaagtagctgggattacaggtgcgtgccaccacgctgggccgatttttgtgttttcggtagagacggggtttcactgtgttggccaggctggtcttgaactcctgacctcaagtgatcctcccaccttggcctcccaaggtgctgggattgcaggcgtgagccaccgtgcccggcctcagagtagttcttaaatggtaatctagagacaactcttttaggccatcttgctttttctaataattgatgtgtagatgttccttttatattctgtttactcattgttgacgattataggttgcaatacctgctcccggatttagtttatcaccttacttttaattgaattatttgatatataagaaattccaagtccaagcatggtggctagcgcctgcaatcccagcactttgggaggccatggtgggaggatcgcttgggtccaggagtttgagaccagctgaagcaacatattgggactctgtctctaccaaaaatacaaataagttggctgggcatggtgatgcactcctataatcccagctactcaggaggctgaggtgggaggatcacttgagctctagaggccaaggctgcagtgagccgtgatcatgcctctgcaccctagtctgggtgacagaaggagaccctgtctcaaaaaaaaaaaaaaaaaaaaaaaaaaaaa

SEQ ID NO: 46 - DNA, 1548 bp, Homo sapiens
ctgatgcatgacctctttgacagttgcctttgttcatgattcctagtttcctggtttattttattggggtacagggtgaggtctggaagacctggaaaggaaactcacttttttggtttgacagtattaatgcaatcatctagttcataccttgtaagcccacttattatttcctctgcgtgtgttttttctggtttagcggattagctgcactgtctcttcaaaggctgtccaatcaaggaggggttattaaaaccagggcgatttatgactgagaattaattagagaagcattttcatgcacaacatccaattttttgattagcaatggagcagggccgcaattaacactcgaggaagcttaaatttccagctttttgattctcaggaaatgagattatcaaaccagggtcagacacttgacagcaaagtgggagtgggggagtgtgaaattatatgtaaaaaaaaaaaaaaaaaaaaaaaattccagagtctagaaattcggtcatttttctctttatgtaatggtaagaattaagaatcctcacatctgcaaccaaaaatacaagcctggtgtgtaacactaaagggtaaaacagggatagatataagctgttatgctattcttcaggcaacactgtggataagtgacattcagatgtttactgtaaagaaaaatttgaatacatttgtattgaagggcttttagaaaagagcattactacacagaactgagagttgaaaatatgaaggtgtggaagattaagagaacgcgtttactttaaaagataatttaaaagttatctttccaggctgacacggtggcccactcctctagtcataacactctgggaggctgtggtgggacgatccctcgagcccaggagttcatagaccagcctgggtaatatagggagaccccatctctatctaaaattttttttaaaagaaaataaatatctttacagttatttttcttagtcctatgttctttattttggtgttttccattggatacctgcatgccaagtgttgtgctacagtattactgaagagtataatggaagtaatgtcctgctgaaaattttctttgagatattaatcattaataatttatatattgctatttaatacttacataggtctttagccttttaaaggatttctgtttgacagcttttataattgaaagttattccattttttttttaattttgcatgcttgaaaaagatgaaaacagtgatttaaattatgaagtatggggccaggtgcagtggctcatgctggtaatcccagcactttgagaggctaaggcaagtgggtcacttgagcccaagagttcaagaccagcctggccaacatggtgtgaaaccccgtctgtactaaaaatacaaaaattagccaggcgtggtggtgcatgcctgtaatcccaggtacttgggaggctgaggcacaggaattgcgtgaacccaggaggcagaggttgcagtgagccaagatcacatcaccgcactgcagcctgggtgacagagtaagactctgtctcc

SEQ ID NO: 47 - DNA, 648 bp, Homo sapiens
  modified_base (578)..(578): a, c, t, g, unknown or other
gcaggactgaacagagagaactcaccatggagttgggctgagctggctttttctgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgacccacccggtgatggctgactggggccaggaaccctggtcaccgtctcctcagcacccaccaaggctccggatgtgttccccatcatatcagggtgcagacacccaaaggataacagccctgtggtcctggcatgcttgataactgggtaccacccaacgtccgtgactgtcacctggtacatggggacacagagccgagcccangagaacttccctgagatacaaagacgggacagtactacatgacaagcagccagtctccanccccctccag

SEQ ID NO: 48 - DNA, 453 bp, Homo sapiens
agagctagcaccatgagcttcaacaccctgtacctactacaatttcagaagcagagctgcctgctacagcaaactggaaaatgtgatgccttcgtgaaatttcccgtcgctaaagatactctacacatgccccttgaccaagaaatagcacttatgggaactctacagatggatttctacacatctgaagtgatgcattggccacactattcttaatggcattgtttgtaatggccccaaattggaaatcacctaaagtccatccctacaggacttgtttaataaattatgaattatccatactttacatctattaaaaaatgaatacttgggccaggctcacatctgtaatcccagcattttgggaggccaaggcaggtggatcgcttgagttcgagaccagcctgagcaacatggtgaaactctgtctctaccaaaaatgcaaaaaaaaaa

SEQ ID NO: 49 - DNA, 1680 bp, Homo sapiens
atgccgttcccatttgggaagtctcacaaatctccagcagacattgtgaagaatctgaaggagagcatggctgttctggaaaagcaagacatttctgataaaaaagcagaaaaggctacagaagaagtttccaaaaatctggttgccatgaaagaaattctgtatggcacaaatgaaaaagagcctcagacagaagcagtagctcaacttgctcaagaactctataatagtgggctccttagcaccctggtagctgatttacagctcattgactttgagggcaaaaaagacgtggctcaaattttcaacaatattctcagaagacaaattggtacgagaactcctactgttgaatacatctgcacccaacagaatattttgttcatgttattgaaagggtatgaatctccagaaatagctctaaattgtggaataatgttaagagaatgcatcagacatgaaccacttgcaaaaatcattttgtggtcggaacagttttatgatttcttcagatatgtcgaaatgtcaacatttgacatagcttcagatgcatttgccacattcaaggatttacttacaagacataaattgctcagtgcagaatttttggaacagcattatgatagatttttcagtgaatatgagaagttacttcattcagaaaattatgtgacaaaaagacagtcactgaagcttctcggtgaactactactagatagacacaacttcacaattatgacaaaatacatcagtaaacctgagaacctcaaattaatgatgaacctgctgcgagacaaaagtcgcaacatccagtttgaggcctttcacgtttttaaggtgtttgtagccaatcctaacaagacgcagcccatcctagacatcctcctcaagaaccaggccaaactcatagagttcctcagcaagtttcagaacgacaggacggaggatgagcagtttaacgacgagaagacctatttagttaaacagatcagggatttgaagagaccagctcagcaagaagcttaatctccaataaacatctatgttaaatccaaattcagcatttgctgttagctattcagcatcaggcactcttattgattcatgaggaacattactgctaatctgctgttaagtgaacggtttttcattttacccttttgtttttcagtccaggttggagatcgtagctgctgctgcttgcacactagggcacatgtgggctttctcttgatctttgtgtcatttcagaattcaaagactgtgctacgggagttctgaacatggctgggttcatgaaggcaaatgtatggatgagagtttggtttaggaaagagggcactgatatcagattagacctatgtgtttgcacccatctttgttggcgatctgagtgcagtgtggcaagtgcacacctggcatccctgcgtcagatcgcgcaccttcaggtcgcgcaccttcgctgaaggaagatgacgcagagctttatctgaaatcagaggggagctatccaaaatgggagtttgggggcagctaaagttgacatgcgaataaattgatactgaaaaacaaaagggtaaaatgaaaaaccgttcacttaacagcagattagcagtaatgttcctcatgaatcaataagagtgcctgatgctgtatagctaacagcaaatgctgaatttt

SEQ ID NO: 50 - DNA, 353 bp, Homo sapiens
ggctgcaggattcggcacgagagatgcaaggtctatctctcccatcagggctgccaaagctgggctttgtttttcccagcagaatgatgccattctcacaaaccaatgctctatattgcttgaagtctgcatctaaatattgatttcacgttttaaagaaattctcttaaattacaattgtgcccaatgcagggtggctctggggggcaagtaggtggtacaggggattggaaacatgctccgcgcctccagaggttgggctggtccttagactgggtgcttatgattgggtcttggttagcccactttccctctccatgtggagatggaaggtagagggatacagtgtcttt

SEQ ID NO: 51 - DNA, 623 bp, Homo sapiens
tttttttttttttttacaataaattattcattctaaccttagcaaaggtacatggattctgtctaccaaatattaattttacagtgaccacaaaaggttatttggttgttacaaagactacaatttgaagatactagataggcaaaaattttgccattattaatatcgaccaaacacagaaaagacaacagatgtgtttctaaggcacgatttacatactaaattcccttgaaattcaatgcaatttcacttcgctcatgattttcaatcacaagacacctgtgacaaatgaggaacaaatccagaagagcccatctaatctcgtcttaagtggtcccttaaatacgttaaaagcccaccaatataggcatttacaaggcttttctaagacttccagctcctgaaaaaatcacaccttcacagtttcaaatcaaccacctaagttgctaaggggcagcgaaggatgctgatctgctgcgtatgcaagtctgactcccatctttcaagtgactaattcacttgataacacccacaccccaaacaaaacaaccctctgaaacaggctgtcttggccaccaggaggaagctggtaaagacatttcccctgcctgtgagatggcatc

SEQ ID NO: 52 - DNA, 1966 bp, Homo sapiens
caggagaatcaattaaagccattgtgaaagatgtcatgtatatctgcccatttatgggagcagtgagtggaaccctgacagtgacggactttaagctgtacttcaaaaatgtcgagagggacccgcattttatccttgatgttccccttggagtgatcagcagagtggagaagattggagcacagagccatggagacaattcctgtggtatagagatagcgtgcaaggatatgaggaacttgcggcttgcttataaacaggaagaacagagtaaactagggatatttgaaaacctcaacaaacatgcatttcctctttctaacggacaggcactatttgcattcagctataaagaaaaatttccaattaatggctggaaagtttatgatccagtatctgaatataagagacagggcttgccaaatgagagttggaaaatatccaaaataaacagtaattatgagttctgtgacacctaccctgccatcattgttgtgccaactagtgtaaaagatgatgacctttcaaaagtggcagcttttcgagcaaaaggcagagtccctgtgttgtcatggattcatccggaaagtcaagcaacgattacccgttgcagccagccacttgtgggtcccaatgataagcgctgcaaagaggatgaaaaatacttgcaaacaataatggatgctaacgcacagtcacacaagcttatcatctttgatgctcgacaaaacagtgtcgctgataccaacaagatgctgcttgctggggcagtaagaattgctgataaaatagaatctgggaaaacatctgtggtggtgcattgcagcgacggttgggaccgaacagcccagctcacatctctggctatgctaatgttggacagttactacaggaccattaaaggatttgaaactctcgtagaaaaggagtggataagctttggacacaggtttgcactgcgagtgggccatggtaatgacaaccatgcggatgctgaccgatctcccatatttctgcagtttgttgattgtgtttggcaaatgacaaggcagtttccttcagcattcgagtttaatgagctattcttgattacaattttggatcacctttatagctgtctttttgggacctttttgtgcaactgtgaacagcagcgattcaaagaggcatggggggctgggacccagcgagcccgtggctctctcaggagtagatgagtgctagaccctggacctcctgactccgagttcatgtagtctccgctgcaccaactccaaaaccgtgggattctgcttttcatcctctgtggtgttggccagatcatacttgaaggccttggaaatggagtctgctgattagccaaaaagcccactttaggatgtatatacaaagacgatatctttatggtcgtatatcaatagccagctagacgagttttctaatcccttctttgtgaattatgaaaaccacgtgttatatcctgttgctagtctgagtcatttggaattgtgggtaaattattatgtacgatggaatccacggatgagacctcagaaatatgtaatcccctggctgactaggactgttaaacatagtgtggactggatgatgccttcgacaaaccagagaagccaagttggggggagctggtgcctggagtgggccctgtgcacctcacctggcggaggctgggggggctctgtcagcaggaccctagaggagactctcattcgattttaaagaagcacaacgggtcattttcctttgtatgttcctagcgcagaactgtttctaaaacaacttgaagtatagttttgttatctaagcaatttttgttttaagtaagtaagtgtactagaatgcgaagccgttatggttcaggtttttaaaaactggtacagtattgtatctgtctcatctgttgcactgtatttcaatcatctgtaattaaaatgatcatatgttt

SEQ ID NO: 53 - DNA, 6860 bp, Homo sapiens
gccctgggagaaatcctcccttcactgggagagaacttctctcccagggcggtgcgacccggagctccagcgcccgagtctccacttcgtttgctgaaacttgctttctaccagctaagaaccatgctgcgagtgattgtggaatctgccagcaatatccctaaaacgaaatttggcaagccggatcctattgtttctgtcatttttaaggatgagaaaaagaaaacaaagaaagttgataatgaattgaaccctgtctggaatgagattttggagtttgacttgaggggtataccactggacttttcatcttcccttgggattattgtgaaagattttgagacaattggacaaaataaattaattggcacggcgactgtagccctgaaggacctgactggtgaccagagcagatccctgccgtacaagctgatctccctgctaaatgaaaaagggcaagatactggggccaccattgacttggtgatcggctatgatccgccttctgctccacatccaaatgacctgagcgggcccagcgtgccaggcatgggaggagatggggaagaagatgaaggtgatgaagacaggttggacaatgcagtcaggggccctgggcccaaggggccagttgggacggtgtcggaagctcagcttgctcggaggctcaccaaagtaaagaacagccggcggatgctgtcaaataagccacaggacttccagatccgcgtccgagtgattgagggccgacagttaagtggtaacaacataaggcctgtggtcaaagttcacgtctgtggccagacacaccgaacaagaatcaagagaggaaacaaccctttttttgatgagttgtttttctacaatgtcaacatgaccccttctgaattgatggatgagatcatcagcatccgggtttataattctcactctctgcgggcagattgtctgatgggggaatttaagattgatgttggatttgtttatgatgaacctggccatgctgtcatgagaaagtggcttcttctcaatgacccggaagataccagttcaggttctaaaggttatatgaaagtcagcatgtttgtcctgggaaccggagatgagcctcctcctgagagacgagatcgtgataatgacagtgatgatgtggagagtaatttgttactccctgctggcattgccctccggtgggtgaccttcttgctgaaaatctaccgagctgaggacatcccccagatggatgatgccttctcacagacagtaaaggaaatatttggaggcaatgcagataagaaaaatctcgtggatccttttgtagaagtttcctttgctggaaaaaaggtttgtacaaacataattgagaaaaatgcaaacccagagtggaatcaggtcgtcaatcttcagatcaagtttccttcagtgtgtgaaaaaataaaactaacaatatatgactgggaccgtcttactaaaaatgatgtagttggaacaacatatctacacctctctaaaattgctgcctctggtggggaagtggaagatttctcatcttcgggaactggggctgcatcatatacagtaaacacaggagaaacagaggtaggctttgttccaacgtttggaccttgttacctgaatctttatggaagccccagagagtacacgggattcccagacccctatgatgagctgaatactggaaagggggaaggagttgcctacagaggcaggatcttggttgaattagccacttttcttgagaagacaccaccagataaaaagcttgagcccatttcaaatgatgacctgctggttgttgagaaataccagcgaaggcggaagtacagcctgtctgccgtgtttcattcagccaccatgttgcaagatgttggtgaggccattcagtttgaagtcagcattgggaactatggcaacaagtttgacaccacctgtaagcctttggcatcaacaactcagtacagccgtgctgtatttgatggcaactactattattacttgccttgggcccacaccaagccagttgttaccctgacttcatactgggaggatattagtcatcgcctggatgcggtgaacactctcctagctatggcagaacggctgcaaacaaatatagaagctctaaaatcagggatacaaggtaaaattcctgcaaaccagctggctgaattgtggctgaagctgatagatgaagttatagaagacacgagatacacgttgcctctcacagaaggaaaagccaacgtcacagttctcgatactcagatccgaaagctgcggtccaggtctctctcccaaatacatgaggcggctgtgaggatgaggtcggaagccacagatgtgaagtccacactggcagaaattgaggactggcttgataaattaatgcagctgactgaagagccacagaacagcatgcctgacatcatcatctggatgatccggggagagaagagactggcctatgcacgaattcccgcacatcaggtcttgtactccaccagtggtgagaatgcatctggaaaatactgtgggaaaacccaaaccatctttctgaagtatccacaggagaaaaacaacgggccaaaggtgcctgtggagttgcgagtgaacatctggctaggcttaagtgctgtggagaagaagtttaacagcttcgcagaaggaactttcaccgtctttgctgaaatgtatgaaaatcaagctctcatgtttggaaaatggggtacttctggattagtaggacgtcataagttttctgatgtcacaggaaaaataaaactcaagagggaattttttctgcctccaaaaggctgggaatgggaaggagagtggatagttgatcctgaaagaagcttgctgactgaggcagatgcaggtcacacggagttcactgatgaagtctatcagaacgagagccgctaccccgggggcgactggaagccggccgaggacacctacacggatgcgaacggcgataaagcagcatcacccagcgagttgacttgtcctccaggttgggaatgggaagatgatgcatggtcttatgacataaatcgagcggtggatgagaaaggctgggaatatggaatcaccattcctcctgatcataagcccaaatcctgggttgcagcagagaaaatgtaccacactcatagacggcgaaggctggtccgaaaacgcaagaaagatttaacacagactgcttcaagcaccgcaagggccatggaggaattgcaagaccaagagggctgggaatatgcttctctaattggctggaaatttcactggaaacaacgtagttcagataccttccgccgcagacgctggaggagaaaaatggctccttcagaaacacatggtgcagctgccatctttaaacttgaaggtgcccttggggcagacactaccgaagatggggatgagaagagcctggagaaacagaagcacagtgccaccactgtgttcggagcaaacacccccattgtttcctgcaattttgacagagtctacatctaccatctgcgctgctatgtctatcaagccagaaacctcttggctttagataaggatagcttttcagatccatatgctcatatctgtttcctccatcggagcaaaaccactgagatcatccattcaaccctgaatcccacgtgggaccaaacaattatattcgatgaagttgaaatctatggggaaccccaaacagttctacagaatccacccaaagttatcatggaactttttgacaatgaccaagtgggcaaagatgaatttttaggacgaagcattttctctcctgtggtgaaactgaactcagaaatggacatcacacccaaacttctctggcacccagtaatgaatggagacaaagcctgcggggatgttcttgtaactgcagagctgattctgaggggcaaggatggctccaaccttcccattcttccccctcaaagggcgccaaatctatacatggtcccccaggggatcaggcctgtggtccagctcactgccattgagattctagcttggggcttaagaaatatgaaaaacttccagatggcttctatcacatcccccagtcttgttgtggagtgtggaggagaaagggtggaatcggtggtgatcaaaaaccttaagaagacacccaactttccaagttctgttctcttcatgaaagtgttcttgcccaaggaggaattgtacatgcccccactggtgatcaaggtcatcgaccacaggcagtttgggcggaagcctgtcgtcggccagtgcaccatcgagcgcctggaccgctttcgctgtgacccttatgcagggaaagaggacatcgtcccacagctcaaagcctcccttctgtctgccccaccatgccgggacatcgttatcgaaatggaagacaccaaaccattactggcttctaagctgacagaaaaggaggaagaaatcgtggactggtggagtaaattttatgcttcctcaggggaacatgaaaaatgcggacagtatattcagaaaggctattccaagctcaagatatataattgtgaactagaaaatgtagcagaatttgagggcctgacagacttctcagatacgttcaagttgtaccgaggcaagtcggatgaaaatgaagatccttctgtggttggagagtttaagggctcctttcggatctaccctctgccggatgaccccagcgtgccagcccctcccagacagtttcgggaattacctgacagcgtcccacaggaatgcacggttaggatttacattgttcgaggcttagagctccagccccaggacaacaatggcctgtgtgacccttacataaaaataacactgggcaaaaaagtcattgaagaccgagatcactacattcccaacactctcaacccagtctttggcaggatgtacgaactgagctgctacttacctcaagaaaaagacctgaaaatttctgtctatgattatgacacctttacccgggatgaaaaagtaggagaaacaattattgatctggaaaaccgattcctttcccgctttgggtcccactgcggcataccagaggagtactgtgtttctggagtcaatacctggcgagatcaactgagaccaacacagctgcttcaaaatgtcgccagattcaaaggcttcccacaacccatcctttccgaagatgggagtagaatcagatatggaggacgagactacagcttggatgaatttgaagccaacaaaatcctgcaccagcacctcggggcccctgaagagcggcttgctcttcacatcctcaggactcaggggctggtccctgagcacgtggaaacaaggactttgcacagcaccttccagcccaacatttcccagggaaaacttcagatgtgggtggatgttttccccaagagtttggggccaccaggccctcctttcaacatcacaccccggaaagccaagaaatactacctgcgtgtgatcatctggaacaccaaggacgttatcttggacgagaaaagcatcacaggagaggaaatgagtgacatctacgtcaaaggctggattcctggcaatgaagaaaacaaacagaaaacagatgtccattacagatctttggatggtgaagggaattttaactggcgatttgttttcccgtttgactaccttccagccgaacaactctgtatcgttgcgaaaaaagagcatttctggagtattgaccaaacggaatttcgaatcccacccaggctgatcattcagatatgggacaatgacaagttttctctggatgactacttgggtttcctagaacttgacttgcgtcacacgatcattcctgcaaaatcaccagagaaatgcagattggacatgattccggacctcaaagccatgaacccccttaaagccaagacagcctccctctttgagcagaagtccatgaaaggatggtggccatgctacgcagagaaagatggcgcccgcgtaatggctgggaaagtggagatgacattggaaatcctcaacgagaaggaggccgacgagaggccagccgggaaggggcgggacgaacccaacatgaaccccaagctggacttaccaaatcgaccagaaacctccttcctctggttcaccaacccatgcaagaccatgaagttcatcgtgtggcgccgctttaagtgggtcatcatcggcttgctgttcctgcttatcctgctgctcttcgtggccgtgctcctctactctttgccgaactatttgtcaatgaagattgtaaagccaaatgtgtaacaaaggcaaaggcttcatttcaagagtcatccagcaatgagagaatcctgcctctgtagaccaacatccagtgtgattttgtgtctgagaccacaccccagtagcaggttacgccatgtcaccgagccccattgattcccagagggtcttagtcctggaaagtcaggccaacaagcaacgtttgcatcatgttatctcttaagtattaaaagttttattttctaaagtttaaatcatgtttttcaaaatatttttcaaggtggctggttccatttaaaaatcatctttttatatgtgtcttcggttctagacttcagcttttggaaattgctaaatagaattcaaaaatctctgcatcctgaggtgatatacttcatatttgtaatcaactgaaagagctgtgcattataaaatcagttagaatagttagaacaattcttatttatgcccacaaccattgctatattttgtatggatgtcataaaagtctatttaacctctgtaatgaaactaaataaaaatgtttcacctttaaaaaaaaaaaaaaaaaa

SEQ ID NO: 54 - DNA, 715 bp, Homo sapiens
  modified_base (376)..(376): a, c, t, g, unknown or other
cggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgttcagtggcatctttctctaacccccagcctcccttcccacagaggccaccgtcatggccagttgctgcagtttctttccagagaacctgtgtatgtgtaaagctgtacaggcgtgggtacaccacacagnctgtcttgcactgtggactgttgagttactagtacatctaggtaagcaccgcatatctgtattcatgtctgccttggccttttaacatctgtgtggtagccgtgtttgaatacccattccctttttgggaacccattagttgttcagcaatttttactgtagataaggctataccgatatctgtgtcatgggttttatgacatgggcagatatctgggagagaaagttcctcagaagaatctgggcacacatgtgtaattctaatatgatgacacccagtttcacctaagaggtggcccatgacttcccaacttaccagctggcctaaactgtttggca

SEQ ID NO: 55 - DNA, 4356 bp, Homo sapiens
cgaagaggagagagcgcgggcctctaggaaggtatggcctcacaagtcttggtctacccaccatatgtttatcaaactcagtcaagtgccttttgtagtgtgaagaaactcaaagtagagccaagcagttgtgtattccaggaaagaaactatccacggacctatgtgaatggtagaaactttggaaattctcatcctcccactaagggtagtgcttttcagacaaagataccatttaagagacctcgaggacacaacttttcattgcagacaagtgctgttgttttgaaaaacactgcaggtgctacaaaggtcatagcagctcaggcacagcaagctcacgtgcaggcacctcagattggggcgtggcgaaacagattgcatttcctagaaggcccccagcgatgtggattgaagcgcaagagtgaggagttggataatcatagcagcgcaatgcagattgtcgatgaattgtccatacttcctgcaatgttgcaaaccaacatgggaaatccagtgacagttgtgacagctaccacaggatcaaaacagaattgtaccactggagaaggtgactatcagttagtacagcatgaagtcttatgctccatgaaaaatacttacgaagtccttgattttcttggtcgaggcacgtttggccaggtagttaaatgctggaaaagagggacaaatgaaattgtagcaatcaaaattttgaagaatcatccttcttatgcccgtcaaggtcaaatagaagtgagcatattagcaaggctcagtactgaaaatgctgatgaatataactttgtacgagcttatgaatgctttcagcaccgtaaccatacttgtttagtctttgagatgctggaacaaaacttgtatgactttctgaaacaaaataaatttagtcccctgccactaaaagtgattcggcccattcttcaacaagtggccactgcactgaaaaaattgaaaagtcttggtttaattcatgctgatctcaagccagagaatattatgttggtggatcctgttcggcagccttacagggttaaagtaatagactttgggtcggccagtcatgtatcaaagactgtttgttcaacatatctacaatctcggtactacagagctccagagattatattggggttgccattttgtgaagccatagacatgtggtcattgggatgtgtgattgcagaattatttcttggatggccgctctacccaggagccttggagtatgatcagattcgatacatttctcagactcaaggtttgccaggagaacagttgttaaatgtgggtactaaatccacaagatttttttgcaaagaaacagatatgtctcattctggttggagattaaagacattggaagagcatgaggcagagacaggaatgaagtctaaagaagccagaaaatacattttcaacagtctggatgatgtagcgcatgtgaacacagtgatggatttggaaggaagtgatcttttggctgagaaagctgatagaagagaatttgttagtctgttgaagaaaatgttgctgattgatgcagatttaagaattactccagctgagaccctgaaccatccttttgttaatatgaaacatcttctagatttccctcatagcaaccatgtaaagtcctgttttcatattatggatatttgtaagtcccacctaaattcatgtgacacaaataatcacaacaaaacttcacttttaagaccagttgcttcaagcagtactgctacactgactgcaaattttactaaaatcggaacattaagaagtcaggcattgaccacatctgctcattcagttgtgcaccatggaatacctctgcaggcaggaactgctcagtttggttgtggtgatgcttttcagcagacattgattatctgtcccccagctattcaaggtattcctgcaacacatggtaaacccaccagttattcaataagggtagataatacagttccacttgtaactcaggccccagctgtgcagccactacagatccgaccaggagttctttctcagacgtggtctggtagaacacagcagatgctggtgcctgcctggcaacaggtgacacccctggctcctgctactactacactaacttctgagagtgtggctggttcacacaggcttggagactgggggaagatgatttcatgcagcaatcattataactcagtgatgccgcagcctcttctgaccaatcagataactttatctgcccctcagccagttagtgtggggattgcacatgttgtctggcctcagcctgccactaccaagaaaaataaacagtgccagaacaggagtaattcattacagaataccaatatcccacattcagcatttatttctccaaagataattaatgggaaagatgtcgaggaagtaagttgtatagaaacacaggacaatcagaactcagaaggagaggcaagaaattgctgtgaaacatctatcagacaggactctgattcatcagtttcagacaaacagcggcaaaccatcattattgccgactccccgagtcctgcagtgagtgtcatcactatcagcagtgacactgatgaggaagagacttcccagagacattcactcagagaatgtaaaggtagtctagattgtgaagcttgccagagcactttgaatattgatcggatgtgttcattaagtagtcctgatagtactctgagtaccagctcctcagggcagtccagcccatccccctgcaagagaccgaatagtatgtcagatgaagagcaagaaagtagttgtgatacggtggatggctctccgacatctgactcttccgggcatgacagtccatttgcagagagcacttttgtggaggacactcatgaaaacacagaattggtatcctctgctgacacagaaaccaagccagctgtctgttctgttgtggtgccaccagtggaactagaaaatggcttaaatgccgatgagcatatggcaaacacagattctatatgccagccattaataaaaggacgatctgcccctggaagattaaaccagccttctgcagtgggtactcgtcagcaaaaattgacatcagcattccagcagcagcatttgaacttcagtcaggttcagcactttggatctgggcatcaagagtggaatggaaactttgggcacagaagacagcaagcttatattcctactagtgttaccagtaatccattcactctttctcatggaagtcccaatcacacagcagtgcatgcccacctggctggaaatacacacctcggaggacagcctactctacttccatacccatcatcagccaccctcagtagtgctgcaccagtggcccacctgttagcctctccgtgtacctcaagacctatgttaaagcatccaacttataatatctcccatcccagtggcatagttcaccaagtcccagtgggcttaaatccccgtctgttaccatccccaaccattcatcagactcagtacaaaccaatcttcccaccacattcttacattgcagcatcacctgcatatactggatttccactgagtccaacaaaactcagccagtatccatatatgtgaaaaacagtatattggggaagctcaatgatacaaacatttgattaaaaataaaaacatggtatttaatattagccatggcacaagaaaattatttttggaatcatgtagacttgggtgcaatttaaacaactttgagctttaaaaactcacttttgatgtgttttgcacatttggtataacttgtctttggtcatgttatcttcttatgtagtaactctagacaggtgacttatgggagcagaagtccagttttgctcctgctattttttataaattgccttctaactagtgcaagacacgtctacatttgggaagccattctgtgtacagacttagagcaacagatgcacatatgtcagaattacagcatacaagtgaattgtattatccgtgtcttagtgtataaatgttgggtcacttacctaagaaattgagctattgttctttacatttgcatgtgtcttttgcatgggcaaaatgttgcctagactttgctcttaaatgttgttctaataatctcagctgcattgtaaaccgttcctacacatagtgccttaaatatttgaggttgttaatgttattacctatatataaatgttgaggactgcagcacttaaaattcagacctactatttagtttccttttgatagcgtaatgttcatttttgtttttgtgtggtatgatttcaggtagtagctgtttttttccttattaagagggcagcatgtttgctatag